(19) United States
(12) Patent Application Publication
(10) Pub. No.: US 2023/0192899 A1
(43) Pub. Date: Jun. 22, 2023

(54) ACTIVATABLE BISPECIFIC ANTIBODIES COMPRISING A LINKER BETWEEN THE TWO BINDING DOMAINS WHICH IS A HUMAN IMMUNOGLOBULIN HINGE REGION, OR A VARIANT THEREOF, AND USES THEREOF

(71) Applicant: LOCKBODY THERAPEUTICS LTD, Sandwich (GB)

(72) Inventor: William James Jonathan Finlay, Sandwich (GB)

(21) Appl. No.: 17/610,899

(22) PCT Filed: May 13, 2020
(86) PCT No.: PCT/EP2020/063362

§ 371 Date: Nov. 12, 2021

(30) Foreign Application Priority Data
May 13, 2019 (GB) 1906685.1
Jul. 17, 2019 (GB) 1910254.0
Dec. 4, 2019 (GB) 1917678.3
Jan. 28, 2020 (GB) 2001196.1

(51) Int. Cl. C07K 16/46; A61P 35/00; C12N 15/63; A61K 47/68

(52) U.S. Cl. CPC C07K 16/468; A61K 47/6801; A61P 35/00; C12N 15/63

(57) ABSTRACT

Described herein are protein molecules that exhibit activatable target binding in diseased tissues and related nucleic acid molecules, vectors and host cells. Also described herein are medical uses for such protein molecules.

### CROSS REFERENCE TO RELATED APPLICATIONS

This application is a 35 U.S.C. § 371 national phase application of International Patent Application No. PCT/EP2020/063362, filed on May 13, 2020, which claims the benefit of GB Patent Application No. 2001196.1, filed on Jan. 28, 2020, GB Patent Application No. 1917678.3, filed on Dec. 4, 2019, GB Patent Application No. 1910254.0, filed on Jul. 17, 2019, and GB Patent Application No. 1906685.1, filed on May 13, 2019, the disclosure of each of which is hereby incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: ULSL_002_04US_SubSeqList_ST25.txt, date created: May 3, 2022, file size ~400,272 bytes).

### FIELD OF THE INVENTION

The invention relates to protein molecules that exhibit activatable target binding in diseased tissues and medical uses therefor.

### BACKGROUND OF THE INVENTION

In the use of antibodies and other binding proteins to treat disease, many potential drug targets have been described, but few are exclusively expressed in diseased tissue. The majority of potential targets in this space are, in fact, also expressed in non-diseased tissue.

In addition, the majority of drug mechanisms of action employed in challenging areas of therapy such as cancer employ highly potent cell-killing mechanisms of action. As a result, engagement of the target by the drug in non-diseased tissue often causes unwanted side effects. There is a need for engineered forms of binding proteins that are partially, or even fully, inactive in healthy tissue but become highly activated in diseased tissue.

### SUMMARY OF THE INVENTION

Provided herein is a protein comprising a first moiety and a second moiety and a peptide linker between the first moiety and the second moiety, wherein the peptide linker comprises an amino acid sequence from a human immunoglobulin hinge region or an amino acid sequence or an amino acid sequence having from 1 to about 7 amino acid substitutions compared to a human immunoglobulin hinge region; wherein the peptide linker is cleavable by a protease expressed in a diseased tissue; wherein the second moiety is capable of specifically binding to a molecule expressed in the diseased tissue; and wherein the binding of the second moiety to the molecule expressed in the diseased tissue is reduced or inhibited when the peptide linker is uncleaved. In some embodiments, the peptide linker is between about 5 and about 15 amino acids in length. In some embodiments, the peptide linker comprises or consists of the amino acid sequence of SEQ ID NO:1, SEQ ID NO:2, SEQ ID NO:3, SEQ ID NO:4, SEQ ID NO:5, SEQ ID NO:69, SEQ ID NO:70, SEQ ID NO:71, SEQ ID NO:72, SEQ ID NO:81, SEQ ID NO:82, SEQ ID NO:83, SEQ ID NO:84, SEQ ID NO:85, SEQ ID NO:86, or SEQ ID NO:87.

In some embodiments, the protease is a human matrix metalloprotease (MMP), a human cathepsin, human enterokinase, human thrombin, human tPA, human Granzyme B, human uPA, or human ADAMTs-5. In some embodiments, the peptide linker comprises a human MMP cleavage site, a human cathepsin, human enterokinase, human thrombin, human tPA, human Granzyme B, human uPA, or human ADAMTs-5 cleavage site. In some embodiments, the human MMP is MMP-2, MMP-3, MMP-7, MMP-8, MMP-9, MMP-10, MMP-12, MMP-13 or MMP14. In some embodiments, the level or the activity of the human MMP is elevated in the diseased tissue compared to the level or the activity of the human MMP in a non-diseased tissue. In some embodiments, the human cathepsin is Cathepsin A, Cathepsin C, Cathepsin D, Cathepsin G, Cathepsin L or Cathepsin K. In some embodiments, the level or the activity of the human cathepsin is elevated in the diseased tissue compared to the level or the activity of the human cathepsin in a non-diseased tissue.

In some embodiments, the first moiety comprises an antibody, an antigen-binding portion of an antibody or a receptor ectodomain. In some embodiments, the first moiety is a Fab, a single-chain Fab, a VH domain, a VL domain, an immunoglobulin new antigen receptor (IgNAR), a single-chain variable fragment (scFv), a diabody, or a T cell receptor domain. In some embodiments, the first moiety specifically binds to a molecule expressed in a diseased tissue.

In some embodiments, the first moiety specifically binds to a first molecule expressed in a diseased tissue and the second moiety is capable of specifically binding to a second molecule expressed in a diseased tissue, wherein the first molecule expressed in a diseased tissue and the second molecule expressed in a diseased tissue are different molecules. In some embodiments, the first molecule expressed in a diseased tissue and the second molecule expressed in a diseased tissue are expressed by the same cell. In some embodiments, the first molecule expressed in a diseased tissue and the second molecule expressed in a diseased tissue are expressed by different cells. In some embodiments, the first molecule expressed in a diseased tissue and/or the second molecule expressed in a diseased tissue is expressed on the surface of a cell. In some embodiments, the first molecule expressed in a diseased tissue and/or the second molecule expressed in a diseased tissue is a soluble molecule.

In some embodiments, the first moiety binds specifically to human EGFR, human HER2, human HER3, human CD105, human C-KIT, human PD1, human PD-L1, human PSMA, human EpCAM, human Trop2, human EphA2, human CD20, human BCMA, human GITR, human OX40, human CSF1R, human Lag3 or human cMET.

In some embodiments, the second moiety specifically binds to a molecule expressed by a human immune cell. In some embodiments, the molecule expressed by a human immune cell is human CD3, human CD16A, human CD16B, human CD28, human CD89, human CTLA4, human NKG2D, human SIRPα, human SIRPγ, human PD1, human Lag3, human 4-1BB, human OX40, or human GITR.

In some embodiments, the first moiety comprises a heavy chain variable (VH) region and a light chain variable (VL) region. In some embodiments, the first moiety comprises an immunoglobulin constant region or a portion of an immunoglobulin constant region. In some embodiments, the immunoglobulin constant region is IgG, IgE, IgM, IgD, IgA, or IgY. In some embodiments, the immunoglobulin constant region is IgG1, IgG2, IgG3, IgG4, IgA1, or IgA2. In some embodiments, the immunoglobulin constant region is immunologically inert. In some embodiments, the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A and L235A, a human IgG1 constant region comprising the amino acid substitutions L234A, L235A and G237A, a human IgG1 constant region comprising the amino acid substitutions L234A, L235A, G237A and P331S, or a wild-type human IgG2 constant region.

In some embodiments, the second moiety comprises an antibody, an antigen-binding portion of an antibody or a receptor ectodomain. In some embodiments, the second moiety is a Fab, a single-chain Fab, a VH domain, a VL domain, an immunoglobulin new antigen receptor (IgNAR), a single-chain variable fragment (scFv), or a T cell receptor domain. In some embodiments, the second moiety binds specifically to human CD47. In some embodiments, the second moiety binds specifically to human CD3 or human PD-L1.

In some embodiments, the second moiety comprises a heavy chain variable (VH) region and a light chain variable (VL) region. In some embodiments, the second moiety comprises an immunoglobulin constant region or a portion of an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region is immunologically inert. In some embodiments, the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A and L235A, a human IgG1 constant region comprising the amino acid substitutions L234A, L235A and G237A, a human IgG1 constant region comprising the amino acid substitutions L234A, L235A, G237A and P331S, or a wild-type human IgG2 constant region.

In some embodiments, the protein has an immune effector function or two, three or more immune effector functions. In some embodiments, the immune effector function is ADCC, CDC orADCP.

In some embodiments, the first moiety prevents or reduces specific binding of the second moiety to the molecule expressed in the diseased tissue. In some embodiments, the peptide linker is cleaved in the vicinity of the diseased tissue or inside the diseased tissue. In some embodiments, the peptide linker is cleaved in the vicinity of the diseased tissue or inside the diseased tissue, wherein the first moiety dissociates from the second moiety in the vicinity of the diseased tissue or inside the diseased tissue and wherein the second moiety specifically binds to the molecule expressed in the diseased tissue in the vicinity of the diseased tissue or inside the diseased tissue. In some embodiments, the diseased tissue is a tumour or an inflamed tissue.

In some embodiments, the first moiety binds specifically to human cMET, wherein the second moiety binds specifically to human CD47, and wherein the protein comprises a first polypeptide chain comprising or consisting of the amino acid sequence of SEQ ID NO:16 and a second polypeptide chain comprising or consisting of the amino acid sequence of SEQ ID NO:17.

In some embodiments, the first moiety binds specifically to human HER2, wherein the second moiety binds specifically to human CD3, and wherein the protein comprises a first polypeptide chain comprising or consisting of the amino acid sequence of SEQ ID NO:26 and a second polypeptide chain comprising or consisting of the amino acid sequence of SEQ ID NO:27.

In some embodiments, the first moiety binds specifically to human HER2, wherein the second moiety binds specifically to human CD47, and wherein the protein comprises a first polypeptide chain comprising or consisting of the amino acid sequence of SEQ ID NO:34 and a second polypeptide chain comprising or consisting of the amino acid sequence of SEQ ID NO:35.

In some embodiments, the first moiety binds specifically to human cMET, wherein the second moiety binds specifically to human CD47, and wherein the protein comprises a first polypeptide chain comprising or consisting of the amino acid sequence of SEQ ID NO:36 and a second polypeptide chain comprising or consisting of the amino acid sequence of SEQ ID NO:37.

In some embodiments, the first moiety binds specifically to human Her2, wherein the second moiety binds specifically to human CD3, and wherein the protein comprises a first polypeptide chain comprising or consisting of the amino acid sequence of SEQ ID NO:38 and a second polypeptide chain comprising or consisting of the amino acid sequence of SEQ ID NO:39.

In some embodiments, the first moiety binds specifically to human Her2, wherein the second moiety binds specifically to human CD3, and wherein the protein comprises a first polypeptide chain comprising or consisting of the amino acid sequence of SEQ ID NO:40 and a second polypeptide chain comprising or consisting of the amino acid sequence of SEQ ID NO:41.

In some embodiments, the first moiety binds specifically to human Her2, wherein the second moiety binds specifically to human CD47, and wherein the protein comprises a first polypeptide chain and a second polypeptide chain, wherein:
(a) the first polypeptide chain comprises or consists of the amino acid sequence of SEQ ID NO:42, and the second polypeptide chain comprises or consists of the amino acid sequence of SEQ ID NO:43; or
(b) the first polypeptide chain comprises or consists of the amino acid sequence of SEQ ID NO:44, and the second polypeptide chain comprises or consists of the amino acid sequence of SEQ ID NO:45; or
(c) the first polypeptide chain comprises or consists of the amino acid sequence of SEQ ID NO:46, and the second polypeptide chain comprises or consists of the amino acid sequence of SEQ ID NO:47; or
(d) the first polypeptide chain comprises or consists of the amino acid sequence of SEQ ID NO:48, and the second polypeptide chain comprises or consists of the amino acid sequence of SEQ ID NO:49; or
(e) the first polypeptide chain comprises or consists of the amino acid sequence of SEQ ID NO:50, and the second polypeptide chain comprises or consists of the amino acid sequence of SEQ ID NO:51; or
(f) the first polypeptide chain comprises or consists of the amino acid sequence of SEQ ID NO:52, and the second polypeptide chain comprises or consists of the amino acid sequence of SEQ ID NO:53; or
(g) the first polypeptide chain comprises or consists of the amino acid sequence of SEQ ID NO:54, and the second polypeptide chain comprises or consists of the amino acid sequence of SEQ ID NO:55; or
(h) the first polypeptide chain comprises or consists of the amino acid sequence of SEQ ID NO:88, and the second polypeptide chain comprises or consists of the amino acid sequence of SEQ ID NO:89; or
(i) the first polypeptide chain comprises or consists of the amino acid sequence of SEQ ID NO:90, and the second polypeptide chain comprises or consists of the amino acid sequence of SEQ ID NO:91; or
(j) the first polypeptide chain comprises or consists of the amino acid sequence of SEQ ID NO:44, and the second polypeptide chain comprises or consists of the amino acid sequence of SEQ ID NO:92; or
(k) the first polypeptide chain comprises or consists of the amino acid sequence of SEQ ID NO:44, and the second polypeptide chain comprises or consists of the amino acid sequence of SEQ ID NO:93; or
(l) the first polypeptide chain comprises or consists of the amino acid sequence of SEQ ID NO:44, and the second polypeptide chain comprises or consists of the amino acid sequence of SEQ ID NO:94; or
(m) the first polypeptide chain comprises or consists of the amino acid sequence of SEQ ID NO:44, and the second polypeptide chain comprises or consists of the amino acid sequence of SEQ ID NO:95; or
(n) the first polypeptide chain comprises or consists of the amino acid sequence of SEQ ID NO:44, and the second polypeptide chain comprises or consists of the amino acid sequence of SEQ ID NO:96; or
(o) the first polypeptide chain comprises or consists of the amino acid sequence of SEQ ID NO:44, and the second polypeptide chain comprises or consists of the amino acid sequence of SEQ ID NO:97.

In some embodiments, the first moiety binds specifically to human Her2, wherein the second moiety binds specifically to human CD3, and wherein the protein comprises a first polypeptide chain comprising or consisting of the amino acid sequence of SEQ ID NO:73 and a second polypeptide chain comprising or consisting of the amino acid sequence of SEQ ID NO:74.

In some embodiments, the first moiety binds specifically to human cMET, wherein the second moiety binds specifically to human cMET, and wherein the protein comprises a first polypeptide chain comprising or consisting of the amino acid sequence of SEQ ID NO:75 and a second polypeptide chain comprising or consisting of the amino acid sequence of SEQ ID NO:76.

In some embodiments, the first moiety binds specifically to human Her2, wherein the second moiety binds specifically to human CD3, and wherein the protein comprises a first polypeptide chain and a second polypeptide chain, wherein:
(a) the first polypeptide chain comprises or consists of the amino acid sequence of SEQ ID NO:98, and the second polypeptide chain comprises or consists of the amino acid sequence of SEQ ID NO:99; or
(b) the first polypeptide chain comprises or consists of the amino acid sequence of SEQ ID NO:100, and the second polypeptide chain comprises or consists of the amino acid sequence of SEQ ID NO:101; or
(c) the first polypeptide chain comprises or consists of the amino acid sequence of SEQ ID NO:102, and the second polypeptide chain comprises or consists of the amino acid sequence of SEQ ID NO:103; or
(d) the first polypeptide chain comprises or consists of the amino acid sequence of SEQ ID NO:104, and the second polypeptide chain comprises or consists of the amino acid sequence of SEQ ID NO:105.

Further provided herein is an immunoconjugate comprising a protein of the invention linked to a therapeutic agent. In some embodiments, the therapeutic agent is a cytotoxin, a radioisotope, a chemotherapeutic agent, an immunomodulatory agent, an anti-angiogenic agent, an antiproliferative agent, a pro-apoptotic agent, a cytostatic enzyme, a cytolytic enzymes, a therapeutic nucleic acid, an anti-angiogenic agent, an anti-proliferative agent, or a pro-apoptotic agent.

Further provided herein is a pharmaceutical composition comprising a protein of the invention or an immunoconjugate of the invention, and a pharmaceutically acceptable carrier, diluent or excipient.

Further provided herein is a nucleic acid molecule encoding a protein or a portion of a protein of the invention. Further provided herein is a nucleic acid molecule encoding the first polypeptide chain, the second polypeptide chain, or both the first polypeptide chain and the second polypeptide chain of a protein of the invention.

Further provided herein is an expression vector comprising a nucleic acid molecule of the invention.

Further provided herein is a recombinant host cell comprising a nucleic acid molecule of the invention or an expression vector of the invention.

Further provided herein is a method of producing a protein, the method comprising culturing a recombinant host cell comprising an expression vector of the invention under conditions whereby the nucleic acid molecule is expressed, thereby producing the protein; and isolating the protein from the host cell or culture.

Further provided herein is a method for enhancing an anti-cancer immune response in a subject, comprising administering to the subject a therapeutically effective amount of a protein of the invention, an immunoconjugate of the invention, or a pharmaceutical composition of the invention.

Further provided herein is a method of treating cancer, an autoimmune disease, an inflammatory disease, a cardiovascular disease or a fibrotic disease in a subject, comprising administering to the subject a therapeutically effective amount of a protein of the invention, an immunoconjugate of the invention, or a pharmaceutical composition of the invention. In some embodiments, the cancer is Gastrointestinal Stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma or cancer of hematological tissues. In some embodiments, the autoimmune disease or the inflammatory disease is arthritis, asthma, multiple sclerosis, psoriasis, Crohn’s disease, inflammatory bowel disease, lupus, Grave’s disease, Hashimoto’s thyroiditis or ankylosing spondylitis. In some embodiments, the cardiovascular disease is coronary heart disease, or atherosclerosis or stroke. In some embodiments, the fibrotic disease is myocardial infarction, angina, osteoarthritis, pulmonary fibrosis, cystic fibrosis, bronchitis or asthma.

Further provided herein is a protein of the invention, an immunoconjugate of the invention, or a pharmaceutical composition of the invention for use in the treatment of cancer, an autoimmune disease, an inflammatory disease, a cardiovascular disease or a fibrotic disease. In some embodiments, the cancer is Gastrointestinal Stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma or cancer of hematological tissues. In some embodiments, the autoimmune disease or the inflammatory disease is arthritis, asthma, multiple sclerosis, psoriasis, Crohn’s disease, inflammatory bowel disease, lupus, Grave’s disease, Hashimoto’s thyroiditis or ankylosing spondylitis. In some embodiments, the cardiovascular disease is coronary heart disease, atherosclerosis, or stroke. In some embodiments, the fibrotic disease is myocardial infarction, angina, osteoarthritis, pulmonary fibrosis, cystic fibrosis, bronchitis or asthma.

Further provided herein is a protein of the invention, an immunoconjugate of the invention, or a pharmaceutical composition of the invention, for use as a medicament.

### BRIEF DESCRIPTION OF THE DRAWINGS

FIG. 1A - FIG. 1B. Challenges in dosing systemically-active antibody drugs into solid tumours - anti-CD47 as an example. CD47 antibodies (FIG. 1A) have significant challenges such as high expression of CD47 in the bloodstream. Erythrocytes and platelets in particular form a ‘sink’ and toxicity risk issue. The tumour is also typically a ‘hostile’ environment with high expression of enzymes such as MMPs which accelerate IgG degradation. Anti-CD47 protein constructs of the invention (FIG. 1B) aim to eliminate CD47 binding in the native protein, which removes the peripheral activity. The tumour-targeting domain then drives high concentration in the tumour environment and the peptide linker system exploits the MMP activity in the tumour to activate the CD47-binding activity in the tumour, rather than the periphery.

FIG. 2A - FIG. 2B. Protein construct IgG² design and activation principles. The protein construct IgG² design (FIG. 2A) may be based on sequences derived from IgG1, IgG2, IgG3, IgG4,IgA, IgE, or IgM and may or may not have effector function capacity. In this construct, four polypeptide chains encode for four Fab domains (2x Fab A, 2X Fab B), two linker sequences, and may or may not have an immunoglobulin hinge region and an Fc domain. Each Fab A-Linker domain blocks the binding activity of Fab B. The choice of linker sequence, such as a lower hinge peptide sequence from an immunoglobulin, creates a structure that will be locked in a non-diseased tissue, but might be quickly cleaved and unlocked in the presence of high concentrations of proteases in the tumour environment (FIG. 2B). The linkers may be sequentially cleaved, creating an intermediate unlocked active state which allows Fabs A and B from a single protein construct to bind their cognate targets. Secondary, potentially slower cleavage of the second linker in each Fab A-Fab B protein construct unit may release the Fab A domains from the structure entirely, making a dissociated form. Cleaved linkers based on immunoglobulin hinge sequences may also recruit increased immune effector function at the cell membrane via endogenous anti-hinge antibodies. Variable regions are indicated in white. Constant regions are indicated in grey.

FIG. 3A - FIG. 3B. Protein construct Fab² design and activation principles. The protein construct Fab² design may be based on sequences derived from IgG1, IgG2, IgG3, IgG4, IgA, IgE, or IgM and may or may not have effector function capacity. In this construct, two (FIG. 3A) or three (FIG. 3B) polypeptide chains may encode for two Fab domains (1x Fab A, 1X Fab B), two or more linker sequences, and may or may not have an immunoglobulin hinge region and an Fc domain in which pairing of heterodimers may or may not be driven by mutations in the Fc. Each Fab A-Linker domain blocks the binding activity of Fab B. The choice of linker sequence, such as a lower hinge peptide sequence, creates a structure that will be locked in a non-diseased tissue but quickly cleaved and unlocked in the presence of high concentrations of proteases in the tumour environment (FIG. 3A). The linkers may be sequentially cleaved, creating an intermediate unlocked active state which allows Fabs A and B from a single protein to bind their cognate targets. Secondary, potentially slower cleavage of the second linker in each Fab A-Fab B protein unit may release the Fab A domains from the structure entirely. Cleaved linkers based on immunoglobulin hinge sequences may also recruit increased immune effector function at the cell membrane via endogenous anti-hinge antibodies. Variable regions are indicated in white. Constant regions are indicated in grey.

FIG. 4. SDS-PAGE analysis of Protein A-purified protein construct IgG² and Fab² proteins from clones 1-15. Multiple construct example proteins were expressed in CHO cells and purified using Protein A affinity chromatography. Purified proteins were then subjected to SDS-PAGE analysis in both unreduced and reduced (r) states, alongside a Molecular Weight Standard (M). Clones 6, 10 and 14 (all containing LHL linkers) were found to contain the highest proportion of expected-size products and lowest higher and lower molecular weight content.

FIG. 5A - FIG. 5I. Size Exclusion Chromatography of Protein A-purified protein construct IgG² and Fab² proteins. Selected construct example proteins were analysed and fully purified using SEC. Clones 1 (FIG. 5A), 2 (FIG. 5B), 3 (FIG. 5C), 4 (FIG. 5D), 5 (FIG. 5E), 6 (FIG. 5F), 12 (FIG. 5G), 14 (FIG. 5H) and 10 (FIG. 5I) were analyzed. This data showed that the highest proportion of expected-size products (e.g. highlighted peak, FIG. 5I) and lowest higher/lower molecular weight content were found in the samples from clones 6, 10 and 14 (all containing LHL linkers).

FIG. 6A - FIG. 6B. SDS-PAGE analysis of SEC-purified protein construct IgG² and Fab² proteins. Protein A affinity-purified key lead protein construct clones were finally purified using SEC. Purified proteins from clones 1, 2, 4, 5, 6 and non-SEC purified 15 (FIG. 6A) were then subjected to SDS-PAGE analysis in unreduced state. Purified proteins from clones 7, 8, 10, 11, 12, 13 and 14 (FIG. 6B) were also subjected to SDS-PAGE analysis in both unreduced and reduced (r) states. All proteins were loaded at approximately 1 µg per lane. Clones 6, 10 and 14 (all containing LHL linkers) were found to contain the highest proportion of expected-size products and lowest higher and lower molecular weight content.

FIG. 7A - FIG. 7B. Direct titration ELISA for purified, intact protein constructs and control antibodies binding to human target proteins. Control antibodies A-D5 anti-CD47, A-D5 Fab-Fc (a monovalent version of the A-D5 antibody), MH7.1 anti-C-MET, and anti-Her2 Trastuzumab, all in human IgG1 format, were titrated (in µg/ml) in a direct binding ELISA against human CD47, C-MET and Her2 proteins (FIG. 7A). The Her2CD47-LH-LH and Her2CD47-LHL-LHL in IgG² format (FIG. 7B) and cMETCD47-L2-L2 and cMETCD47-LHL-LHL in Fab² format (FIG. 7C) were also analyzed in the same fashion.

FIG. 8A - FIG. 8C. Human erythrocyte hemagglutination assay for purified, intact protein constructs and control antibodies. Control antibodies anti-CD235a (murine) and A-D5 anti-CD47, A-D5 Fab-Fc (a monovalent version of the A-D5 antibody, labelled as ‘FabCD47-Only’), MH7.1 anti-C-MET, anti-Her2 Trastuzumab, Her2CD47-LH-LH and Her2CD47-LHL-LHL IgG2, and cMETCD47-L2-L2 and cMETCD47-LHL-LHL in Fab² format, were titrated (in nM) in a human erythrocyte hemagglutination assay using fresh erythrocytes from donor 1 (FIG. 8A), donor 2 (FIG. 8B) and donor 3 (FIG. 8C).

FIG. 9A - FIG. 9C. Direct ELISA for purified, intact and MMP-digested protein constructs binding to human target proteins. Protein constructs were submitted to enzymatic digestion using human MMPs 3, 7 and 12 over a time course of 2, 4, 8 and 24 hours incubation, plus a 24 h incubation in buffer without enzyme as a negative control. Samples from these digest time courses were then applied in direct binding ELISA against human Her2 and CD47 (FIG. 9A, B) or human C-MET and human CD47 (FIG. 9C).

FIG. 10A - FIG. 10C. Functional analyses for purified, intact and MMP-digested Her2CD3 Fab² protein construct binding to human target proteins. Antibodies Her2CD3-L1-LH, Her2CD3-L2-L2 and Her2CD3-LHL-LHL in Fab² format were analyzed by ELISA without MMP digest (FIG. 10A), Flow Cytometry (FIG. 10B) and a CD3 reporter assay (FIG. 10C), with or without MMP digest.

FIG. 11. Alternative structures based on protein construct design and activation principles. The protein construct module (1) that is found in both the Fab² and IgG² designs of FIGS. 2 and 3 may be modified and the functional characteristics of the final molecule altered. In this case, the upper binding unit of the protein construct module or the lower unit, or both, may be an alternative structure to an immunoglobulin Fab domain, allowing alternative molecules based on sequences derived from peptides, receptor ectodomains, binding domains and especially other dimerizing immune recognition receptors such as T cell receptors. These constructs could form many formats and examples are provided here, such as: Four polypeptide chains (2) may encode for an IgG²-like structure containing two full protein construct modules with 4 binding domain units (1x A, 1X B, or 2x A or B), two or more linker sequences, and may or may not have an immunoglobulin hinge region and an Fc domain in which pairing of heterodimers may or may not be driven by mutations in the Fc. In the expression of 3 polypeptides, the Fab² design can be augmented by the addition of a binding domain (3) or peptide that renders the structure potentially trispecific or of altered valency. Trispecificity or altered valency may also be achieved in the Fab² design by the addition of a further one (4) or two (5) protein construct -linker-Fab/receptor structures at the c-terminus. It should be noted also that any of the structures outlined in this figure or in FIGS. 2 and 3 may be further functionalized by the addition of n-terminal of c-terminal fusions of any kind of polypeptide chain, or by chemical conjugation. Variable regions are indicated in white. Constant regions are indicated in grey.

FIG. 12. A ‘passive’ structure based on Fab² protein construct principles. In this case, the upper binding unit of the protein construct module may be placed c-terminally to an Fc domain. These constructs may or may not have an immunoglobulin hinge region and an Fc domain in which pairing of heterodimers may or may not be driven by mutations in the Fc. In this construct, the binding of both Fab or receptor domains to their cognate targets should only become fully active after cleavage of at least one linker. It should be noted also that any of the structures outlined in this figure may be further functionalized by the addition of n-terminal or c-terminal fusions of any kind of polypeptide chain, or by chemical conjugation. Variable regions are indicated in white. Constant regions are indicated in grey.

FIG. 13. An ‘activatable’ antibody drug conjugate (ADC) strategy based on Fab² protein construct principles. In this case, the upper and lower binding units of the protein construct module may contain antibodies to the same internalizing receptor target, or two different targets, that are found on the surface of the same cell. These constructs could be chemically conjugated or fused with a ‘payload’ moiety such as a toxin or other active molecule to form an ADC and may or may not have an immunoglobulin hinge region and an Fc domain in which pairing of heterodimers may or may not be driven by mutations in the Fc. In this construct, the binding of the upper Fab or receptor domain to its cognate target is constitutively active, causing accumulation of the antibody in tissues where its cognate target is expressed. The construct does not initially drive internalization into the target cell as the binding is monovalent and the receptor is known to only be significantly internalized when 2 or more receptor domains are cross-linked by bivalent antibody binding. The activity of the second (lower) Fab or receptor domain should only be engaged after linker cleavage by a disease-related enzyme, which then drives multivalent receptor binding and internalization of the ADC, allowing delivery of the (e.g. cytotoxic or inflammatory) payload moiety. It should be noted also that any of the structures outlined in this figure may be further functionalized by the addition of n-terminal or c-terminal fusions of any kind of polypeptide chain.

FIG. 14. Direct ELISA for purified, intact protein constructs binding to human and murine target proteins. Samples were applied in direct binding ELISA against human Her2 and human and murine CD47.

FIG. 15A - FIG. 15F. Direct ELISA for purified, intact and MMP-digested protein constructs binding to human target proteins. Protein constructs were submitted to enzymatic digestion using human MMPs 7 (FIG. 15A), 8 (FIG. 15B), 10 (FIG. 15C), 12 (FIG. 15D), 13 (FIG. 15E), and Cathepsin S (FIG. 15F), over a time course of 2, 4, 8 and 24 hours incubation, plus a 24 h incubation in buffer without enzyme as a negative control (time 0). Samples from these digest time courses were then applied in direct binding ELISA against human Her2 and CD47.

FIG. 16A - FIG. 16B. Biacore SPR assay for purified, intact and MMP-digested Her47-LHL-LHLF binding to human target proteins. Her47-LHL-LHLF was submitted to enzymatic digestion using human MMP 12 over a time course of 2, 4, 8 and 24 hours incubation, plus a 24 h incubation in buffer without enzyme as a negative control (Undigested). Samples from these digest time courses were then captured on anti-Fc antibody coated Biacore chips and human Her2 (FIG. 16A) or human CD47 (FIG. 16B) flowed in solution. Rmax values were plotted to indicate maximal binding observed at the highest concentration of analyte protein.

FIG. 17. Biacore SPR assay for purified, intact and 24 h MMP-digested Her47-LHL-LHLF binding to human target proteins. Her47-LHL-LHLF was submitted to enzymatic digestion using human MMP 12 for 24 hours incubation, or without enzyme as a negative control (‘before protease treatment’). Samples were then captured on anti-Fc antibody coated Biacore chips and human human CD47 flowed in solution at multiple concentrations. Binding curves indicate no interaction of the undigested (intact) Her47-LHL-LHLF protein to huCD47, even at 400 nM huCD47, while strong binding is evident for the same protein after MMP12 activation, at all concentrations tested.

FIG. 18A - FIG. 18B. Structural modelling of full Her47-LHL-LHL IgG² structure. FIG. 18A, molecular modelling of the full structure of the IgG² molecule, showing the upper (trastuzumab) Fab domains in contact with their Her2 epitope (grey). FIG. 18B, molecular modelling of the full structure of the IgG² molecule, showing the upper (trastuzumab) Fab domains in contact with their Her2 epitope (grey), but the CD47 ectodomain is also superimposed in its likely binding position for the A-D5 lower Fab. This analysis demonstrates that the CD47 epitope cannot be bound when both linkers are intact.

FIG. 19A - FIG. 191. Structural dynamics of Her47 Fab² structure with different linkers. Solvent accessible surface area (SASA) results obtained for 3 of linkers (LHL, LHLF, L2). FIG. 19A, FIG. 19D and FIG. 19G show the absolute SASA values for 9 dynamics runs for LHL and LHLF linkers and 10 runs using L2, all over 6 ns. FIG. 19B, FIG. 19E, FIG. 19H, FIG. 19C, FIG. 19F and FIG. 19l show normalised results representing the difference to the starting SASA value, overthe 6 ns dynamics run time and the first 2.5 ns of the 6 ns dynamics runs. FIG. 19A, FIG. 19B and FIG. 19C depict SEQ ID NO:2. FIG. 19D, FIG. 19E and FIG. 19F depict SEQ ID NO:3. FIG. 19G, FIG. 19H and FIG. 19l depict SEQ ID NO:32.

FIG. 20A - FIG. 20B. Structural dynamics of intact and activated Her47 LHL-LHL Fab² structure. (FIG. 20A) Overlaid Fab² structures in both intact linker and activated (single linker cut by protease) forms. (FIG. 20B) Two poses taken from a molecular dynamics simulation of the Fab² regions in which one LHL or LHLF linker has been cleaved. Anti-CD47 Fab is shown in black and the anti-HER2 Fab is shown in grey. The diagram illustrates the furthest degree of travel of the Her2 domain and the resulting exposure of the anti-CD47 Fab CDRs.

FIG. 21. Flow cytometric analyses of protein binding to ‘Tg32’ mouse erythrocytes. Flow cytometric analyses of binding to erythrocytes was performed using A-D5 IgG1, IgG² Her47 LHL-LHL, IgG² Her47 LHL-LHLF and Fab² Met47 LHL-LHL. Binding was measured using anti-human PE-conjugated secondary antibody. A-D5 IgG1 was tested at 0.1 µg/ml, 1 µg/ml and 10 µg/ml. IgG² Her47 LHL-LHL was tested at 0.1 µg/ml, 1 µg/ml and 10 µg/ml. IgG² Her47 LHL-LHLF was tested at 0.1 µg/ml, 1 µg/ml and 10 µg/ml. Fab² Met47 LHL-LHL was tested at 0.1 µg/ml, 1 µg/ml and 10 µg/ml.

FIG. 22. ‘Tg32’ mouse erythrocyte hemagglutination assay. Agglutination of erythrocytes was performed using A-D5 IgG1, IgG² Her47 LHL-LHL, IgG² Her47 LHL-LHLF and Fab² Met47 LHL-LHL. Proteins were titrated (in nM) using pooled fresh erythrocytes from multiple donor mice.

FIG. 23. Tolerability study in ‘Tg32’ mouse: bodyweight analyses. A tolerability study using A-D5 IgG1, IgG² Her47 LHL-LHL, IgG² Her47 LHL-LHLF and Fab² Met47 LHL-LHL was performed dosing all proteins at 2 mg/kg and 10 mg/kg concentrations in Tg32 mice. Body weights were then monitored for 60 days. A-D5 IgG1 10 mg/kg dose was not tolerated, cohort terminated on day 1.

FIG. 24. Tolerability study in ‘Tg32’ mouse: reticulocyte analyses at Day 5 after dosing. A tolerability study using A-D5 IgG1, IgG² Her47 LHL-LHL, IgG² Her47 LHL-LHLF and Fab² Met47 LHL-LHL was performed dosing all proteins at 2 mg/kg and 10 mg/kg concentrations in Tg32 mice. Blood samples were taken and reticulocyte levels measured. A-D5 IgG1 at 2 mg/kg dose demonstrated significantly elevated reticulocyte levels.

FIGS. 25A - 25K. Tolerability study in ‘Tg32’ mouse: haematology analyses at Days 5, 29 and 60 after dosing. A tolerability study using A-D5 IgG1, IgG² Her47 LHL-LHL, IgG² Her47 LHL-LHLF and Fab² Met47 LHL-LHL was performed dosing all proteins at 2 mg/kg and 10 mg/kg concentrations in Tg32 mice. Blood samples were taken and reticulocyte (FIG. 25A), erythrocyte (RBC, FIG. 25B), haemoglobin (FIG. 25C), mean corpuscular haemoglobin concentration (MCHC) (FIG. 25D), mean corpuscular volume (MCV) (FIG. 25E), leukocyte (FIG. 25F), monocyte (FIG. 25G), lymphocyte (FIG. 25H), basophil (FIG. 25I), eosinophil (FIG. 25J) and neutrophil (FIG. 25K) levels measured.

FIG. 26. Pharmacokinetics study in ‘Tg32’ mouse: data per molecule at two doses. A pharmacokinetic study using IgG² Her47 LHL-LHL, IgG² Her47 LHL-LHLF and Fab² Met47 LHL-LHL was performed dosing all proteins at 2 mg/kg and 10 mg/kg concentrations in Tg32 mice. A-D5 IgG1 was dosed at 2 mg/kg. Serum samples were taken and human IgG levels measured (in µg/ml) from 30 mins out to 42 days after dosing.

FIG. 27. Pharmacokinetics study in ‘Tg32’ mouse: data per dose. A pharmacokinetic study using IgG² Her47 LHL-LHL, IgG² Her47 LHL-LHLF and Fab² Met47 LHL-LHL was performed dosing all proteins at 2 mg/kg and 10 mg/kg concentrations in Tg32 mice. A-D5 IgG1 was dosed at 2 mg/kg. Serum samples were taken and human IgG levels measured (in µg/ml) from 30 mins out to 42 days after dosing. Concentrations were plotted at the 2 mg/kg (FIG. 27A) and 10 mg/kg (FIG. 27B) doses, separately. A-D5 IgG1 2 mg/kg dose was included in both analyses, for reference.

FIG. 28. Pharmacokinetics study in ‘Tg32’ mouse: AUC data per dose. A pharmacokinetic study using IgG² Her47 LHL-LHL, IgG² Her47 LHL-LHLF and Fab² Met47 LHL-LHL was performed dosing all proteins at 2 mg/kg and 10 mg/kg concentrations in Tg32 mice. A-D5 IgG1 was dosed at 2 mg/kg. Serum samples were taken and human IgG levels measured (in µg/ml) from 30 mins out to 42 days after dosing. Concentration measurements over time were used to calculate Area Under the Curve (AUC) for each dose.

FIG. 29A - FIG. 29B. Flow cytometric analyses of binding to NHP and human erythrocytes. Flow cytometric analyses of binding to erythrocytes was performed using A-D5 3 M (effector null) IgG1, IgG² Her47 LHL-LHL, IgG² Her47 LHL-LHLF and Trastuzumab. Binding was measured using anti-human PE-conjugated secondary antibody. A-D5 IgG1 was the only protein that exhibited concentration-dependent binding to both NHP (cynomolgus monkey) erythrocytes (FIG. 29A) and human erythrocytes (FIG. 29B).

FIG. 30A - FIG. 30N. Direct ELISA for purified, intact and MMP-digested protein constructs (digested at pH7.4 and pH6.0) binding to human target proteins. Protein constructs were submitted to enzymatic digestion at either pH7.4 or pH6.0 using human MMPs, over a time course of 2, 4, 8 and 24 hours incubation, plus a 24 h incubation in buffer without enzyme as a negative control (time 0). Samples from these digest time courses were then applied in direct binding ELISA against human Her2 and CD47.

FIG. 31A - FIG. 31B. Direct ELISA for purified, intact and Cathepsin-digested protein constructs (digested at pH7.4 and pH6.0) binding to human target proteins. Protein constructs were submitted to enzymatic digestion at either pH7.4 or pH6.0 using human Cathepsin enzymes, over a time course of 2, 4, 8 and 24 hours incubation, plus a 24 h incubation in buffer without enzyme as a negative control (time 0). Samples from these digest time courses were then applied in direct binding ELISA against human Her2 and CD47.

FIG. 32A - FIG. 32B. Flow cytometric analyses of binding to human cancer cells. Flow cytometric analyses of binding to erythrocytes was performed using anti-CD47, Trastuzumab, IgG1 isotype, and IgG² Her47 LHL-LHL or IgG² Her47 LHL-LHLF which had both been submitted to enzymatic digestion at pH7.4 using human MMP12, over a time course of 2, 4, 8 and 24 hours incubation, plus a 24 h incubation in buffer without enzyme as a negative control (time 0). Binding was measured using anti-human PE-conjugated secondary antibody. Binding was measured on the Her2-high cell line BT-474 (FIG. 32A, FIG. 32B) and Her2-low MCF-7 (FIG. 32C, FIG. 32D).

FIG. 33. SDS-PAGE analysis of MMP12-digested IgG² Her47 LHL-LHL or IgG² Her47 LHL-LHLF. SDS-PAGE was performed on samples of IgG² Her47 LHL-LHL or IgG² Her47 LHL-LHLF which had both been submitted to enzymatic digestion at pH7.4 using human MMP12, over a time course of 2, 4, 8 and 24 hours incubation, plus a 24 h incubation in buffer without enzyme as a negative control (time 0).

FIG. 34A - FIG. 34B. Mass spectrometry analysis of MMP12-digested IgG² Her47 LHL-LHL. Mass spec was performed on samples of IgG² Her47 LHL-LHL which had been submitted to enzymatic digestion at pH7.4 using human MMP12, over a time course of 2, 4, 8 and 24 hours incubation, plus a 24 h incubation in buffer without enzyme as a negative control (time 0). Presence of peptides indicative of the intact LHL linker (FIG. 34A) and MMP12-cleaved linker (FIG. 34B) was measured. FIG. 34A depicts SEQ ID NO: 110. FIG. 34B depicts SEQ ID NO: 111.

FIG. 35. Size Exclusion Chromatography of Protein A-purified Her47 LHLF-LHL IgG1-2hDAA. Her47 LHLF-LHL IgG1-2hDAA protein was expressed in CHO cells, purified by ProA column and analysed by SEC. Two small, larger MW peaks were observed and a large peak of product of expected size (10.30, approx. 250 kDa).

FIG. 36. SDS-PAGE analysis of purified peak fractions from Size Exclusion Chromatography of Her47 LHLF-LHL IgG1-2hDAA. SDS-PAGE was performed on unreduced samples of Her47 LHLF-LHL IgG1-2hDAA: Lane 1 - Mol wt standard, Lane 2 -total ProA-eluted protein, Lane 3 - blank, Lane 4 - peak 1, Lane 5 - peak two, Lane 6 -peak 3 (correct product).

FIG. 37. SDS-PAGE analysis of purified peak fractions from Size Exclusion Chromatography of Her47 LHLF-LHL IgG1-2hDAA. SDS-PAGE was performed on reduced samples of Her47 LHLF-LHL IgG1-2hDAA: Lane 1 - Mol wt standard, Lane 2 -total ProA-eluted protein, Lane 3 - blank, Lane 4 - peak 1, Lane 5 - peak two, Lane 6 -peak 3 (correct product).

FIG. 38A - FIG. 38C. Direct ELISA for purified, intact and MMP12-digested Her47 IgG1-2hDAA proteins. Her47 LHL-LHLF IgG1-2hDAA (FIG. 38A) was submitted to enzymatic digestion at pH7.4 using human MMP12, over a time course of 2, 4, 8 and 24 hours incubation, plus a 24 h incubation in buffer without enzyme as a negative control (time 0, 2, 4, 8, 24 h incubation). Samples from these digest time courses were then applied in direct binding ELISA against human Her2 and murine EpCAM (FIG. 38A). ELISA was then also performed against human CD47 for digested (dark grey) and undigested (light grey) samples (FIG. 38B). SDS-PAGE was also performed for samples: Lane 1 - Mol wt marker, Lane 2 - 0 hr digest, Lane 3 - 2 hr digest, Lane 4 - 8 hr digest and Lane 4 - 24 hr (FIG. 38C).

FIG. 39A - FIG. 39L. Direct ELISA for purified, intact and MMP12-digested IgG² Her47 proteins with alternative linker compositions. Purified protein from clones Her47 LHL-LHL-EK, Her47-LHL-LHL-Thr, Her47-LHL-LHL-tPA, Her47-LHL-LHL-uPA, Her47-LHL-LHL-GrB and Her47-LHL-LHL-A5 were all tested in titration ELISA against human Her2 and CD47 targets (FIG. 39A, FIG. 39C, FIG. 39E, FIG. 39G, FIG. 39L, FIG. 39K. Each protein was then also submitted to a time course enzymatic digest and ELISA binding to Her2 and CD47 targets (FIG. 39B, FIG. 39D, FIG. 39F, FIG. 39H, FIG. 39J, FIG. 39L).

FIG. 40. Multi-dose tolerability study in NOD-SCID mouse: bodyweight analyses. A tolerability study using IgG² Her47 LHL-LHL, IgG² Her47 LHL-LHLF, Fab² Her47 LHL-LHL and Fab² Her47 LHL-LHLF was performed dosing all proteins every 5 days, with 4 total doses.

FIG. 41A - FIG. 41D. Size Exclusion Chromatography of Protein A-purified Her2CD3 Fab² proteins. Fab² Her23 LHL-LHL-S (FIG. 41A), Fab² Her23 LHLF-LHL-S (FIG. 41B), Fab² Her23 LHL-LHL (FIG. 41C) and Fab² Her23 LHLF-LHL (FIG. 41D), was expressed in CHO cells, purified by ProA column and analysed by SEC. Fab² Her23 LHL-LHL (FIG. 41C) and Fab² Her23 LHLF-LHL (FIG. 41D) both exhibited lower molecular weight contaminants (peak 15.38).

FIG. 42A - FIG. 42B. CD3 co-engagement bioassay analyses for purified, intact and MMP-digested Her2CD3 Fab² proteins using Her2-Low MCF-7 cells. Antibodies Fab² Her23 LHLF-LHL-S (FIG. 42A), Fab² Her23 LHL-LHL-S (FIG. 42B), were submitted to enzymatic digestion at pH7.4 using human MMP12, over a time course of 2, 4, 8 and 24 hours incubation, plus a 24 h incubation in buffer without enzyme as a negative control (time 0). Samples from these digest time courses were then applied in the Promega Jurkat cell CD3 reporter assay, using MCF-7 cells as the target cells.

FIG. 43A - FIG. 43C. CD3 co-engagement bioassay analyses for purified, intact and MMP-digested Her2CD3 Fab² proteins using Her2-high BT-474 cells. Control antibodies (FIG. 43A), and Fab² Her23 LHLF-LHL-S (FIG. 43B), or Fab² Her23 LHL-LHL-S (FIG. 43C) [which had both been submitted to enzymatic digestion at pH7.4 using human MMP12, over a time course of 2, 4, 8 and 24 hours incubation, plus a 24 h incubation in buffer without enzyme as a negative control (time 0)], were applied at 0.1 µg/ml in the Promega Jurkat cell CD3 reporter assay, using BT-474 cells as the target cells.

FIG. 44A - FIG. 44B. Charge variant analysis for IgG² and Fab² Her47 proteins -Charge heterogeneity analysis is important in the characterisation of monoclonal antibodies because it provides important information about product quality and stability. Heterogeneity can be caused by enzymatic post-translational modifications (glycosylation, lysine truncation) or chemical modifications during purification and storage (oxidation or deamidation). Charge variant profiling for the provided test articles was performed by a commercial Charge Variant Assay. The charge variant profiles of the IgG² Her47 LHL-LHL (FIG. 44A) and Fab² Her47 LHL-LHL (FIG. 44B) both displayed a homogeneous profile, with one main isoform (50-57% of total), a major acidic isoform (40-48 % of total) and one minor basic isoform (approx. 3%).

FIG. 45A - FIG. 45B. Size Exclusion Chromatography of Her47 proteins after 5 rounds of freeze-thaw. IgG² Her47 LHL-LHL (FIG. 45A) and Fab² Her47 LHL-LHL (FIG. 45B) were both subjected to 5 rounds of freeze-thaw and then SEC performed for samples from rounds 0-5. No aggregation, fragmentation or loss of product was observed for either protein.

FIG. 46. Alternative protein construct designs. This figure depicts illustrative examples of the protein construct Fab² design. This design may be based on sequences that: 1. Remove the upper variable domains. 2. Contain ‘dummy’ non-binding variable domains. 3. Replace the upper Fab with a diabody (or two scFvs). Variable regions are indicated in white. Constant regions are indicated in grey.

FIG. 47A - FIG. 47B. Cell proliferation analyses for purified, intact Her2CD47 proteins using Her2-high BT-474 cells. Trastuzumab, Isotype control IgG1, IgG² Her47 LHL-LHL (FIG. 47A) and Fab² Her47 LHL-LHL (FIG. 47B), were applied to BT-474 cells over a 72h incubation period and cell proliferation measured. Data is represented as % inhibition of cell growth.

FIG. 48A - FIG. 48G. In vivo efficacy analyses of Her47 molecules in NOD-SCID mice (KYSE-410 model). Trastuzumab (FIG. 48A), IgG² Her47 LHL-LHLF (FIG. 48B), IgG² Her47 LHL-LHL (FIG. 48C), Fab² Her47 LHL-LHLF (FIG. 48D) and Fab² Her47 LHL-LHL (FIG. 48E) were each dosed (intravenously, on days 0, 5, 10) in NOD-SCID mice bearing KYSE-410 tumours. Measurement of tumour volumes was performed on days 4, 7 and 11 and are plotted in comparison to vehicle. Fab² Her47 LHL-LHLF and Fab² Her47 LHL-LHL demonstrated differing potencies (FIG. 48F). None of the dosing groups exhibited any weight loss that might indicate toxicity of the molecules dosed (FIG. 48G).

### DETAILED DESCRIPTION OF THE INVENTION

Disclosed herein are recombinant proteins that are conditionally active in diseased human tissues. In some cases, the protein comprises a binding domain that is masked by another portion of the protein in non-diseased tissues. The protein also comprises a peptide linker that is cleaved by one or more proteases expressed in a diseased tissue. The linker cleavage unmasks the binding domain in the diseased tissue, thus allowing binding and/or function of the protein selectively in the diseased tissue. The proteins of the invention are particularly useful for binding to drug targets that are expressed in both diseased tissue and non-diseased tissue.

Provided herein are a number of activatable protein molecules and medical uses thereof. In some aspects, multiple functional properties of the molecules are considered, including target binding specificity, effective limitation of undesired activity in the native protein but full activity in the activated form, maintained conditional affinity to one or more targets from both human and animal test species (e.g., cynomolgus monkey, also known as the crab-eating macaque, i.e. Macaca fascicularis), biophysical stability and/or yield from protein expression platforms used in research, clinical and commercial supply.

In some aspects, there is provided a protein molecule that specifically binds to one or more human drug targets, and optionally also to cynomolgus monkey orthologs of those targets, wherein the protein molecule comprises heavy and light chain regions assembled from one or multiple polypeptides with the following format:
V-C-LINKER-V-C
V-C-LINKER-V-C or
C-LINKER-V-C
C-LINKER-V-C

In some aspects, the protein molecule comprises two polypeptide chains and has the following format:
VH1-C-LINKER-VH2-C
VL1-C-LINKER-VL2-C

In some aspects, the protein molecule comprises two polypeptide chains and has the following format:
VL1-C-LINKER-VH2-C
VH1-C-LINKER-VL2-C

“V” refers to an immunoglobulin or T cell receptor variable region or the ectodomain of a receptor. “VH1” and “VL1” refer to a heavy chain variable region and a light chain variable region that pair with each other to bind an antigen. “VH2” and “VL2” refer to a heavy chain variable region and a light chain variable region that pair with each other to bind an antigen. “C” refers to an immunoglobulin or T cell receptor constant region. In aspects of the invention, the V-C and V-C units on either side of the linker domain form upper and lower immunoglobulin Fab domains with the lower Fab domain exhibiting binding to its cognate target that is reduced or ablated by the presence of the linker domain which is fused to the N-termini of each V domain in the lower Fab. In another aspect the upper or lower Fab domain may be replaced by an Fc fragment, one or two receptor ectodomains, or any domains having or lacking any specific binding function.

In some aspects, provided herein is a protein comprising a first moiety and a second moiety and a peptide linker between the first moiety and the second moiety,
wherein the peptide linker comprises an amino acid sequence from a human immunoglobulin hinge region or an amino acid sequence or an amino acid sequence having 1, 2, 3, 4, 5, 6, 7, 8, 9, 10, 11, 12, or 13 (e.g., from 1 to about 7) amino acid substitutions compared to a human immunoglobulin hinge region;
wherein the peptide linker is cleavable by a protease expressed in a diseased tissue;
wherein the second moiety is capable of specifically binding to a molecule expressed in the diseased tissue; and
wherein the binding of the second moiety to the molecule expressed in the diseased tissue is reduced or inhibited when the peptide linker is uncleaved.

The linker portion may further comprise a peptide linker derived from an immunoglobulin hinge region, with zero, one, or more mutations away from the germline.

In some aspects, the peptide linker comprises or consists of the sequence set forth in GPAPELL (SEQ ID NO:1), GPAPELLGGGS (SEQ ID NO:2), GPAPLGLGGGS (SEQ ID NO:3), PPCPAPELLGGGS (SEQ ID NO:4), PPCPAPLGLGGGS (SEQ ID NO:5) GPAPELLGGPS (SEQ ID NO:69), GPAPLGLGGPS (SEQ ID NO:70), PPCPAPELLGGPS (SEQ ID NO:71), PPCPAPLGLGGPS (SEQ ID NO:72), GPAPEAAGAGS (SEQ ID NO:81), GPADDDDKSGS (SEQ ID NO:82) (cleavable by Enterokinase), GPALVPRGSGS (SEQ ID NO:83) (cleavable by Thrombin), GPGPFGRSAGGP (SEQ ID NO:84) (cleavable by tPA), GPAPLEADAGS (SEQ ID NO:85) (cleavable by Granzyme B), GPAPEARRGGS (SEQ ID NO:86) (cleavable by uPA), or GPAPEGEARGS (SEQ ID NO:87) (cleavable by ADAMTs-5). In some aspects, the peptide linker comprises or consists of two, three or four of the aforementioned sequences.

Also provided is an immunoconjugate comprising the protein of the invention linked to a therapeutic agent.

In another aspect the invention provides a nucleic acid molecule encoding the protein or a portion thereof as defined herein. Further provided is a vector comprising the nucleic acid molecule of the invention. Also provided is a host cell comprising the nucleic acid molecule or the vector of the invention.

In a further aspect there is provided a method of producing an conditionally active protein of the invention, comprising culturing the host cell of the invention under conditions that result in expression and/or production of the protein, and isolating the protein from the host cell or culture.

In another aspect of the invention there is provided a pharmaceutical composition comprising a protein of the invention as defined herein, or a nucleic acid molecule of the invention as defined herein, or a vector of the invention as defined herein, or an immunoconjugate of the invention as defined herein.

Further provided is a method for enhancing an immune response in a subject, comprising administering an effective amount of a protein of the invention as defined herein, or the immunoconjugate of the invention as defined herein, or the nucleic acid molecule of the invention as defined herein, or the vector of the invention as defined herein, or the pharmaceutical composition of the invention as defined herein.

In a further aspect there is provided a method for treating or preventing cancer in a subject, comprising administering an effective amount of a protein of the invention as defined herein, or the immunoconjugate of the invention as defined herein, or the nucleic acid molecule of the invention as defined herein, or the vector of the invention as defined herein, or the pharmaceutical composition of the invention as defined herein.

Further provided is a protein of the invention as defined herein, or the immunoconjugate of the invention as defined herein, or the nucleic acid molecule of the invention as defined herein, or the vector of the invention as defined herein, or the pharmaceutical composition of the invention as defined herein, for use as a medicament.

Further provided is a protein of the invention as defined herein, or the immunoconjugate of the invention as defined herein, or the nucleic acid molecule of the invention as defined herein, or the vector of the invention as defined herein, or the pharmaceutical composition of the invention as defined herein, for use in the treatment of cancer.

Further provided is a protein, or an immunoconjugate, or a nucleic acid molecule, or a vector, or a pharmaceutical composition of the invention as defined herein, for separate, sequential or simultaneous use in a combination with a second therapeutic agent, for example an anti-cancer agent.

In a further aspect there is provided the use of a protein of the invention as defined herein, or an immunoconjugate of the invention as defined herein, or a nucleic acid molecule of the invention as defined herein, or a vector of the invention as defined herein, or a pharmaceutical composition of the invention as defined herein, in the manufacture of a medicament for the treatment of cancer.

Further provided is a method for treating or preventing an autoimmune disease or an inflammatory disease in a subject, comprising administering an effective amount of a protein as defined herein, or an immunoconjugate as defined here, or a nucleic acid molecule as defined herein, or a vector as defined herein, or a pharmaceutical composition as defined herein.

Also provided is a protein as defined herein, or the immunoconjugate as defined herein, or the nucleic acid molecule as defined herein, or the vector as defined herein, or the pharmaceutical composition as defined herein, for use in the treatment of an autoimmune disease or an inflammatory disease.

Further provided is the use of a protein as defined herein, or an immunoconjugate as defined herein, or a nucleic acid molecule as defined herein, or a vector as defined herein, or a pharmaceutical composition as defined herein, in the manufacture of a medicament for the treatment of an autoimmune disease or an inflammatory disease.

Further provided is a method for treating or preventing a cardiovascular disease or a fibrotic disease in a subject, comprising administering an effective amount of a protein as defined herein, or the immunoconjugate as defined here, or the nucleic acid molecule as defined herein, or the vector as defined herein, or the pharmaceutical composition as defined herein.

Further provided is a protein as defined herein, or the immunoconjugate as defined herein, or the nucleic acid molecule as defined herein, or the vector as defined herein, or the pharmaceutical composition as defined herein, for use as a medicament. Also provided is an antibody molecule or antigen-binding portion thereof as defined herein, or the immunoconjugate as defined herein, or the nucleic acid molecule as defined herein, or the vector as defined herein, or the pharmaceutical composition as defined herein, for use in the treatment of a cardiovascular disease or a fibrotic disease.

Further provided is the use of a protein as defined herein, or an immunoconjugate as defined herein, or a nucleic acid molecule as defined herein, or a vector as defined herein, or a pharmaceutical composition as defined herein, in the manufacture of a medicament for the treatment of an autoimmune disease, an inflammatory disease or a fibrotic disease.

In some aspects, the invention provides a protein comprising a first moiety and a second moiety and a peptide linker between the first moiety and the second moiety, wherein the peptide linker comprises an amino acid sequence from a human immunoglobulin hinge region or an amino acid sequence or an amino acid sequence having amino acid substitutions (e.g., 1, 2, 3, 4, 5, 6, 7, 8, 9, 10, 11, 12, or 13 amino acid substitutions) compared to a human immunoglobulin hinge region; wherein the peptide linker is cleavable by a protease expressed in a diseased tissue; wherein the second moiety is capable of specifically binding to a molecule expressed in the diseased tissue; and wherein the binding of the second moiety to the molecule expressed in the diseased tissue is reduced or inhibited when the peptide linker is uncleaved. In some aspects, the amino acid substitution is a conservative amino acid substitution. In some aspects, the peptide linker comprises an amino acid sequence from a human immunoglobulin hinge region or an amino acid sequence or an amino acid sequence having from 1 to about 7 amino acid substitutions compared to a human immunoglobulin hinge region. In some aspects, the peptide linker comprises an amino acid sequence from a human immunoglobulin hinge region or an amino acid sequence or an amino acid sequence having 1-2, 1-3, 1-4, 1-5, 1-6, 1-7, 2-3, 2-4, 2-5, 2-6, 2-7, 3-4, 3-5, 3-6, 3-7, 4-5, 4-6, 4-7, 5-6, 5-7, or 6-7 amino acid substitutions compared to a human immunoglobulin hinge region.

In some aspects, the peptide linker cleavable by a protease expressed in a diseased tissue is cleavable by a human matrix metalloprotease (MMP) or a human cathepsin. In some cases, the peptide linker cleavable by a protease expressed in a diseased tissue is cleavable by human enterokinase (EK), human thrombin (Thr), human tPA (tissue plasminogen activator), human Granzyme B (GrB), human uPA (urokinase-type plasminogen activator), or human ADAMTs-5 (A Disintegrin-like And Metalloproteinase With Thrombospondin Type 1 Motif 5; A5). In some cases, the peptide linker comprises a human MMP cleavage site or a human cathepsin cleavage site. In some cases, the peptide linker comprises a human enterokinase, human thrombin, human tPA, human Granzyme B, human uPA, or human ADAMTs-5 cleavage site. In some cases, the peptide linker comprises the MMP substrate sequence PLGL (SEQ ID NO:12). In some cases, the peptide linker comprises or consists of the amino acid sequence of GPAPELL (SEQ ID NO:1), GPAPELLGGGS (SEQ ID NO:2), GPAPLGLGGGS (SEQ ID NO:3), PPCPAPELLGGGS (SEQ ID NO:4), or PPCPAPLGLGGGS (SEQ ID NO:5), GPAPELLGGPS (SEQ ID NO:69), GPAPLGLGGPS (SEQ ID NO:70), PPCPAPELLGGPS (SEQ ID NO:71), PPCPAPLGLGGPS (SEQ ID NO:72), GPAPEAAGAGS (SEQ ID NO:81), GPADDDDKSGS (SEQ ID NO:82), GPALVPRGSGS (SEQ ID NO:83), GPGPFGRSAGGP (SEQ ID NO:84), GPAPLEADAGS (SEQ ID NO:85), GPAPEARRGGS (SEQ ID NO:86), or GPAPEGEARGS (SEQ ID NO:87).

In some cases, the peptide linker comprises or consists of two, three, or four of the amino acid sequences in Table 1 fused in a single amino acid chain via peptide bonds. In some cases, the peptide linker is between about 5 amino acids and about 15 amino acids, between about 5 amino acids and about 20 amino acids, or between about 5 amino acids and about 25 amino acids in length.

In some cases, the peptide linker between the first moiety and the second moiety comprises the following amino acid sequence at the N-terminus of the peptide linker sequence: X1-proline-X2. In some aspects, X1 is alanine, glycine, serine, proline or threonine. In some aspects, X1 is alanine, glycine, serine, proline or threonine, aspartic acid, asparagine or valine. In some aspects, X2 is alanine, glycine, serine, proline or threonine. In some aspects, X2 is alanine, glycine, serine, proline or threonine, aspartic acid, asparagine or valine. In some aspects, X1 and X2 are the same amino acid. In some aspects, X1 and X2 are different amino acids.

In some cases, the peptide linker cleavable by a protease expressed in a diseased tissue is cleavable by any one of human MMP1, MMP2, MMP3, MMP4, MMP5, MMP6, MMP7, MMP8, MMP9, MMP10, MMP11, MMP12, MMP13, MMP14, MMP15, MMP16, MMP17, MMP18, MMP19, MMP20, MMP21, MMP22, MMP23, MMP24, MMP25, MMP26, MMP27, or MMP28. In some cases, the peptide linker cleavable by a protease expressed in a diseased tissue is cleavable by any one of human MMP-2, MMP-3, MMP-7, MMP-8, MMP-9, MMP-10, MMP-12 orMMP-13. In some cases, the level or the activity of the human MMP is elevated in the diseased tissue compared to the level or the activity of the human MMP in a non-diseased tissue.

In some cases, the peptide linker cleavable by a protease expressed in a diseased tissue is cleavable by any one of human Cathepsin A, Cathepsin B, Cathepsin C, Cathepsin D, Cathepsin E, Cathepsin F, Cathepsin G, Cathepsin H, Cathepsin K, Cathepsin L1, Cathepsin V, Cathepsin O, Cathepsin S, Cathepsin W, or Cathepsin Z. In some cases, the peptide linker cleavable by a protease expressed in a diseased tissue is cleavable by any one of human Cathepsin D, Cathepsin G, or Cathepsin K. In some cases, the level or the activity of the human cathepsin is elevated in the diseased tissue compared to the level orthe activity of the human cathepsin in a non-diseased tissue. In some cases, the level or the activity of the human cathepsin is elevated in tissues with pH < 7.0 compared to the level or the activity of the human cathepsin in a tissue with pH ≧ 7.4.

In some aspects, the first moiety of any of the proteins of the invention comprises an antibody, an antigen-binding portion of an antibody or a receptor ectodomain. In some cases, the first moiety is a Fab, a single-chain Fab, a VH domain, a VL domain, an immunoglobulin new antigen receptor (IgNAR), a single-chain variable fragment (scFv), a diabody, or a T cell receptor domain. An IgNAR is a homodimeric heavy chain-only antibody produced by sharks and other cartilaginous fishes (Feige et al., PNAS, 2014, 111(22):8155-8160).

In some cases, the first moiety specifically binds to a molecule expressed in a diseased tissue. In some embodiments, the first moiety specifically binds to a tumour-associated antigen (TAA). In some cases, the first moiety binds specifically to human EGFR, human HER2, human HER3, human CD105, human C-KIT, human PD1, human PD-L1, human PSMA, human EpCAM, human Trop2, human EphA2, human CD20, human BCMA, human GITR, human OX40, human CSF1R, human Lag3 or human cMET. In some embodiments, the first moiety also binds the cynomolgus ortholog of any of these molecules.

In some aspects, the first moiety comprises a heavy chain variable (VH) region and a light chain variable (VL) region. In some cases, the first moiety further comprises an immunoglobulin constant region or a portion of an immunoglobulin constant region. In some cases, the immunoglobulin constant region is IgG, IgE, IgM, IgD, IgA or IgY.

In some aspects, the anti-HER2 variable region sequences used in the protein constructs disclosed herein are the variable region sequences of trastuzumab. In some aspects, the anti-CD3 variable region sequences used in the protein constructs disclosed herein are the variable region sequences of OKT3 or SP34. In some aspects, the anti-cMET variable region sequences used in the protein constructs disclosed herein are provided in WO 2019/175186. In some aspects, the anti-CD47 variable region sequences used in the protein constructs disclosed herein are provided in WO 2019/034895.

In some aspects, the second moiety of any of the proteins of the invention comprises an antibody, an antigen-binding portion of an antibody or a receptor ectodomain. In some cases, the second moiety is a Fab, a single-chain Fab, a VH domain, a VL domain, an immunoglobulin new antigen receptor (IgNAR), a single-chain variable fragment (scFv), a diabody, or a T cell receptor domain.

In some cases, the second moiety specifically binds to a molecule expressed in a diseased tissue. In some embodiments, the second moiety specifically binds to a tumour-associated antigen (TAA). In some cases, the second moiety binds specifically to human CD47. In some cases, the second moiety binds specifically to human PD-L1. In some cases, the second moiety specifically binds to a molecule expressed by a human immune cell. In some cases, the molecule expressed by a human immune cell is human CD3, human CD16A, human CD16B, human CD28, human CD89, human CTLA4, human NKG2D, human SIRPα, human SIRPγ, human PD1, human Lag3, human 4-1BB, human OX40, or human GITR. In some embodiments, the second moiety also binds the cynomolgus ortholog of any of these molecules.

In some aspects, the second moiety comprises a heavy chain variable (VH) region and a light chain variable (VL) region. In some cases, the second moiety further comprises an immunoglobulin constant region or a portion of an immunoglobulin constant region. In some cases, the immunoglobulin constant region is IgG, IgE, IgM, IgD, IgA or IgY.

In some aspects, the first moiety and/or the second moiety of a protein of the invention may comprise an immunoglobulin constant region. In some embodiments, the immunoglobulin constant region is IgG1, IgG2, IgG3, IgG4,IgA1, IgA2, IgE, or IgM. In additional embodiments, the immunoglobulin constant region is IgG1, IgG2, IgG3, lgG1null, IgG4(S228P), IgA1, IgA2, IgE, or IgM. In some embodiments, the first moiety and/or the second moiety of a protein of the invention may comprise an immunologically inert constant region. In some aspects, the first moiety and/or the second moiety of a protein of the invention may comprise an immunoglobulin constant region comprising a wild-type human IgG1 constant region, a human IgG1 constant region comprising the amino acid substitutions L234A and L235A, a human IgG1 constant region comprising the amino acid substitutions L234A, L235A and G237A or a human IgG1 constant region comprising the amino acid substitutions L234A, L235A, G237A and P331S. In some aspects, the first moiety and/or the second moiety of a protein of the invention may comprise an immunoglobulin constant region comprising a wild-type human IgG² constant region or a wild-type human IgG4 constant region. In some aspects, the first moiety and/or the second moiety of a protein of the invention may comprise an immunoglobulin constant region comprising any one of the amino acid sequences in Table 10. The Fc region sequences in Table 10 begin at the CH1 domain. In some aspects, the first moiety and/or the second moiety of a protein of the invention may comprise an immunoglobulin constant region comprising an amino acid sequence of an Fc region of human IgG4,human IgG4(S228P), human IgG2, human IgG1, human IgG1-3M or human IgG1-4M. For example, the human IgG4(S228P) Fc region comprises the following substitution compared to the wild-type human IgG4 Fc region: S228P. For example, the human IgG1-3M Fc region comprises the following substitutions compared to the wild-type human IgG1 Fc region: L234A, L235A and G237A, while the human IgG1-4M Fc region comprises the following substitutions compared to the wild-type human IgG1 Fc region: L234A, L235A, G237A and P331S. In some aspects, a position of an amino acid residue in a constant region of an immunoglobulin molecule is numbered according to EU nomenclature (Ward et al., 1995 Therap. Immunol. 2:77-94). In some aspects, an immunoglobulin constant region may comprise an RDELT (SEQ ID NO:65) motif or an REEM (SEQ ID NO:66) motif (underlined in Table 10). The REEM (SEQ ID NO:66) allotype is found in a smaller human population than the RDELT (SEQ ID NO:65) allotype. In some aspects, the first moiety and/or the second moiety of a protein of the invention antibody may comprise an immunoglobulin constant region comprising any one of SEQ ID NOS:56-62. In some aspects, the first moiety and/or the second moiety of a protein of the invention may comprise the heavy chain amino acid sequence and the light chain amino acid sequence of any one of the clones in Tables 3-9 and any one of the Fc region amino acid sequences in Table 10. In some aspects, the first moiety and/or the second moiety of a protein of the invention may comprise an immunoglobulin heavy chain constant region comprising any one of the Fc region amino acid sequences in Table 10 and an immunoglobulin light chain constant region that is a kappa light chain constant region or a lambda light chain constant region.

In some aspects, a protein of the invention comprises an IgG1 isotype constant region. The IgG1 isotype constant region potently activates all FcyR signalling types, thus driving maximal opsonizing effector function.

In some aspects, the immunoglobulin constant region comprises a hinge region or a truncated hinge region. In some embodiments, a hinge region may comprise one, two, three, four or more amino acid substitutions compared to a wild-type human hinge region amino acid sequence. In some embodiments, the immunoglobulin constant region does not comprise a hinge region.

In some aspects, the first moiety specifically binds to a first molecule expressed in a diseased tissue and the second moiety is capable of specifically binding to a second molecule expressed in a diseased tissue, wherein the first molecule expressed in a diseased tissue and the second molecule expressed in a diseased tissue are different molecules. In some embodiments, the first molecule expressed in a diseased tissue and the second molecule expressed in a diseased tissue are expressed by the same cell. In some embodiments, the first molecule expressed in a diseased tissue and the second molecule expressed in a diseased tissue are expressed by different cells. In some embodiments, the first molecule expressed in a diseased tissue, the second molecule expressed in a diseased tissue, or both the first molecule expressed in a diseased tissue and the second molecule expressed in a diseased tissue is expressed on the surface of a cell. In some embodiments, the first molecule expressed in a diseased tissue and/or the second molecule expressed in a diseased tissue is a soluble molecule.

In some aspects, the first moiety binds specifically to human cMET and the second moiety binds specifically to human CD47. In some aspects, the first moiety binds specifically to human HER2 and the second moiety binds specifically to human CD47. In some aspects, the first moiety binds specifically to human cMET and the second moiety binds specifically to human CD47. In some aspects, the first moiety binds specifically to human HER2 and the second moiety binds specifically to human CD3. In some aspects, the first moiety binds specifically to human cMET and the second moiety binds specifically to human cMET.

In some aspects, a protein of the invention has an immune effector function or two, three or more immune effector functions. For example, an immune effector function may be antibody-dependent cellular cytotoxicity (ADCC), complement dependent cytotoxicity (CDC), or antibody-dependent cellular phagocytosis (ADCP).

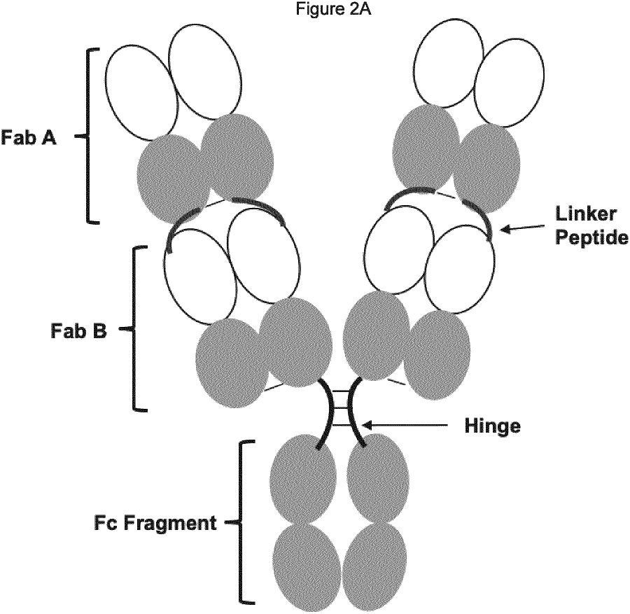

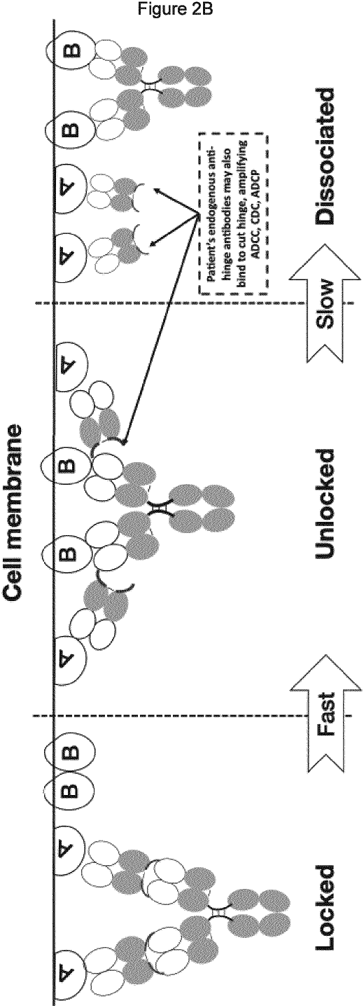

In some aspects, the first moiety of a protein of the invention prevents or reduces specific binding of the second moiety to the molecule expressed in the diseased tissue. In some embodiments, the peptide linker of a protein of the invention is cleaved in the vicinity of the diseased tissue or inside the diseased tissue. In some cases, the peptide linker is cleaved in the vicinity of the diseased tissue or inside the diseased tissue, wherein the first moiety dissociates from the second moiety in the vicinity of the diseased tissue or inside the diseased tissue and wherein the second moiety specifically binds to the molecule expressed in the diseased tissue in the vicinity of the diseased tissue or inside the diseased tissue. In some cases, the cleaved peptide linker comprises a binding site or a target site for anti-hinge antibodies (e.g., a subject’s endogenous anti-hinge antibodies), whereas the uncleaved (e.g., intact) peptide linker does not comprise a binding site or a target site for anti-hinge antibodies. Binding of the anti-hinge antibodies to the cleaved peptide linker may increase ADCC, CDC, and/or ADCP in the presence of an activated protein of the invention (see, e.g., FIG. 2B).

In some aspects, a protein of the invention stimulates inflammatory signalling in a diseased tissue. Increased inflammatory signalling may increase immune recruitment to the diseased tissue. In some cases, a protein of the invention increases antigen presentation in a diseased tissue. In some cases, a protein of the invention increases tumour-associated-antigen-specific T cell proliferation.

In some aspects, the diseased tissue may be a tumour, a necrotic tissue, a fibrotic tissue, a tissue undergoing the clotting cascade or an inflamed tissue.

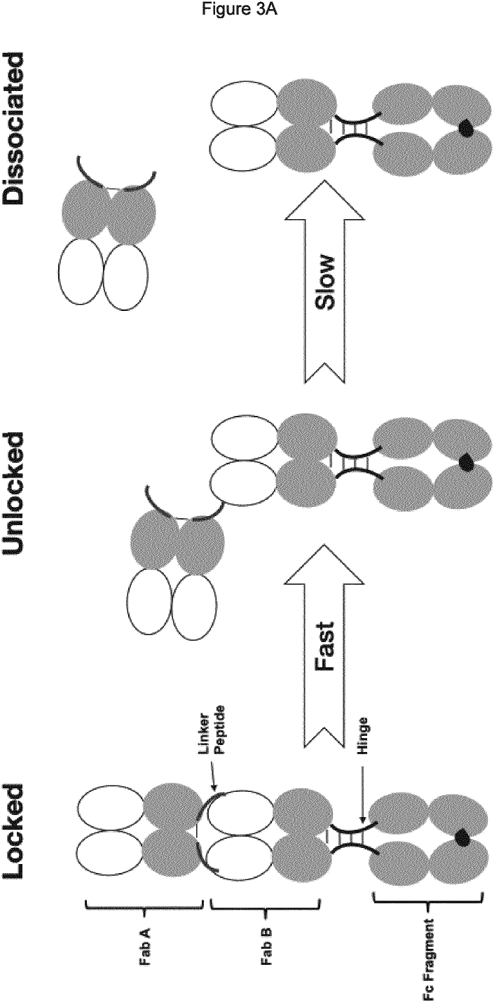

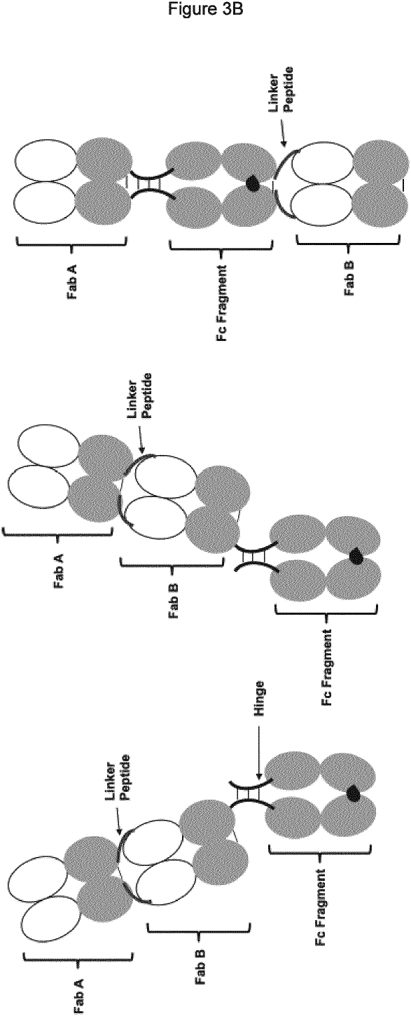

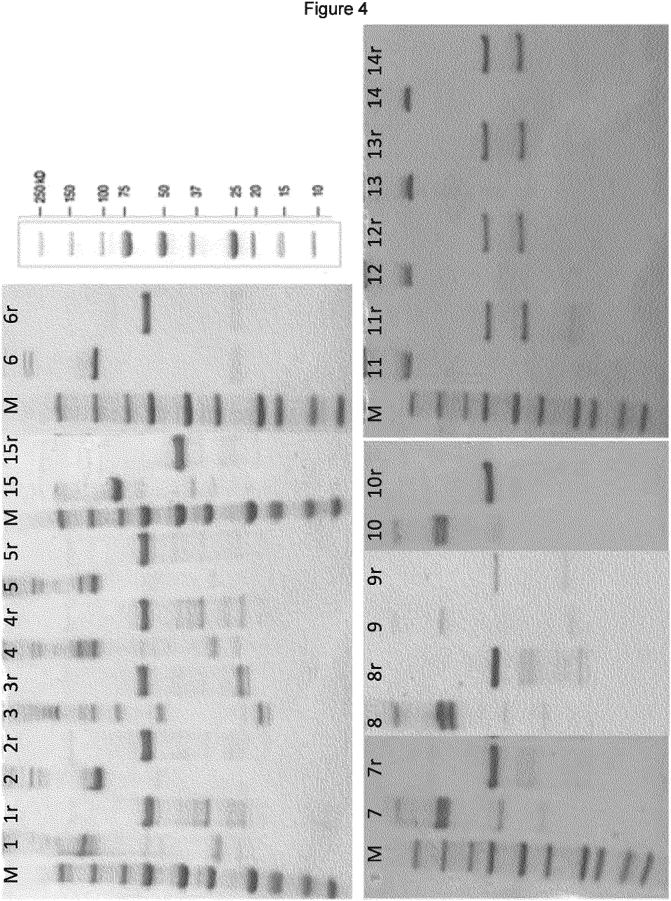

In some aspects, provided herein is a protein comprising a first moiety and a second moiety, and a peptide linker between the first moiety and the second moiety, wherein the first moiety binds specifically to human cMET, wherein the second moiety binds specifically to human CD47, and wherein the protein comprises a first polypeptide chain comprising or consisting of the amino acid sequence of SEQ ID NO:16 and a second polypeptide chain comprising or consisting of the amino acid sequence of SEQ ID NO:17. The protein comprises only one copy of the first polypeptide chain and only one copy of the second polypeptide chain. The peptide linker of this protein comprises two copies of the LHL sequence (see Table 1), each fused at the n-terminus to the first moiety and at the c-terminus to the second moiety, via a peptide bond. This protein is referred to as “Fab2 cMetCD47-LHL-LHL” or “Met47-LHL-LHL″. The amino acid sequences are provided in Table 3. The structure of this protein is depicted in FIG. 3A. The second moiety of this protein is linked via a G4S linker (SEQ ID NO:15) and a truncated hinge region to KIH IgG1-Fc.

In some aspects, provided herein is a protein comprising a first moiety and a second moiety, and a peptide linker between the first moiety and the second moiety, wherein the first moiety binds specifically to human HER2, wherein the second moiety binds specifically to human CD3, and wherein the protein comprises a first polypeptide chain comprising or consisting of the amino acid sequence of SEQ ID NO:26 and a second polypeptide chain comprising or consisting of the amino acid sequence of SEQ ID NO:27. The protein comprises only one copy of the first polypeptide chain and only one copy of the second polypeptide chain. The peptide linker of this protein comprises two copies of the LHL sequence (see Table 1) fused via a peptide bond. This protein is referred to as “Fab2 Her2CD3-LHL-LHL” or “Her23-LHL-LHL”. The amino acid sequences are provided in Table 4. The structure of this protein is depicted in FIG. 3A. The second moiety of this protein is linked via a G4S linker (SEQ ID NO:15) and a truncated hinge region (3M) to KIH IgG1-Fc.

In some aspects, provided herein is a protein comprising a first moiety and a second moiety, and a peptide linker between the first moiety and the second moiety, wherein the first moiety binds specifically to human HER2, wherein the second moiety binds specifically to human CD47, and wherein the protein comprises a first polypeptide chain comprising or consisting of the amino acid sequence of SEQ ID NO:34 and a second polypeptide chain comprising or consisting of the amino acid sequence of SEQ ID NO:35. The protein comprises two identical copies of the first polypeptide chain and two identical copies of the second polypeptide chain. The peptide linker of this protein comprises two copies of the LHL sequence (see Table 1) fused via a peptide bond. This protein is referred to as “IgG2 Her2CD47-LHL-LHL” or “Her47-LHL-LHL”. The amino acid sequences are provided in Table 5. The structure of this protein is depicted in FIG. 2A. The second moiety of this protein may be linked via a hinge region or a truncated hinge region to a human IgG1 Fc sequence (see Table 10).

In some aspects, provided herein is a protein comprising a first moiety and a second moiety, and a peptide linker between the first moiety and the second moiety, wherein the first moiety binds specifically to human cMET, wherein the second moiety binds specifically to human CD47, and wherein the protein comprises a first polypeptide chain comprising or consisting of the amino acid sequence of SEQ ID NO:36 and a second polypeptide chain comprising or consisting of the amino acid sequence of SEQ ID NO:37. The first polypeptide chain further comprises a human IgG1 amino acid sequence (see Table 10). The second polypeptide chain further comprises a human kappa light chain amino acid sequence. The peptide linker of this protein comprises two copies of the LHL sequence (see Table 1) fused via a peptide bond. This protein is referred to as “Fab2 CMET/CD47 ‘One Arm’ style” or “Met47-LHL-LHL”. The amino acid sequences are provided in Table 6. The structure of this protein is depicted in FIG. 3B. The construct is a ‘Knob into hole’ One-Arm Fab2 construct that has Fab2 on the knob side and a hinge-hole Fc stump on the other. This construct may comprise a human IgG1 Fc sequence that is not effector null (see Table 10).

In some aspects, provided herein is a protein comprising a first moiety and a second moiety, and a peptide linker between the first moiety and the second moiety, wherein the first moiety binds specifically to human Her2, wherein the second moiety binds specifically to human CD3, and wherein the protein comprises a first polypeptide chain comprising or consisting of the amino acid sequence of SEQ ID NO:38 and a second polypeptide chain comprising or consisting of the amino acid sequence of SEQ ID NO:39. The first polypeptide chain further comprises a human IgG1-3M amino acid sequence (see Table 10). The second polypeptide chain further comprises a human kappa light chain amino acid sequence. The peptide linker of this protein comprises two copies of the LHL sequence (see Table 1) fused via a peptide bond. This protein is referred to as “Fab2 Her2/CD3 ‘One Arm’ style” or “Her23-LHL-LHL”. The amino acid sequences are provided in Table 7. The structure of this protein is depicted in FIG. 3B. The construct is a ‘Knob into hole’ One-Arm Fab2 construct that has Fab2 on the knob side and a hinge-hole Fc stump on the other. This construct is also effector null (IgG1-3M; see Table 10).

In some aspects, provided herein is a protein comprising a first moiety and a second moiety, and a peptide linker between the first moiety and the second moiety, wherein the first moiety binds specifically to human Her2, wherein the second moiety binds specifically to human CD3, and wherein the protein comprises a first polypeptide chain comprising or consisting of the amino acid sequence of SEQ ID NO:40 and a second polypeptide chain comprising or consisting of the amino acid sequence of SEQ ID NO:41. The first polypeptide chain further comprises a human IgG1-3M amino acid sequence (see Table 10). The second polypeptide chain further comprises a human lambda light chain amino acid sequence. The peptide linker of this protein comprises two copies of the LHL sequence (see Table 1) fused via a peptide bond. This protein is referred to as “Fab2 Her2/CD3(34) “One Arm” style” or “Her23(34)-LHL-LHL”. The amino acid sequences are provided in Table 8. The structure of this protein is depicted in FIG. 3B. The construct is a ‘Knob into hole’ One-Arm Fab2 construct that has Fab2 on the knob side and a hinge-hole Fc stump on the other. This construct is also effector null (IgG1-3M; see Table 10).

In some aspects, provided herein is a protein comprising a first moiety and a second moiety, and a peptide linker between the first moiety and the second moiety, wherein the first moiety binds specifically to human Her2, wherein the second moiety binds specifically to human CD47, and wherein the protein comprises a first polypeptide chain and a second polypeptide chain, wherein:
(a) the first polypeptide chain comprises or consists of the amino acid sequence of SEQ ID NO:42, and the second polypeptide chain comprises or consists of the amino acid sequence of SEQ ID NO:43 (referred to as “Her47-LHLF-LHL”); or
(b) the first polypeptide chain comprises or consists of the amino acid sequence of SEQ ID NO:44, and the second polypeptide chain comprises or consists of the amino acid sequence of SEQ ID NO:45 (referred to as “Her47-LHL-LHLF”); or
(c) the first polypeptide chain comprises or consists of the amino acid sequence of SEQ ID NO:46, and the second polypeptide chain comprises or consists of the amino acid sequence of SEQ ID NO:47 (referred to as “Her47 -LHLF-LHLF”); or
(d) the first polypeptide chain comprises or consists of the amino acid sequence of SEQ ID NO:48, and the second polypeptide chain comprises or consists of the amino acid sequence of SEQ ID NO:49 (referred to as “Her47-LHLM-LHLM”); or
(e) the first polypeptide chain comprises or consists of the amino acid sequence of SEQ ID NO:50, and the second polypeptide chain comprises or consists of the amino acid sequence of SEQ ID NO:51 (referred to as “Her47-LHLM-LHLMF”); or
(f) the first polypeptide chain comprises or consists of the amino acid sequence of SEQ ID NO:52, and the second polypeptide chain comprises or consists of the amino acid sequence of SEQ ID NO:53 (referred to as “Her47-LHLMF-LHLM”); or
(g) the first polypeptide chain comprises or consists of the amino acid sequence of SEQ ID NO:54, and the second polypeptide chain comprises or consists of the amino acid sequence of SEQ ID NO:55 (referred to as “Her47-LHLMF-LHLMF”); or
(h) the first polypeptide chain comprises or consists of the amino acid sequence of SEQ ID NO:44, and the second polypeptide chain comprises or consists of the amino acid sequence of SEQ ID NO:92 (referred to as “Her47 LHL-LHL-EK”);
(i) the first polypeptide chain comprises or consists of the amino acid sequence of SEQ ID NO:44, and the second polypeptide chain comprises or consists of the amino acid sequence of SEQ ID NO:93 (referred to as “Her47-LHL-LHL-Thr”);
(j) the first polypeptide chain comprises or consists of the amino acid sequence of SEQ ID NO:44, and the second polypeptide chain comprises or consists of the amino acid sequence of SEQ ID NO:94 (referred to as “Her47-LHL-LHL-tPA”);
(k) the first polypeptide chain comprises or consists of the amino acid sequence of SEQ ID NO:44, and the second polypeptide chain comprises or consists of the amino acid sequence of SEQ ID NO:95 (referred to as “Her47-LHL-LHL-GrB”);
(l) the first polypeptide chain comprises or consists of the amino acid sequence of SEQ ID NO:44, and the second polypeptide chain comprises or consists of the amino acid sequence of SEQ ID NO:96 (referred to as “Her47-LHL-LHL-uPA”); or
(m) the first polypeptide chain comprises or consists of the amino acid sequence of SEQ ID NO:44, and the second polypeptide chain comprises or consists of the amino acid sequence of SEQ ID NO:97 (referred to as “Her47-LHL-LHL-A5”). These proteins are generally referred to as “lgG2Her2/CD47”. The amino acid sequences are provided in Table 9 and

Table 20. The structure of this protein is depicted in FIG. 2A. The peptide linker sequences for the LHLF, the LHLM and the LHLMF linkers are provided in Table 1. The peptide linker sequences for the EK, Thr, tPA, GrB, uPA, and A5 linkers are provided in Table 21.

In some aspects, provided herein is a protein comprising a first moiety and a second moiety, and a peptide linker between the first moiety and the second moiety, wherein the first moiety binds specifically to human Her2, wherein the second moiety binds specifically to human CD47, and wherein the protein comprises a first polypeptide chain and a second polypeptide chain, wherein:
(a) the first polypeptide chain comprises or consists of the amino acid sequence of SEQ ID NO:88, and the second polypeptide chain comprises or consists of the amino acid sequence of SEQ ID NO:89 (referred to as “Her47 LHLF-LHL IgG1-2hDAA”); or
(b) the first polypeptide chain comprises or consists of the amino acid sequence of SEQ ID NO:90, and the second polypeptide chain comprises or consists of the amino acid sequence of SEQ ID NO:91 (referred to as “Her47 LHL-LHLF IgG1-2hDAA”). These proteins are generally referred to as “IgG2 ‘IgG1-DAA’ Her2/CD47”. The amino acid sequences are provided in Table 19. These proteins comprise stabilizing mutations in the hinge. The structure of this protein is depicted in FIG. 2A. The peptide linker sequences for the LHLF and the LHL linkers are provided in Table 1.

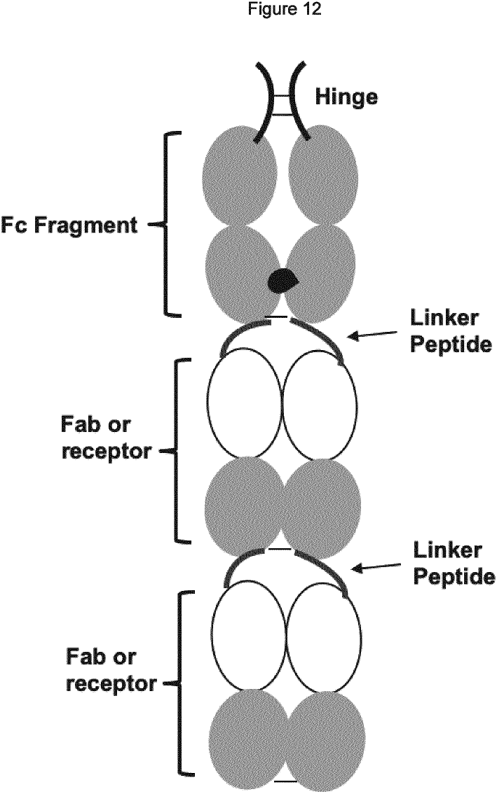

In some aspects, provided herein is a protein comprising a first moiety and a second moiety, and a peptide linker between the first moiety and the second moiety, wherein the first moiety binds specifically to human Her2, wherein the second moiety binds specifically to human CD3, and wherein the protein comprises a first polypeptide chain comprising or consisting of the amino acid sequence of SEQ ID NO:73 and a second polypeptide chain comprising or consisting of the amino acid sequence of SEQ ID NO:74. The first polypeptide chain further comprises a human IgG1-3M Fc amino acid sequence (e.g. containing ‘hole’ mutations’ to enable heterodimerization with the second polypeptide chain), followed by a linker sequence, the VH and CH1 domains of the first binding moiety, another linker sequence, then the VH and CH1 domains of the second binding moiety (see Table 13). The second polypeptide chain further comprises a human IgG1-3M Fc amino acid sequence (e.g. containing ‘knob’ mutations’ to enable heterodimerization with the second polypeptide chain), followed by a linker sequence, the VL and CL domains of the first binding moiety, another linker sequence, then the VL and CL domains of the second binding moiety (see Table 13). The peptide linkers of this protein comprise four sequences (see Table 1) fused via a peptide bond, which reside between the Fc and first moiety and between the first and second binding moieties. This protein is referred to as “Fc-Her2/CD3(34)” or “Fc-Her23(34)”. The amino acid sequences are provided in Table 13. The structure of this protein is depicted in FIG. 12. The construct is a ‘Knob into hole’ Fc-Fab2 construct that has light chain polypeptides on either the knob or hole side and heavy chain polypeptides on the other. This construct is also effector null (IgG1-3M; see Table 10).

In some aspects, provided herein is a protein comprising a first moiety and a second moiety, and a peptide linker between the first moiety and the second moiety, wherein the first moiety binds specifically to human cMET, wherein the second moiety binds specifically to human cMET, and wherein the protein comprises a first polypeptide chain comprising or consisting of the amino acid sequence of SEQ ID NO:75 and a second polypeptide chain comprising or consisting of the amino acid sequence of SEQ ID NO:76. The first polypeptide chain further comprises a human IgG1 or human IgG1-3M amino acid sequence (see Table 10). The second polypeptide chain further comprises a human kappa light chain amino acid sequence. The peptide linker of this protein comprises two copies of the LHL sequence (see Table 1) fused via a peptide bond. This protein is referred to as “Fab2 CMET/CMET ‘One Arm’ style” or “MetMet-LHL-LHL”. The amino acid sequences are provided in Table 14. The structure of this protein is depicted in FIG. 3B. The construct is a ‘Knob into hole’ One-Arm Fab2 construct that has Fab2 on the knob side and a hinge-hole Fc stump on the other. This construct may comprise a human IgG1 Fc sequence that is or is not effector null (see Table 10).

In some aspects, provided herein is a protein comprising a first moiety and a second moiety, and a peptide linker between the first moiety and the second moiety, wherein the first moiety binds specifically to human Her2, wherein the second moiety binds specifically to human CD3, and wherein the protein comprises a first polypeptide chain and a second polypeptide chain, wherein:
(a) the first polypeptide chain comprises or consists of the amino acid sequence of SEQ ID NO:98, and the second polypeptide chain comprises or consists of the amino acid sequence of SEQ ID NO:99 (referred to as “Fab2 Her23 LHL-LHLF ”); or
(b) the first polypeptide chain comprises or consists of the amino acid sequence of SEQ ID NO:100, and the second polypeptide chain comprises or consists of the amino acid sequence of SEQ ID NO:101 (referred to as “Fab2 Her23 LHL-LHL”); or
(c) the first polypeptide chain comprises or consists of the amino acid sequence of SEQ ID NO:102, and the second polypeptide chain comprises or consists of the amino acid sequence of SEQ ID NO:103 (referred to as “Fab2 Her23 LHLF-LHL-S”); or
(d) the first polypeptide chain comprises or consists of the amino acid sequence of SEQ ID NO:104, and the second polypeptide chain comprises or consists of the amino acid sequence of SEQ ID NO:105 (referred to as “Fab2 Her23 LHL-LHL-S”). This protein is referred to as “Fab2 Her2/CD3”. The amino acid sequences are provided in Table 22. The structure of this protein is depicted in FIG. 3A. The protein comprises only one copy of the first polypeptide chain and only one copy of the second polypeptide chain. The peptide linker of this protein comprises two copies of the LHL sequence (see Table 1), or one copy of the LHL sequence and one copy of the LHLF sequence, fused via a peptide bond.

In some aspects, provided herein is an immunoconjugate comprising the protein of the invention as defined herein linked to an additional therapeutic agent.

Examples of suitable therapeutic agents include cytotoxins, radioisotopes, chemotherapeutic agents, immunomodulatory agents, anti-angiogenic agents, antiproliferative agents, pro-apoptotic agents, and cytostatic and cytolytic enzymes (for example RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include the taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., ADEPT), radioisotopes, photosensitizers may also be used in immunoconjugates. In addition, conjugates can be made using secondary carriers as the cytotoxic agent, such as liposomes or polymers, Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Suitable immunomodulatory agents include anti-hormones that block hormone action on tumours and immunosuppressive agents that suppress cytokine production, down-regulate self-antigen expression, or mask MHC antigens.

Also provided is a nucleic acid molecule encoding the protein or a portion of the protein of the invention as defined herein. Further provided herein is a nucleic acid molecule encoding the first polypeptide chain, the second polypeptide chain, or both the first polypeptide chain and the second polypeptide chain of a protein of the invention that comprises multiple non-identical polypeptide chains. In some aspects, the nucleic acid molecule as defined herein may be isolated.

Further provided is a vector comprising the nucleic acid molecule of the invention as defined herein. The vector may be an expression vector.

Also provided is a host cell comprising the nucleic acid molecule or the vector of the invention as defined herein. The host cell may be a recombinant host cell.

In a further aspect there is provided a method of producing a protein of the invention, comprising culturing the host cell of the invention under conditions that result in expression and/or production of the protein, and isolating the protein from the host cell or culture.

In some aspects, provided herein is a pharmaceutical composition comprising the protein of the invention as defined herein, or the nucleic acid molecule of the invention as defined herein, or the vector of the invention as defined herein.

Further provided is a method for enhancing an immune response in a subject, comprising administering to the subject an effective amount of the protein the invention as defined herein, or the immunoconjugate of the invention as defined herein, or the nucleic acid molecule of the invention as defined herein, or the vector of the invention as defined herein, or the pharmaceutical composition of the invention as defined herein.

In a further aspect there is provided a method for treating or preventing cancer in a subject, or ameliorating a symptom of cancer in a subject, comprising administering to the subject an effective amount of the protein of the invention as defined herein, or the immunoconjugate of the invention as defined herein, or the nucleic acid molecule of the invention as defined herein, or the vector of the invention as defined herein, or the pharmaceutical composition of the invention as defined herein.

In some aspects, the cancer is a solid tumour. In some cases, the cancer is a hematologic malignancy. For example, the cancer may be Gastrointestinal Stromal cancer (GIST), pancreatic cancer, skin cancer (e.g., melanoma), breast cancer, lung cancer, bronchial cancer, colorectal cancer, prostate cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, or cancer of hematological tissues. In some cases, a cancer of haematological tissues is a lymphoma.

In some aspects, provided herein is a protein of the invention as defined herein, or the immunoconjugate of the invention as defined herein, or the nucleic acid molecule of the invention as defined herein, or the vector of the invention as defined herein, or the pharmaceutical composition of the invention as defined herein, for use in the treatment of cancer, or for use in or ameliorating a symptom of cancer.

In some aspects, provided herein is a protein, or an immunoconjugate, or a nucleic acid molecule, or a vector for use, or the method of treatment of the invention as defined herein, for separate, sequential or simultaneous use in a combination combined with a second therapeutic agent, for example an anti-cancer agent.

In a further aspect there is provided the use of a protein of the invention as defined herein, or an immunoconjugate of the invention as defined herein, or a nucleic acid molecule of the invention as defined herein, or a vector of the invention as defined herein, or a pharmaceutical composition of the invention as defined herein, in the manufacture of a medicament for the treatment of cancer or for ameliorating a symptom of cancer.

The invention also provides a method for treating or preventing an autoimmune disease or an inflammatory disease in a subject, comprising administering to the subject an effective amount of the protein as defined herein, or the immunoconjugate as defined here, or the nucleic acid molecule as defined herein, or the vector as defined herein, or the pharmaceutical composition as defined herein.

For example, the autoimmune disease or inflammatory disease may be arthritis, asthma, multiple sclerosis, psoriasis, Crohn’s disease, inflammatory bowel disease, lupus, Grave’s disease and Hashimoto’s thyroiditis, or ankylosing spondylitis.

Also provided is a protein as defined herein, or the immunoconjugate as defined herein, or the nucleic acid molecule as defined herein, or the vector as defined herein, or the pharmaceutical composition as defined herein, for use in the treatment of an autoimmune disease or an inflammatory disease.

Further provided is the use of a protein as defined herein, or an immunoconjugate as defined herein, or a nucleic acid molecule as defined herein, or a vector as defined herein, or a pharmaceutical composition as defined herein, in the manufacture of a medicament for the treatment of an autoimmune disease or an inflammatory disease.

The invention also provides a method for treating or preventing a cardiovascular disease or a fibrotic disease in a subject, comprising administering to the subject an effective amount of the protein as defined herein, or the immunoconjugate as defined here, or the nucleic acid molecule as defined herein, or the vector as defined herein, or the pharmaceutical composition as defined herein.

Also provided is a protein as defined herein, or the immunoconjugate as defined herein, or the nucleic acid molecule as defined herein, or the vector as defined herein, or the pharmaceutical composition as defined herein, for use in the treatment of a cardiovascular disease or a fibrotic disease.

Further provided is the use of a protein as defined herein, or an immunoconjugate as defined herein, or a nucleic acid molecule as defined herein, or a vector as defined herein, or a pharmaceutical composition as defined herein, in the manufacture of a medicament for the treatment of a cardiovascular disease or a fibrotic disease.

The cardiovascular disease in any aspect of the invention may for example be coronary heart disease, atherosclerosis, or stroke.

For example, the fibrotic disease in any aspect of the invention may be myocardial infarction, angina, osteoarthritis, pulmonary fibrosis, asthma, cystic fibrosis or bronchitis.

In some aspects, provided herein is a protein comprising the amino acid sequences disclosed herein and in the format disclosed herein for use in therapy.

In some aspects, the pharmaceutical composition may comprise a pharmaceutically acceptable excipient, carrier or diluent. A pharmaceutically acceptable excipient may be a compound or a combination of compounds entering into a pharmaceutical composition which does not provoke secondary reactions and which allows, for example, facilitation of the administration of the protein as defined herein, an increase in its lifespan and/or in its efficacy in the body or an increase in its solubility in solution. These pharmaceutically acceptable vehicles are well known and will be adapted by the person skilled in the art as a function of the mode of administration of the protein as defined herein.

In some aspects, the protein as defined herein may be provided in a lyophilised form for reconstitution prior to administration. For example, lyophilised protein molecules may be reconstituted in sterile water and mixed with saline prior to administration to an individual.

The protein as defined herein will usually be administered in the form of a pharmaceutical composition, which may comprise at least one component in addition to the protein molecule. Thus pharmaceutical compositions may comprise, in addition to the protein as defined herein, a pharmaceutically acceptable excipient, carrier, buffer, stabilizer or other materials well known to those skilled in the art. Such materials should be non-toxic and should not interfere with the efficacy of the protein. The precise nature of the carrier or other material will depend on the route of administration, which may be by bolus, infusion, injection or any other suitable route, as discussed below.

For parenteral, for example sub-cutaneous or intra-venous administration, e.g. by injection, the pharmaceutical composition comprising the protein as defined herein may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringe’s Injection, Lactated Ringer’s Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or nonionic surfactants, such as TWEEN™, PLURONICS™ or polyethylene glycol (PEG).

A pharmaceutical composition comprising a protein as defined herein may be administered alone or in combination with other treatments, either simultaneously or sequentially dependent upon the condition to be treated.

A protein as defined herein may be used in a method of treatment of the human or animal body, including prophylactic or preventative treatment (e.g. treatment before the onset of a condition in an individual to reduce the risk of the condition occurring in the individual; delay its onset; or reduce its severity after onset). The method of treatment may comprise administering the protein as defined herein to an individual in need thereof.

Administration is normally in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are well known in the art (Ledermann J.A. et al., 1991, Int. J. Cancer 47: 659-664; Bagshawe K.D. et al., 1991, Antibody, Immunoconjugates and Radiopharmaceuticals 4: 915-922). Specific dosages may be indicated herein or in the Physician’s Desk Reference (2003) as appropriate for the type of medicament being administered may be used. A therapeutically effective amount or suitable dose of the protein as defined herein may be determined by comparing its in vitro activity and in vivo activity in an animal model. Methods for extrapolation of effective dosages in mice and other test animals to humans are known. The precise dose will depend upon a number of factors, including whether the protein is for prevention or for treatment, the size and location of the area to be treated, the precise nature of the protein (e.g. Fab2, IgG) and the nature of any detectable label or other molecule attached to the protein.

A typical protein dose will be in the range 100 µg to 1 g for systemic applications, and 1 µg to 1 mg for topical applications. An initial higher loading dose, followed by one or more lower doses, may be administered. In some aspects, the protein will comprise a whole antibody, e.g., the IgG1 or IgG4 isotype. This is a dose for a single treatment of an adult patient, which may be proportionally adjusted for children and infants, and also adjusted for other protein construct formats in proportion to molecular weight. Treatments may be repeated at daily, twice-weekly, weekly or monthly intervals, at the discretion of the physician. The treatment schedule for an individual may be dependent on the pharmacokinetic and pharmacodynamic properties of the protein composition, the route of administration and the nature of the condition being treated.

Treatment may be periodic, and the period between administrations may be about two weeks or more, e.g. about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some aspects, proteins as defined herein may be administered as sub-cutaneous injections. Sub-cutaneous injections may be administered using an auto-injector, for example for long or short-term prophylaxis/treatment.

In some aspects, the therapeutic effect of the protein as defined herein may persist for several multiples of the protein half-life in serum, depending on the dose. For example, the therapeutic effect of a single dose of the protein as defined herein may persist in an individual for 1 month or more, 2 months or more, 3 months or more, 4 months or more, 5 months or more, or 6 months or more.

As used herein, the term “CD47” refers to IAP (Integrin Associated Protein) and variants thereof that retain at least part of the biological activity of CD47. As used herein, CD47 includes all mammalian species of native sequence CD47, including human, rat, mouse and chicken. In some embodiments, the term “CD47” is used to include variants, isoforms and species homologs of human CD47. In some cases, as used herein, CD47 includes all mammalian and non-mammalian species of native sequence CD47, including human, monkey, rat, mouse and chicken. In some embodiments, the term “CD47” refers only to wild-type CD47. Proteins of the invention may cross-react with CD47 from species other than human, in particular CD47 from cynomolgus monkey (Macaca fascicularis). Examples of human and cynomolgus CD47 amino acid sequences are provided in Table 11. In certain embodiments, the proteins of the invention may be completely specific for human CD47 and may exhibit no non-human cross-reactivity.

As used herein, the term “cMET” refers to the MET protein and variants thereof that retain at least part of the biological activity of cMET. In some cases, as used herein, cMET includes all mammalian species of native sequence cMET, including human, rat, mouse and chicken. In some embodiments, the term “cMET” may be used to include variants, isoforms and species homologs of human cMET. In some cases, as used herein, cMET includes all mammalian and non-mammalian species of native sequence cMET, including human, monkey, rat, mouse and chicken. In some embodiments, the term “cMET” refers only to wild-type cMET. Antibodies of the invention may cross-react with cMET from species other than human, in particular cMET from cynomolgus monkey (Macaca fascicularis). Examples of human and cynomolgus cMET amino acid sequences are provided in Table 12. In certain embodiments, the antibodies may be completely specific for human cMET and may exhibit no non-human cross-reactivity.

As used herein, the term “Her2” refers to the human epidermal growth factor receptor 2 protein and variants thereof that retain at least part of the biological activity of Her2. Her2 is also known as HER2/neu, ErbB2, c-erbB-2 and human EGF receptor 2. In some embodiments, the term “Her2” may be used to include variants, isoforms and species homologs of human Her2. In some cases, as used herein, Her2 includes all mammalian and non-mammalian species of native sequence Her2 (also known as ErbB2), including human, monkey, rat, mouse and chicken. In some embodiments, the term “Her2” refers only to wild-type Her2. Antibodies of the invention may cross-react with Her2 from species other than human, in particular Her2 from cynomolgus monkey (Macaca fascicularis). Examples of human and cynomolgus Her2/ErbB2 amino acid sequences are provided in Table 15. In certain embodiments, the antibodies may be completely specific for human Her2 and may exhibit no non-human cross-reactivity.

As used herein, the term “CD3” refers to the “cluster of differentiation 3” multimeric protein complex and variants thereof that retain at least part of the biological activity of CD3. The CD3 complex comprises four distinct polypeptide chains; epsilon (ε), gamma (y), delta (δ) and zeta (ζ). These polypeptide chains assemble and function as three pairs of dimers (ε_{ʏ}, εδ, ζζ). In some embodiments, the term “CD3” may be used to include variants, isoforms and species homologs of human CD3. In some cases, as used herein, CD3 includes all mammalian and non-mammalian species of native sequence CD3, including human, monkey, rat, mouse and chicken. In some embodiments, the term “CD3” refers only to wild-type CD3. Antibodies of the invention may cross-react with CD3 from species other than human, in particular CD3 from cynomolgus monkey (Macaca fascicularis). Examples of human and cynomolgus CD3 epsilon amino acid sequences are provided in Table 16. In certain embodiments, the antibodies may be completely specific for human CD3 and may exhibit no non-human cross-reactivity.

As used herein, an “antagonist” as used in the context of the protein of the invention refers to a protein which is able to bind to a molecule expressed in a diseased tissue and inhibit the molecule’s biological activity and/or downstream pathway(s) mediated by the molecule. For example, an “anti-CD47 antagonist protein” (interchangeably termed “anti-CD47 protein”) refers to a protein which is able to bind to CD47 and inhibit CD47 biological activity and/or downstream pathway(s) mediated by CD47 signalling. An anti-CD47 antagonist protein encompasses proteins that can block, antagonize, suppress or reduce (including significantly) CD47 biological activity, including downstream pathways mediated by CD47 signalling, such as receptor binding and/or elicitation of a cellular response to CD47. For the purposes of the present invention, it will be explicitly understood that the term “anti-CD47 antagonist protein” encompass all the terms, titles, and functional states and characteristics whereby CD47 itself, and CD47 biological activity (including but not limited to its ability to enhance the activation of phagocytosis by cells of the myeloid lineage), or the consequences of the activity or biological activity, are substantially nullified, decreased, or neutralized in any meaningful degree.

A protein of the invention “specifically binds” “specifically interacts”, “preferentially binds”, “binds” or “interacts” with an a molecule (e.g., human CD47, human Her2, human CD3, human cMET, or human PD-L1) if it binds with greater affinity, avidity, more readily and/or with greater duration than the protein binds to other molecules.

An “antibody molecule” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody molecule” encompasses not only intact polyclonal or monoclonal antibodies, but also any antigen binding fragment (for example, an “antigen-binding portion”) or single chain thereof, fusion proteins comprising an antibody, and any other modified configuration of the immunoglobulin molecule that comprises an antigen recognition site including, for example without limitation, scFv, single domain antibodies (for example, shark and camelid antibodies), maxibodies, minibodies, intrabodies, diabodies, triabodies, tetrabodies, v-NAR and bis-scFv.

An “antibody molecule” encompasses an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), for example IgG1, IgG2, IgG3, IgG4,IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody molecule, as used herein, refers to one or more fragments of an intact antibody that retain the ability to specifically bind to an antigen. Antigen binding functions of an antibody molecule can be performed by fragments of an intact antibody. Examples of binding fragments encompassed within the term “antigen binding portion” of an antibody molecule include Fab; Fab′; F(ab′)2; an Fd fragment consisting of the VH and CH1 domains; an Fvfragment consisting of the VL and VH domains of a single arm of an antibody; a single domain antibody (dAb) fragment, and an isolated complementarity determining region (CDR).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, CH2 and CH3. As is known in the art, an Fc region can be present in dimer or monomeric form.

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, and contribute to the formation of the antigen binding site of antibodies. When choosing FR to flank CDRs, for example when humanizing or optimizing an antibody, FRs from antibodies which contain CDR sequences in the same canonical class are preferred.

As used herein the term “conservative substitution” refers to replacement of an amino acid with another amino acid which does not significantly deleteriously change the functional activity. A preferred example of a “conservative substitution” is the replacement of one amino acid with another amino acid which has a value ≥ 0 in the following BLOSUM 62 substitution matrix (see Henikoff & Henikoff, 1992, PNAS 89: 10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | -1 | -2 | -2 | 0 | -1 | -1 | 0 | -2 | -1 | -1 | -1 | -1 | -2 | -1 | 1 | 0 | -3 | -2 | 0 |
| R | -1 | 5 | 0 | -2 | -3 | 1 | 0 | -2 | 0 | -3 | -2 | 2 | -1 | -3 | -2 | -1 | -1 | -3 | -2 | -3 |
| N | -2 | 0 | 6 | 1 | -3 | 0 | 0 | 0 | 1 | -3 | -3 | 0 | -2 | -3 | -2 | 1 | 0 | -4 | -2 | -3 |
| D | -2 | -2 | 1 | 6 | -3 | 0 | 2 | -1 | -1 | -3 | -4 | -1 | -3 | -3 | -1 | 0 | -1 | -4 | -3 | -3 |
| C | 0 | -3 | -3 | -3 | 9 | -3 | -4 | -3 | -3 | -1 | -1 | -3 | -1 | -2 | -3 | -1 | -1 | -2 | -2 | -1 |
| Q | -1 | 1 | 0 | 0 | -3 | 5 | 2 | -2 | 0 | -3 | -2 | 1 | 0 | -3 | -1 | 0 | -1 | -2 | -1 | -2 |
| E | -1 | 0 | 0 | 2 | -4 | 2 | 5 | -2 | 0 | -3 | -3 | 1 | -2 | -3 | -1 | 0 | -1 | -3 | -2 | -2 |
| G | 0 | -2 | 0 | -1 | -3 | -2 | -2 | 6 | -2 | -4 | -4 | -2 | -3 | -3 | -2 | 0 | -2 | -2 | -3 | -3 |
| H | -2 | 0 | 1 | -1 | -3 | 0 | 0 | -2 | 8 | -3 | -3 | -1 | -2 | -1 | -2 | -1 | -2 | -2 | 2 | -3 |
| I | -1 | -3 | -3 | -3 | -1 | -3 | -3 | -4 | -3 | 4 | 2 | -3 | 1 | 0 | -3 | -2 | -1 | -3 | -1 | 3 |
| L | -1 | -2 | -3 | -4 | -1 | -2 | -3 | -4 | -3 | 2 | 4 | -2 | 2 | 0 | -3 | -2 | -1 | -2 | -1 | 1 |
| K | -1 | 2 | 0 | -1 | -3 | 1 | 1 | -2 | -1 | -3 | -2 | 5 | -1 | -3 | -1 | 0 | -1 | -3 | -2 | -2 |
| M | -1 | -1 | -2 | -3 | -1 | 0 | -2 | -3 | -2 | 1 | 2 | -1 | 5 | 0 | -2 | -1 | -1 | -1 | -1 | 1 |
| F | -2 | -3 | -3 | -3 | -2 | -3 | -3 | -3 | -1 | 0 | 0 | -3 | 0 | 6 | -4 | -2 | -2 | 1 | 3 | -1 |
| P | -1 | -2 | -2 | -1 | -3 | -1 | -1 | -2 | -2 | -3 | -3 | -1 | -2 | -4 | 7 | -1 | -1 | -4 | -3 | -2 |
| S | 1 | -1 | 1 | 0 | -1 | 0 | 0 | 0 | -1 | -2 | -2 | 0 | -1 | -2 | -1 | 4 | 1 | -3 | -2 | -2 |
| T | 0 | -1 | 0 | -1 | -1 | -1 | -1 | -2 | -2 | -1 | -1 | -1 | -1 | -2 | -1 | 1 | 5 | -2 | -2 | 0 |
| W | -3 | -3 | -4 | -4 | -2 | -2 | -3 | -2 | -2 | -3 | -2 | -3 | -1 | 1 | -4 | -3 | -2 | 11 | 2 | -3 |
| Y | -2 | -2 | -2 | -3 | -2 | -1 | -2 | -3 | 2 | -1 | -1 | -2 | -1 | 3 | -3 | -2 | -2 | 2 | 7 | -1 |
| V | 0 | -3 | -3 | -3 | -1 | -2 | -2 | -3 | -3 | 3 | 1 | -2 | 1 | -1 | -2 | -2 | 0 | -3 | -1 | 4 . |

The term “monoclonal antibody” (Mab) refers to an antibody, or antigen-binding portion thereof, that is derived from a single copy or clone, including for example any eukaryotic, prokaryotic, or phage clone, and not the method by which it is produced. Preferably, a monoclonal antibody of the invention exists in a homogeneous or substantially homogeneous population.

A “humanized” antibody molecule refers to a form of non-human (for example, murine) antibody molecules, or antigen-binding portion thereof, that are chimeric immunoglobulins, immunoglobulin chains, or fragments thereof (such as Fv, Fab, Fab′, F(ab′)2 or other antigen-binding sub-sequences of antibodies) that contain minimal sequence derived from non-human immunoglobulin. Humanized antibodies may be human immunoglobulins (recipient antibody) in which residues from a CDR of the recipient are replaced by residues from a CDR of a non-human species (donor antibody) such as mouse, rat, or rabbit having the desired specificity, affinity, and capacity.

“Human antibody” or “fully human antibody” refers to an antibody molecule, or antigen-binding portion thereof, derived from transgenic mice carrying human antibody genes or from human cells.

The term “chimeric antibody” is intended to refer to an antibody molecule, or antigen-binding portion thereof, in which the variable region sequences are derived from one species and the constant region sequences are derived from another species, such as an antibody molecule in which the variable region sequences are derived from a mouse antibody and the constant region sequences are derived from a human antibody.

The term “immunoconjugate” refers to a protein of the invention that is conjugated, fused or linked to at least one cytotoxic, cytostatic, or therapeutic agent.

Proteins of the invention can be produced using techniques well known in the art, for example recombinant technologies, phage display technologies, synthetic technologies or combinations of such technologies or other technologies readily known in the art.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by a protein of the invention, an antibody molecule, or antigen-binding portion thereof, at one or more of the protein’s or antibody molecule’s antigen-binding regions. Epitopes can consist of defined regions of primary secondary or tertiary protein structure and includes combinations of secondary structural units or structural domains of the target recognised by the antigen binding regions of the protein, the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which a protein of the invention or an antibody molecule can specifically bind as determined by any method well known in the art, for example, by conventional immunoassays, antibody competitive binding assays or by x-ray crystallography or related structural determination methods (for example NMR).

The term “binding affinity” or “KD” refers to the dissociation rate of a particular antigen-binding protein interaction or antigen-antibody interaction. The KD is the ratio of the rate of dissociation, also called the “off-rate (k_{off})”, to the association rate, or “on-rate (kₒₙ)”. Thus, K_{D} equals kₒᵣᵣ/ kₒₙ and is expressed as a molar concentration (M). It follows that the smaller the K_{D}, the stronger the affinity of binding. Therefore, a K_{D} of 1 µM indicates weak binding affinity compared to a K_{D} of 1 nM. KD values for binding proteins or antibodies can be determined using methods well established in the art. One method for determining the KD of a binding protein or an antibody is by using surface plasmon resonance (SPR), typically using a biosensor system such as a Biacore® system.

The term “potency” is a measurement of biological activity and may be designated as IC₅₀, or effective concentration of a protein of an immunoconjugate of the invention to its binding partner (e.g., a molecule expressed in a diseased tissue) or antigen to inhibit 50% of activity of the binding partner or the antigen measured in an activity assay as described herein.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The term “inhibit” or “neutralize” as used herein with respect to bioactivity of a protein of the invention means the ability of the protein to substantially antagonize, prohibit, prevent, restrain, slow, disrupt, eliminate, stop, reduce or reverse for example progression or severity of that which is being inhibited including, but not limited to, a biological activity or binding interaction of a molecule expressed in a diseased tissue.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the invention.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as defined above. The term “treating” also includes adjuvant and neoadjuvant treatment of a subject. For the avoidance of doubt, reference herein to “treatment” includes reference to curative, palliative and prophylactic treatment. For the avoidance of doubt, references herein to “treatment” also include references to curative, palliative and prophylactic treatment.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

The practice of the present invention will employ, unless otherwise indicated, conventional techniques of molecular biology (including recombinant techniques), microbiology, cell biology, biochemistry and immunology, which are within the skill of the art.

Particular non-limiting embodiments of the present invention will now be described with reference to accompanying drawings.

### EXAMPLES

### Example 1. Generation of Optimized Conditionally Active Therapeutic Antibodies

### Introduction

In this example, we successfully generated a panel of optimized conditionally active antibodies. These conditionally active antibodies were well expressed, biophysically stable, highly soluble and of maximized amino acid sequence identity to preferred human germlines.

### Materials and Methods

### Cloning, Transient Expression, Purification and Characterization of Proteins

Antibody-encoding DNA sequences were cloned via restriction-ligation cloning into separate human IgG1 heavy and light-chain encoding expression cassettes in separate plasmid vectors, to create IgG or IgG² format constructs for expression. Similar cassettes were also cloned into ‘knob into hole’ human IgG1 heterodimerising Fc vectors to create Fab and Fab² constructs. Fab² cMETCD47 and Her2CD3 protein constructs were constructed using knob-into-hole (KIH) heavy chain expression vectors (CH3 domain T366W and T366S/L368A/Y407V mutations). Fab² Her2-CD3 constructs also included effector function-ablating mutations L234A/L235A/G237A. IgG² Her2CD47 protein constructs were constructed using “wildtype” IgG1 heavy & kappa light chain expression vectors. IgGs were expressed in CHO cells after transient transfection with endotoxin-free IgG expression plasmid preparations, per manufacturer’s protocols.

Produced antibodies were captured from clarified supernatants using a HiTrap MabSelect Sure Protein A 5 mL column on an ÄKTA Pure 150 L FPLC system. Eluted protein peaks were immediately buffer exchanged into 1x PBS pH 7.4 by directly loading the eluted protein A peak fractions onto a HiPrep 26/10 Desalting column. Protein concentration was determined by measuring the absorbance at 280 nm and 1 µg of each purified protein was analysed by SDS-PAGE under reducing and/or non-reducing conditions using 4-20% TGX polyacrylamide gradient gels (BioRad, Cat. nr. 456-1093) with 1x Tris/glycine/SDS buffer, separated by 120V field for 1 hour. In order to test for the presence of non-covalently bound aggregates and to complement the SDS PAGE analysis, analytical size-exclusion chromatography was performed. Aliquots of selected clones were analyzed by analytical Size Exclusion Chromatography (SEC) using a Superdex 200 Increase 10/300 SEC column and 1x PBS pH 7.4 as running buffer, in isocratic mode.

Selected proteins were further purified using preparative SEC. Antibody samples up to 1 ml were loaded onto a Superdex 200 Increase 10/300 SEC column or a HiLoad 26/600 Superdex 200 pg column equilibrated in 1x PBS pH 7.4. 1 ml fractions of peak of interest were collected, and main peak fractions were pooled. After size exclusion chromatography, the samples were again analyzed by SDS-PAGE, as above.

### Haemagglutination

Red blood cells (RBC) were isolated from fresh uncoagulated human blood (minimum of 3 different donors), diluted to 2% in PBS and incubated for 60-90 minutes in a U bottomed 96 well plate with a titration of IgG or protein construct. In the absence of haemagglutination, cells sedimented to the bottom of the well forming a red pellet. Haemagglutination was observed as non-settled RBC solution. Images of each plate were recorded and the data expressed for each sample as titre for the last well in which haemagglutination was observed.

### Metalloprotease Digestion

Protein constructs were incubated for 16 hour at 37° C. with either individual human Matrix Metalloprotease (MMP) enzymes or a mix of active MMP3, MMP7 and MMP12 (equal parts of each) at a ratio of 1% total MMP to protein construct (wt/wt) in Tris buffered saline (pH7.4) containing 5 mM CaCl₂. The reactions were stopped by the addition of 20 mM EDTA and then samples tested for binding or functional activity as described.

### IgG Titration Binding ELISAs

To coat ELISA plates, target proteins were diluted to 1 µg/ml in PBS pH7.4 and added at 100 µl per well, at 4° C., o/n. Coated plates were washed 3x with PBS pH7.4, blocked with 4% Skim Milk Protein in PBS (380 µl/ well) for 1 hr at RT, then washed 5x with PBS-Tween 20 (PBST). Antibodies (100 µl/well; diluted in PBST) were then added and then incubated 1 hr at RT. Plates were then washed 3x with PBS and goat anti-human IgG-HRP added (100 µl/well) at RT, for 1 hr. Plates were then washed 3x with PBST and twice with PBS before the addition of 100 µl TMB per well. Reactions were stopped by adding 100 µl 2 M H₂SO₄/well and OD was read on a plate reader at 450 nm.

### Flow Cytometry Binding

Binding of protein constructs (+/- pre-digestion with MMP3/7/12) and control lgG to Jurkat cells and BT-474 cells was assessed by flow cytometry. Viable cells were identified using Zombie UV™ Fixable Viability Dye (Biolegend). Binding of human IgG and protein constructs was detected with a FITC conjugated goat anti human (H+L) secondary antibody. The mouse monoclonal anti-CD3 control antibody binding was detected using Alexa-Fluor-488 goat anti mouse IgG. Results were analysed by measurement of the Mean Fluorescence Intensity (MFI) of viable cells in the FITC channel detector of a BD Fortessa flow cytometer.

### T-Cell Activation Bioassay

Functional activity of Her2CD3 protein constructs was assessed in a co-culture assay using BT474 cells and an NFAT-RE- luciferase Jurkat reporter cell line (Promega - TCR/CD3 Effector cells NFAT). BT-474 cells (40000 cell/well) were seeded into 96-well white clear bottomed tissue culture treated plates in Hybri-Care medium (ATCC) supplemented with 10% FBS and incubated at 37° C. overnight in a CO₂ incubator. Medium was removed and control antibodies or protein constructs (+/- pre-digestion with MMP3/7/12) prepared in assay medium (RPMI supplemented with 10% FBS) were added to the cells. TCR/CD3 Effector cells (NFAT) were thawed and diluted according to the manufacturer’s protocol, then added to the assay wells. Following a 6 hour incubation at 37° C. in a CO₂ incubator the plates were re-equilibrated to room temperature and luciferase activity determined by addition of Bio-Glo reagent for 5-10 minutes and measurement of luminescent signal (RLU). Fold induction was determined by calculating the ratio of the sample RLU/RLU in the absence of antibody following subtraction of background luminescence signal.

### Molecular Dynamics Simulations

Eight systems (linker sequences X1, X2, X3 and X4 and the equivalent sequences with a covalent bond broken in one of the linkers at the GS sequence position XC1, XC2, XC3 and XC4) were modelled and optimised using the AMBER10:EHT forcefield in MOE (Chemical Computing Group Inc). Histidine charges were assigned using protonate3D tool in MOE. Three successive rounds of minimisation were undertaken with a final gradient of 0.001 allowing 10,000 steps without constraints. The individual systems were constrained once again and gradually minimized using NAMD 2.13 with the CHARMM27 forcefield to reduce the total potential energy in a series of three steps of energy minimization. The first step kept all heavy atoms constrained, and only hydrogen atoms were allowed mobility. The second step removed the constraints on the sidechains whilst the third steps freed all atoms, allowing them to move without restriction. Minimisation within CHARMM27 was facilitated since this is the forcefield at work within NAMD molecular dynamics simulations.

For dynamics runs, Generalised Born (GB) solvation was used to describe solvent effects. MD simulations were set up and performed afterwards using NAMD 2.13. Four steps of equilibration were performed, gradually loosening harmonic constraints in 125 ps increments, for a total equilibration time of 500 ps. The first step heated the system to 310 K while applying a force of 4 kcal/mol to hold the backbone in place. The remaining three steps gradually lifted the backbone constraint force from 4 kcal/mol to 1 kcal/mol with an NVT ensemble (constant number (N), volume (V), and temperature (T)). A switching function was applied at 10 Å to abridge the van der Waals potential function. Electrostatic and vdW cutoff was set at 14 Å as recommended in NAMD. Periodic boundary conditions were not used as they were not compatible with the implicit solvent approach. The constraints applied during equilibration were removed for the free simulation of the antibody complexes. Production runs were conducted for either 6, 15, 20 or 100 ns at 310 K. 100 ns runs were applied to cleaved linker proteins XC1-4 in order to fully explore range of movement whilst the shorter runs (up to 20 ns) were found to be sufficient to map the range of movement for non-cleaved antibody constructs X1-4.

### In Vivo Analyses of PK and Tolerability

TOLERABILITY STUDY - Twenty eight (28) 6 - 8 week old male B6.Cg-Fcgrt^{tm1Dor} Tg(FCGRT)32DcrJ (‘Tg32’ homozygous human FcRn transgenic, JAX stock# 014565) mice were assigned into 7 groups with 4 mice per group. Body weights were measured the day of antibody administration. At 0 hours, test articles were administrated as IV injections at 2 mg/kg or 10 mg/kg, and at a dose volume of 10 ml/kg. 200 µL whole blood samples were collected into EDTA, on days 5, 29, and 60 (terminal bleed). Blood was used for CBC/Dif/Retic analysis, including Leucocytes, Neutrophils, Eosinophils, Basophils, Lymphocytes, Monocytes, Hemoglobin, Erythrocytes, Reticulocytes, MCHC and MCV. Body weight were then monitored weekly for the first month and then monthly until the end of the experiment.

PHARMACOKINETICS STUDY - Thirty two 6 - 8 week old male B6.Cg-Fcgrt^{tm1Dor} Tg(FCGRT)32DcrJ (‘Tg32’ homozygous human FcRn transgenic, JAX stock #014565) mice were assigned into 8 groups with 4 mice per group. The day before test article administration, 35 µL blood samples from three (3) Tg32 mice were collected into EDTA to test the binding of the test articles to the mouse erythrocytes using flow cytometry. Body weights were measured the day of antibody administration and weekly until the end of the experiment. At 0 hours, the test articles were administrated as IV injections at 2 mg/kg or 10 mg/kg, at a dose volume of 10 ml/kg. Blood samples were collected from each mouse according to the bleeding schedule: 30 min, 4 h, 1, 3, 5, 7, 10, 14, 21, 28 and 42 days. 25 µL blood samples were collected from each mouse according to the bleeding schedule. The blood samples were collected into K₃EDTA, processed to plasma, and stored at -20° C. Plasma samples were then assessed in triplicate by and ELISA for estimation of human IgG concentration.

TOLERABILITY STUDY IN NOD-SCID MICE - NOD-SCID mice were assigned into 4 groups with 3 mice per group. Body weights were measured daily. At Day 0, test articles were administrated as IV injections at 8 mg/kg or 14 mg/kg, with 3 subsequent doses at 4 or 7 mg/kg at 5 day intervals.

### Flow Cytometry Analyses of Binding to Monkey and Human Erythrocytes

Erythrocytes were isolated from 3 Cyno (Cynomolgus) monkeys and 3 human donors. Per sample, 5×10⁵ cells (diluted in DMEM + 5% FBS) were stained for one hour with A-D5 IgG1: 0.0032; 0.016; 0.03; 50 µg/mL. Trastuzumab: 0.0032; 0.016; 0.03; 50 µg/mL. IgG² Her47 LHL-LHLF 0 h: 0.016; 0.08; 0.4; 2; 10; 50 µg/mL. IgG² Her47 LHL-LHL 0 h: 0.016; 0.08; 0.4; 2; 10; 50 µg/mL. Binding of test samples to erythrocytes was measured using FITC AffiniPure Goat Anti-Human IgG (1:200 dilution; 1-hour incubation time), followed by Flow cytometry measurement of FITC fluorescence intensity (BD LSR FortessaX-20 cell analyser).

### Metalloprotease and Cathepsin Digestion at pH6.0 and 7.4

Protein constructs were placed in TBS (containing 5 mM CaCl₂, pH6.0 or 7.4) then incubated at 37° C. with individual human Matrix Metalloprotease (MMP) or Cathepsin enzymes, at a ratio of 1% total enzyme to protein construct (wt/wt), for 0, 2, 4, 8 and 24 h. The reactions were stopped by the addition of 20 mM EDTA and then samples frozen before testing for binding or functional activity as described.

### In Vitro Protein Stability Analyses

Forced Oxidation - For forced oxidation analysis, test articles in PBS were treated with 0.5 % H2O2 at room temperature for 2 hours and then stored at -80° C. prior to SEC and RP analysis (intact antibodies and subunits, tryptic peptides) on a Dionex Ultimate 3000RS HPLC system (ThermoFisher Scientific, Hemel Hempstead, UK). For intact antibody reduction, DTT was added to a final concentration of 0.33 M and samples were incubated for 1 hour at 22° C. and immediately analysed by RP.

SEC Analysis - Chromatographic separation was performed using an Acquity UPLC Protein BEH SEC Column, 200 Å, 1.7 µm, 4.6 mm × 150 mm (Waters, Elstree, UK) and an Acquity UPLC Protein BEH SEC guard column 30 × 4.6 mm, 1.7 µm, 200 Å (Waters, Elstree, UK) connected to a Dionex Ultimate 3000RS HPLC system (ThermoFisher Scientific, Hemel Hempstead, UK). The method consisted of an isocratic elution over 10 minutes and the mobile phase was 0.2 M potassium phosphate pH 6.8, 0.2 M potassium chloride. The flow rate was 0.35 mL/minute. Detection was carried out by UV absorption at 280 nm.

Reverse Phase analysis of intact antibodies and subunits - Chromatographic separation was performed using a PLRP-S 1000, 5 µm, 2.1 mm × 50 mm column (Agilent Technologies, Stockport, UK) connected to a Dionex Ultimate 3000RS HPLC system (ThermoFisher Scientific, Hemel Hempstead, UK). The method consisted of a linear gradient from 75 % buffer A (0.02 % TFA, 7.5 % acetonitrile in H2O) to 45 % buffer B (0.02 % TFA, 7.5 % H2O in acetonitrile) over 14 minutes. The flow rate was 0.5 mL/minute and the temperature was maintained at 70° C. throughout the analysis. Detection was carried out by UV absorption at 280 nm.

HIC Analysis - Chromatographic separation was performed using a TSKgel Butyl-NPR 4.6 mm × 35 mm HIC column (TOSOH Bioscience Ltd., Reading, UK) connected to a Dionex Ultimate 3000RS HPLC system (ThermoFisher Scientific, Hemel Hempstead, UK). The method consisted of a linear gradient from 60 % Buffer A (100 mM sodium phosphate pH 7.0, 2 M ammonium sulphate) to 90 % Buffer B (100 mM sodium phosphate pH 7.0) over 9 minutes. The flow rate was 1.2 mL/minute. Detection was carried out by UV absorption at 280 nm.

Charge Variant Assay - Charge variant profiling of test articles was determined by Protein Charge Variant Assay on a LabChip GXII Touch HT (PerkinElmer), according to the manufacturer’s protocol.

### Biacore® Analyses of Fc Affinity for Human Fc Receptors

Interaction affinities for antibody proteins were determined by surface plasmon resonance using a BIACORE® T200 instrument. For most analyses, His6-tagged FcγRl, FcyRlla (167R and 167H variants), FcyRllb, FcyRllla (176F and 176V variants), and FcyRlllb receptors (all Sino Biological) were captured on a CM5 sensor chip coated with an anti-HIS antibody by standard amine coupling. Receptor-specific formats of analyses were then applied, as below.

FcγRl is a high-affinity receptor for IgG1 monomers, so 1:1 kinetic analysis was performed under the following conditions: ‘single cycle’ analysis using flow rate 30 µl/min, receptor protein loaded to ~30 RU at 10 µl/min (diluted 0.25 µg/ml in HBS-P+), 5 point three-fold dilution of purified antibodies titrated 0.411 nM to 33.33 nM applied with an association time of 200 s, dissociation time of 300 s. Regeneration with 2x injections glycine pH 1.5 and analysis using 1:1 fit.

The interactions between monomeric IgG and FcγRII and FcγRIII receptors are relatively low affinity interactions, so ‘steady state’ affinity analyses were performed under the following conditions: flow rate 30 µl/min, receptor protein loaded to ~60 RU at 10 µl/min (diluted 0.25 µg/ml in HBS-P+), 5 point three-fold dilution series of purified antibodies titrated between 33 nM and 24000 nM applied with an association time of 30 s, dissociation time of 25 s. Regeneration with 2x injections glycine pH 1.5 and analysis using steady state affinity calculation.

### Results and Discussion

### Protein Construct Design Principles

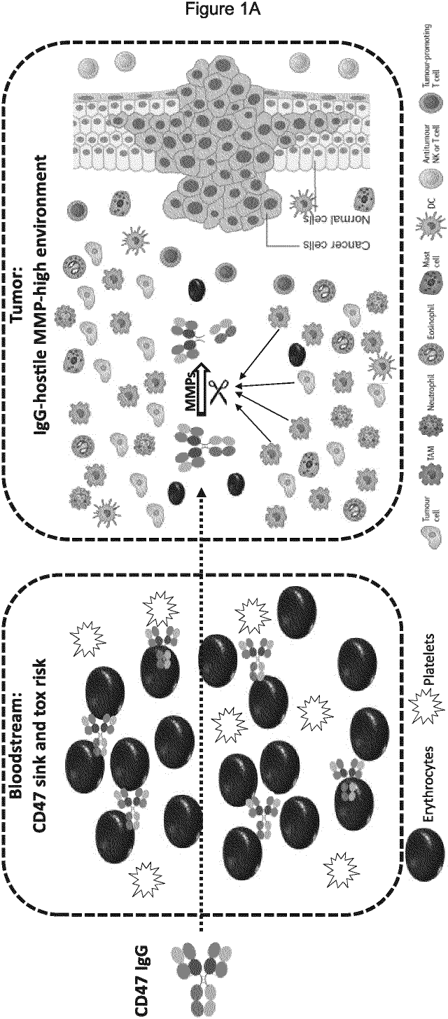

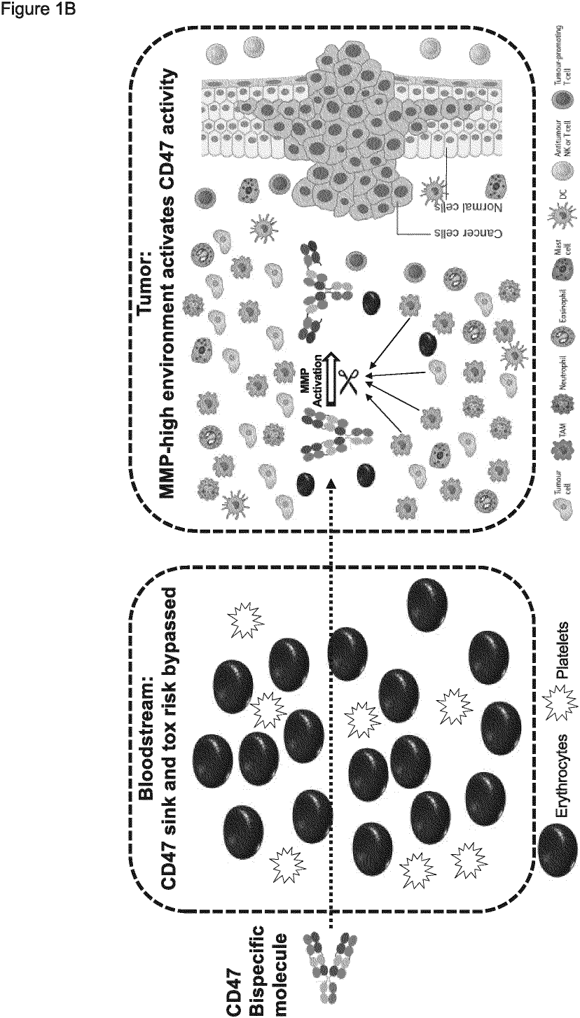

Standard anti-cancer antigen antibodies suffer from significant pharmacological challenges in the treatment of solid tumours. A key issue that restricts efficacy in this class of potential drugs is that the antigens targeted by the antibodies are not found exclusively in the tumour and are merely highly overexpressed in the tumour. This off-tumour target expression often leads to dose-limiting side effect risks and antigen ‘sink’ effects where large doses of the antibody must be given to ensure sufficient antibody penetrates the tumour to have a therapeutic effect. One such example is the class of antibodies that target the antigen CD47 (FIG. 1A), where challenges include: High expression of CD47 in the bloodstream (e.g. on erythrocytes and platelets in particular) is a ‘sink’ that is bound by intravenously-dosed antibody, minimizing the amount of drug penetrating the tumour (even when large doses of IgG are given). The binding of blood cells by anti-CD47 is also a significant toxicity risk. Indeed, anti-CD47 antibodies are known to be capable of causing anaemia and even the cross-linking of human erythrocytes, creating a hemagglutination risk in patients. In addition, the tumour is typically a ‘hostile’ environment with high expression rates of enzymes such as MMPs which can accelerate IgG degradation. These factors all conspire to minimise the potential safety and efficacy of anti-CD47 antibodies and many other types of anti-tumour target antibodies where target expression is not limited solely to the tumour environment.

Anti-CD47 protein constructs (FIG. 1B) aim to overcome the peripheral sink and toxicity issues experienced with anti-tumour antigen IgGs by eliminating binding of the high risk (but potentially strong mechanism of action) target, in the native protein. This effect is achieved by adding low-risk upper domains (e.g. Fab domains that target another tumour antigen such as Her2) and linkers above (n-terminal to) the binding domains of the high-risk lower domains (such as CD47). The use of appropriate upper domain/linker combinations results in a configuration that fully blocks binding activity in the lower CD47 domain. The tumour-targeting domain (e.g. Her2) then drives high concentration in the tumour environment and the protein construct linker system exploits the elevated MMP activity in the tumour to cleave the linker peptides, exposing the CD47 binding domains and thereby conditionally activating the CD47-binding activity in the tumour, rather than the periphery. This design principle has the potential to be applicable in many different structural formats, examples of which are outlined below.

The protein construct IgG² design (FIG. 2A) may be based on sequences derived from IgG1, IgG2, IgG3, IgG4, IgE, IgM, or IgA and may or may not have effector function capacity. In this construct, four polypeptide chains encode for four Fab domains (2x Fab A, 2X Fab B), four linker sequences, and may or may not have an immunoglobulin hinge region and an Fc domain. Each Fab A-Linker domain (upper) blocks the binding activity of Fab B (lower). The target binding specificity of the upper and lower domains may be different, to drive bispecific function, or the same, to drive polyvalent target interaction. The choice of linker sequence, such as a lower hinge peptide sequence, creates a structure that will be locked in a non-diseased tissue, but quickly cleaved and unlocked in the presence of high concentrations of proteases in the tumour environment (FIG. 2B). The linkers in protein construct designs are all proteolytically cleavable and may be sequentially cleaved, with a first ‘fast’ cleavage taking the ‘Locked’ intact structure and creating an intermediate ‘Unlocked’ active state which allows Fabs A and B from a single protein construct to bind their cognate targets. Secondary, potentially slower cleavage of the second linker in each Fab A-Fab B protein construct unit may release the Fab A domains from the structure entirely, creating a ‘Dissociated’ form where the lower Fab domains are fully liberated for non-targeted (but likely still localized) activity. Cleaved linkers based on immunoglobulin hinge sequences may also recruit increased immune effector function (ADCC, CDC and ADCP) at the cell membrane via endogenous anti-hinge antibodies, which are a known phenomenon in human patients with (and even without) underlying autoreactive disease.

The protein construct Fab² design may be based on sequences derived from IgG1, IgG2, IgG3, IgG4, IgE, IgM, or IgA and may or may not have effector function capacity. In this construct, two (FIG. 3A) or three (FIG. 3B) polypeptide chains encode for two Fab domains (1x Fab A, 1X Fab B), two or more linker sequences, and may or may not have an immunoglobulin hinge region and an Fc domain in which pairing of heterodimers may or may not be driven by mutations in the Fc. Each Fab A-Linker domain again blocks the binding activity of Fab B and the choice of linker sequence, such as a lower hinge peptide sequence, creates a structure that will be locked in a non-diseased tissue, but quickly cleaved and unlocked in the presence of high concentrations of linker-cleaving proteases in the tumour environment, eventually becoming dissociated (FIG. 3A).

### Protein Construct Cloning and Expression

To produce and purify 15 bispecific, conditionally-active protein constructs formatted as Fab² or IgG² molecules with different Linker domains (Table 1), DNA cassettes for each construct type (Table 2) were synthesised and cloned into expression vectors encoding human IgG1 heavy and light chain or ‘Knob into hole’ heterodimeric Fc. Proteins were named using the nomenclature (format)-(target names [upper domain/lower domain)-(heavy chain linker type)-(light chain linker type). All proteins were produced by transient transfection of CHO cells, then purified by protein A affinity chromatography.

The anti-HER2 variable region sequences used in the protein constructs disclosed herein are the variable region sequences of trastuzumab. The anti-CD3 variable region sequences used in the protein constructs disclosed herein are the variable region sequences of OKT3 or SP34. The anti-cMET variable region sequences used in the protein constructs disclosed herein are provided in WO 2019/175186. The anti-CD47 variable region sequences used in the protein constructs disclosed herein are provided in WO 2019/034895.

### Analysis of Protein Construct Expression and Purification Characteristics

The protein A purified proteins were quantified, showing that using different linker types affected expression yield (Table 2). Protein preparations in 1x PBS pH 7.4 were also examined in analytical size exclusion chromatography for quantification of the percentage desired product. Across all 3 classes of bispecific proteins generated (cMETCD47, Her2CD47 and Her2CD3), constructs containing the LHL linker generated the most optimal combination of the highest yield and production of % main peak of desired product by analytical SEC (Table 2). SDS-PAGE analysis of the protein A-purified proteins (FIG. 4) also demonstrated that clones containing the short linker domains L1 and LH, plus the long linker domain L3 produced highly heterogeneous products, with significant HMW and LMW content in the unreduced samples and LMW content in the reduced samples. This suggested that suboptimal linker types can lead to significant formation of undesirable multimers and breakdown products. For all constructs, HMW impurities were mostly absent in the reduced lanes, suggesting that these HMW impurities are disulfide-bonded dimers or higher, which are not reduced by SDS alone. For the IgG² design, clones 12 and 14 both exhibited high yields (Table 2), but clone 14 (containing the LHL linker) demonstrated highest yield and high uniformity of the desired product by SEC (90%) and SDS-PAGE (FIG. 4).

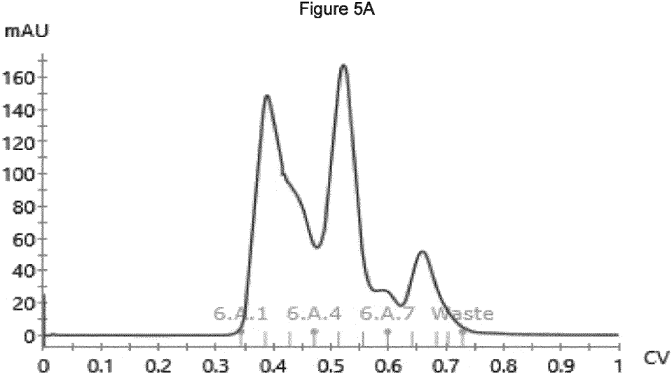

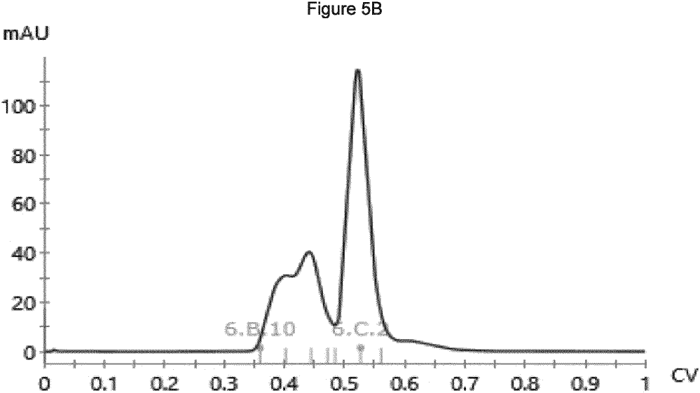

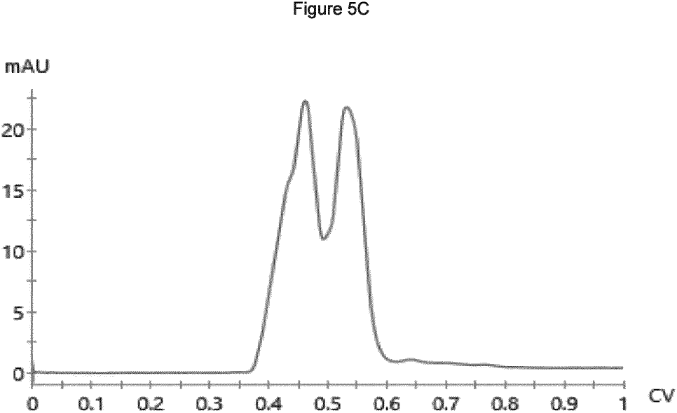

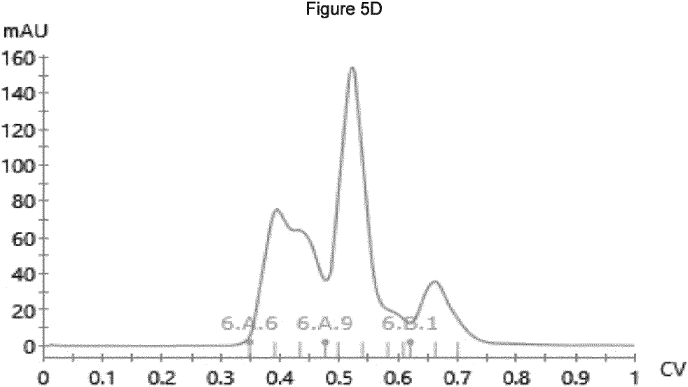

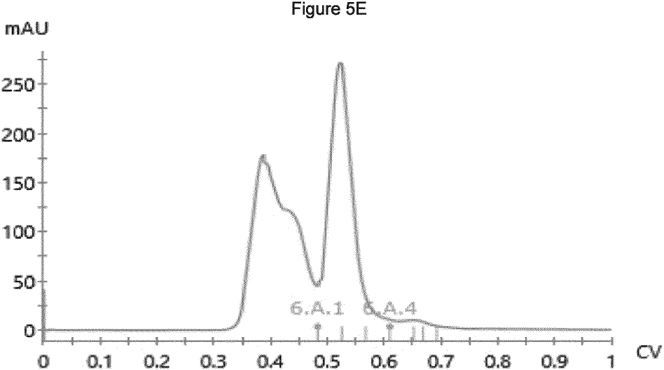

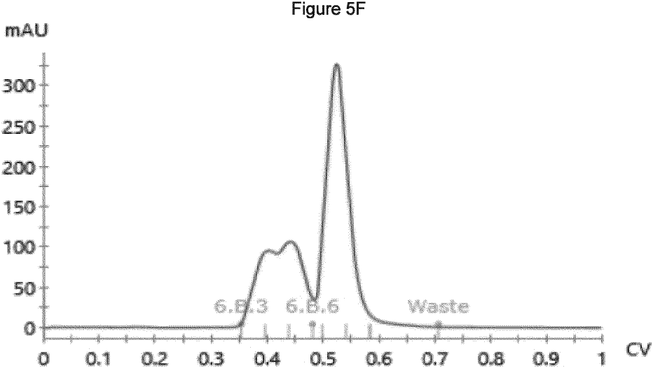

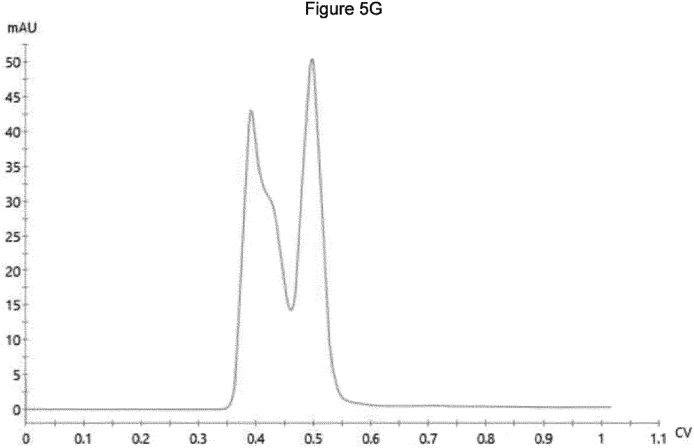

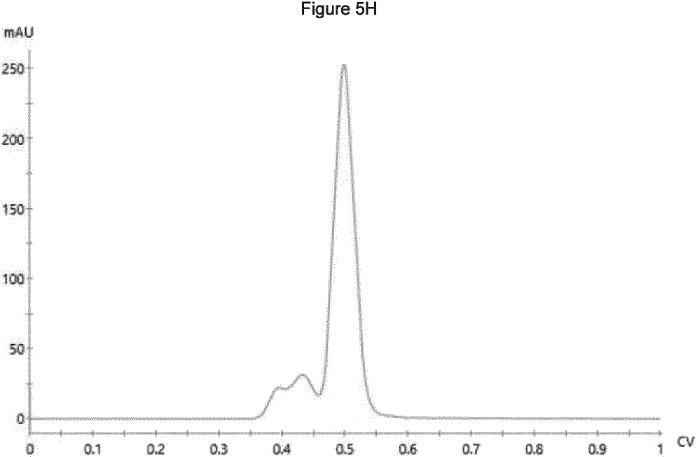

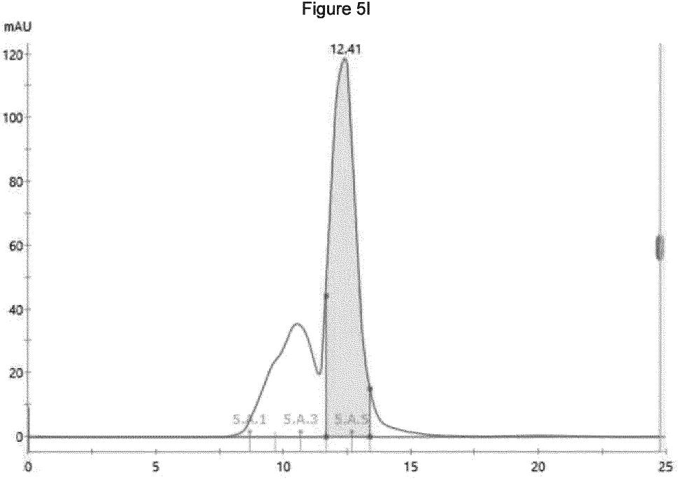

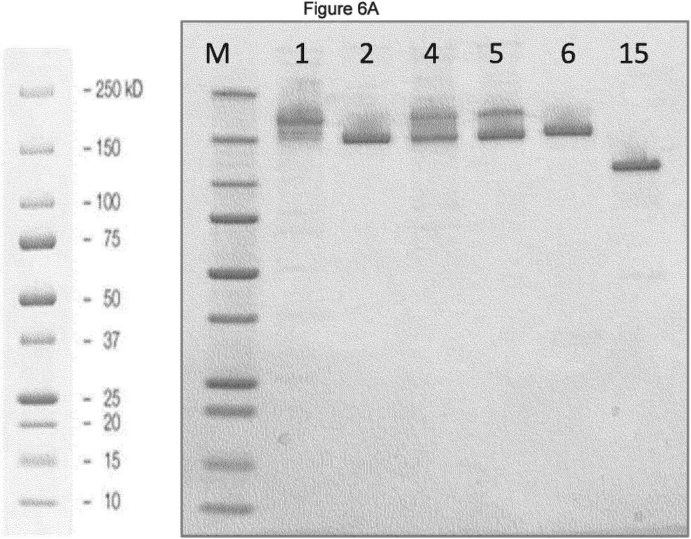

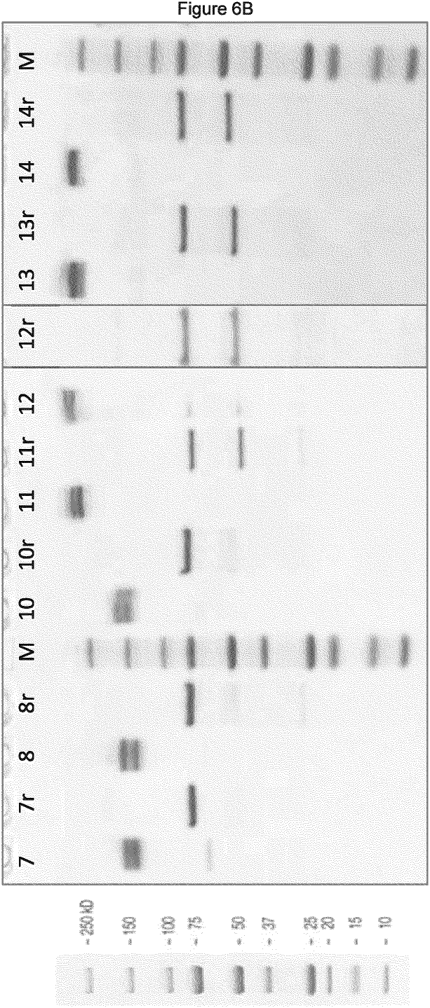

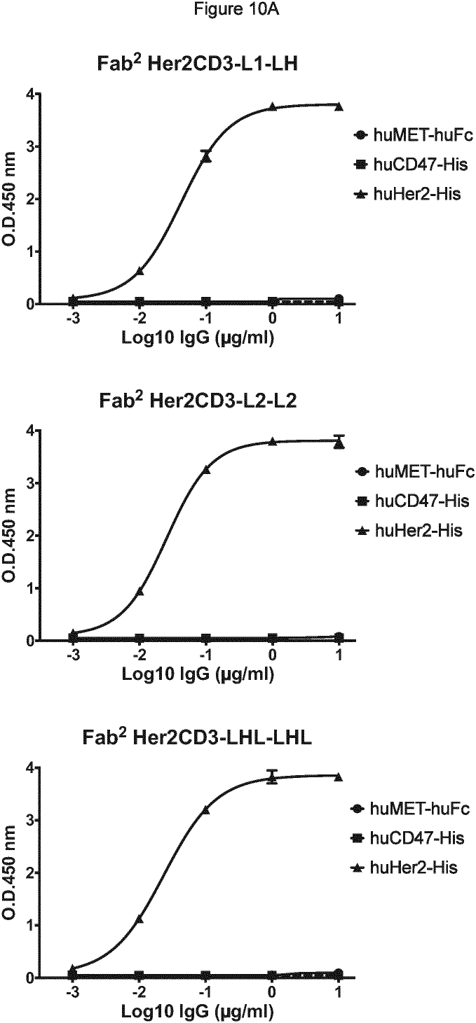

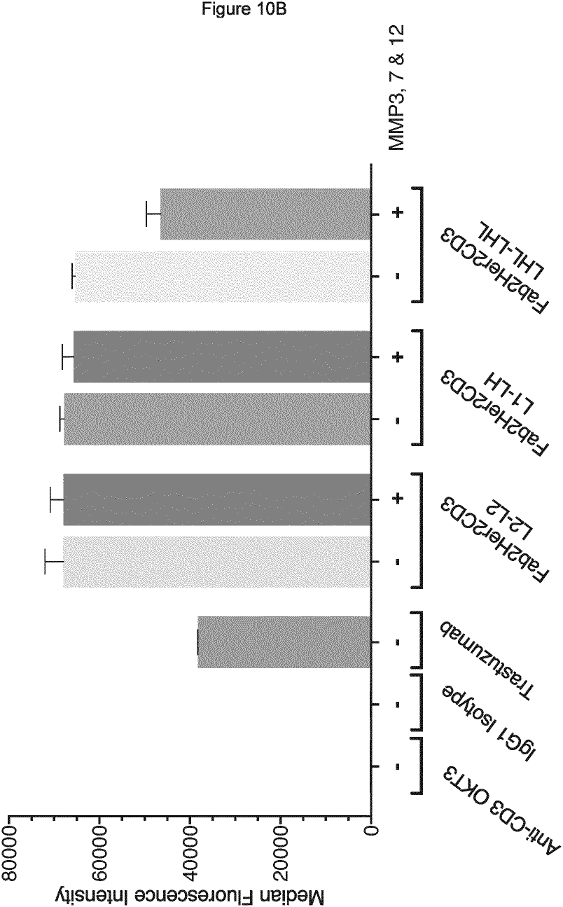

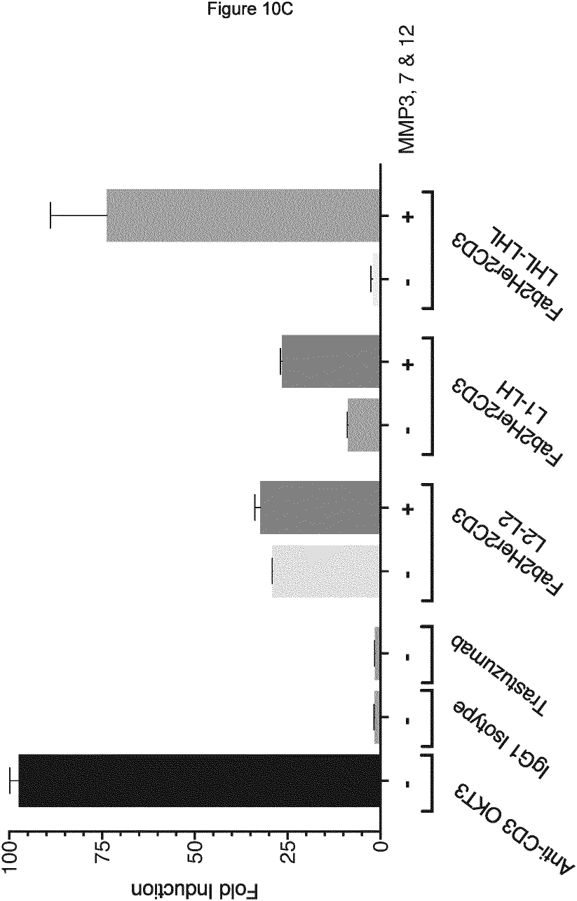

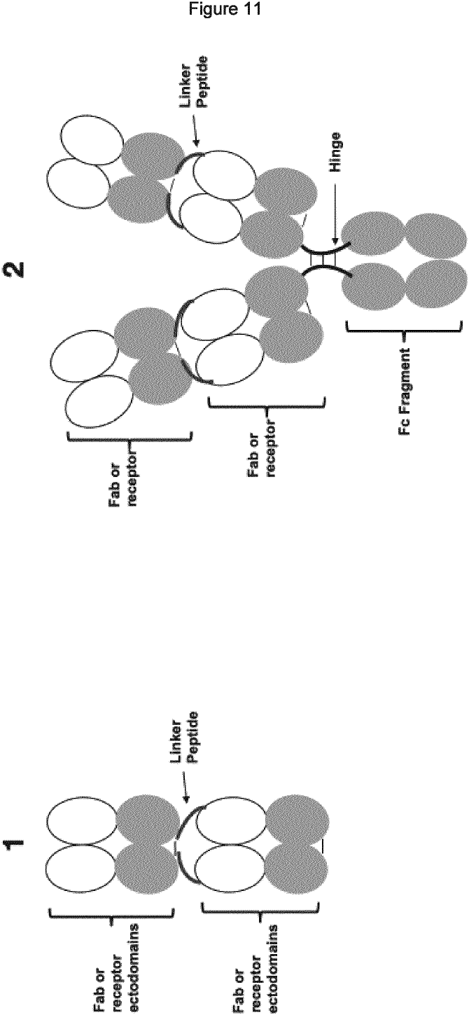

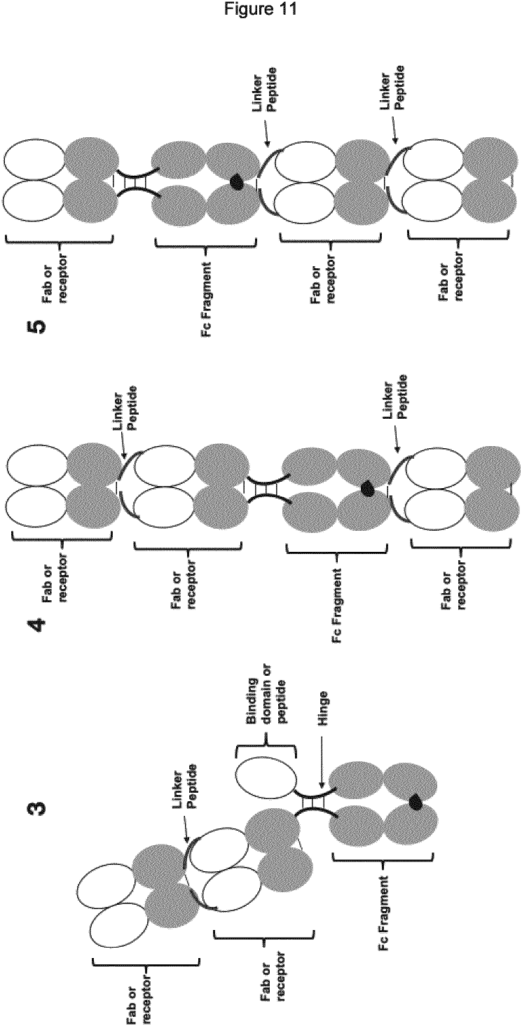

SEC chromatography was then performed on the remaining protein samples from a subset of clones in an attempt to produce fully purified monomeric protein. Chromatography traces for clones 1, 2, 3, 4, 5, 6, 10, 12 and 14 in preparative SEC are shown in FIG. 5. These analyses further demonstrated that the clones 1 (FIG. 5A), 3 (FIG. 5C), 4 (FIG. 5D), 5 (FIG. 5E) and 12 (FIG. 5G) contained a large percentage of higher and often lower molecular weight products around the main peak of desired product (eluting at a CV of approximately 0.5-0.55 in FIGS. 5A-5H). In contrast, clones 6 (FIG. 5F), 14 (FIG. 5H) and 10 (FIG. 5I) exhibited a dominant, well-defined major product peak. For clones 2 and 6, this allowed effective SEC purification of a predominantly monomeric protein, as demonstrated by SDS-PAGE of unreduced samples (FIG. 6A). Attempted SEC purification of clones 1, 4, and 5 failed, producing samples that were still heterogeneous (FIG. 6A). SEC-purified protein from Her2CD3 Fab² clones 7, 8 and 10 exhibited improved homogeneity, while of the Her2CD47 IgG² clones, only clone 14 achieved full homogeneity and monomeric status as evidenced in SDS-PAGE of unreduced and reduced samples (FIG. 6B). Again, these findings demonstrated that clones 6, 10 and 14, which all contained the LHL linker on both chains, exhibited the most reproducibly beneficial characteristics.

### Functional Characterization of Protein Constructs Containing CD47 Antibody V-Domains in the Lower Fab

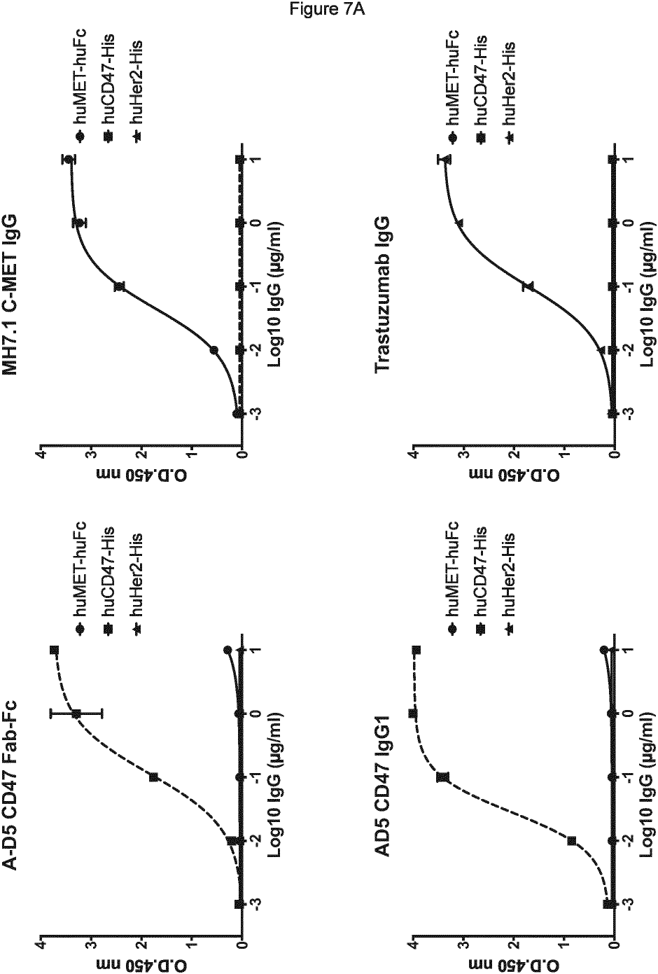

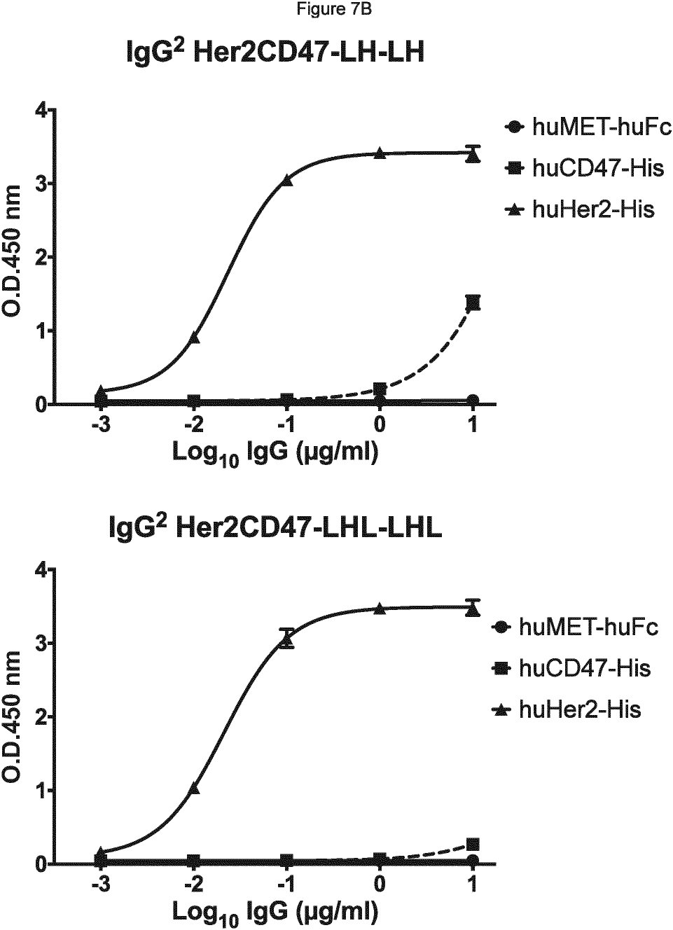

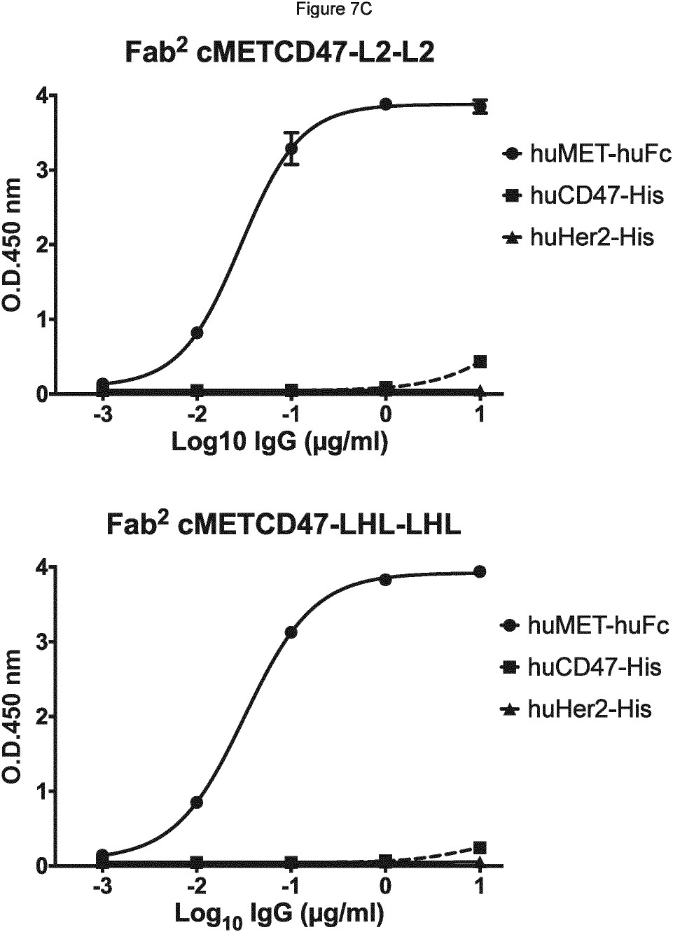

Control IgG antibodies A-D5 anti-CD47, MH7.1 anti-C-MET, anti-Her2 Trastuzumab, and A-D5 Fab-Fc (a monovalent version of the A-D5 antibody containing a single Fab domain), were titrated (in µg/ml) in a direct binding ELISA against human CD47, C-MET and Her2 proteins (FIG. 7A). The Her2CD47-LH-LH and Her2CD47-LHL-LHL clones in IgG² format (FIG. 7B) and cMETCD47-L2-L2 and cMETCD47-LHL-LHL in Fab² format (FIG. 7C) were also analyzed in the same fashion. The control antibodies demonstrated the expected strong binding activity against their cognate targets, with little or no background to any other target, even at the highest concentration (FIG. 7A).

Importantly, the very strong monovalent binding of A-D5 Fab-Fc (FIG. 7A) demonstrated the intrinsic affinity of the A-D5 anti-CD47 domains and the significant potency that would need to be locked into the protein construct format of the invention, for it to be successful. Protein constructs Her2CD47-LHL-LHL (FIG. 7B) and cMETCD47-LHL-LHL (FIG. 7C) also showed similarly strong, highly specific binding to the cognate target of their upper Fab domains, but no binding signal against CD47, demonstrating that the binding activity of the CD47 v-domains was indeed fully inhibited when this linker combination was used. Importantly, high background signal on human CD47 was observed for Her2CD47-LH-LH and to a lesser extent for cMETCD47-L2-L2, suggesting that the ablation of binding affinity in the lower fab is critically controlled by linker choice. These findings suggested that the protein constructs Her2CD47-LHL-LHL (FIG. 7B) and cMETCD47-LHL-LHL (FIG. 7C) containing the anti-CD47 binding domains of antibody A-D5 that were tested in this assay have reduced ability to bind CD47 by >1000-fold in comparison to A-D5 IgG1, where a binding signal OD of 1.0 is achieved at approximately 1 × 10⁻² µg/ml, while neither protein construct exhibits signal above 0.2 at the top concentration tested, of 10 µg/ml.

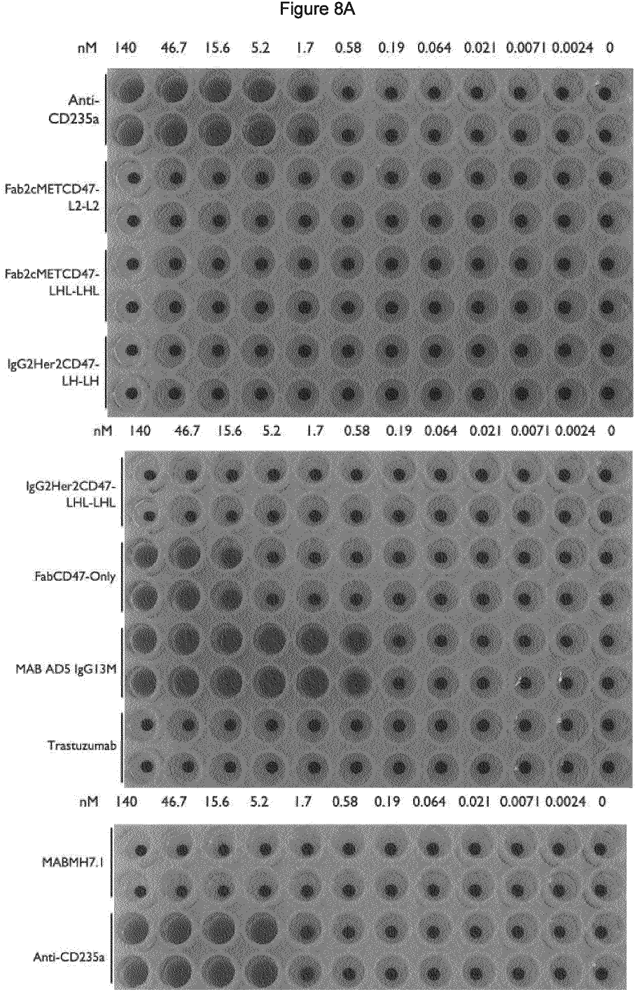

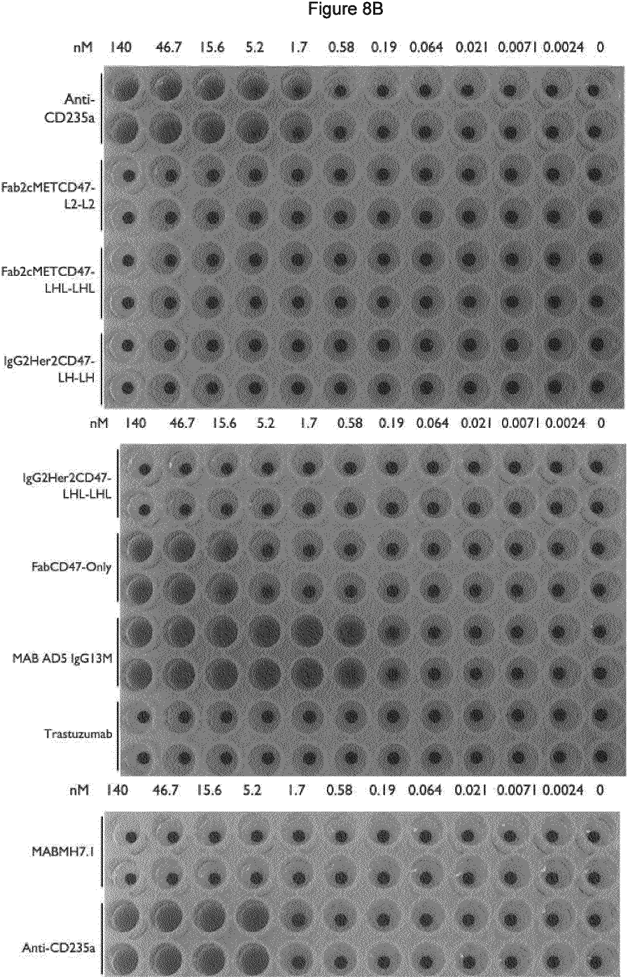

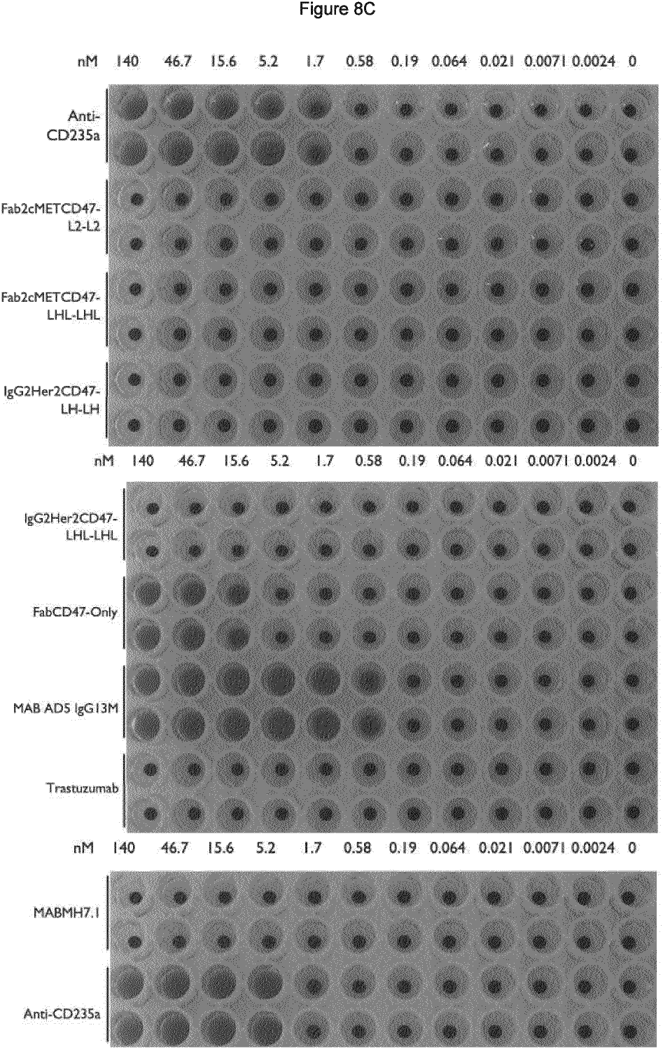

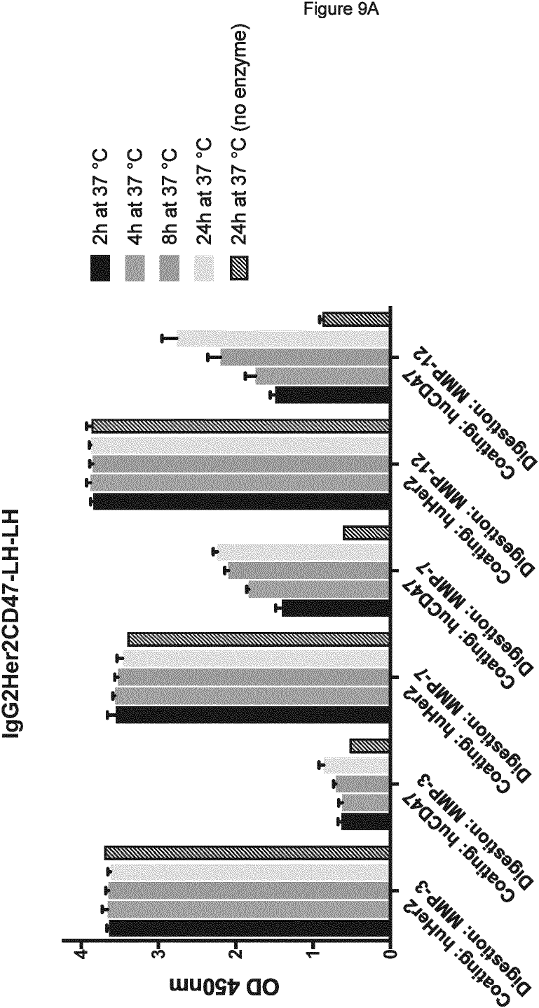

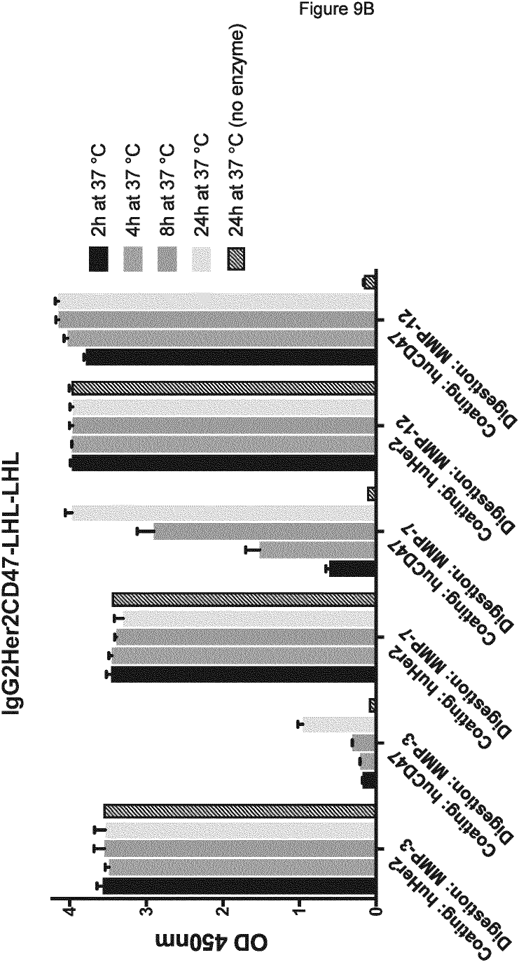

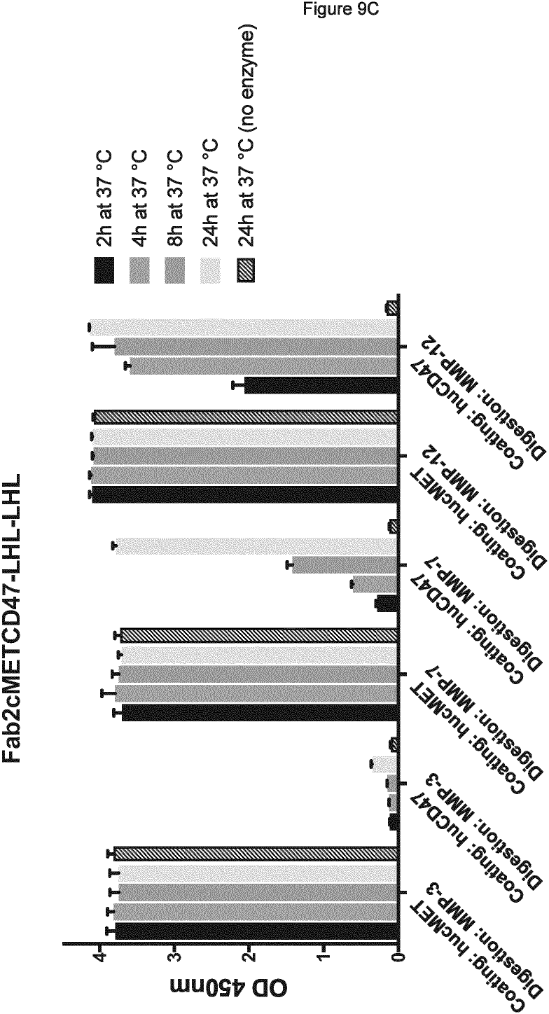

As hemagglutination is a primary toxicity risk of anti-CD47 antibodies, the prioritized protein constructs were then examined in a human erythrocyte-based hemagglutination assay. Control antibodies anti-CD235a (murine) and A-D5 anti-CD47, A-D5 Fab-Fc, MH7.1 anti-C-MET, anti-Her2 Trastuzumab, Her2CD47-LH-LH and Her2CD47-LHL-LHL IgG2, and cMETCD47-L2-L2 and cMETCD47-LHL-LHL in Fab² format, were titrated (in nM) in a human erythrocyte hemagglutination assay using fresh erythrocytes from healthy donor 1 (FIG. 8A), donor 2 (FIG. 8B) and donor 3 (FIG. 8C). For all 3 donors, control antibodies anti-CD235a, A-D5 anti-CD47 and A-D5 Fab-Fc demonstrated potent concentration-dependent hemagglutination, due to cross-linking their respective surface antigens on adjacent erythrocytes. The low-potency hemagglutination effects observed for A-D5 Fab-Fc (clone 15) may be due to the presence of small amounts of functional dimer in this protein preparation as it was only purified by Protein A column and not fully purified to monomeric state by SEC. Importantly, none of the protein construct samples exhibited any ability to induce hemagglutination effects, even at a highest concentration of 140 nM. This finding suggests that the IgG² and Fab² format protein constructs containing the anti-CD47 binding domains of antibody A-D5 that were tested in this assay had reduced ability to induce agglutination by >241-fold in comparison to A-D5 IgG1, which exhibited a titre of 0.58 nM.

Prioritized protein constructs were submitted to enzymatic digestion using human MMPs 3, 7 and 12 over a time course of 2, 4, 8 and 24 hours incubation, plus a 24 h incubation in buffer without enzyme as a negative control. Samples from these digest time courses were then applied in direct binding ELISA against human Her2 and CD47 (FIGS. 9A, B) or human C-MET and CD47 (FIG. 9C). In each case, no loss of binding was observed over time towards either Her2 or C-MET, suggesting that the addition of proteases does not reduce the functional binding capacity of the upper Fab domains. In contrast, the CD47 binding capacity of all three protein constructs clearly increased over time, in a MMP-dependent fashion. The clone IgG² Her2CD47-LH-LH (FIG. 9A) again exhibited high background binding to CD47 and the lowest increase in CD47 binding overtime. In contrast, the clones IgG² Her2CD47-LHL-LHL (FIG. 9B) and Fab² cMETCD47-LHL-LHL (FIG. 9C) again exhibited low background binding to CD47 (OD 450 nM < 0.2) and rapidly increasing binding to CD47 from 2 hours incubation onwards with both MMPs 7 and 12, reaching full saturation (OD 450 nM approximately 4.0) by 24 hours incubation. MMP3 appeared to be the slowest-activating of the 3 MMPs, showing increased CD47 binding signal after 24 hours for all 3 protein construct examples.

### Functional Characterization of Protein Constructs Containing CD3 Antibody V-Domains in the Lower Fab

Antibodies Fab² Her2CD3-L1-LH, Fab² Her2CD3-L2-L2 and Fab² Her2CD3-LHL-LHL were analyzed by ELISA (FIG. 10A), Flow Cytometry (FIG. 10B) and a CD3 reporter assay (FIG. 10C). In ELISA analysis, all 3 proteins demonstrated the expected strong binding activity against Her2 (Upper Fab domain), with little or no background binding to any other target, even at the highest concentration (FIG. 10A). All 3 protein construct Fab² proteins were then submitted to incubation overnight with, or without mixed MMPs 3, 7 and 12 before further analysis. In flow cytometric binding to the HER2+ human cell line BT474, anti-HER2 Trastuzumab exhibited strong binding, Fab² Her2CD3-L1-LH and Fab² Her2CD3-L2-L2 showed similarly potent binding before and after MMP digest, while Fab² Her2CD3-LHL-LHL exhibited partially reduced binding after MMP digest, suggestive of a proportion of the protein losing the upper fab in the ‘dissociated’ state (FIG. 10B). As this finding suggested that the MMP digest process was active in the case of Fab² Her2CD3-LHL-LHL, the same samples were applied in a reporter assay where Her2+ BT474 cells were mixed with human CD3+ Jurkat cells engineered to provide a measurable signal if CD3 is activated. The data from this assay demonstrated that the bivalent anti-CD3 antibody OKT3 directly activated the CD3 signal in the reporter cells, as expected (FIG. 10C). The protein constructs each exhibited distinct profiles: The Her2CD3-L2-L2 protein, which contains 2xG4S linkers (not cleavable by MMP proteases) showed high background in the assay with no increase in CD3 activation signal after MMP digest, suggesting that the flexible linkers of this construct allow binding of Her2 by the upper Fab and partial activity of the lower fab allowing some background, but non-activating CD3 co-ligation (FIG. 10C). The Fab² Her2CD3-L1-LH protein exhibited lower background activity in the assay, with a moderate increase in signal after MMP digest. The Fab² Her2CD3-LHL-LHL exhibited no measurable background signaling in the absence of MMP digest, showing minimal activation of CD3 similar to the negative control Trastuzumab and IgG1 Isotype control antibodies in the undigested sample, but potent activation in the MMP digested sample. The data in FIGS. 10A-C therefore suggest that the Her2CD3-LHL-LHL Fab² format had the best combination of properties as it was simply expressed and purified, has high intrinsic Her2 binding activity, low background CD3-ligating activity, and high CD3 co-ligation activation only when activated by MMP cleavage of the LHL linker.

### Second Generation Construct Cloning and Expression

The performance of the above multispecific Fab² and IgG² clones containing the LHL linkers encouraged a second series of constructs to experimentally explore the potential functional sequence space in both tertiary structure and linker sequence content. The following clones were synthesized and assembled to sample these parameters:

1. Clone ‘Met47-LHL-LHL’, which was a Fab² protein in the ‘One Arm’ style (Table 6, FIG. 3B).

2. Clone ‘Her23-LHL-LHL’, which was a remodeled ‘Clone 10’ Fab² protein in the ‘One Arm’ style (Table 7, FIG. 3B).

3. Clone ‘Her23(34)-LHL-LHL’, which was a Fab² protein in the ‘One Arm’ style (Table 8, FIG. 3B).

4. A series of clones of the IgG² type incorporating the Her2 binding domains of Trastuzumab in the upper fab and the CD47 binding domains of clone A-D5 in the lower Fab (Table 9). These clones contained mutated LHL-based linker sequences that might be more sensitive to enzymatic cleavage by a broad family of MMPs (LHLF linkers), linkers of moderately increased length that added a portion of the human IgG1 middle hinge sequence (LHLM linkers), or both (LHLMF linkers).

5. Clone ‘Fc-Her23(34)’, which was a Fab² protein (Table 13, FIG. 12) in which the Fab² module is placed at the c-terminal end of the KIH-Fc.

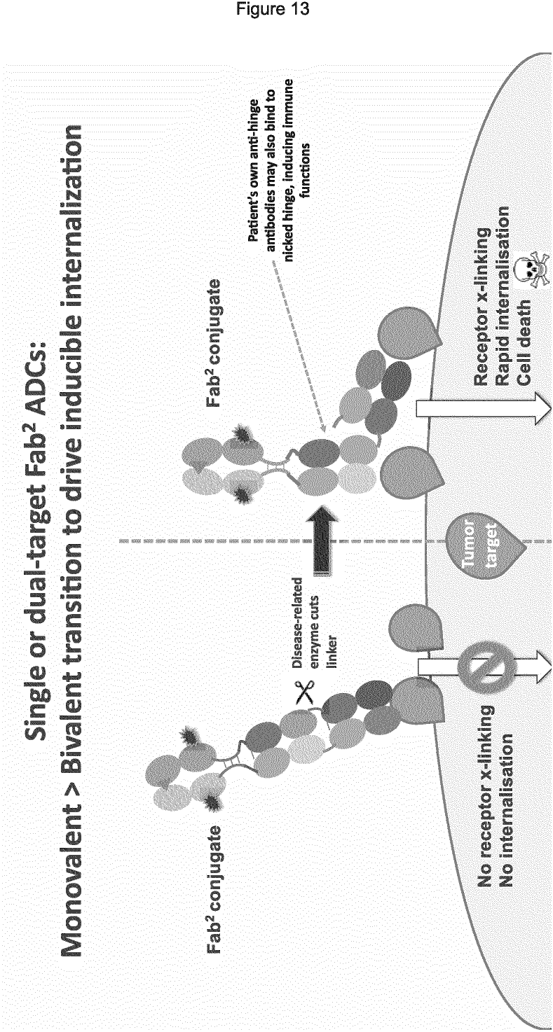

6. Clone ‘MetMet-LHL-LHL, which was a Fab² protein in the ‘One Arm’ style (Table 14, FIG. 3B, FIG. 13) containing two copies of the C-Met Fab. In this structural format the binding to the Met receptor in bivalent form can only occur after a single LHL linker has been cleaved by a protease.

These constructs were successfully expressed and purified by protein A and size exclusion chromatography, as described above.

### Second Generation IgG² Construct Analyses

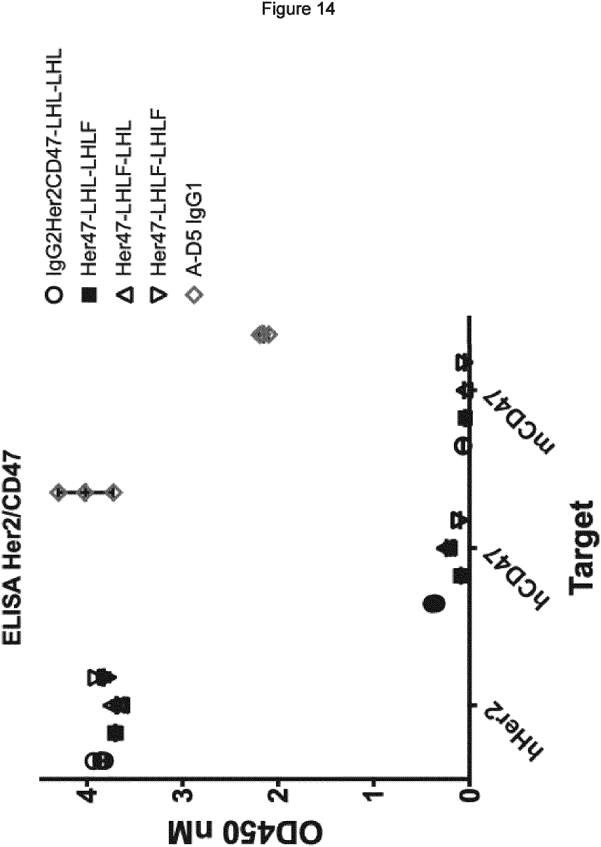

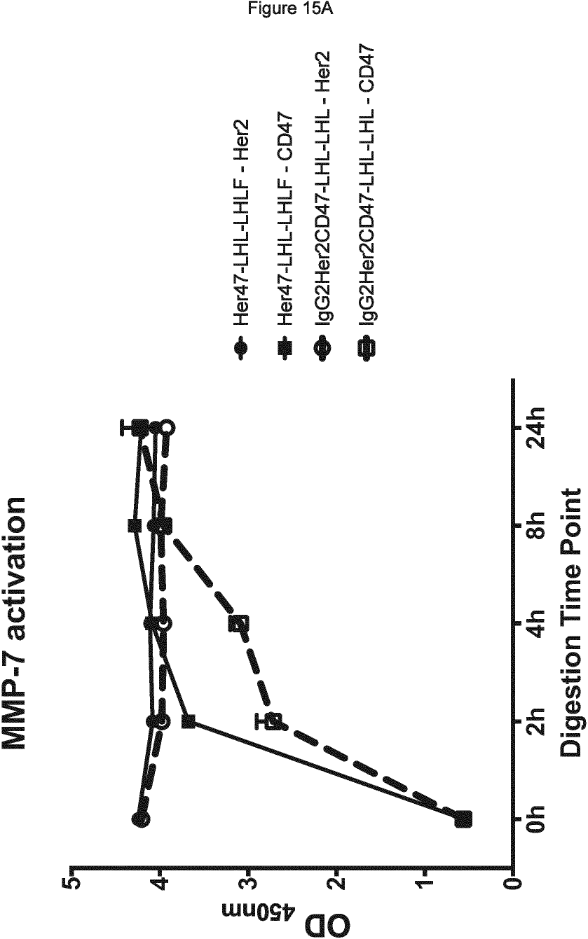

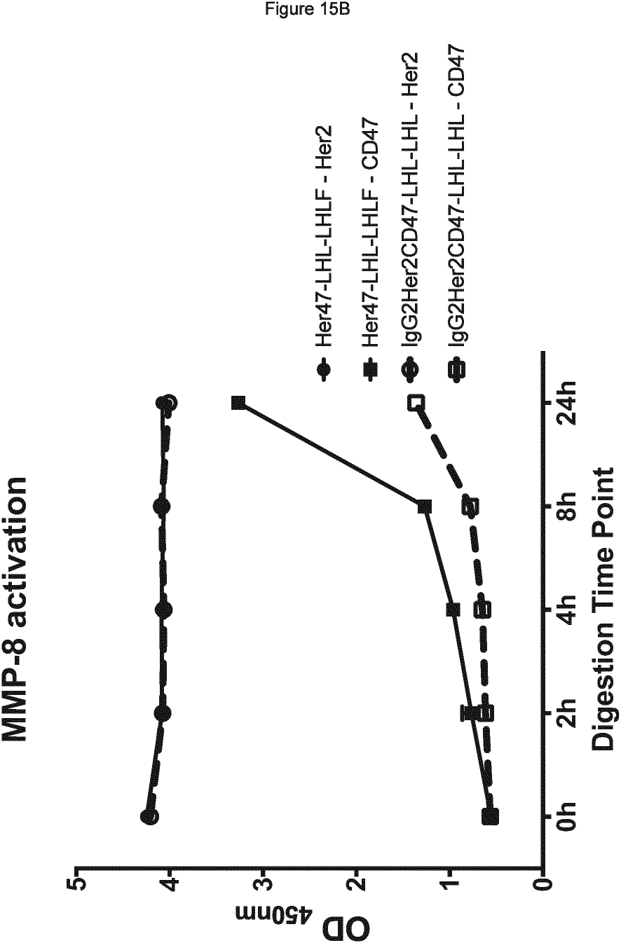

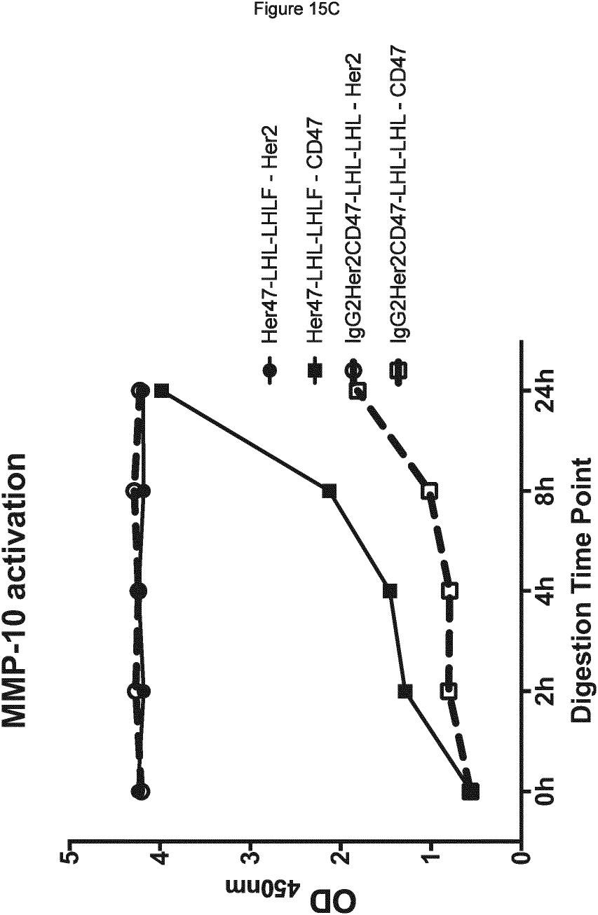

The performance of the above purified IgG² clones was examined in a series of further analyses. Example clones A-D5 IgG1, IgG² Her2CD47-LHL-LHL, IgG² Her47-LHLF-LHL, IgG² Her47-LHL-LHLF and IgG² Her47-LHLF-LHLF (Table 9) were firstly examined for binding to human Her2, and human and mouse CD47 (FIG. 14). This analysis demonstrated that: 1) The A-D5 anti-CD47 IgG1 protein exhibited high binding signal for both hCD47 and mCD47. 2) All 4 IgG² proteins retained high binding signal for hHer2, but no/background binding signal for either hCD47 or mCD47 (FIG. 14).

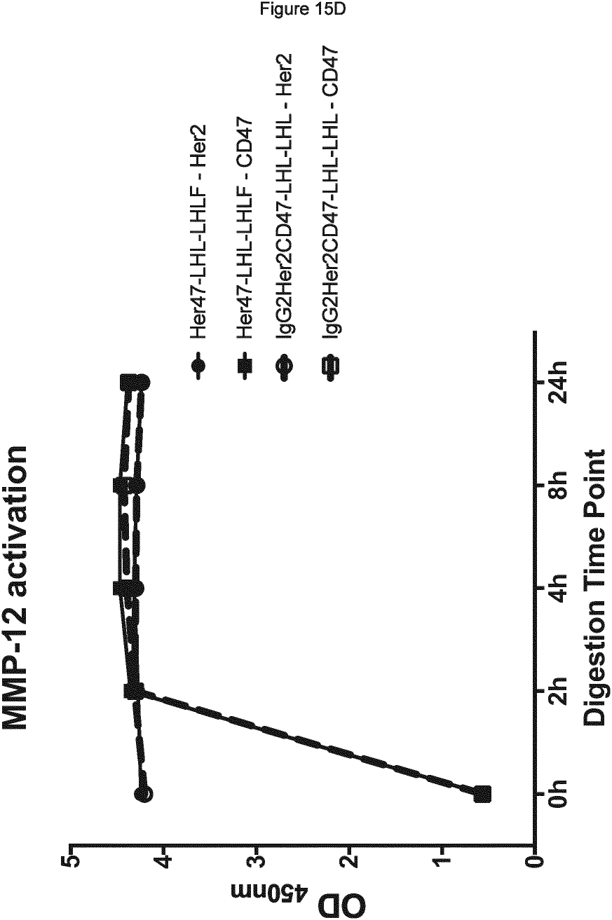

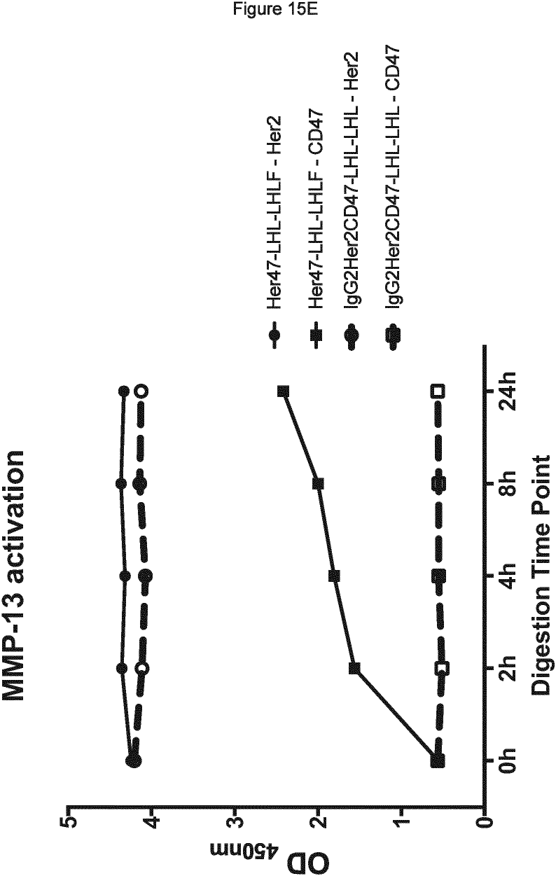

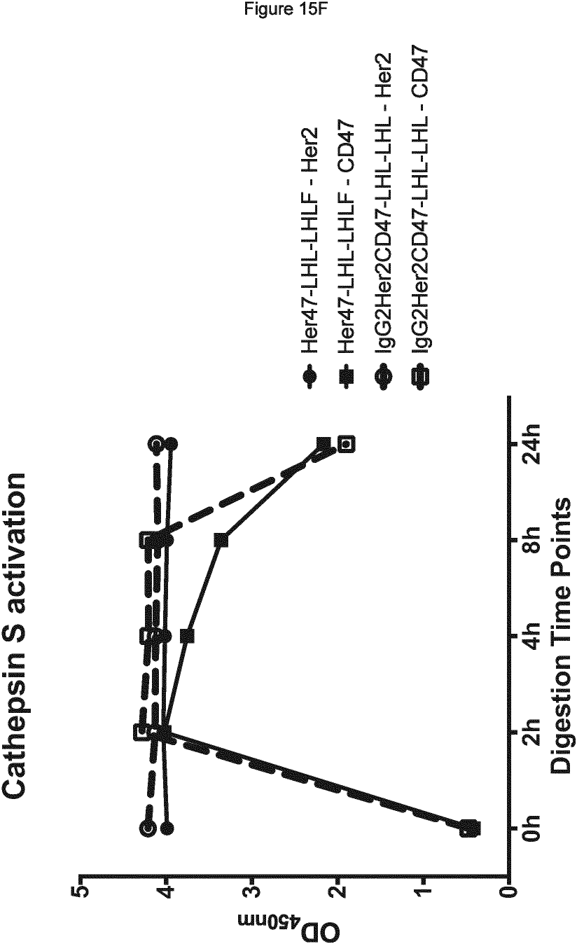

Clones IgG² Her2CD47-LHL-LHL and IgG² Her47-LHL-LHLF were examined for sensitivity to enzymatic activation (at pH7.4) by multiple proteases that are known to be over-expressed in human tumours (FIG. 15): Incubation with MMP7 (FIG. 15A), MMP8 (FIG. 15B), and MMP10 (FIG. 15C) demonstrated that both proteins could be activated for hCD47 binding by each enzyme, but IgG² Her47-LHL-LHLF was activated more rapidly in each case. Incubation with MMP12 demonstrated that both proteins could be activated for hCD47 binding at an equal rate for this enzyme (FIG. 15D). Unexpectedly, incubation with MMP13 demonstrated that IgG² Her47-LHL-LHLF could be activated for hCD47 binding by this enzyme, while IgG² Her2CD47-LHL-LHL could not (FIG. 15E). Importantly, incubation with the cysteine protease Cathepsin S also demonstrated that both proteins could be activated for hCD47 binding at an equal rate for this enzyme (FIG. 15F). These findings demonstrated that the peptide linker content of the Fab² modules contained in IgG² proteins can be ‘tuned’ to broaden the number of potential activating enzymes and even to make the proteolytic activation process more rapid. To sample this potential, further linker designs in the appropriate length and content to drive sensitivity to specific classes of enzymes were envisioned, where amino acid sequences which show proteolytic sensitivity to the activity of other disease-associated metalloproteinases such as ADAMS, Cysteine, Aspartate and serine proteases, etc., would be used to provide either broader or more selective activation.

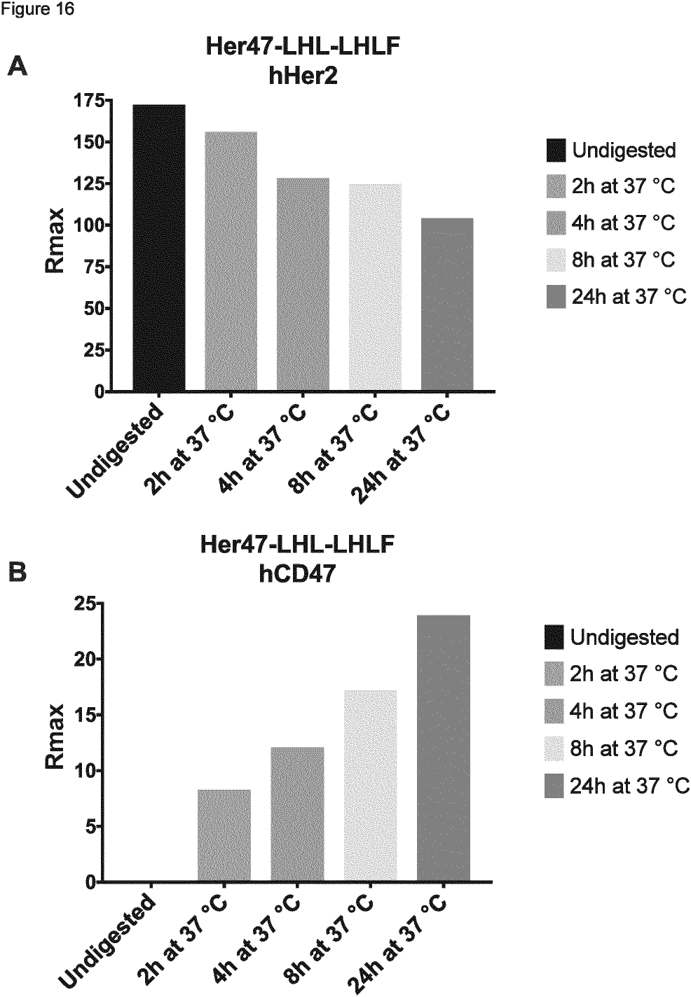

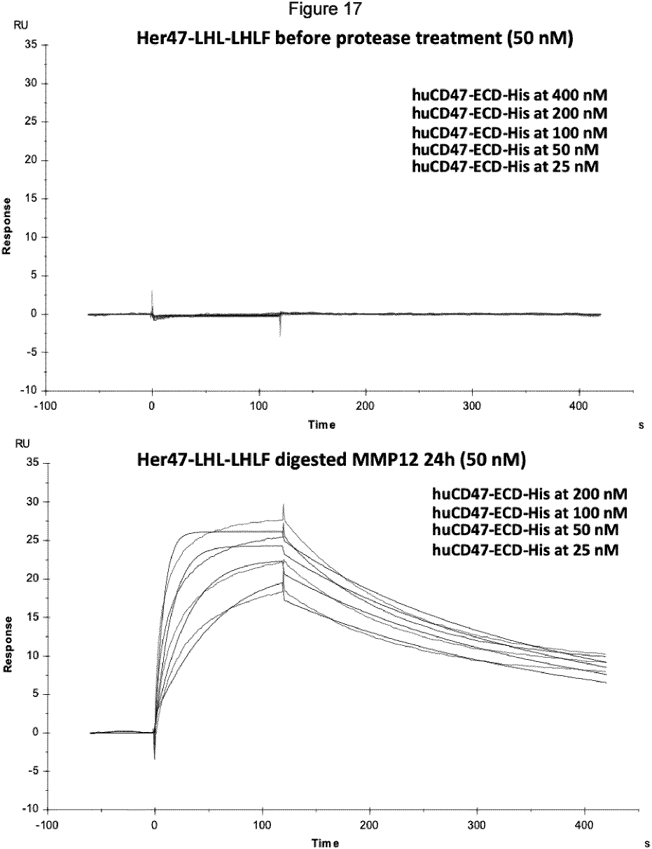

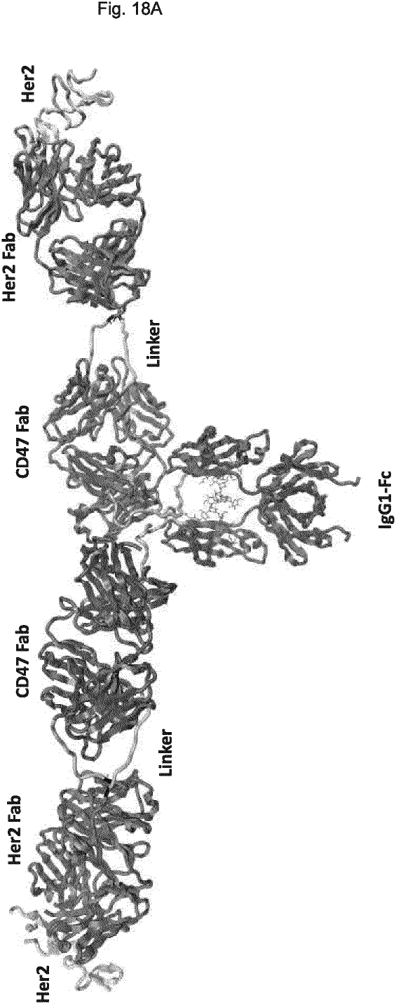

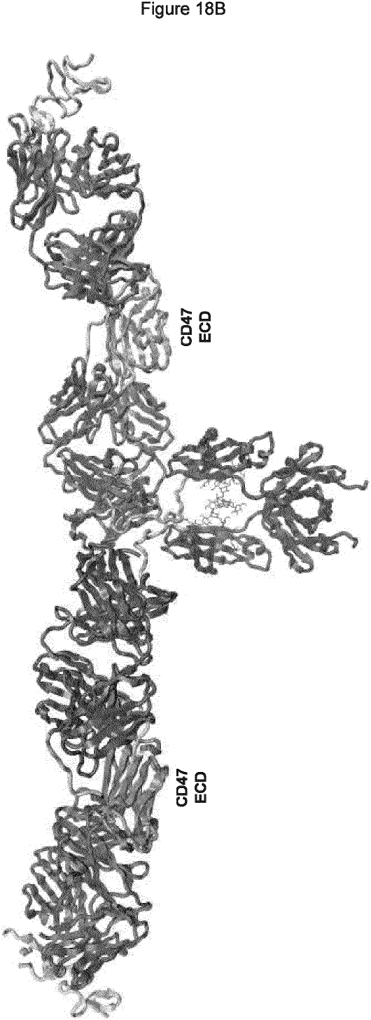

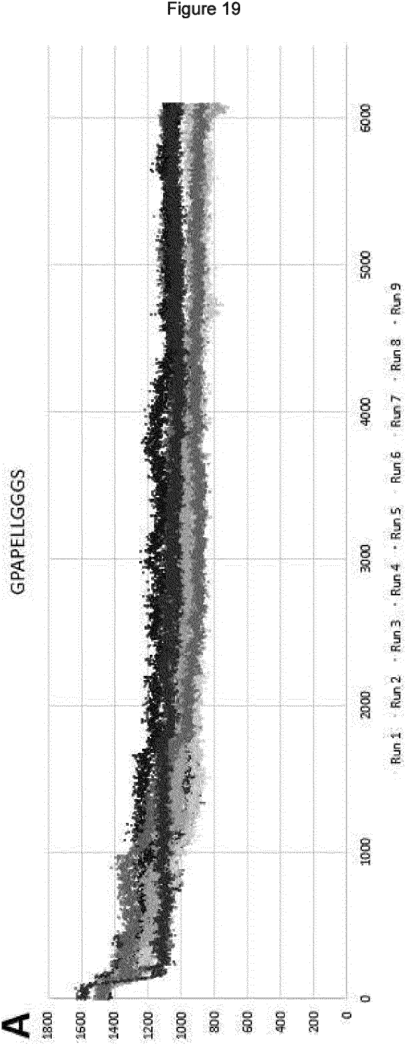

To examine the effect of activation on IgG² binding affinity, a Biacore assay was established which could sample both Her2 and CD47 binding. In this assay, control antibodies and IgG² Her47-LHL-LHLF protein (undigested or activated for 2, 4, 8 or 24 hours with MMP12) were captured on a chip surface via anti-Fc antibody and then binding affinity was measured for soluble Her2 and CD47 ectodomain proteins. Binding analyses were repeated for Trastuzumab IgG1 and A-D5 IgG1 (no enzyme digest) at the beginning and end of experimental runs, with the data being shown in Table 17. These analyses showed that the calculated KD values for each antibody were highly similar in each run. Importantly however, the Rmax values (maximum binding signal at maximal analyte concentration) dropped significantly between runs at the beginning and end of the experiments (e.g. Trastuzumab Rmax 265.80 RU at beginning, 144.13 RU at the end), suggesting that the activity of the anti-Fc antibody capture surface is reduced after the many rounds of regeneration that are inherent to the Biacore method. To examine the effects of MMP12 activation on target reactivity, Rmax and KD values for both Her2 (FIG. 16A, Table 18) and CD47 (FIG. 16B, Table 18) were calculated. This analysis showed that Her2 binding and affinity was maintained over 24 hours of MMP12 activation. Importantly, no CD47 binding (Rmax = 0) was observed in the 0 hour sample (no MMP12 digest), but both Rmax and apparent affinity for CD47 rose rapidly over the activation time course, beginning at 2 hours incubation (FIG. 16B, Table 18). These findings demonstrated that lower Fabs in a Fab² module are truly inert and incapable of target interaction in the intact molecule until protease activation occurs. This observation is further illustrated in FIG. 17, where even at 400 nM concentration of CD47 analyte, no binding activity is observed, but high binding to CD47 is evident after 24 h of MMP12 treatment.

The above findings also demonstrated that the cleavage of the linker peptides in the Fab² module (and therefore the activation of the lower Fab), is temporal. This may be a pharmacological benefit to molecules containing the Fab² module, as the activation is likely to be strongly biased towards diseased tissues in which both the target antigen of the upper Fab and enzymes capable of activating the lower fab are highly over-expressed.

This would lead to rapid drug accumulation, long residence time and high levels of activation in such diseased tissues.

### In Silico Modelling of Fab² Module Structure and Molecular Dynamics

To understand the mechanisms by which the Fab² module might function, we performed structural modelling and molecular dynamics analyses. The crystal structures of human CD47 ECD bound to Fab domains of IgG1 C47B161 (Protein Data Bank identifier 5TZT), IgG1 C47B222 (Protein Data Bank identifier 5TZ2) and IgG1 B6H12.2 (Protein Data Bank identifier 5TZU) were used as templates to generate a model of the anti-CD47 A-D5 Fab bound to CD47 ECD using the protein modelling suite of MOE software. The structure of trastuzumab Fab in complex with the Her2 extracellular domain was taken from the PDB structure 1N8Z.

The LHL and LHLF linkers between the upper trastuzumab Fab and the lower anti-CD47 Fab were then modelled using the protein modelling suite of MOE software. To assist modelling of the linkers, the C-termini of Fab structures available in Protein Data Bank were examined to help define the conformations with which the LB linkers could exit from each of the heavy and light chain domains of the Trastuzumab Fab. The modelling predicted that the trastuzumab Fab heavy and light chain C-termini can optionally possess the native inter-chain disulphide bond normally present in an IgG1 Fab domain.

A full-length IgG1-based model incorporating the aforementioned, modelled anti-CD47 Fab was constructed using the structure of IgG1 b12 (Protein Data Bank identifier 1HZH) as a template. Structural errors in 1HZH such as missing structural regions were remodelled and corrected. The IgG1 b12 Fab were replaced by the anti-CD47 Fab and the Fc-hinge were attached using the protein modelling suite of MOE software. This model illustrated the likely tertiary structure of an IgG² molecule based on Her2 and CD47 with IgG1 (FIG. 18A). In the model, the binding of the Her2 epitope is constitutively active (FIG. 18A), while the binding of the CD47 ECD is fully occluded by both the linker itself and the proximity of the anti-CD47 Fab to the anti-HER2 Fab (FIG. 18B). The model is consistent with a Fab² module that could bind HER2 via the upper fab with no observed hindrance, but that is prevented from binding CD47 via the lower fab until the linker is degraded (FIG. 16A, FIG. 16B).

To gain further insights into how the Fab² module moves in solution, we performed molecular dynamics simulations using multiple linker compositions, including LHL-LHL, LHL-LHLF and L2L2 (G4SG4S (SEQ ID NO:32) amino acid sequence, Table 1). An RMSD for each residue was calculated for each run. In addition a custom descriptor dSASA was written in the SVL language (MOE) which calculated the change in solvent accessible surface area (SASA) of each residue, and also an aggregate of only the linker sequence in each case. As such it provided the basis to compare how different uncleaved linkers perform in terms of conformational changes and associated effects on solvent accessibility of the linkers which is expected to impact sequence-specific cleavage potential (FIG. 18).

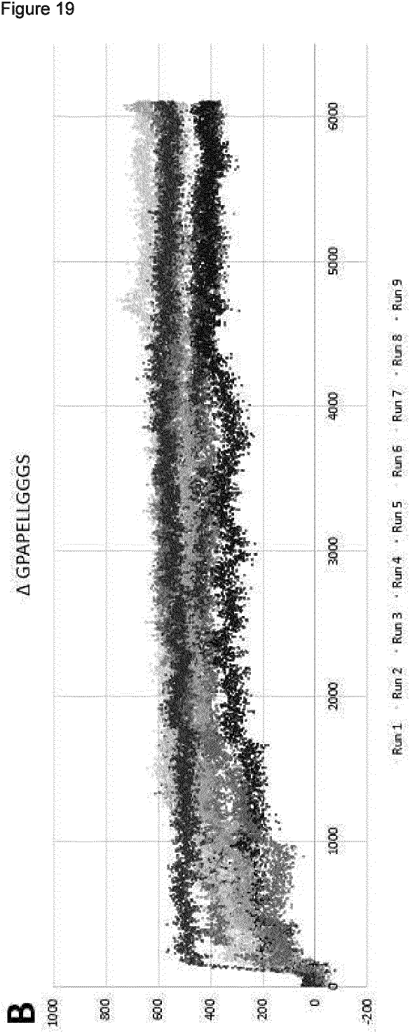

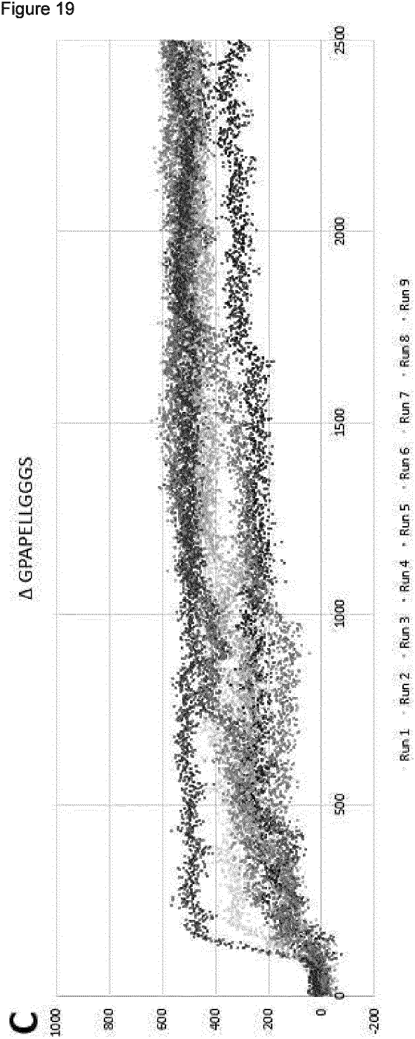

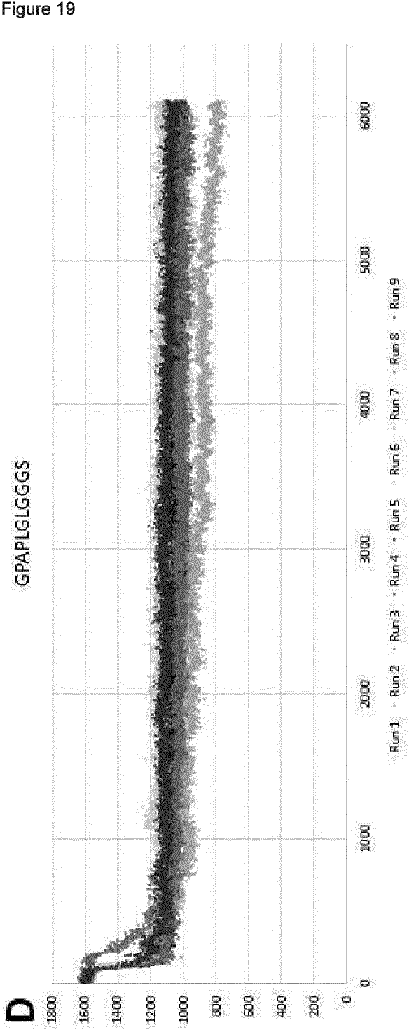

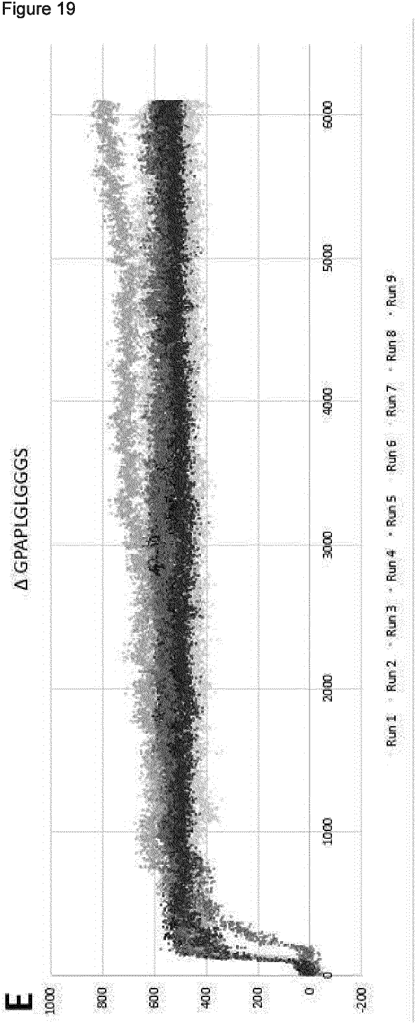

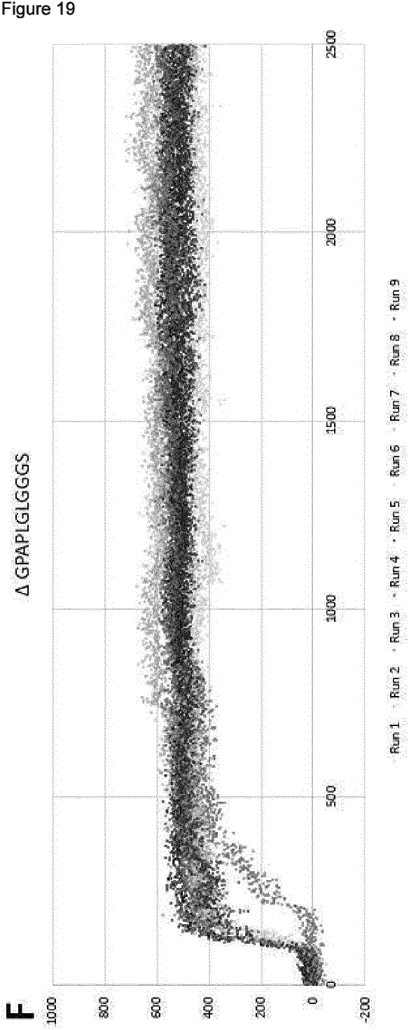

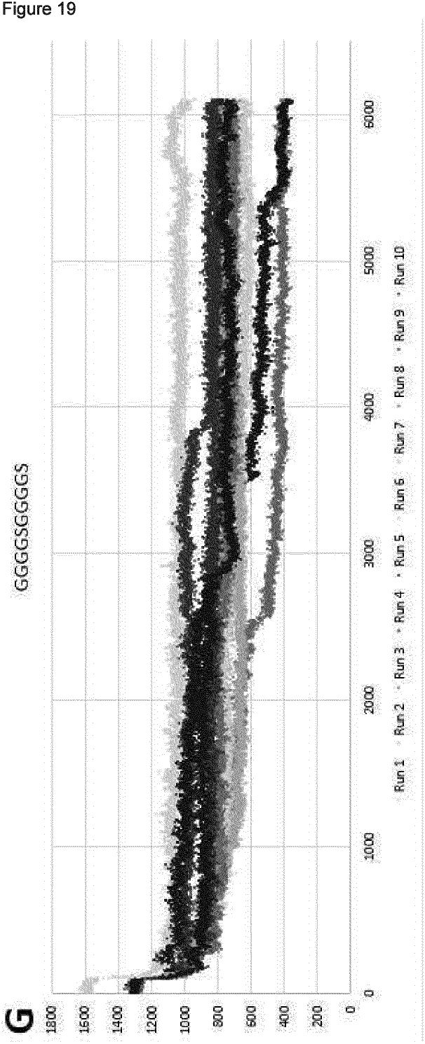

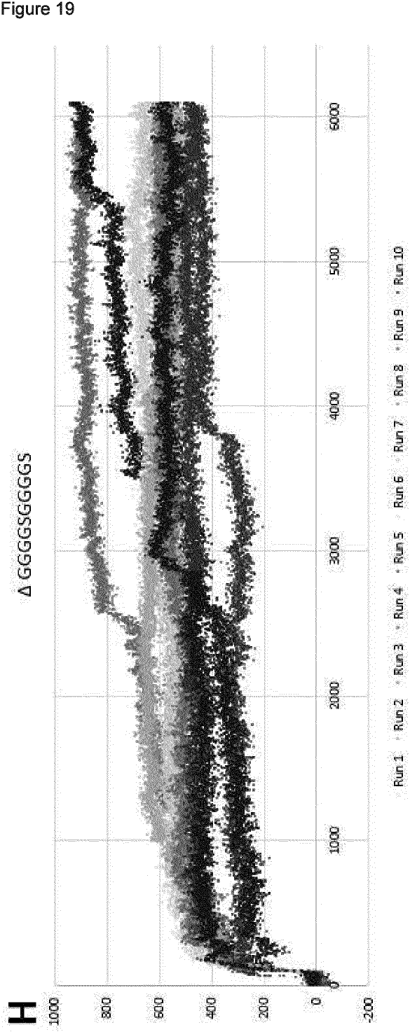

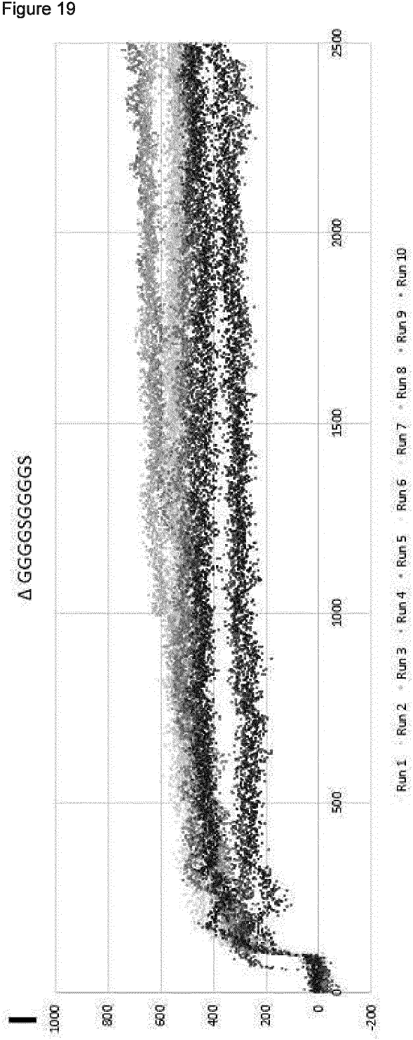

FIG. 19A - FIG. 19I show 9 graphs, corresponding to the solvent accessible surface area (SASA) results obtained for the 3 linkers tested. The first analyses sampled the absolute SASA values (over 6 ns) for 9 dynamics runs for each of LHL and LHLF linkers and 10 runs using L2 (FIG. 19A, FIG. 19D and FIG. 19G, respectively). This data demonstrated that the L2 linker clearly had the greatest propensity to structural heterogeneity over time (FIG. 19G). Since the starting SASA value at was not the same for each run, secondary analyses normalized results representing the difference to the starting SASA value, calculated by subtracting all SASA values from the starting SASA value. Results over the 6 ns dynamics run time (FIG. 19B, FIG. 19E and FIG. 19H, respectively) and the first 2.5 ns of the 6 ns dynamics runs (FIG. 19C, FIG. 19F and FIG. 19I, respectively) illustrated that the LHL and LHLF linkers were clearly less structurally dynamic than the L2 linker. In particular, the 6 ns molecular dynamics runs show that the L2 exhibits highest flexibility, thus underscoring the concept that different linker sequences will give rise to different flexibilities and, therefore, CDR solvent exposure profiles for the lower Fab in the Fab² module.

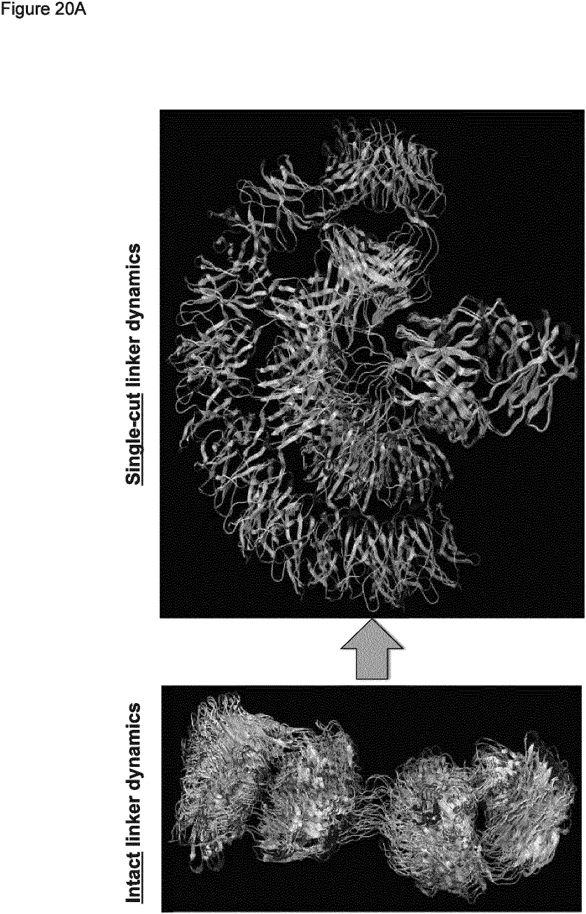

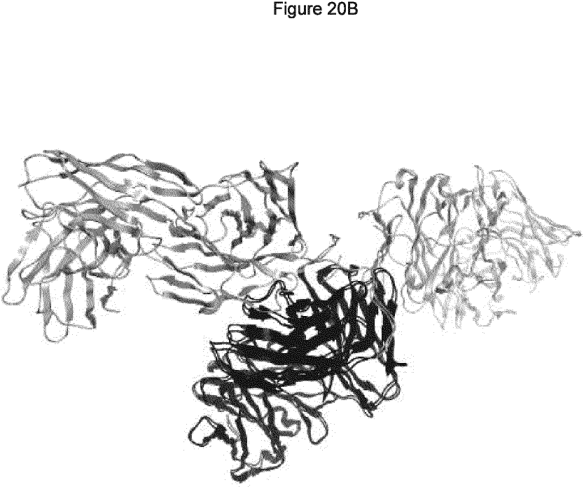

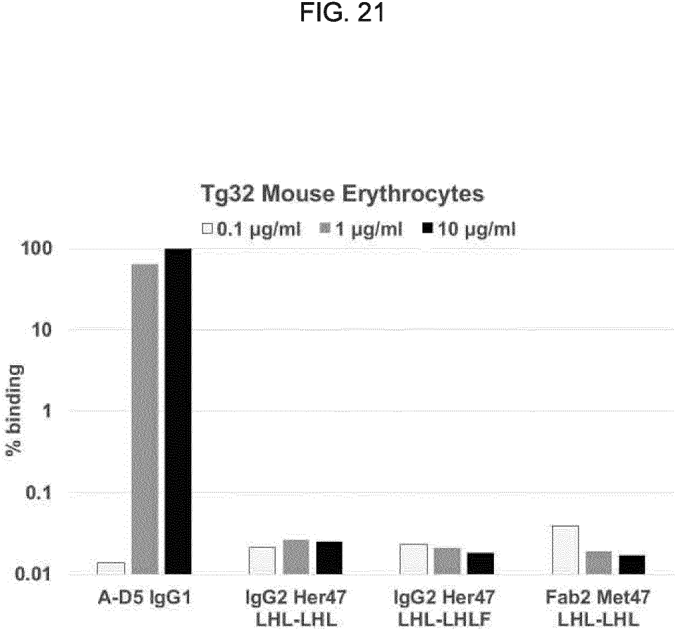

Comparisons between cleaved and uncleaved linkers show dramatic increases in flexibility for the cleaved linkers compared to uncleaved linkers, consistent with biological observations in FIGS. 14-17. For example, FIG. 20A shows the limited movement in the Fab² module when both linkers are intact, followed by dramatic movement of the upper Fab domain in the context of a single cleaved LHL linker (second linker intact) during a 100 ns dynamics run. This analysis showed that the upper fab domain gains a significant increase in degrees of freedom, leading to multiple positions in which it can move fully out of the way of the lower fab domain, fully exposing its CDRs to allow unfettered interaction with e.g. CD47 (FIG. 20B).

### In Vitro and in Vivo Analyses of Tolerability and Pharmacokinetics

To examine the tolerability and pharmacokinetics of IgG2 and Fab2 in the context of CD47 as the lower Fab domain, multiple example molecules (A-D5 IgG1, IgG² Her47 LHL-LHL, IgG² Her47 LHL-LHLF and Fab² Met47 LHL-LHL) were studied in 6 - 8 week old male B6.Cg-Fogrt^{tm1Dor} Tg(FCGRT)32DcrJ mice. These ‘Tg32’ mice are human FcRn homozygous transgenic animals, with pharmacokinetics characteristics for human IgGs that mimic those of humans and primates. As the CD47 binding domains contained in A-D5 IgG1 and in the lower Fab domain of IgG² Her47 LHL-LHL, IgG² Her47 LHL-LHLF and Fab² Met47 LHL-LHL are all known to be capable of binding to recombinant mouse CD47 protein, they were first tested for reactivity to mouse membrane-presented CD47 on erythrocytes, by flow cytometry (FIG. 21). This study showed that A-D5 IgG1 exhibited no signal on mouse erythrocytes at 0.1 µg/ml, but clear concentration-dependent binding signal at both 1 and 10 µg/ml (FIG. 21). Importantly, the binding to mouse erythrocytes was sub-saturating at 1 µg/ml (63% binding, FIG. 21) and fully saturating at 10 µg/ml (98% binding, FIG. 21). In contrast, none of the proteins IgG² Her47 LHL-LHL, IgG² Her47 LHL-LHLF nor Fab² Met47 LHL-LHL exhibited any binding at any concentration, indicating that the anti-CD47 variable domains of the lower Fab domains are not capable of interacting with CD47 on mouse erythrocytes (FIG. 21).

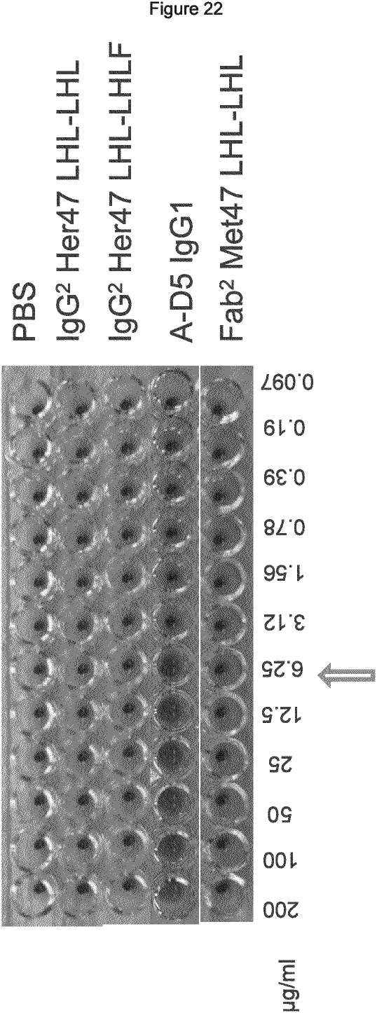

Using isolated erythrocytes from the Tg32 mice, hemagglutination assays were also performed. This analysis demonstrated that only A-D5 IgG1 was capable of driving concentration-dependent agglutination of the mouse erythrocytes (at > 3.12 µg/ml), while none of the proteins IgG² Her47 LHL-LHL, IgG² Her47 LHL-LHLF nor Fab² Met47 LHL-LHL exhibited agglutination at any concentration up to 200 µg/ml (FIG. 22). These findings illustrated that the in vitro examinations of human CD47 protein and erythrocyte binding outlined above (i.e. full binding to human CD47 by A-D5 IgG1, but no measurable binding for IgG² or Fab² proteins) are recapitulated in the mouse system, making mice a viable model to study the effects of CD47 binding on both pharmacokinetics and tolerability.

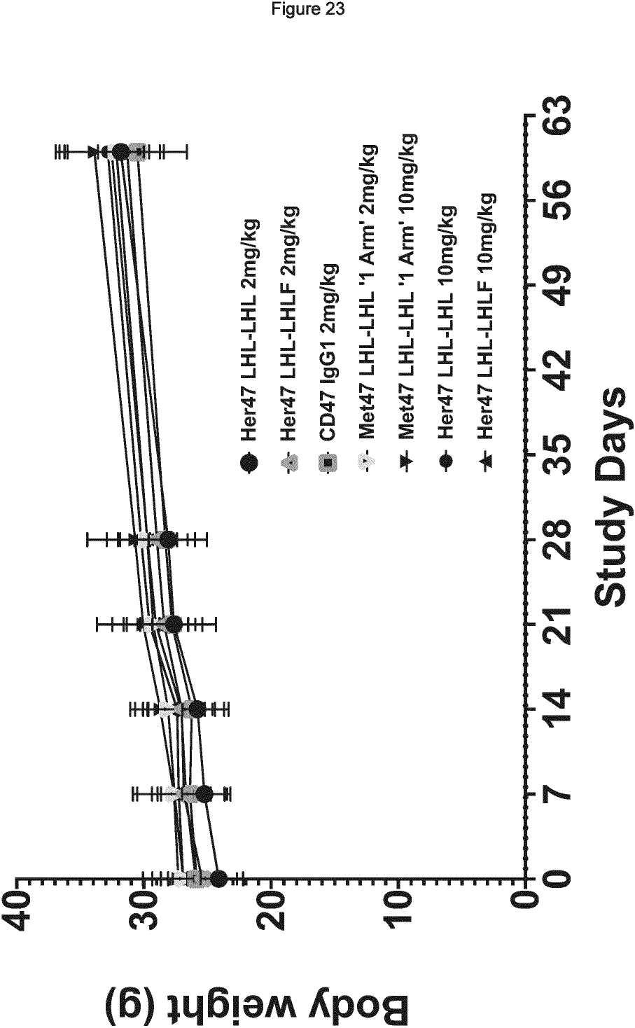

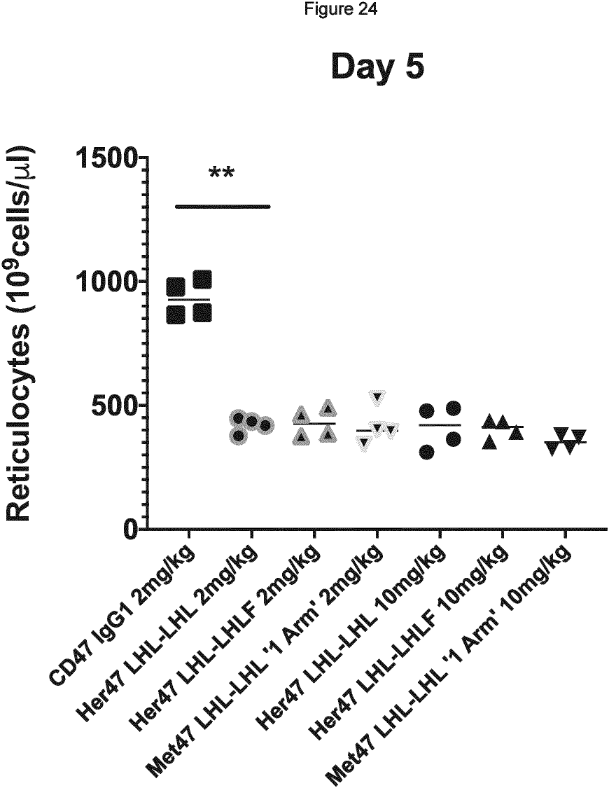

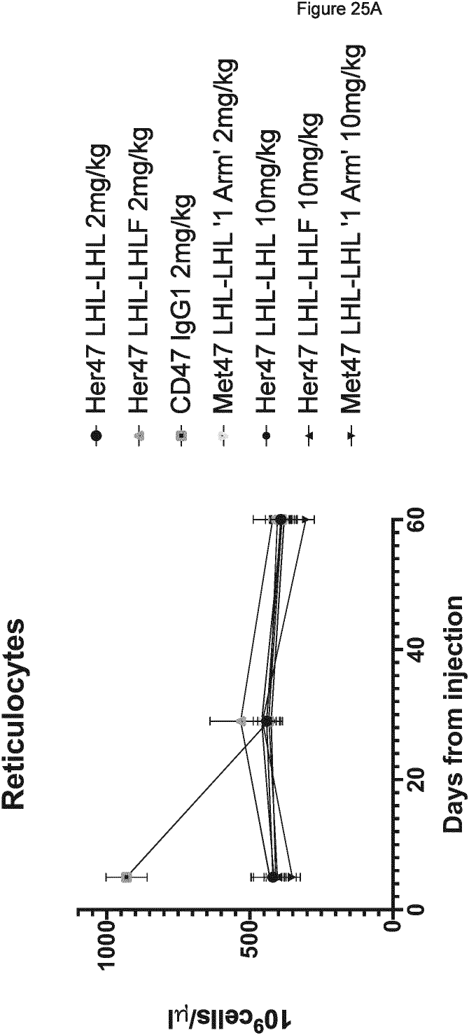

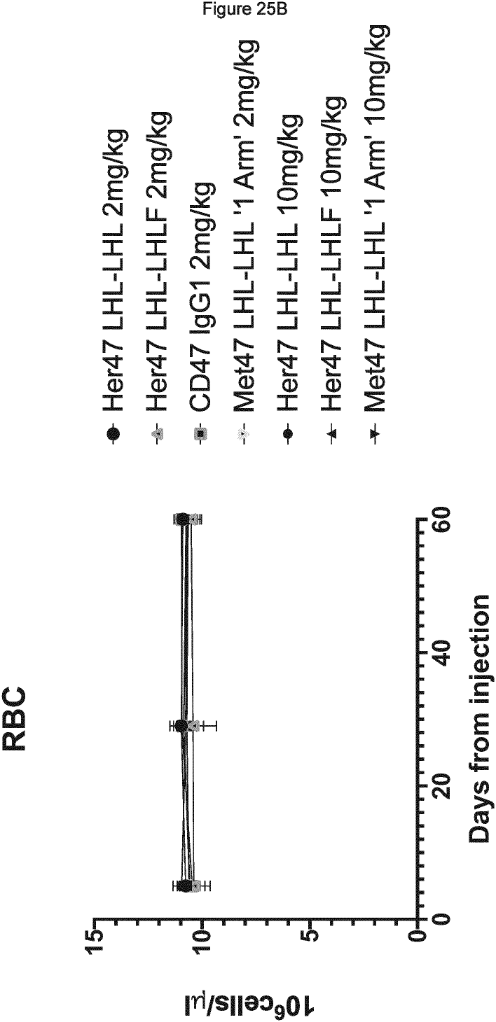

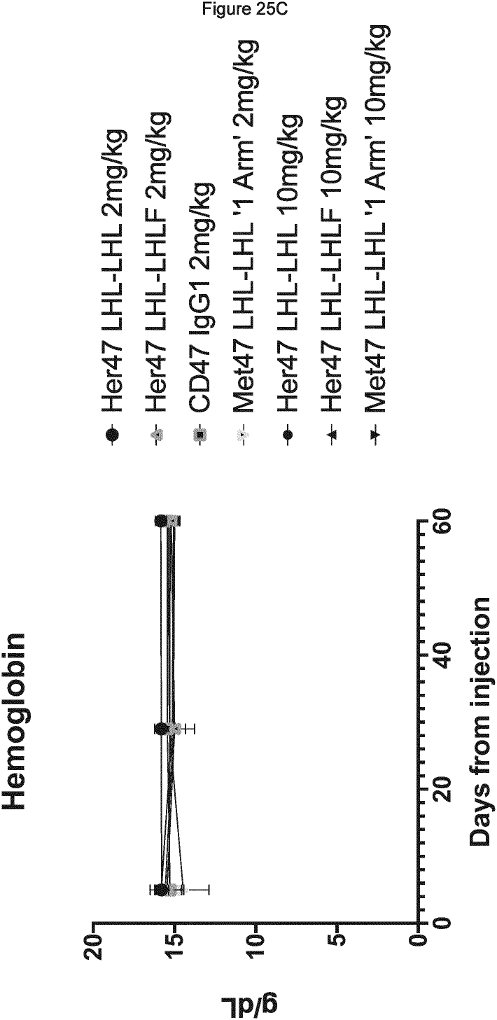

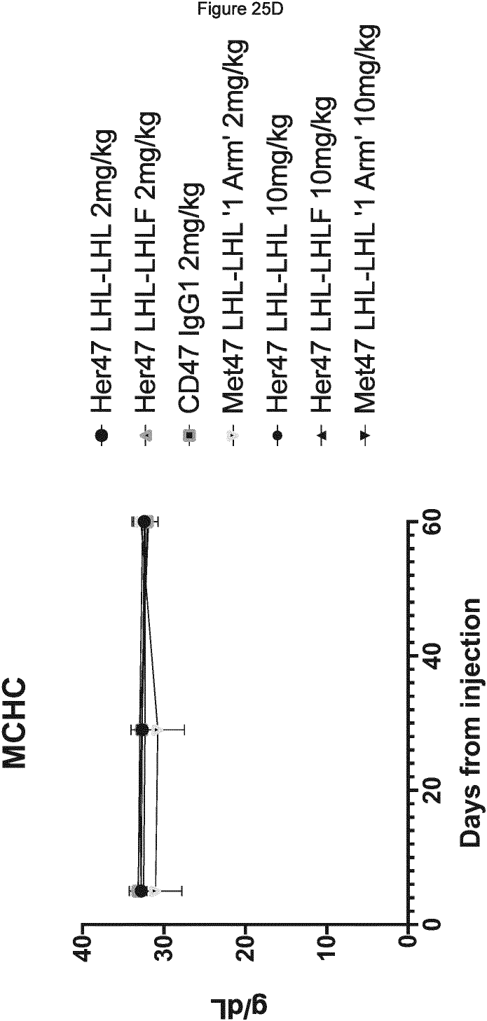

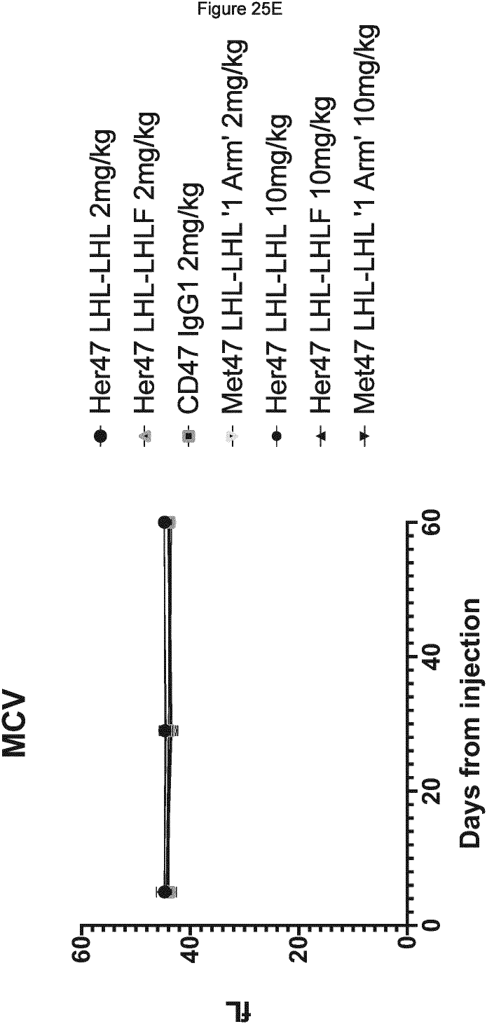

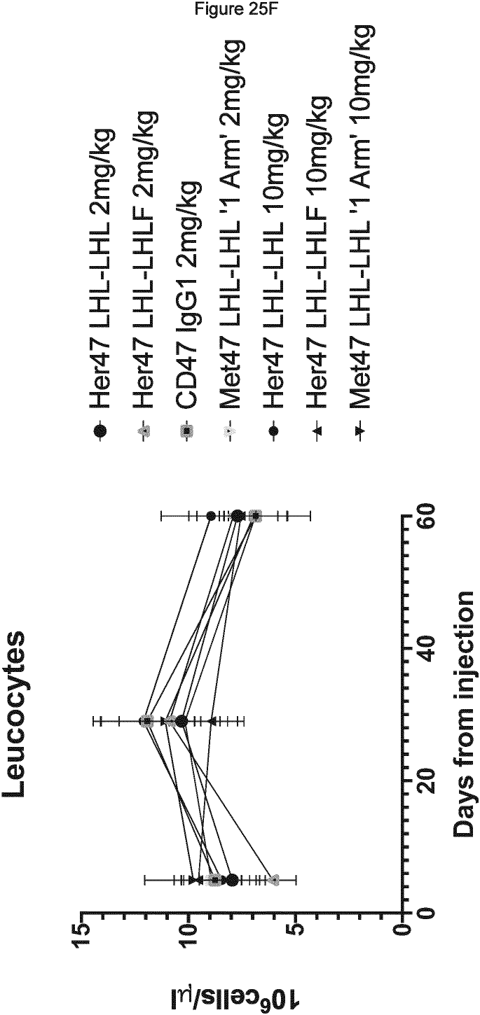

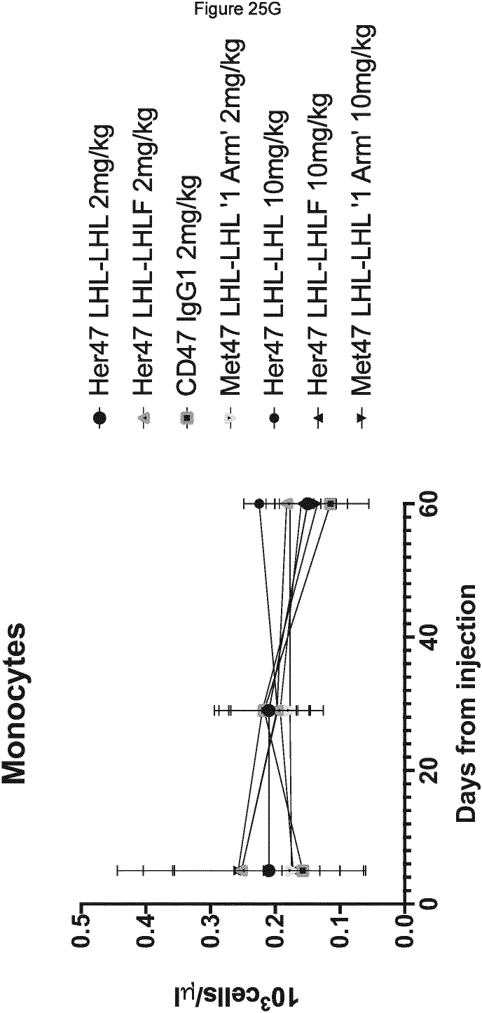

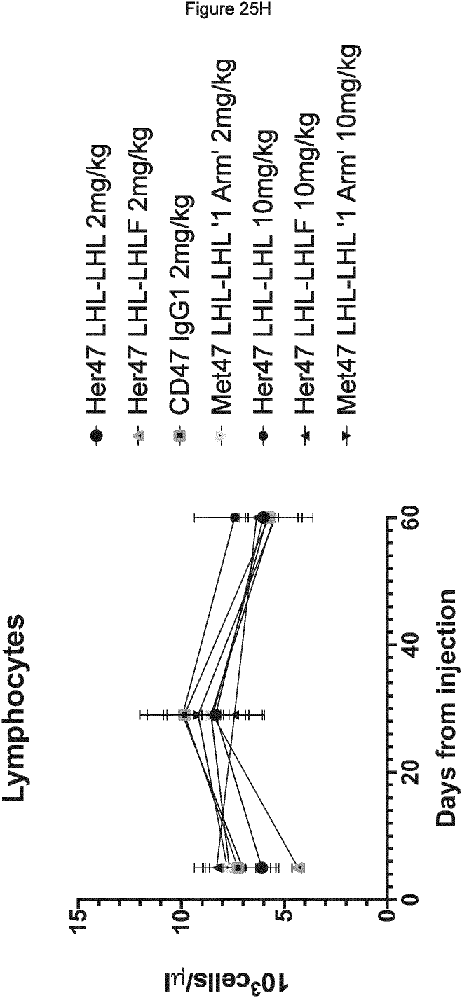

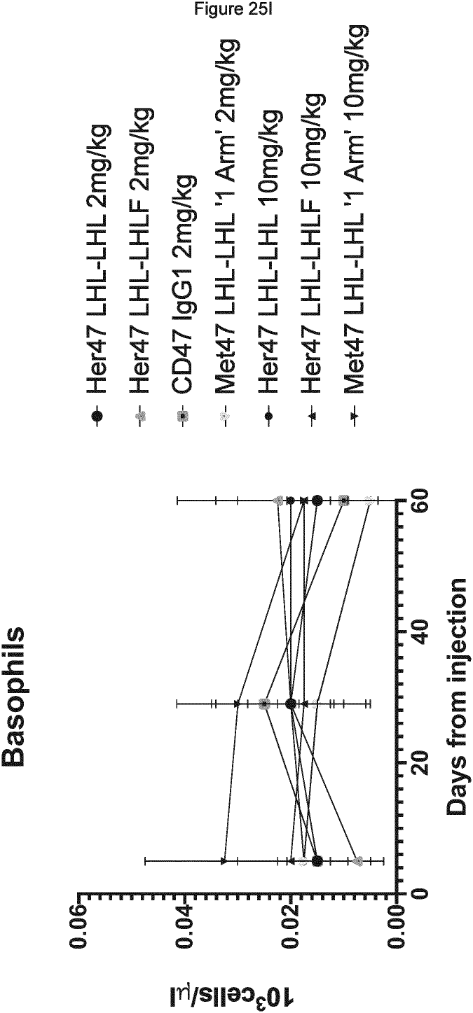

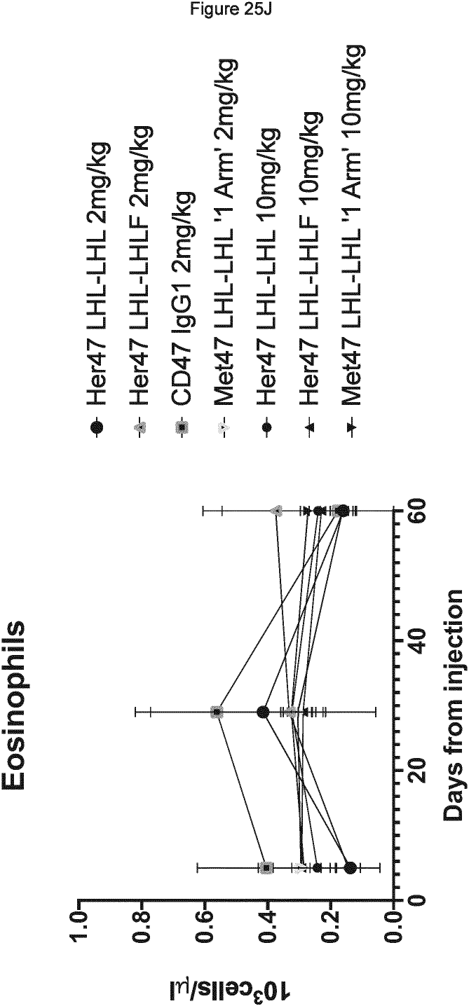

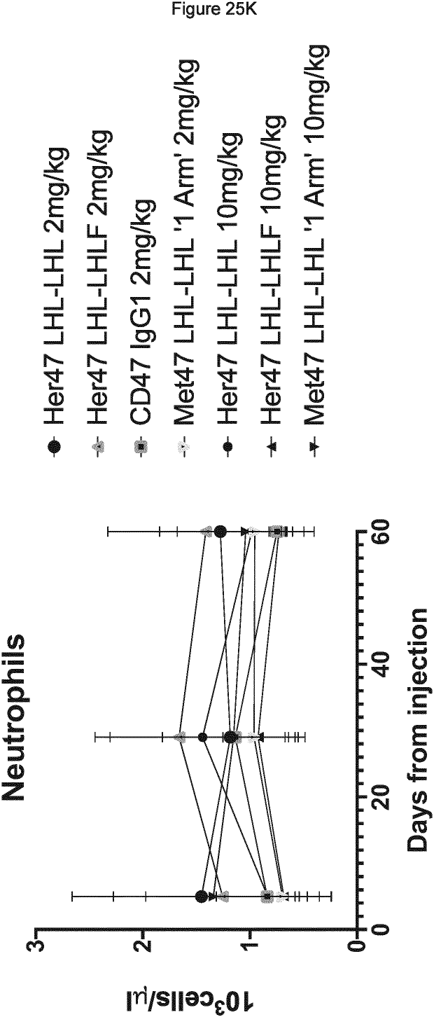

In an in vivo tolerability study, A-D5 IgG1, IgG² Her47 LHL-LHL, IgG² Her47 LHL-LHLF and Fab² Met47 LHL-LHL were each dosed once (intravenously) at concentrations of 2 mg/kg or 10 mg/kg in Tg32 mice. The 2 mg/kg dose of A-D5 IgG was tolerated, while the 10 mg/kg dose was poorly tolerated, causing overt toxicity on day 0 that caused the study in this dosing cohort to be terminated. In contrast, the 2 mg/kg and 10 mg/kg doses of IgG² Her47 LHL-LHL, IgG² Her47 LHL-LHLF and Fab² Met47 LHL-LHL were all well tolerated across 60 days of body weight observation (FIG. 23). While the 2 mg/kg dose of A-D5 IgG was tolerated overall, at 5 days after dosing, reticulocytes were observed to be significantly upregulated in this group (FIG. 24). In contrast, neither the 2 mg/kg nor 10 mg/kg doses of IgG² Her47 LHL-LHL, IgG² Her47 LHL-LHLF nor Fab² Met47 LHL-LHL were associated with reticulocyte upregulation at day 5 or later (FIG. 24, FIG. 25). Reticulocyte upregulation is a known response to rapid erythrocyte clearance, which suggested that IV dosing of the A-D5 IgG1 antibody led to accelerated clearance of CD47-high erythrocytes.

To sample the effects of the dosed proteins more broadly, full haematological panels were examined at days 5, 29 and 60 after dosing (FIG. 25A - FIG. 25K). These analyses demonstrated that the reticulocyte upregulation effect for A-D5 IgG1 was transient, returning to baseline by day 29 (FIG. 25A). No significant effects were observed for A-D5 IgG1, IgG² Her47 LHL-LHL, IgG² Her47 LHL-LHLF and Fab² Met47 LHL-LHL on days 5, 29 or 60 on erythrocyte (RBC) count, haemoglobin, mean corpuscular haemoglobin concentration (MCHC), mean corpuscular volume (MCV), leukocyte, monocyte, lymphocyte, basophil, eosinophil or neutrophil levels (FIG. 25B - FIG. 25K).

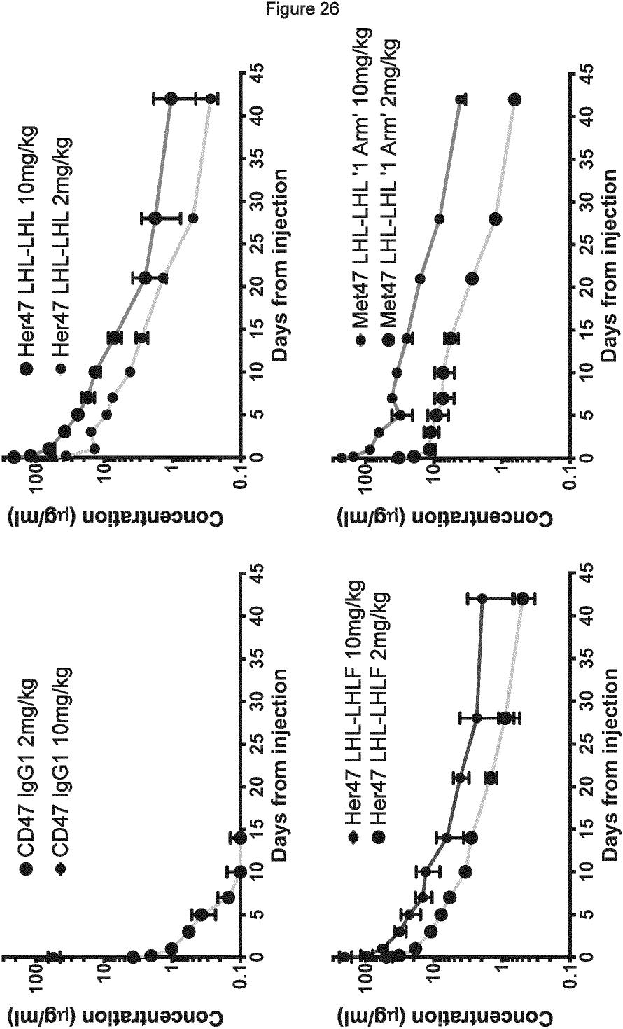

In an in vivo pharmacokinetics study, IgG² Her47 LHL-LHL, IgG² Her47 LHL-LHLF and Fab² Met47 LHL-LHL were each dosed once (intravenously) at concentrations of 2 or 10 mg/kg and A-D5 IgG1 was dosed at the previously-tolerated concentration of 2 mg/kg, in Tg32 mice. Blood samples were collected from each mouse according to the bleeding schedule: 30 min, 4h, 1 day, 3, 5, 7, 10, 14, 21, 28 and 42 days. Analyses of serum antibody concentration demonstrated that A-D5 IgG1 at 2 mg/kg was very rapidly removed from circulation, reaching mean concentrations < 0.5 µg/ml within 5 days (FIG. 26). This rapid drug clearance (known as Tissue-Mediated Drug Disposition, or TMDD) was likely due to the previously-observed strong binding to the mouse erythrocytes, which are then rapidly cleared from the system by phagocytosis. This finding further explains the return of reticulocyte levels to normal by day 29 in A-D5 IgG1 dosing (FIG. 25A), as the molecule has been essentially eliminated by day 10 (FIG. 26).

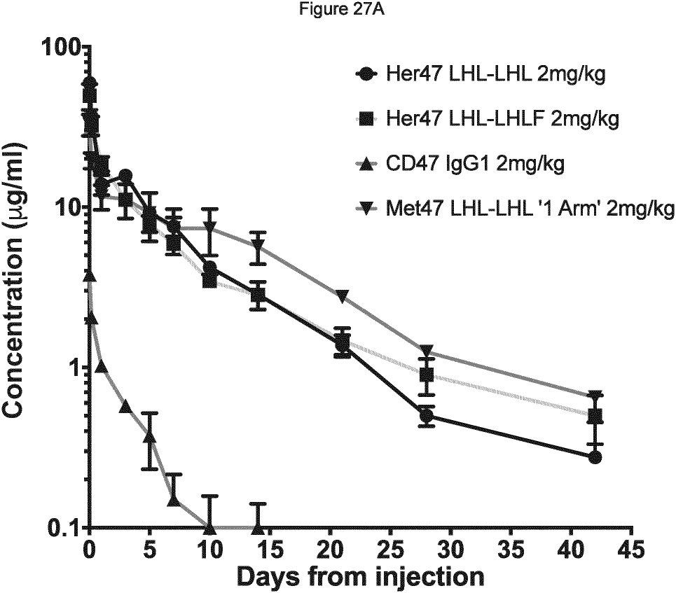

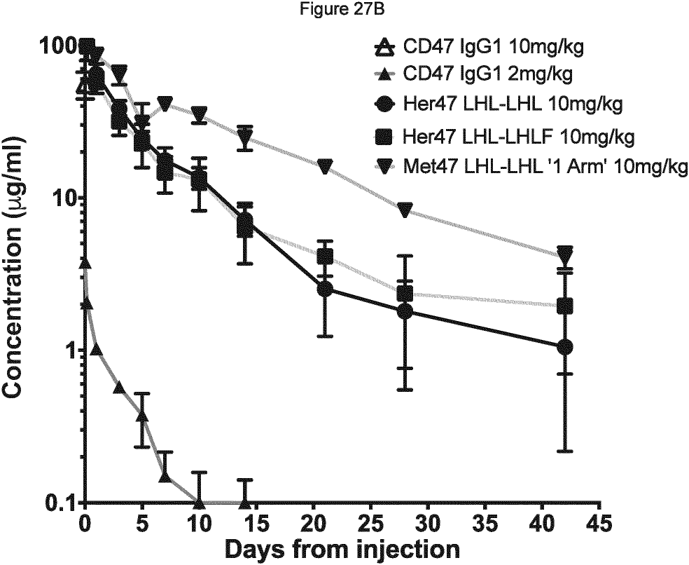

In contrast, IgG² Her47 LHL-LHL, IgG² Her47 LHL-LHLF and Fab² Met47 LHL-LHL all exhibited slow clearance at both 2 and 10 mg/kg doses (FIG. 26). Importantly, the 2 mg/kg doses of each protein took > 25 days to achieve concentrations < 1.0 µg/ml (FIG. 27A) and the 10 mg/kg doses of each protein maintained concentrations > 1.0 µg/ml at 42 days (FIG. 27B). Single samples taken from the mice dosed with 10 mg/kg of A-D5 IgG1 during the tolerance study on day 0 were also analysed, showing that the maximal serum IgG concentrations achieved (but not tolerated) were similar to those achieved for the IgG² and Fab² proteins that were fully tolerated (> 50 µg/ml).

IgG² Her47 LHL-LHL, IgG² Her47 LHL-LHLF and Fab² Met47 LHL-LHL at 2 mg/kg all showed normal ‘Alpha’ phase (when IV doses of immunoglobulins quickly distribute from the bloodstream into tissue), followed by a long ‘Beta’ phase of antibody circulating in the serum (FIGS. 26, 27A). These proteins also exhibited normal distribution profiles at 10 mg/kg, but with even longer circulation (FIGS. 26, 27B). Importantly, the Beta phases of each protein, at both concentrations, exhibited linearity and parallel curves, suggesting that the CD47 domains of these proteins did not lead to the TMDD observed for A-D5 IgG1 (FIG. 26). TMDD would have been expected to show up strongly if the IgG² Her47 LHL-LHL, IgG² Her47 LHL-LHLF or Fab² Met47 LHL-LHL proteins were undergoing activation in the periphery, as activation would lead to high affinity erythrocyte, endothelium and platelet binding, leading to clearance that would change the Beta phase into a sharp downward trajectory, as seen for the A-D5 IgG1 (FIG. 26). These observations, coupled with lack of reticulocyte amplification in the doses of IgG² Her47 LHL-LHL, IgG² Her47 LHL-LHLF and Fab² Met47 LHL-LHL, suggests that peripheral activation levels are low. This is despite the long pharmacokinetics for these molecules meaning that they have been through FcRn recycling multiple times over the >25 day circulation.

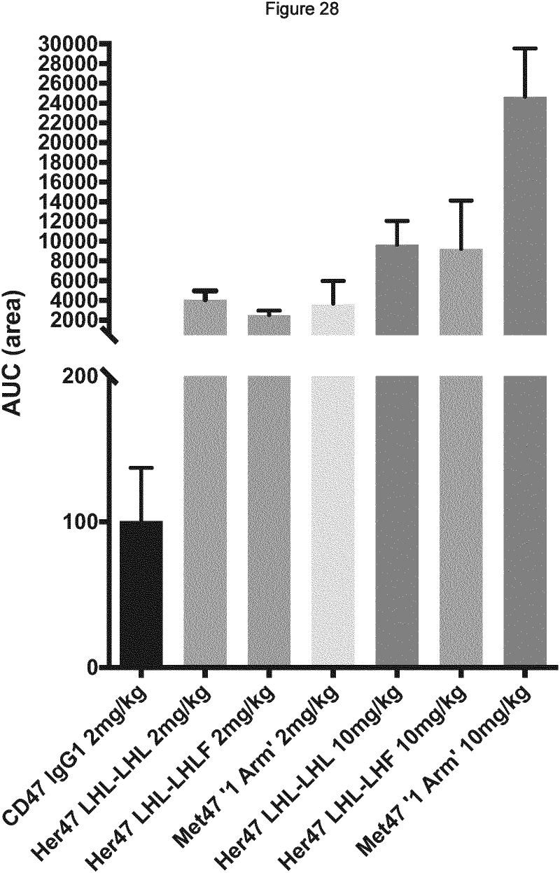

The pharmacokinetic and tolerability findings outlined above for IgG² Her47 LHL-LHL, IgG² Her47 LHL-LHLF and Fab² Met47 LHL-LHL are indicative of normal, FcRn-mediated, antibody-like, half-life extension via the human IgG1 Fc domain. This effect resulted in significant increases in Area Under the Curve (AUC) values for these 3 proteins in comparison to A-D5 IgG1 (FIG. 28). At 2 mg/kg, AUC was improved 25-40 fold for IgG² Her47 LHL-LHL, IgG² Her47 LHL-LHLF and Fab² Met47 LHL-LHL over A-D5 IgG1. At 10 mg/kg, which is not a safely-achievable dose for A-D5 IgG1, AUC was improved by approximately 100-250 fold for IgG² Her47 LHL-LHL, IgG² Her47 LHL-LHLF and Fab² Met47 LHL-LHL over A-D5 IgG1 (FIG. 28). These improvements in AUC are significant as they suggest the use of CD47 binding domains within the IgG² and Fab² structure may be used at high concentration from the first dose, maximising distribution towards tumour tissue.

### Flow Cytometry Analyses of Binding to Monkey and Human Erythrocytes

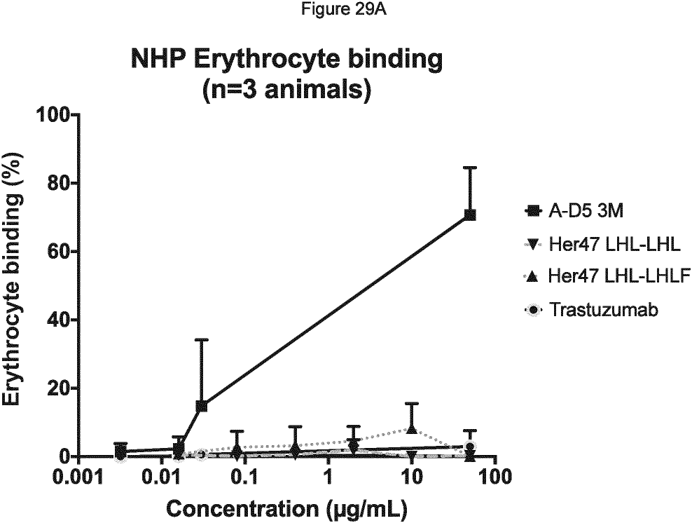

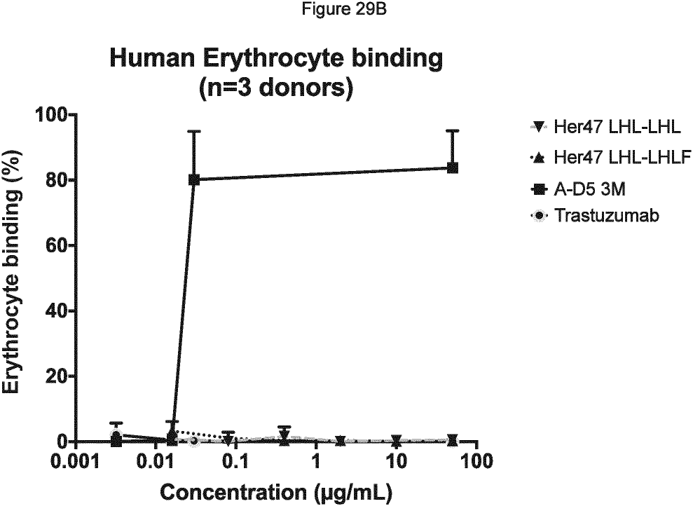

Erythrocytes were isolated from 3 cyno NHP (monkeys) and 3 human donors and stained with A-D5 IgG1, Trastuzumab, IgG² Her47 LHL-LHLF or IgG² Her47 LHL-LHL. Both sets of analyses showed that only A-D5 IgG1 exhibited concentration-dependent binding to either cyno (FIG. 29A), or human (FIG. 29B) erythrocytes. These findings confirmed the above observations that the CD47 binding domains within the IgG² (and, therefore, Fab²) structure are restricted from binding mouse, monkey and human CD47.

### Activation by MMPs and Cathepsins at pH6.0 and 7.4

MMPs and Cathepsins have been shown to have the potential to enzymatically cleave the peptide sequences found in either the LHL or LHLF linkers outlined above. Importantly, however, both of these classes of enzyme can exhibit sensitivity to changes in pH conditions that raise or lowering their enzymatic activity. This can be of critical importance as the pH of solid tumours is frequently observed to deviate away from the normal physiological pH in man, pH7.4. In particular, solid tumours (and highly inflamed tissues) may develop acidic pH conditions as low as pH6.0.

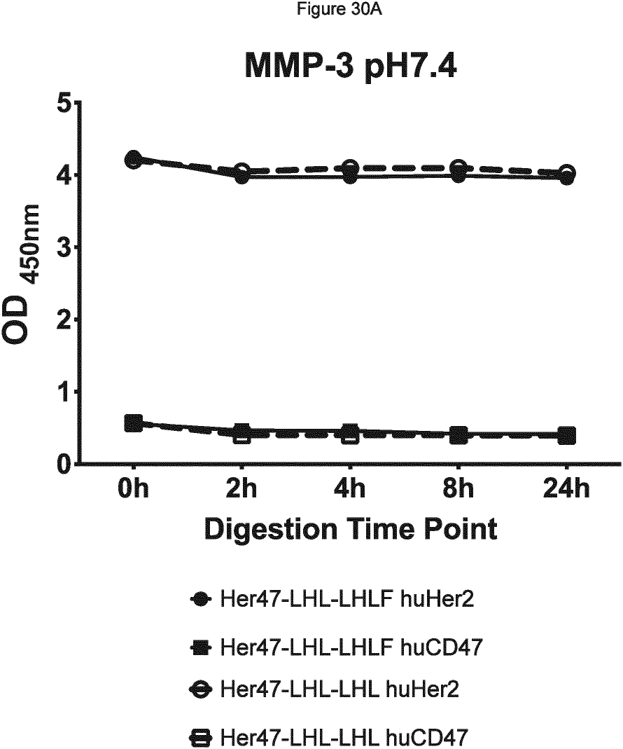

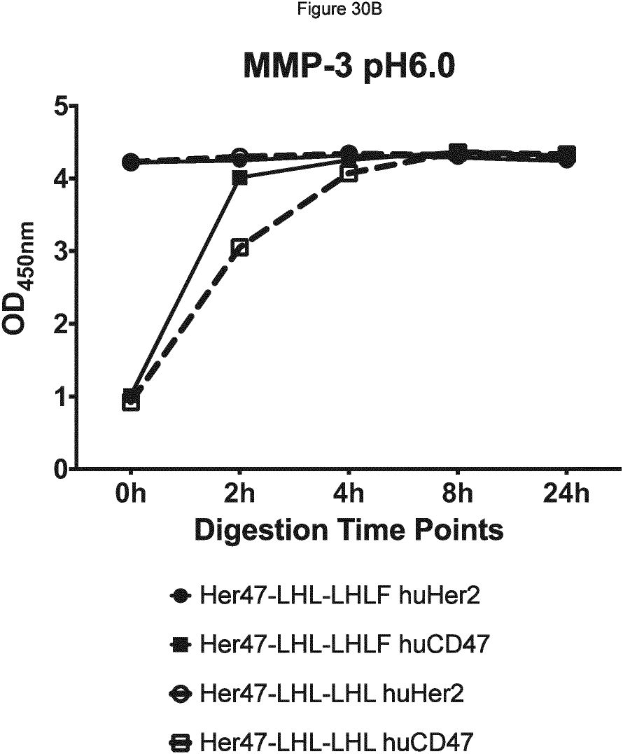

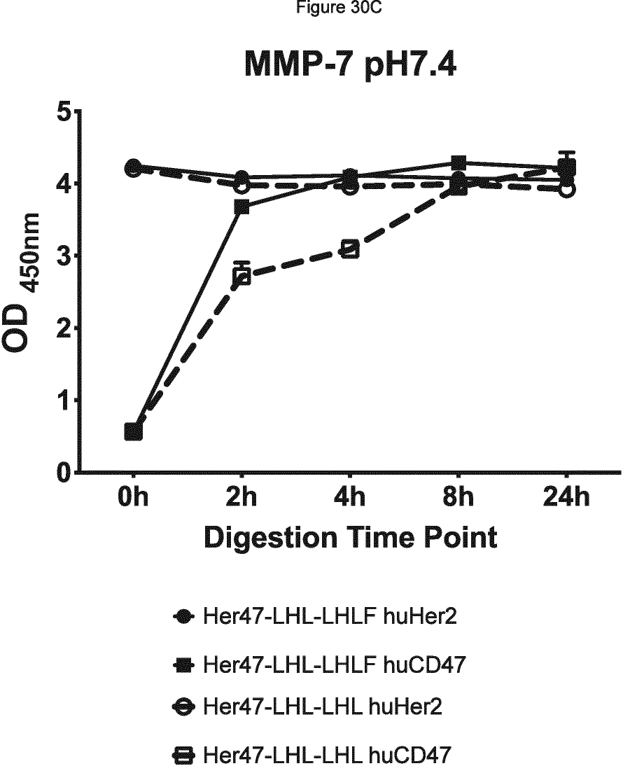

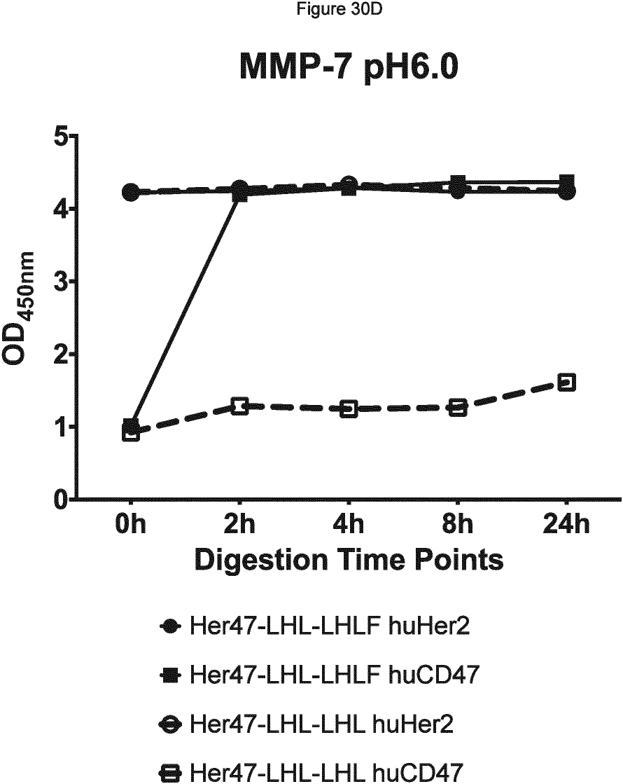

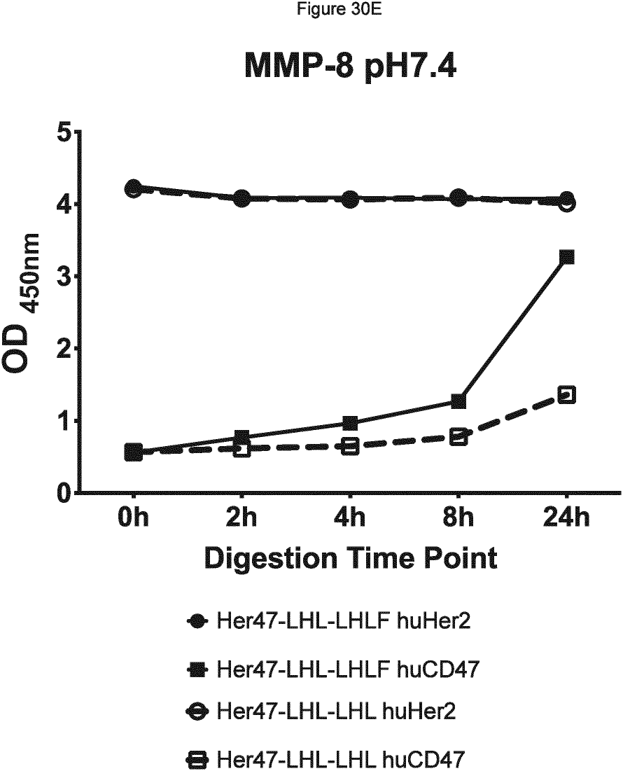

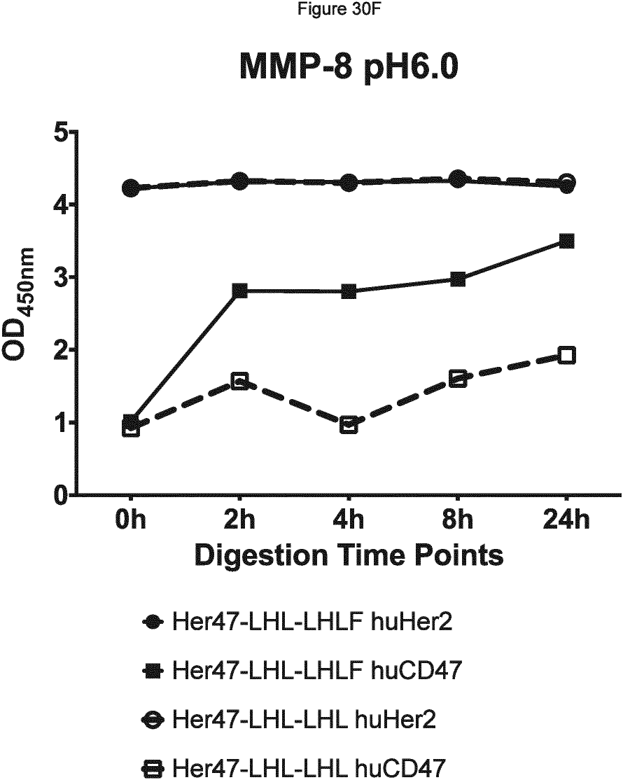

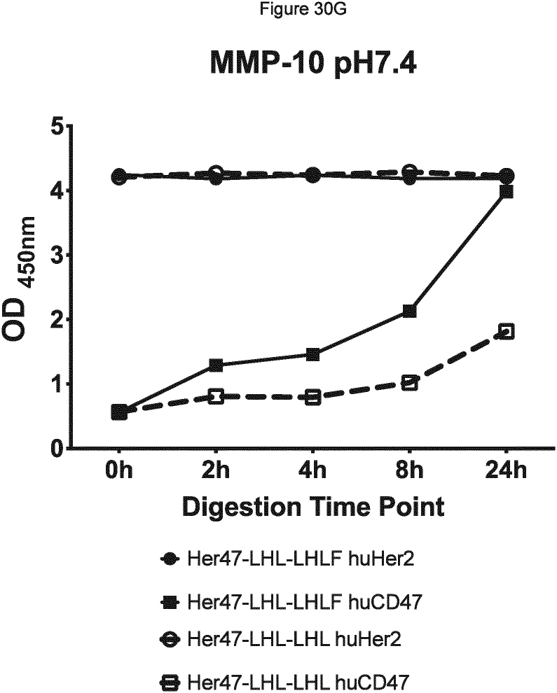

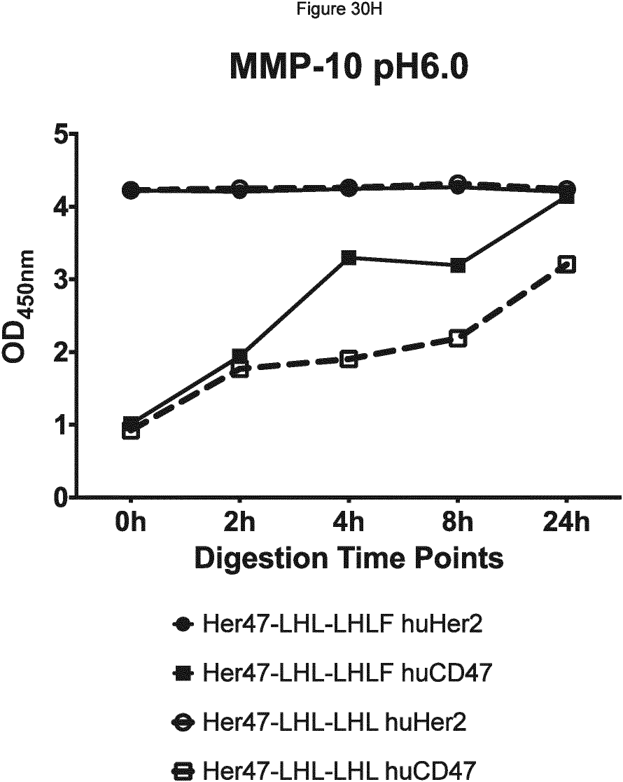

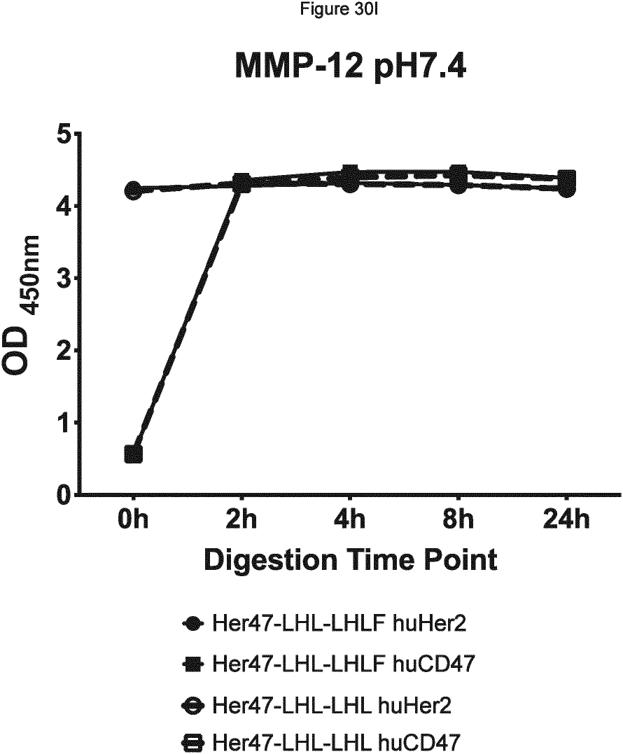

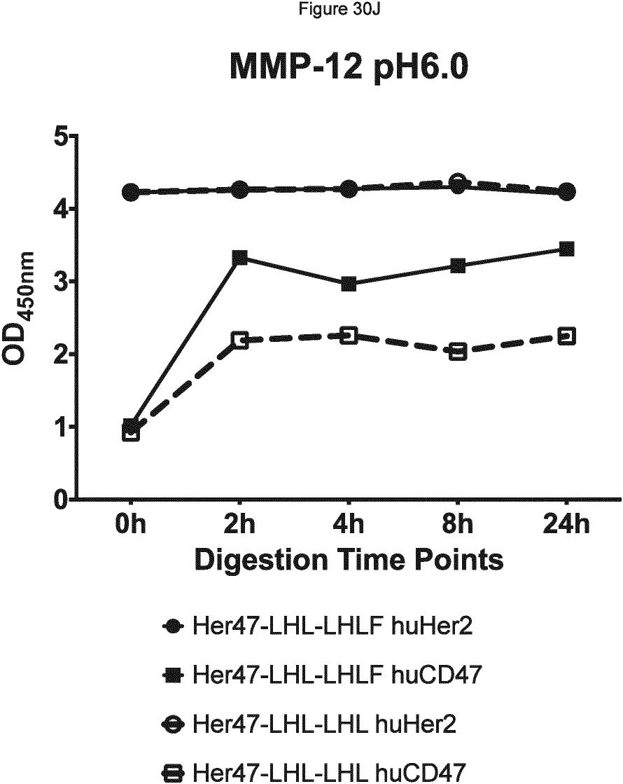

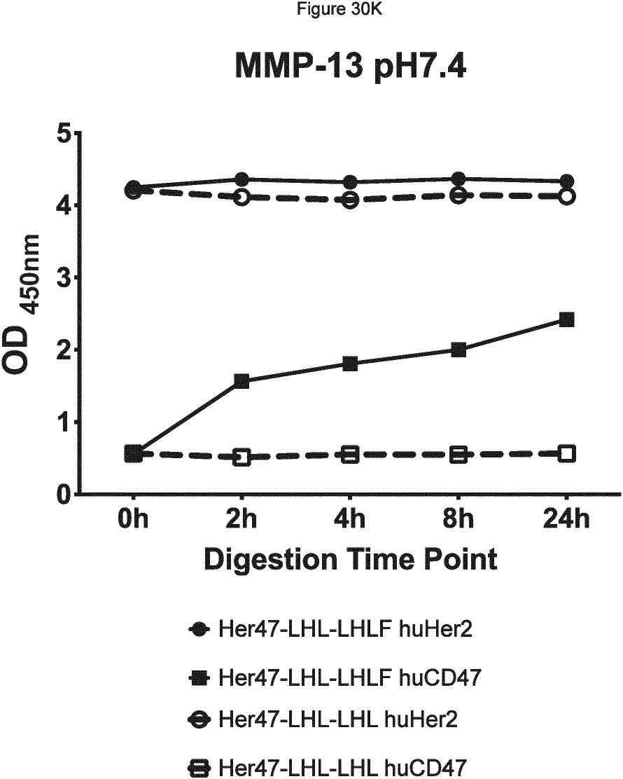

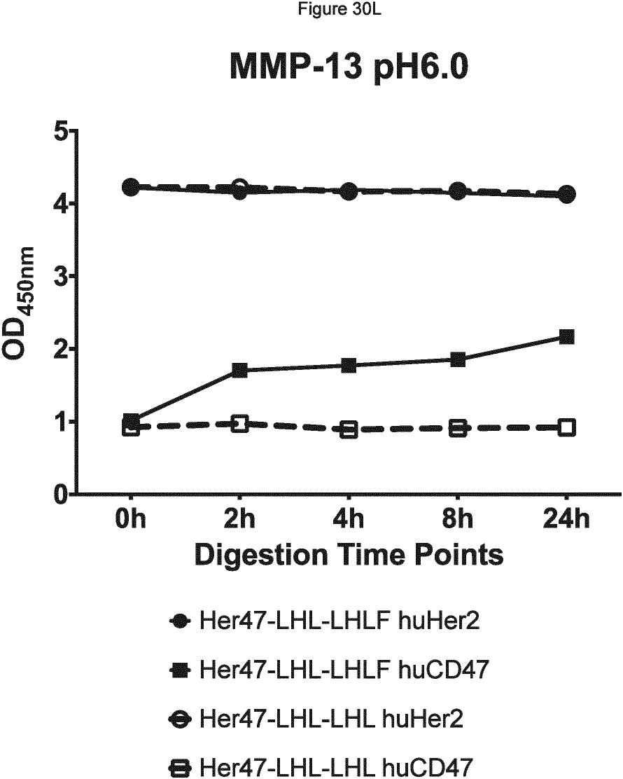

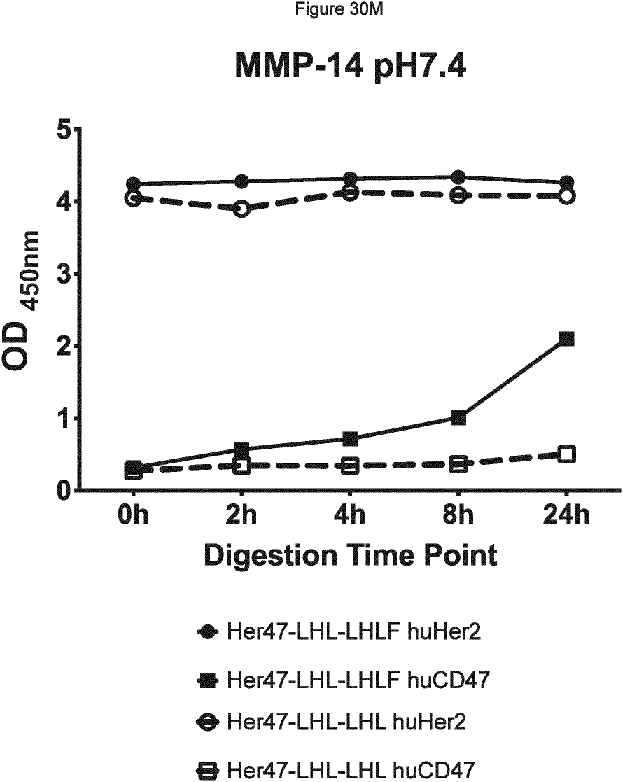

Having proven above that multiple MMP enzymes have the ability to activate lower fab binding at pH7.4, we examined the activity of multiple MMP and Cathepsin enzymes (that are all associated with elevated activity in solid tumours) at both pH 6.0 and 7.4. Activation of IgG² Her47 LHL-LHL or IgG² Her47 LHL-LHLF were examined for MMP3 (FIGS. 30A, B), MMP7 (FIGS. 30C, D), MMP8 (FIGS. 4Fig. 30E, F), MMP10 (FIGS. 30G, H), MMP12 (FIGS. 30I, J), MMP13 (FIGS. 30K, L) and MMP14 (FIGS. 30M, N). MMP-treated samples for IgG² Her47 LHL-LHL and IgG² Her47 LHL-LHLF were then applied in direct binding to both human Her2 and human CD47. These analyses demonstrated that both proteins exhibited measurable activation of CD47 binding by MMPs 8, 10 and 12 at both pH7.4 and pH6.0. In contrast, only IgG² Her47 LHL-LHLF was activated by MMP13 at both pH7.4 and pH6.0 (FIGS. 30K, L), and MMP7 at pH6.0 (FIG. 30D). IgG² Her47 LHL-LHLF also exhibited a relatively higher level of activation, at the majority of time points, than IgG² Her47 LHL-LHL by the majority of MMPs tested (FIGS. 30A-N). These findings confirmed that the inclusion of the LHLF linker leads to more rapid activation of binding by the lower Fab, by a broader range of MMP enzymes, at both pH6.0 and/or pH7.4.

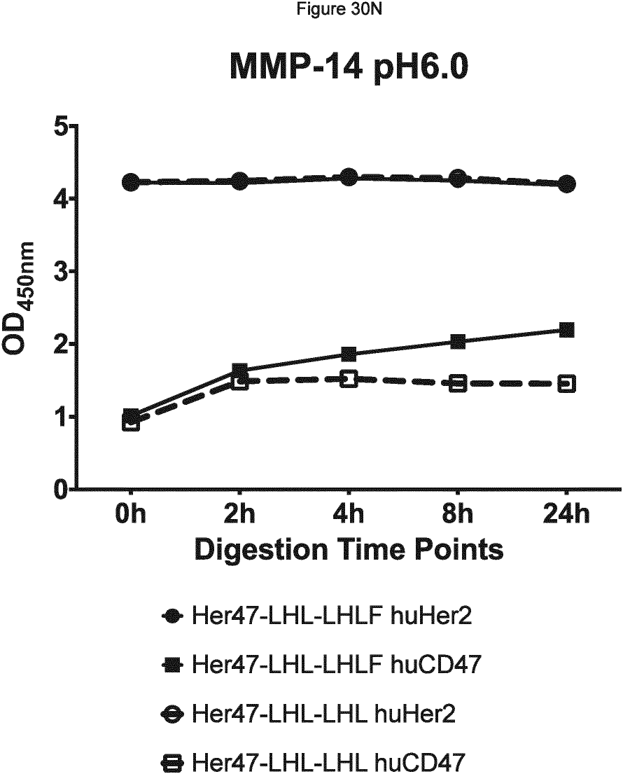

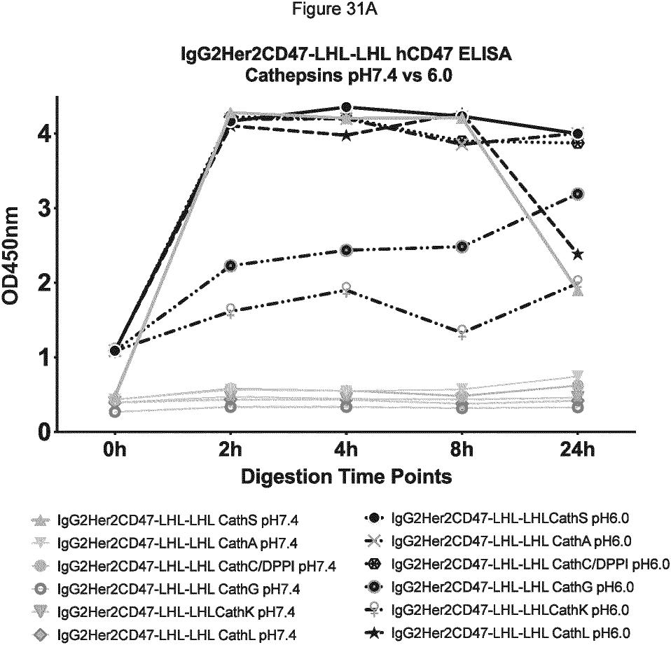

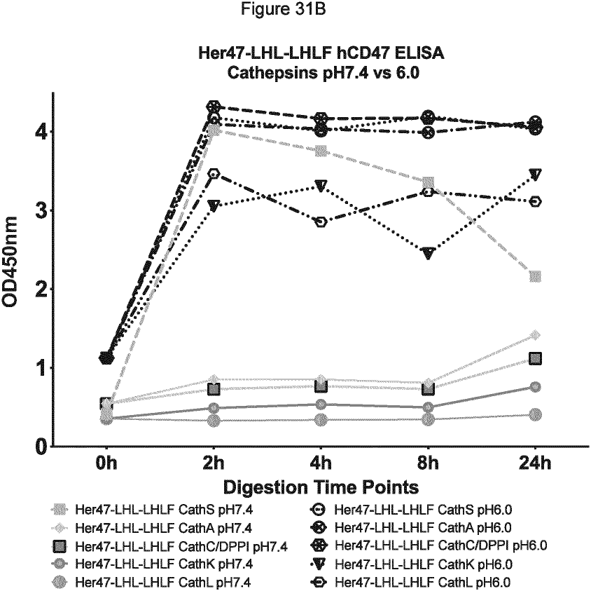

Cathepsins were also examined as potential activation enzymes. For these enzymes, a clear relationship between pH and activity was observed. Activation of IgG² Her47 LHL-LHL (FIG. 31A) or IgG² Her47 LHL-LHLF (FIG. 31B) both demonstrated that only Cathepsin S was capable of activating CD47 binding at both pH7.4 and 6.0. In contrast, treatment with Cathepsins A, C, G, K and L showed little or no activation of CD47 binding at pH7.4 even after 24 hours, but strong activation signals were rapidly generated at pH6.0 (FIGS. 31A, 31B). These findings suggested that the LHL and LHLF linkers could allow accelerated activation in acidified tissues in comparison to those at pH7.4, via more rapid activation by MMPs and by pH-selective activation by a series of Cathepsins. This may further improve the therapeutic index of IgG² and Fab² proteins by minimising the activation of lower Fab binding at pH7.4 (pH of non-diseased tissue, where active extracellular MMP and Cathepsin levels are low) and maximising it at pH6.0 (pH of diseased tissue, where MMP and Cathepsin levels are high and Cathepsin activity is potentiated).

### Activation of Lower Fab Binding to Her2/CD47+ Cells

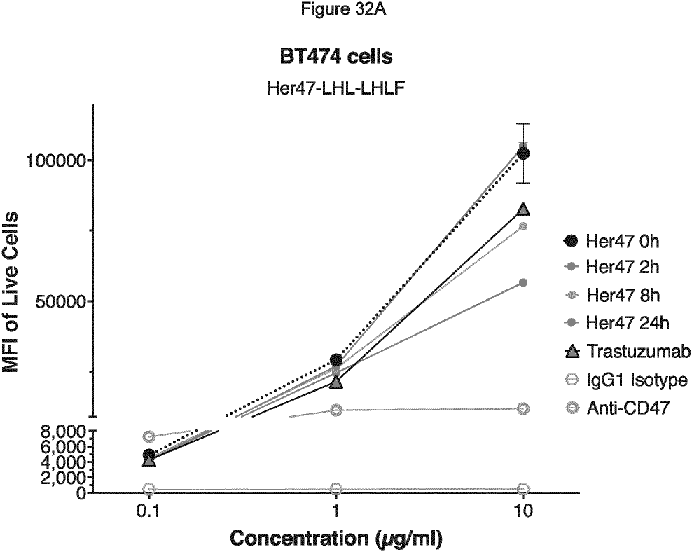

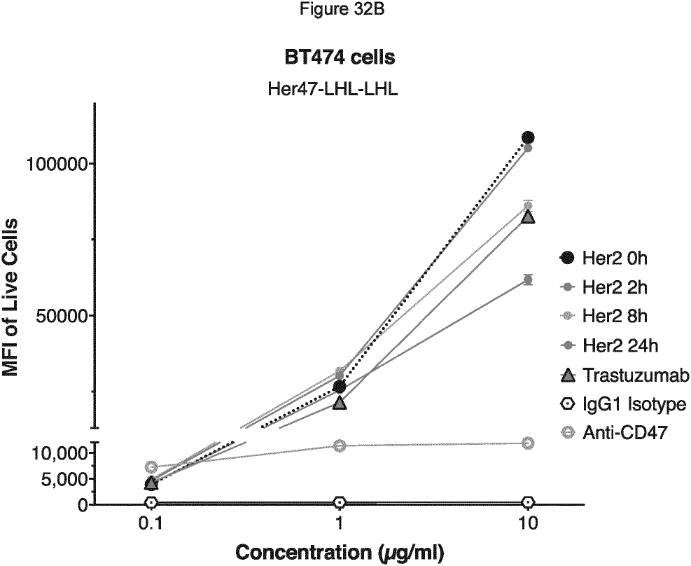

Flow cytometry was performed to examine the binding characteristics of IgG² Her47 LHL-LHL and IgG² Her47 LHL-LHLF to cells expressing different levels of CD47 and Her2 on their cell surface. Staining with Trastuzumab, anti-CD47 and Isotype control IgGs demonstrated that BT474 cells expressed high levels of Her2 and lower levels of CD47 (FIGS. 32A, B), whereas MCF7 cells expressed higher levels of CD47 and low levels of Her2 (FIGS. 32C, D). Staining both cell types with IgG² Her47 LHL-LHLF (FIGS. 32A, C) and IgG² Her47 LHL-LHL (FIGS. 32B, D) after activation with MMP12 for 0, 2, 8 and 24 h, was also performed. Both IgG² Her47 LHL-LHLF and IgG² Her47 LHL-LHL exhibited binding profiles similar to Trastuzumab on BT474 cells at 0, 2 and 8h time points, but slightly reduced binding after 24 h (FIGS. 32A, B). In contrast, IgG² Her47 LHL-LHLF and IgG² Her47 LHL-LHL both exhibited low level binding profiles similar to Trastuzumab on MCF7 cells at 0 h of activation, but significantly higher binding at 2, 8 and 24 h activation time points, mimicking the anti-CD47 control (FIGS. 32C, D). These findings experimentally confirmed that the activation model proposed in FIG. 2B is valid, with Trastuzumab-like binding of Her2 being maintained after activation and CD47 binding profile only becoming highly active after activation. Importantly, these findings also suggested that IgG² (and Fab²) proteins may have the beneficial ability to drive the function of the lower fab binding domains (e.g. CD47) to cells expressing low levels of the upper Fab target (e.g. Her2).

### Analysis of MMP12 Activation by SDS-PAGE and Mass Spectrometry

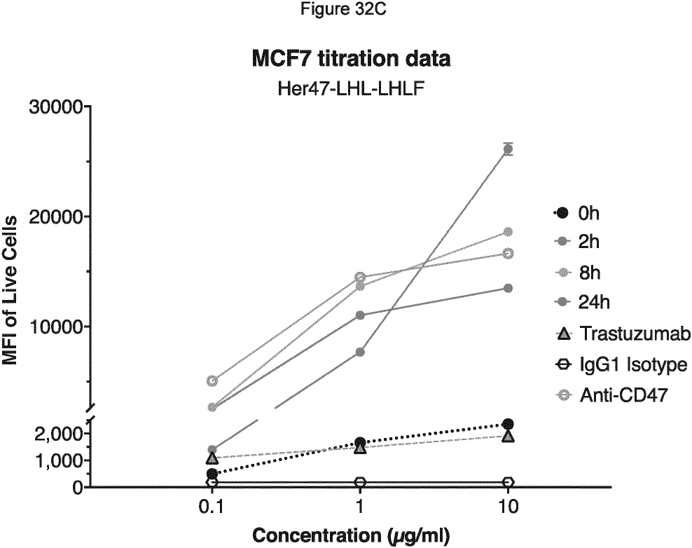

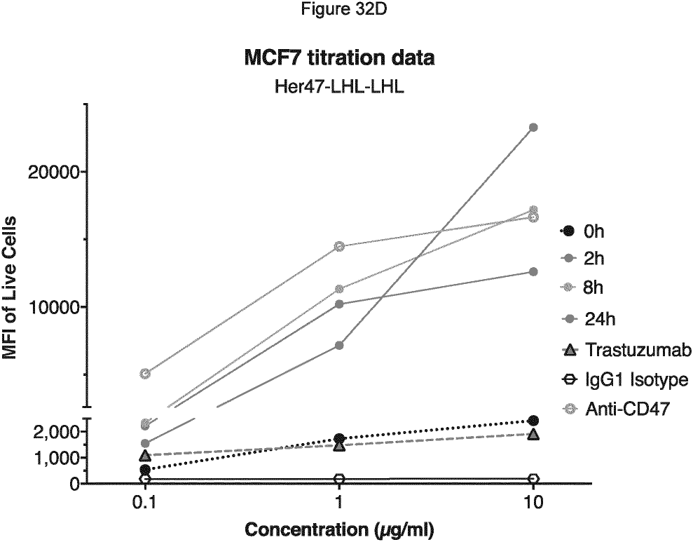

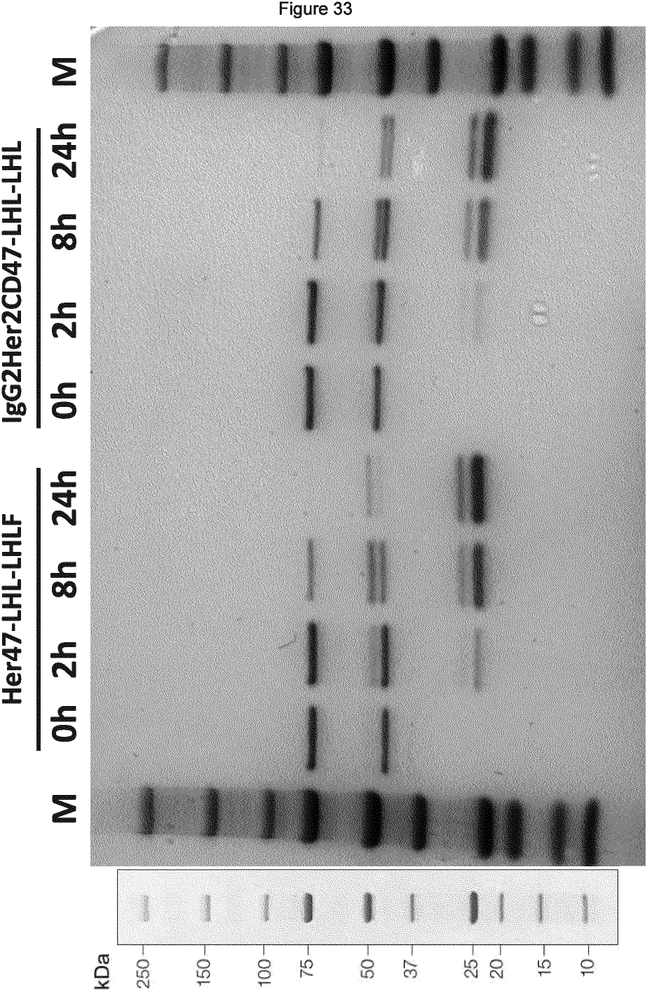

IgG² Her47 LHL-LHLF and IgG² Her47 LHL-LHL proteins were treated with MMP12 as above, over 0, 2, 8 and 24 h. These protein samples were then analysed by SDS-PAGE (FIG. 33). This analysis generated findings that correlate clearly with observations of activation ELISAs (FIGS. 15A-15F, 7A-7C, 30A-30N) and flow cytometry (FIG. 32): At 0 h -two intact chains were observed with the heavy chain running at 75 kDa and light chain running just below the 50 kDa marker (sub-50 kDa). At the 2 h time point, for both IgG² Her47 LHL-LHLF and IgG² Her47 LHL-LHL, a new ~25 kDa product was observed, plus faint bands that are slightly larger than 25 kDa and at ~50 kDa (above the intact light chain). The molecular weights of these new fragments correspond to upper Fab Fd or light chain fragments (1 v domain + 1 c domain = ~25 kDa), Fc (2 x c domains + 1 n-link glycosylation = 28-30 kDa), and intact Fd-hinge-Fc (standard IgG1 heavy chain = 50 kDa), respectively. At 8 and 24 h time points, a progressive reduction in intact chains and increase in fragments that were approximately 25 kDa smaller than the intact chains, was observed. In particular, at 8h the 25 kDa (upper fab chain) product became predominant, with a significant (but reduced compared to the 0 h sample) amount of both the intact heavy chain (75 kDa) and intact light chain (sub-50 kDa) remaining. This SDS-PAGE analysis (FIG. 33) therefore demonstrated that both linkers between the upper and lower Fabs are actively cleaved by the MMP12 enzyme, whether of the LHL or LHLF sequence.

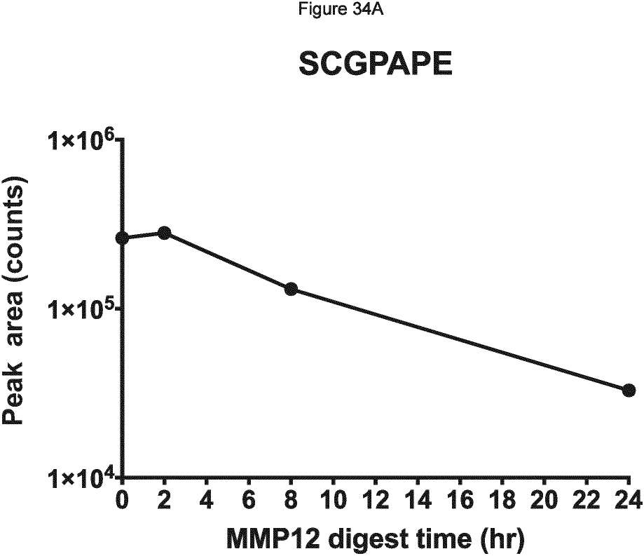

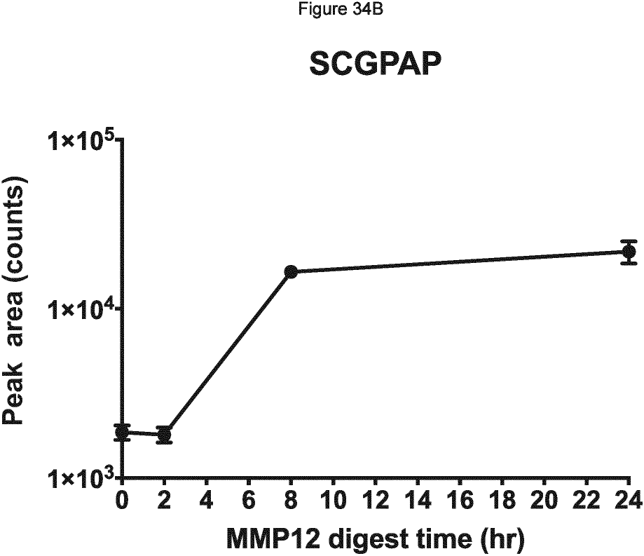

To examine precisely where in the Fab² structure the MMP12 enzyme was activating the IgG² molecules, mass spectrometry was carried out using 0, 2, 8 and 24 h samples from IgG² Her47 LHL-LHL. Peptide samples were prepared by reduction, alkylation and proteolytic digestion using a combination of trypsin and Glu-C. These samples were analysed by LC-MS/MS. Efficiency of cleavage by MMP was determined by comparing the MS responses of peptide derived from the intact MMP-cleavage site (SCGPAPE (SEQ ID NO: 110)) to those from cleaved protein (SCGPAP (SEQ ID NO: 111)). This analysis successfully identified peptides that mapped to both the uncleaved (SCGPAPE (SEQ ID NO: 110)) and cleaved (SCGPAP (SEQ ID NO: 111)) linkers, proving that in the LHL linker, MMP12 cleaves between the second proline (P) and the glutamic acid (E). Relative signal for the uncleaved (SCGPAPE (SEQ ID NO: 110)) peptide progressively reduced from 0 to 8 to 24 h (FIG. 34A), whereas signal for the cleaved (SCGPAP (SEQ ID NO: 111)) peptide progressively increased (FIG. 34B).

### Generation and Analysis of IgG² in Hinge-Stabilized ‘IgG1-DAA’ Form

US Patent No. 8871204B2 teaches protease-resistant IgG1 antibody variants which maintained lower hinge and Fc stability in the presence of MMP enzymes. In these mutated IgG1 antibodies, the sequence of E233-L234-L235-G236 (SEQ ID NO: 112) in the hinge is replaced with P233-V234-A235 (with G236 deleted); and the CH2 domain comprises at least one substitution selected from S239D/1332E, K326A/E333A, H268F/S324T/1332E, F243L/R292P/Y300L, S239D/H268F/S324T/1332E, S267E/H268F/S324T/1332E, K326A/1332E/E333A, S239D/K326A/E333A, S267E/I332E and G237X/S239D/1332E where X is A, D, P, Q or S; wherein amino acid residues are numbered according to EU numbering.

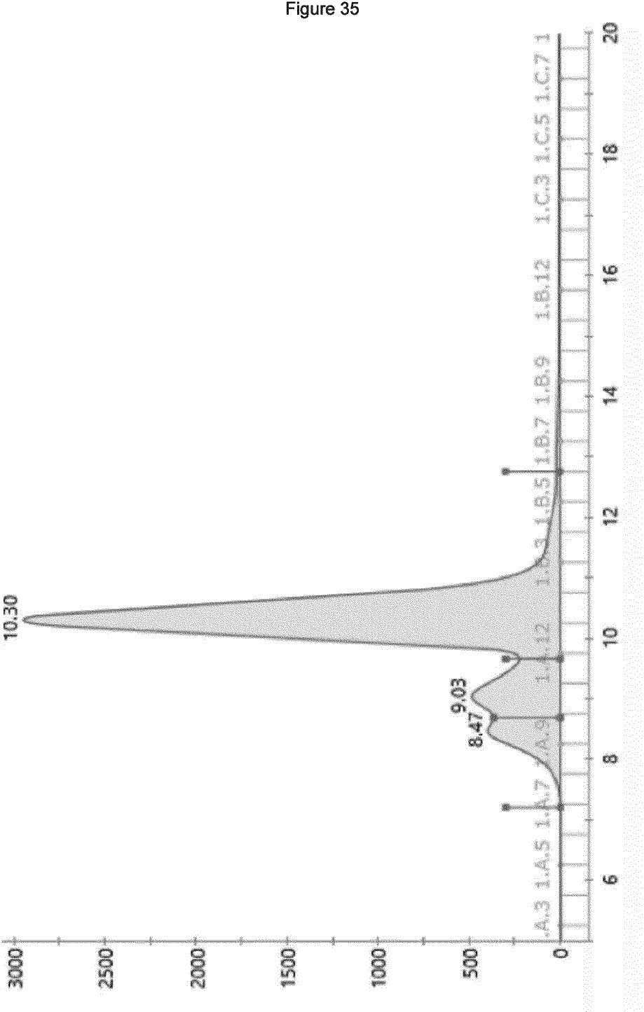

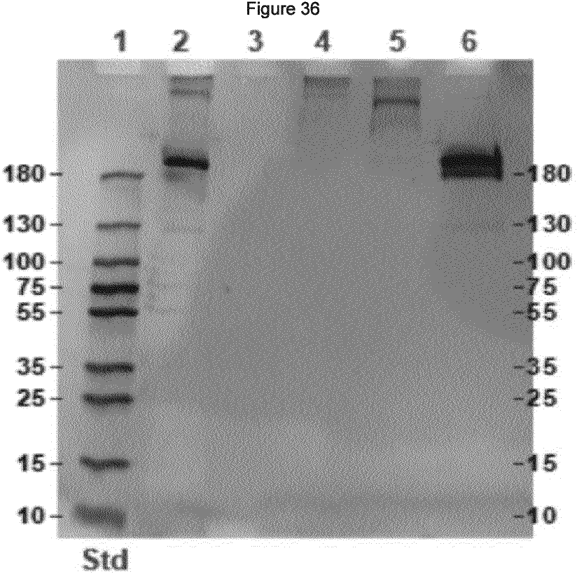

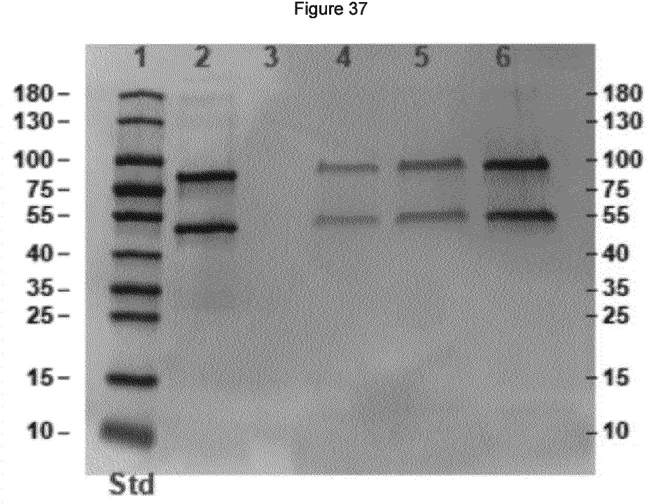

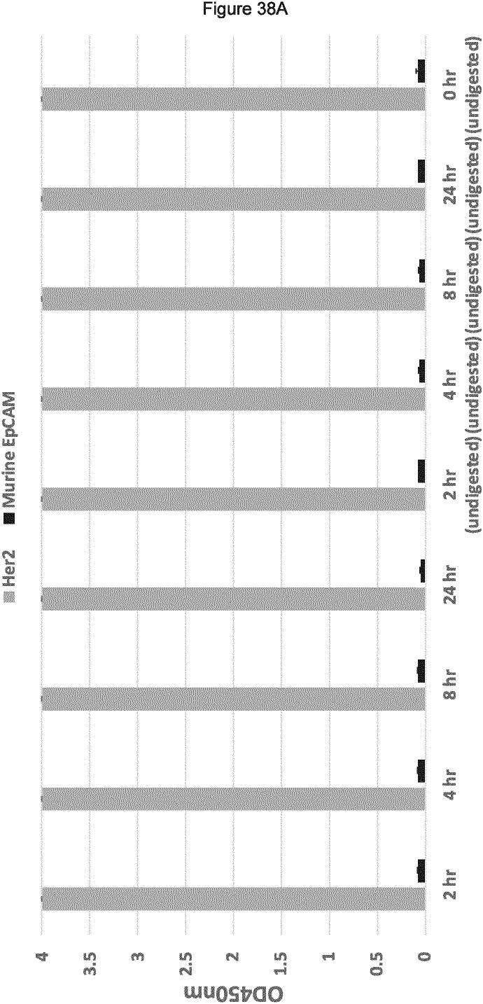

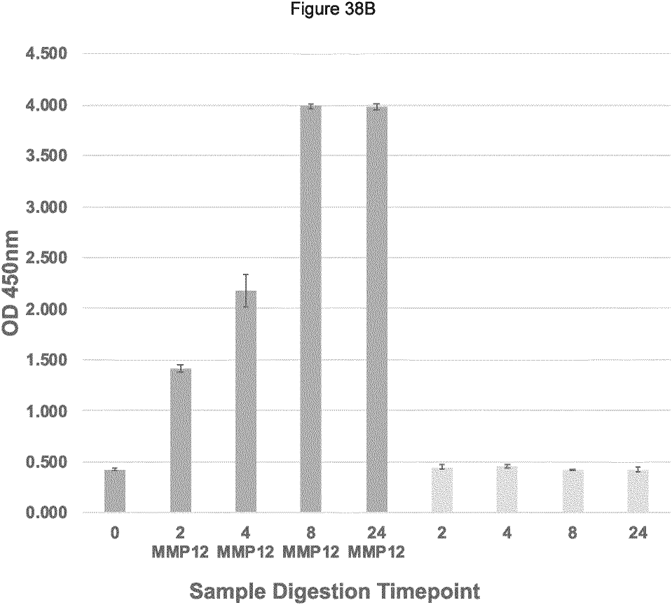

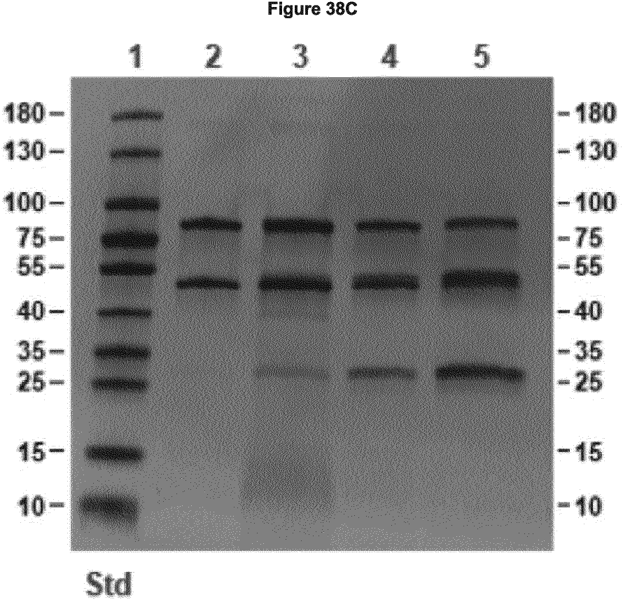

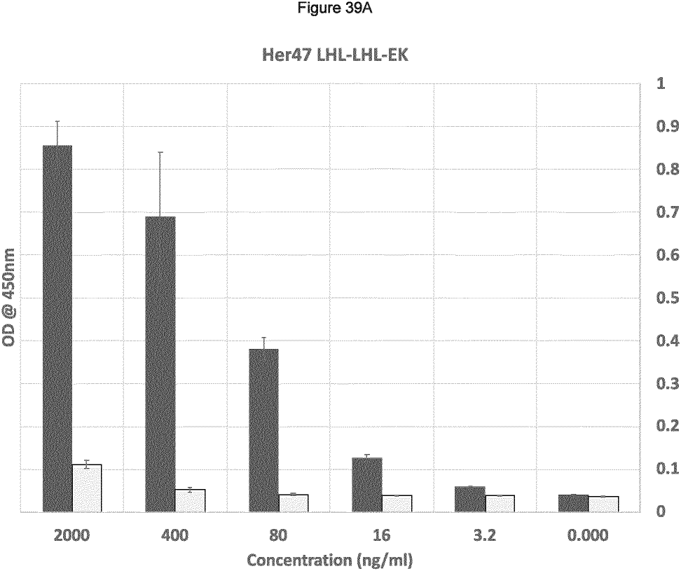

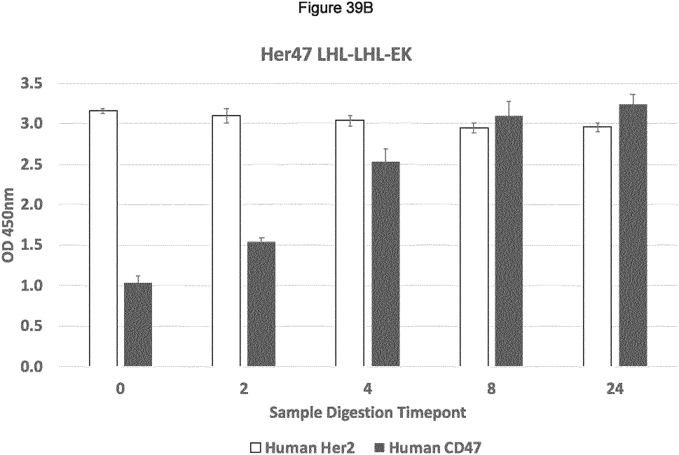

To examine the use of the Fab² structure in the context of such a protease-stabilised IgG1 Fc, two example constructs were generated (Table 19). These two constructs (Her47 LHLF-LHL IgG1-2hDAA and Her47 LHL-LHLF IgG1-2hDAA) placed the Her47 Fab² structure in an IgG² built on the ‘2hDAA’ structure (IgG1 containing P233-V234-A235-ΔG236 and S239D/K326A/E333A mutations). Both constructs were readily expressed in transient CHO cell transfections, with Protein A purified protein exhibiting > 80% product of expected molecular weight. Example analytical SEC data for Her47 LHLF-LHL IgG1-2hDAA shows 80% product at 10.30 ml (FIG. 35). SDS-PAGE analysis of unreduced Protein A purified protein and SEC peaks 8.47, 9.03 and 10.30 ml (FIG. 36) demonstrated that peaks 8.47 and 9.03 contained higher molecular weight aggregates, while peak 10.30 contained product of the expected size (approx. 250 kDa). SDS-PAGE analysis of reduced Protein A purified protein and SEC peaks 8.47, 9.03 and 10.30 ml (FIG. 37) demonstrated that all peaks 8.47, 9.03 and 10.30 ml contained heavy and light chain products of the expected size (approx. 80 and 50 kDa, respectively). Intact monomer (250 kDa) product for Her47 LHL-LHLF IgG1-2hDAA was purified by SEC and submitted to enzymatic digestion at pH7.4 using human MMP12, over a time course of 2, 4, 8 and 24 hours incubation, plus a 24 h incubation in buffer without enzyme as a negative control (time 0, 2, 4, 8, 24 h incubation). In ELISA analysis, all samples exhibited strong binding signal to human Her2, but no measurable binding to control protein murine EpCAM (FIG. 38A). In CD47 binding ELISA, binding signals were increased after 2, 4, 8 and 24 h incubations at 37° C. in the presence or absence of MMP12 enzyme, but no signal above background was observed at 0, 2, 4, 8, or 24 hours without MMP12 (FIG. 38B).

Analysis of 0 hr (undigested), and 2, 8 and 24 h MMP12-digested samples By SDS-PAGE confirmed that the purified protein contained the expected-size heavy and light chains. Over 2, 8 and 24 hr MMP12 digest, a 25 kDa band was generated (FIG. 38C), indicating linker peptide cleavage, but demonstrably less breakdown in heavy chain was observed in comparison with IgG² Her47 LHL-LHL or IgG² Her47 LHL-LHLF (FIG. 33), indicating that the ‘2hDAA’ mutations do indeed stabilise the Fc region to resist MMP12 digest.

### Generation and Analysis of IgG² with alternative enzyme activation

As the LHL and LHLF linkers were shown above to be susceptible to cleavage by multiple MMP and Cathepsin enzymes, six Fab² construct types were examined in IgG² format (Table 20). These constructs used a series of linker designs (Table 21) that were designed to be susceptible to cleavage by enzymes of several classes that are associated with upregulated activity in solid tumours and highly inflamed tissues, such as: enterokinase (EK), thrombin (Thr), tPA, Granzyme B (GrB), uPA and ADAMTs-5 (A5). All 6 constructs were readily expressed by CHO cells and purified by ProA chromatography.

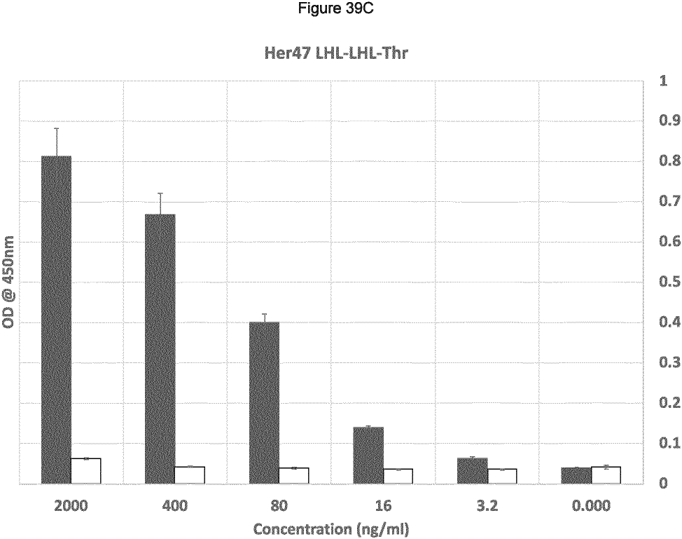

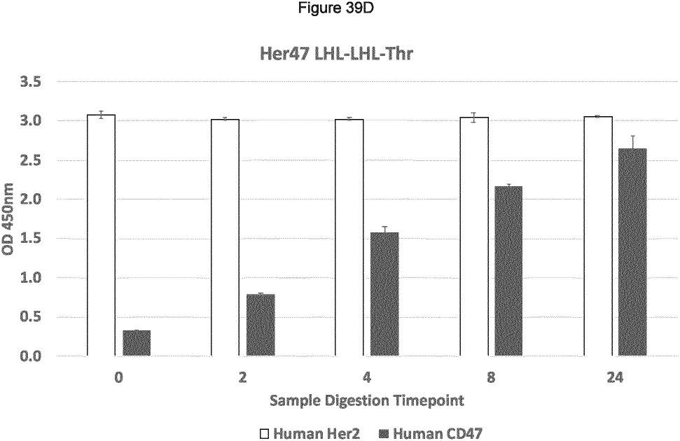

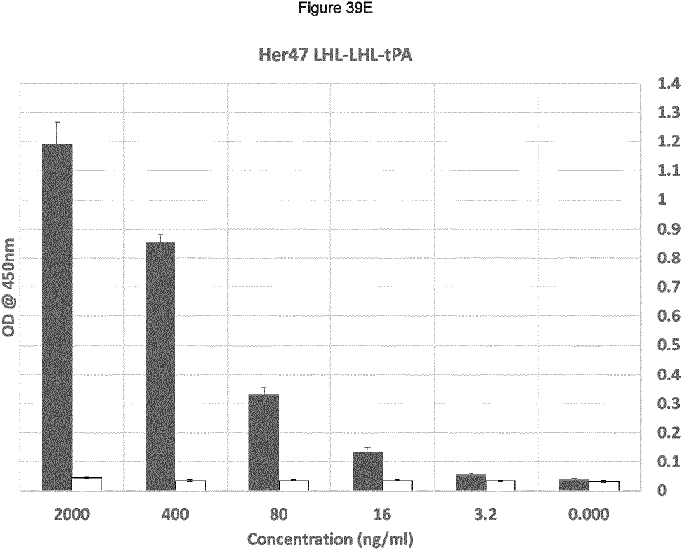

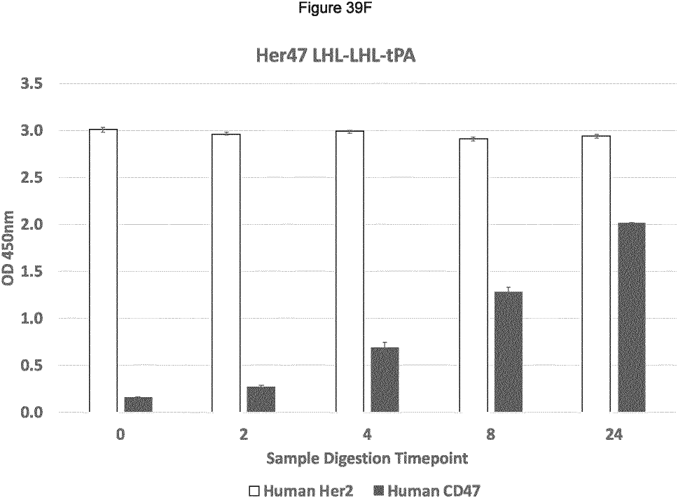

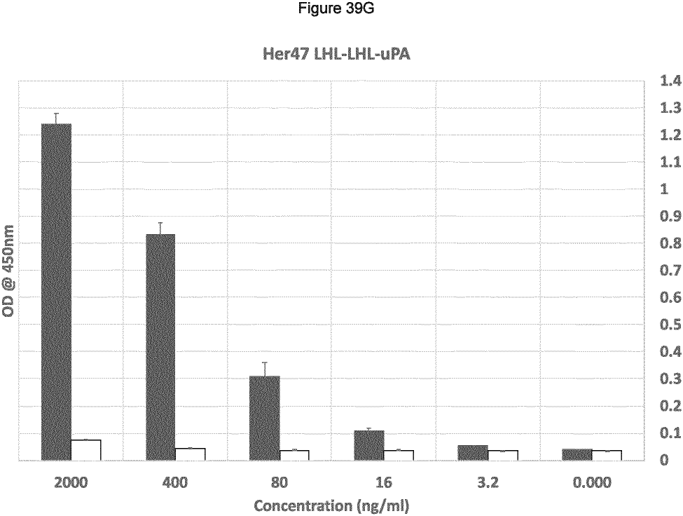

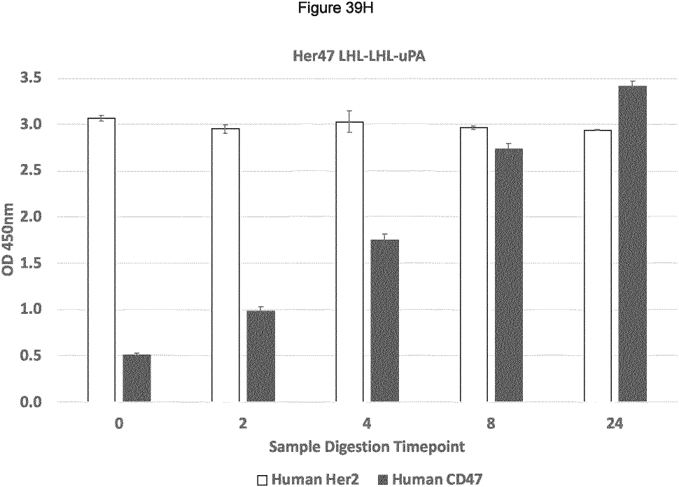

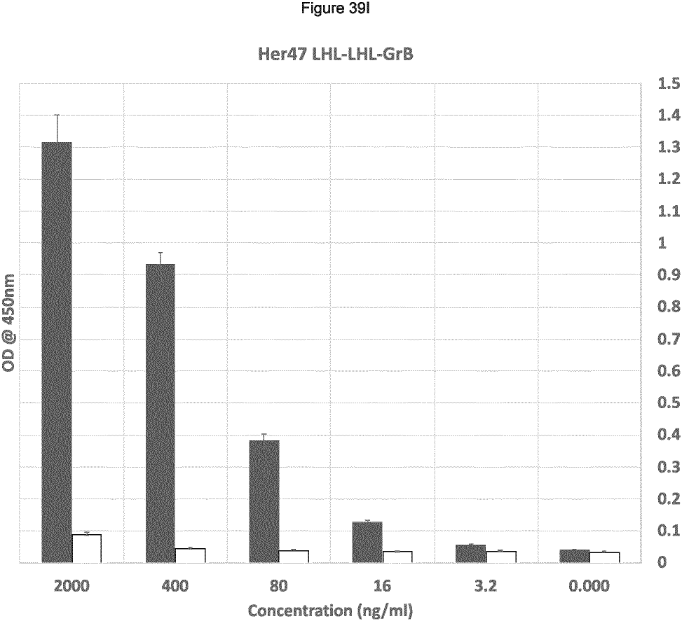

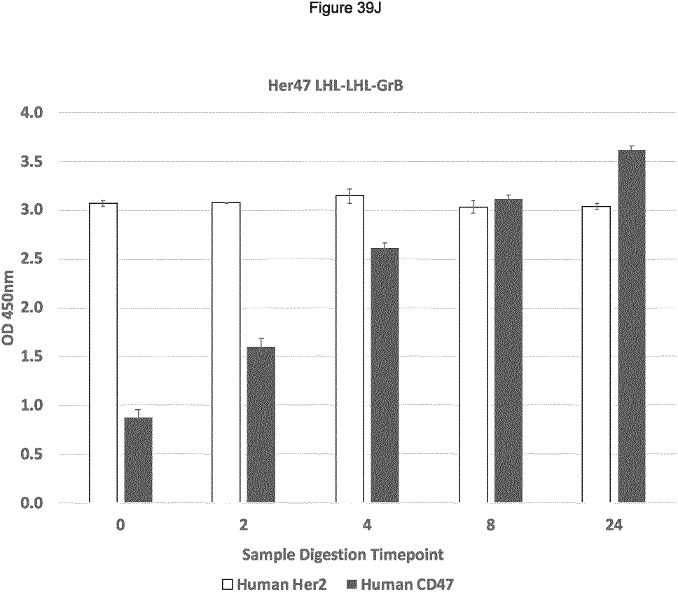

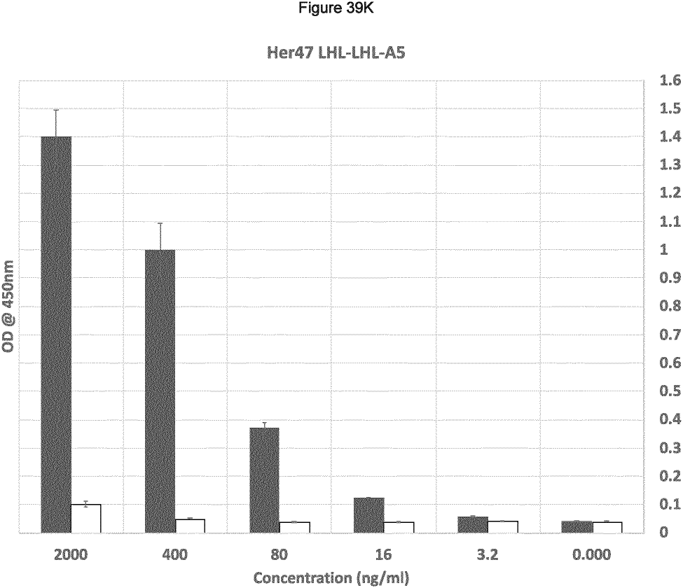

Purified protein from clones Her47 LHL-LHL-EK, Her47-LHL-LHL-Thr, Her47-LHL-LHL-tPA, Her47-LHL-LHL-uPA, Her47-LHL-LHL-GrB and Her47-LHL-LHL-A5 were all tested in titration ELISA against human Her2 and CD47 targets (FIGS. 39A, 39C, 39E, 39G, 39I, 39K). In all cases, these proteins exhibited low/background binding on CD47 at all concentrations tested (white bars), but concentration-dependent binding to Her2 (grey bars). Each protein was then also submitted to a time course enzymatic digest with MMP12, and ELISA binding to Her2 and CD47 targets (FIGS. 39B, 39D, 39F, 39H, 39J, 39L). These findings showed that all proteins retained Her2 binding and exhibited increased activation of CD47 binding, across the time course of enzymatic activation. The functional activities of clones Her47 LHL-LHL-EK, Her47-LHL-LHL-Thr, Her47-LHL-LHL-tPA, Her47-LHL-LHL-uPA, Her47-LHL-LHL-GrB and Her47-LHL-LHL-A5 demonstrated that the Fab2 structure is capable of utilising linker peptide sequences with a wide variety of protease-recognition sites to tailor activation to a given application. For example, in this case, the retention of MMP activation potential in the LHL linker, combined with any of 6 different disease-associated enzyme cleavage motifs in the accompanying linker, would allow tailoring to environments in which MMP and/or Cathepsin enzymes are active, with added activation potential when Enterokinase, thrombin, tPA, Granzyme B, uPA, ADAMTs-5 or other protease enzymes are associated with disease status.

### In Vivo Multi-Dose Tolerability of Her47 Molecules in NOD-SCID Mice

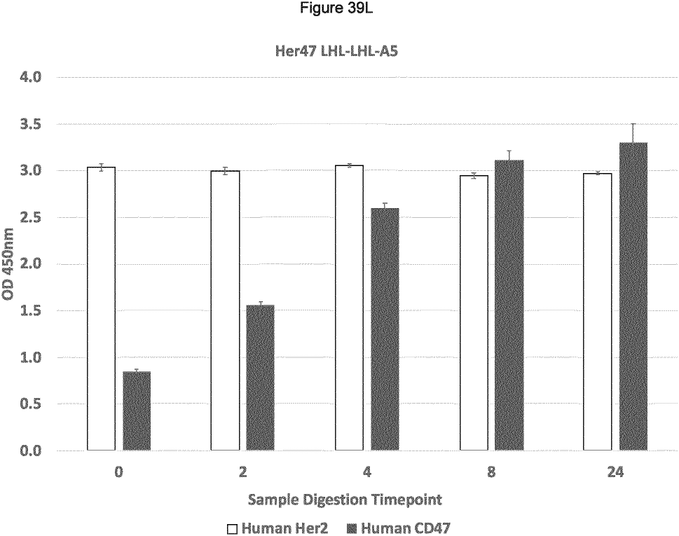

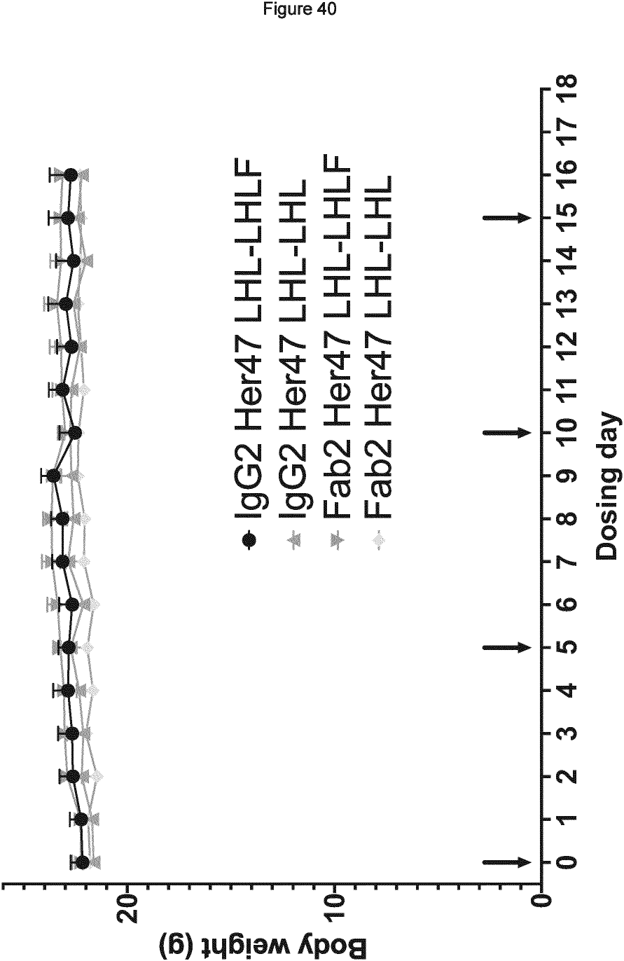

In an in vivo multi-dose tolerability study, IgG² Her47 LHL-LHL, IgG² Her47 LHL-LHLF, Fab² Her47 LHL-LHL and Fab² Her47 LHL-LHLF were each dosed four times (intravenously) in NOD-SCID mice. IgG² proteins were dosed at 14 mg/kg on day 0 and 7 mg/kg on days 5, 10 and 15. Fab² proteins were dosed at 8 mg/kg on day 0 and 4 mg/kg on days 5, 10 and 15. All proteins were fully tolerated, with no clinical signs of toxicity for any individual animals and no loss of bodyweight (FIG. 40). These findings corroborated and expanded upon the tolerability findings for Her47 Fab²-based molecules in single doses in Tg32 mice, outlined above. In the Tg32 mouse study even a single dose of the A-D5 CD47 IgG was not tolerated at 10 mg/kg. In this NOD-SCID mouse study, all four doses of IgG² Her47 LHL-LHL, IgG² Her47 LHL-LHLF, Fab² Her47 LHL-LHL and Fab² Her47 LHL-LHLF were fully tolerated. This findings confirmed that the CD47 binding domains of the Her47 molecules were not being activated in the circulation, even after repeated dosing, as significant activation would have induced the CD47-driven toxicity signals observed for A-D5 IgG1.

### Generation and Analysis of Additional 2-Chain Her2CD3 Fab² ‘One Arm’ Constructs

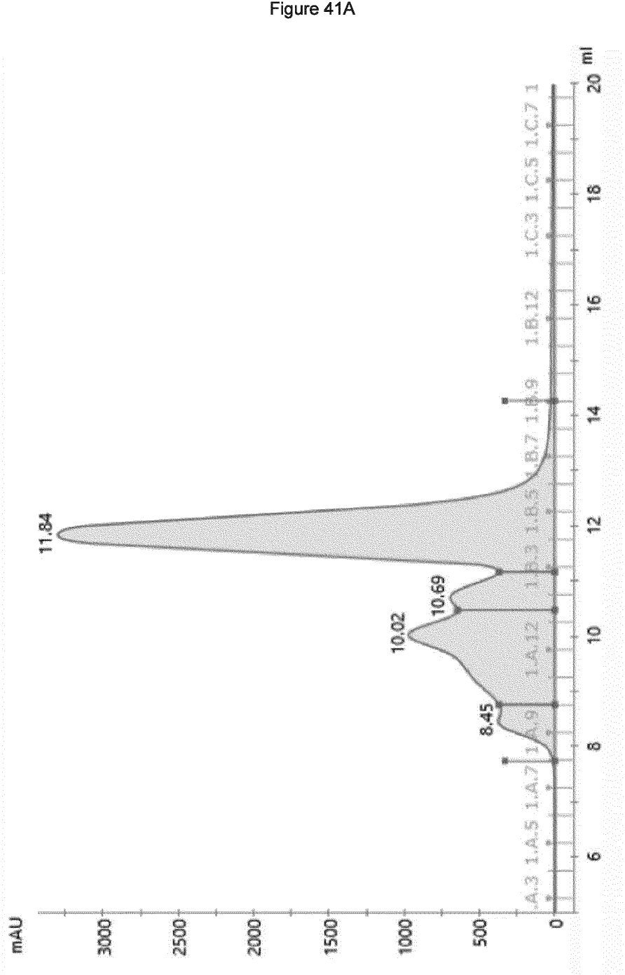

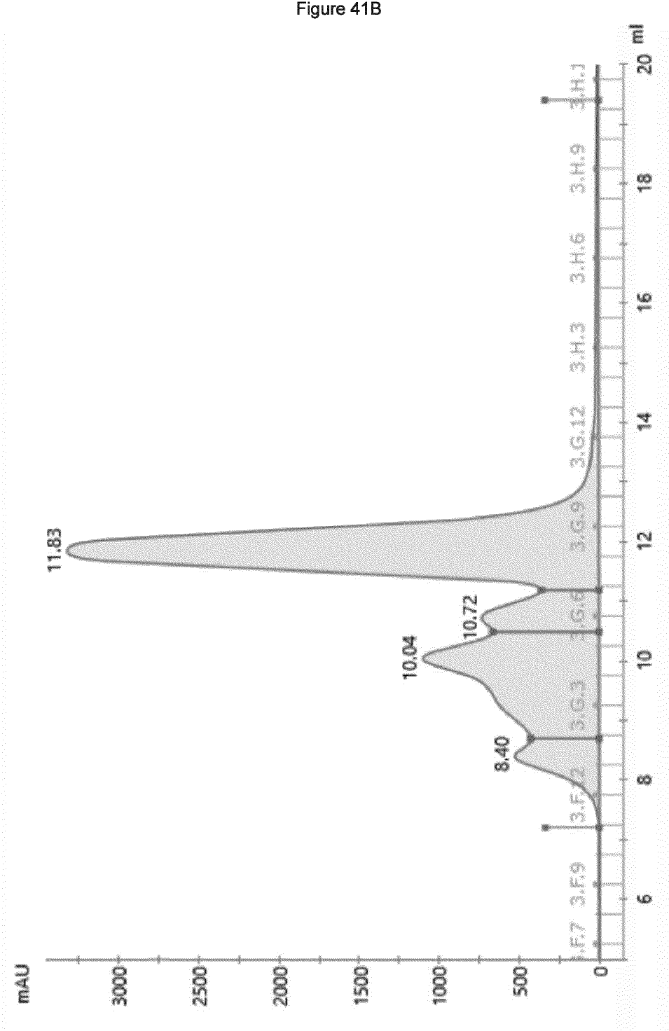

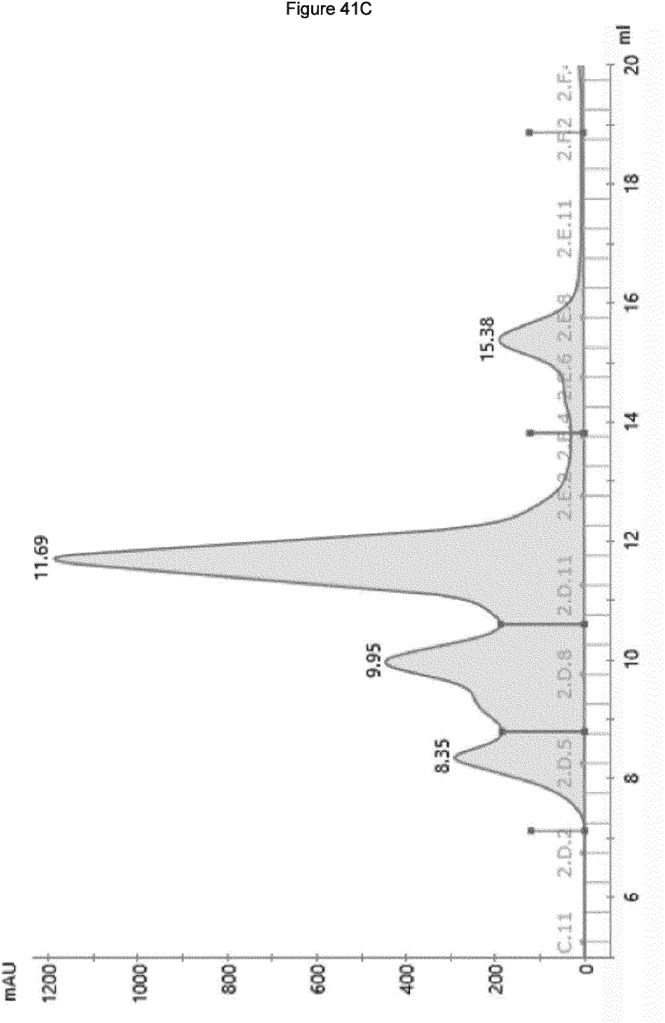

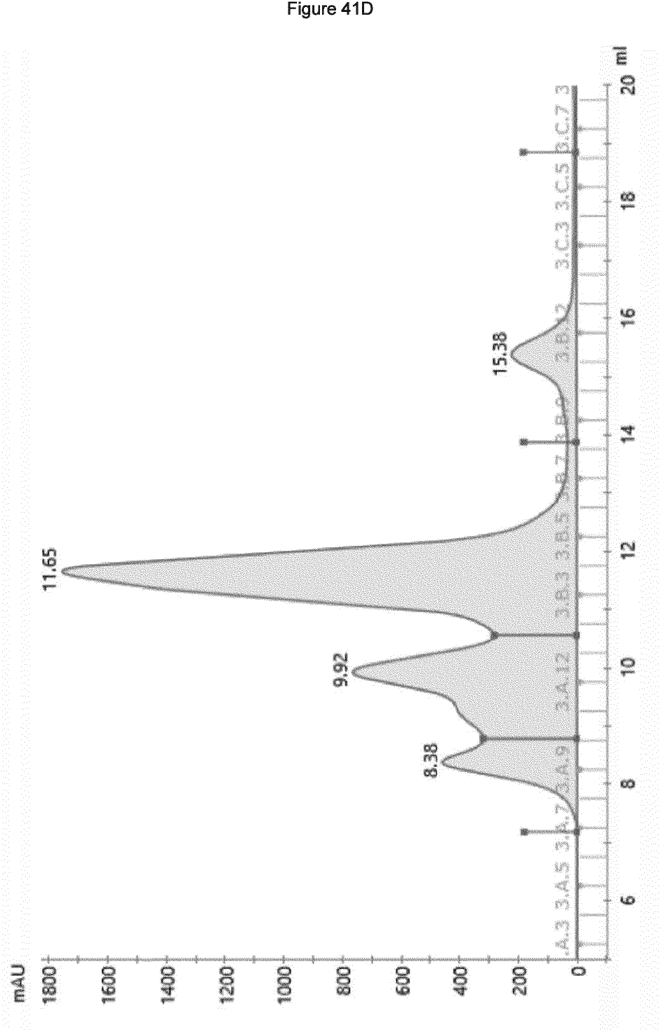

Further Her2CD3 constructs were generated to examine novel sequences for their ability to improve product uniformity and activity (Table 22). All Her2CD3 constructs were expressed by CHO cells, purified from supernatant by ProA and then analysed by SEC. These analyses showed that Fab² Her23 LHL-LHL-S and Fab² Her23 LHLF-LHL-S both exhibited greater uniformity of the main product (FIG. 41 A, FIG. 41B) than did Fab² Her23 LHL-LHL and Fab² Her23 LHL-LHLF, which both exhibited a greater proportion of higher and lower molecular weight products (FIG. 41C, FIG. 41D). These findings suggested that improved uniformity had been created by swapping the orientation upper fabs in constructs Fab² Her23 LHL-LHL-S and Fab² Her23 LHLF-LHL-S so that the chains are [VL-CL-Linker-VH-CH-Fc] plus [VH-CH-Linker-VL-CL-Fc]. This is in contrast to Fab² Her23 LHL-LHL and Fab² Her23 LHL-LHLF, where the two chains are both [VL-CL-Linker-VL-CL-Fc] plus [VH-CH-Linker-VH-CH-Fc].

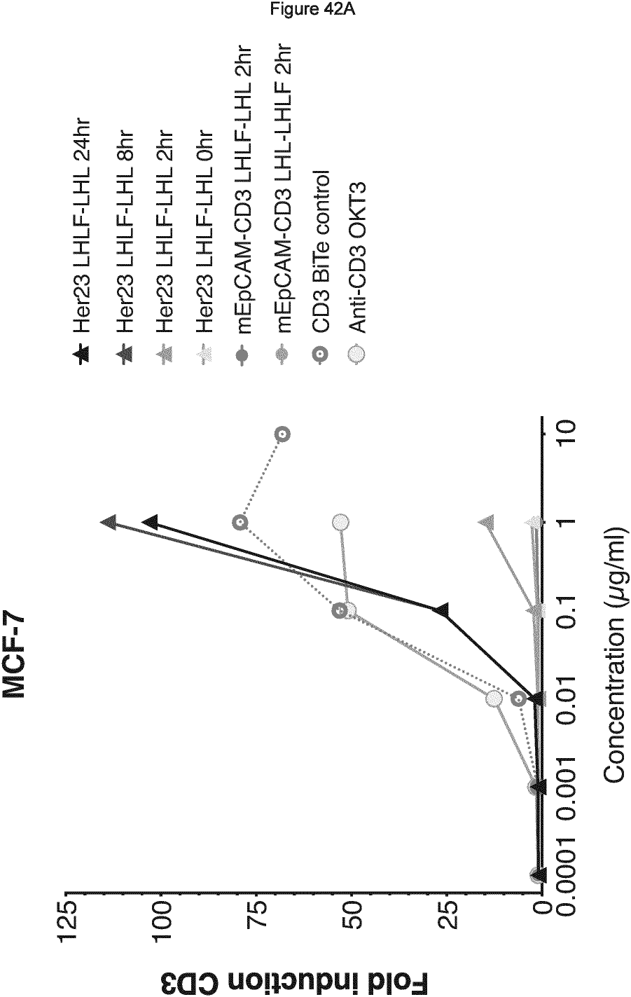

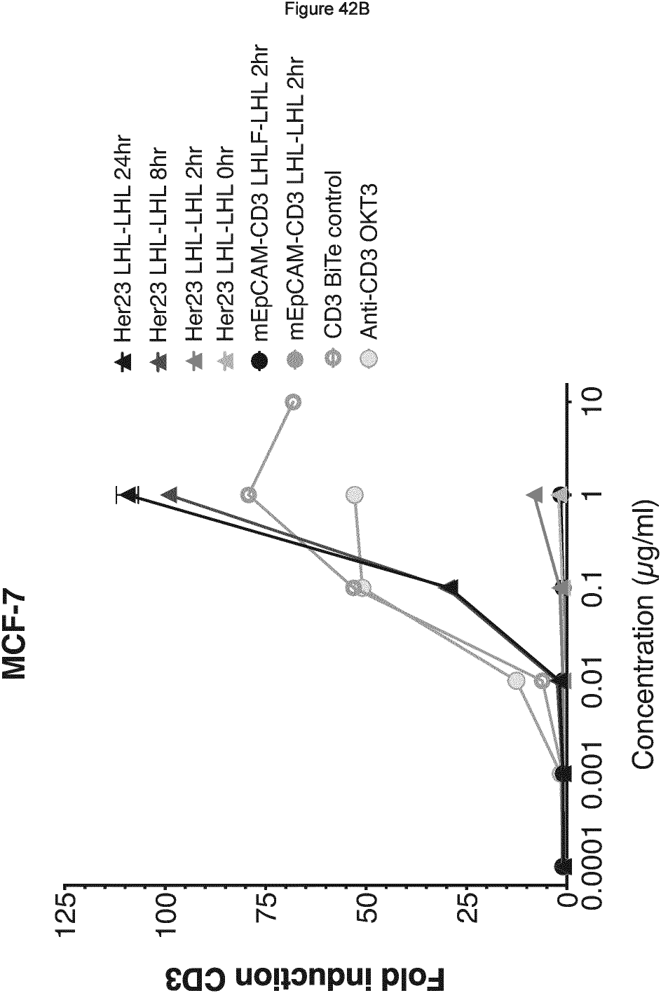

Control proteins Fab² mEpCam3 LHLF-LHL-S and Fab² mEpCam3 LHL-LHL-S (upper Fab contains the v domains of the anti-murine EpCAM antibody G8.8) were then also designed and expressed (Table 22). The correct MW products for clones Fab² Her23 LHL-LHL-S, Fab² Her23 LHLF-LHL-S, Fab² mEpCam3 LHLF-LHL-S and Fab² mEpCam3 LHL-LHL-S were isolated by preparative SEC and analysed in a Promega Jurkat cell-based CD3 ligation reporter cell bioassay, using either MCF-7 or BT-474 as human Her2+ target cells (according to manufacturer’s instructions). Primary analyses using MCF-7 as (very low Her2-expressing) target cells demonstrated that positive control Her2-CD3 BITE protein and OKT3 IgG1 both generated strong positive, concentration-dependent, CD3 activation signal. Clones Fab² mEpCam3 LHLF-LHL-S and Fab² mEpCam3 LHL-LHL-S, treated with MMP12 for 2h, generated no signal at any concentration (FIGS. 42 A, B). In contrast, clones Fab² Her23 LHLF-LHL-S (FIG. 42A) and Fab² Her23 LHL-LHL-S (FIG. 42B) both showed no measurable signal at 0 h, but gradually increasing signal at 2, 8 and 24 h, in similar concentration ranges to the Her2-CD3 BITE, but with maximal signal in both cases being higher than that achieved for the positive controls.

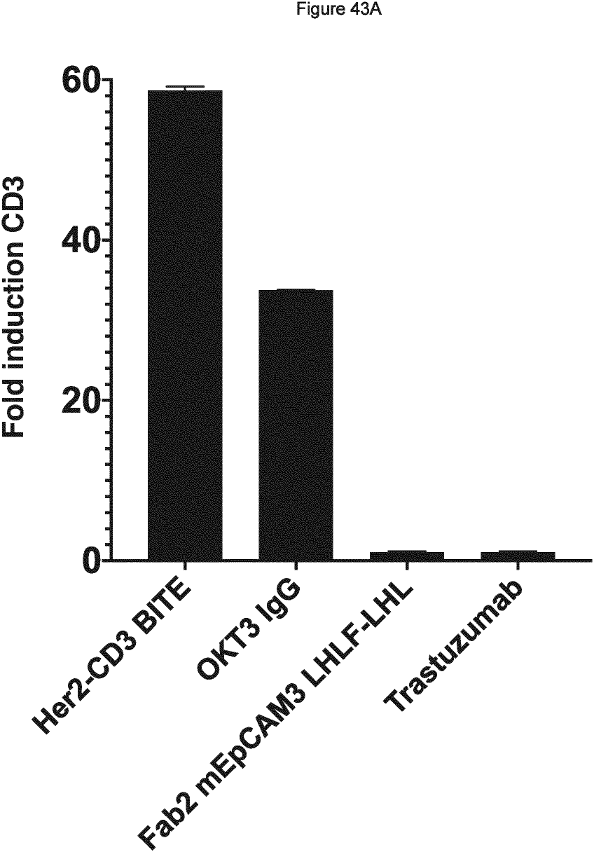

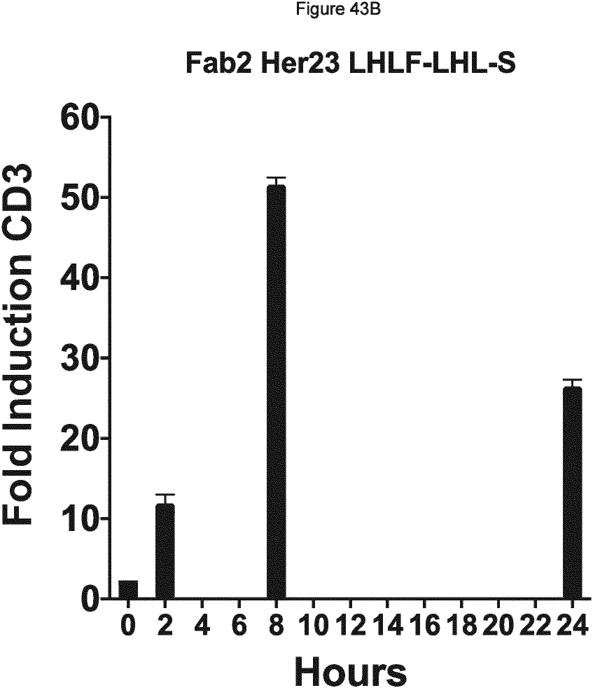

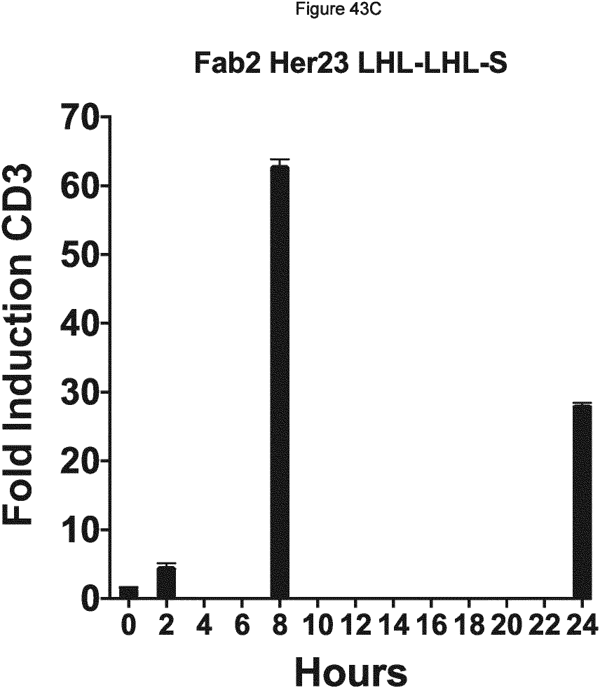

In a secondary assay using Her2 high-expressing BT-474 cells as target cells, all of the above test samples and controls were assayed at 0.1 µg/ml (FIGS. 43A-C). Positive control proteins Her2-CD3 BITE and OKT3 IgG1 both induced strong CD3 activation signaling, while negative controls Fab² mEpCam3 LHLF-LHL-S and Trastuzumab did not. Clones Fab² Her23 LHLF-LHL-S (FIG. 43B) and Fab² Her23 LHL-LHL-S (FIG. 43C) both showed no measurable signal at 0 h, but gradually increasing signal at 2 hours and maximal at 8. Importantly, at 24 h signal significantly decreased in comparison to 8 h. This finding suggests that progressive enzyme activity, which may cleave both linkers (FIG. 33) may lead to eventual separation of the upper and lower Fabs. This may be a beneficial characteristic for a CD3-targeting molecule, as it would be ideal for a such a molecule to be preferentially activated in the highly proteolytic tumour environment, but also to be gradually deactivated there, minimising the risk of the molecule leaking out into healthy tissue, in an active form.

### In Vitro Measurements of Molecular Stability of Her2-CD47 IgG² and Fab² Proteins

Historically, engineered antibody formats have often suffered from structural heterogeneity, leading to manufacturing issues. To examine the stability of the IgG² Her47 LHL-LHL and Fab² Her47 LHL-LHL proteins, a series of in vitro measurements were made:

Forced Oxidation - Oxidation of exposed amino acid residues, such as tryptophan and methionine, is a common degradation pathway for mAbs, with impact on their biological activity. In this study, forced oxidation with 0.5 % H2O2 in PBS for 2 hours at room temperature was applied to the IgG² Her47 LHL-LHL and Fab² Her47 LHL-LHL proteins. As oxidation can alter overall hydrophobicity of an antibody, either by increasing the polarity of the oxidised form or through conformational changes, potential changes induced by forced oxidation were analysed by Reverse Phase (RP) and Size Exclusion (SEC) chromatographies. SEC analysis revealed no changes in the proportion of monomeric species, in either test sample (IgG² Her47 LHL-LHL and Fab² Her47 LHL-LHL both exhibited 99.5 and 97.4 % monomeric species, respectively, before and after oxidation). In RP analysis of intact antibodies, a decrease of 0.5 min in the retention time of IgG² Her47 LHL-LHL and 0.4 min in the retention time of Fab² Her47 LHL-LHL was observed for the intact (non-reduced) antibodies after forced oxidation with 0.5 % H2O2. In RP analysis of subunits, a decrease of 0.5 min in the retention time of the heavy chain and no shift in the retention time of the light chain was observed for IgG² Her47 LHL-LHL after forced oxidation. The reduced Fab² Her47 LHL-LHL showed a different profile on RP analysis, with 3 peaks observed, representing light chain, heavy chain and truncated hinge-Fc stump. Upon oxidation 0.4-0.5 min shifts were observed for the heavy chain and truncated hinge-Fc stump, but not the light chain. The shifts in the retention time, after H2O2 treatment, observed for both reduced IgG² Her47 LHL-LHL and Fab² Her47 LHL-LHL samples, suggest minor oxidation of the exposed amino acids (limited to the Fc regions of the proteins).

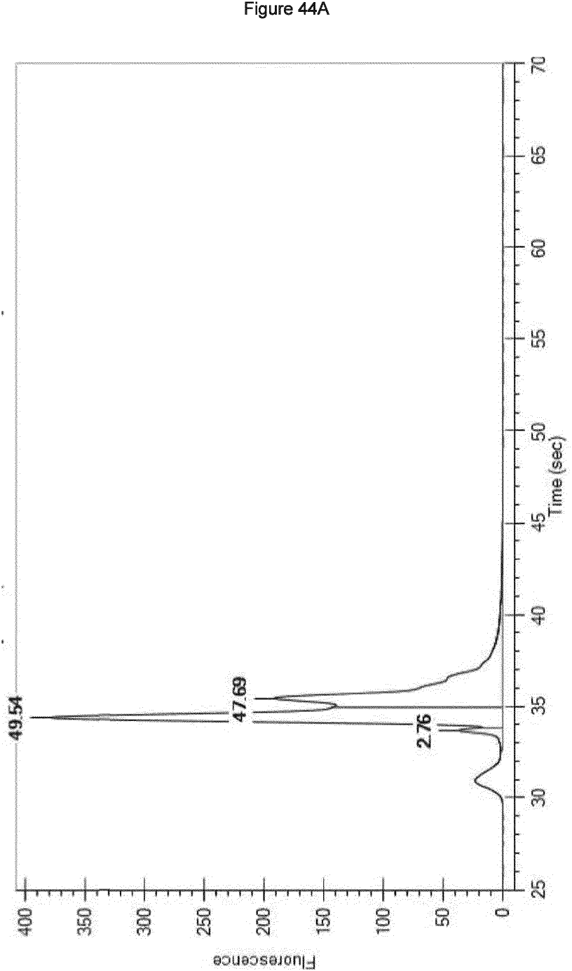

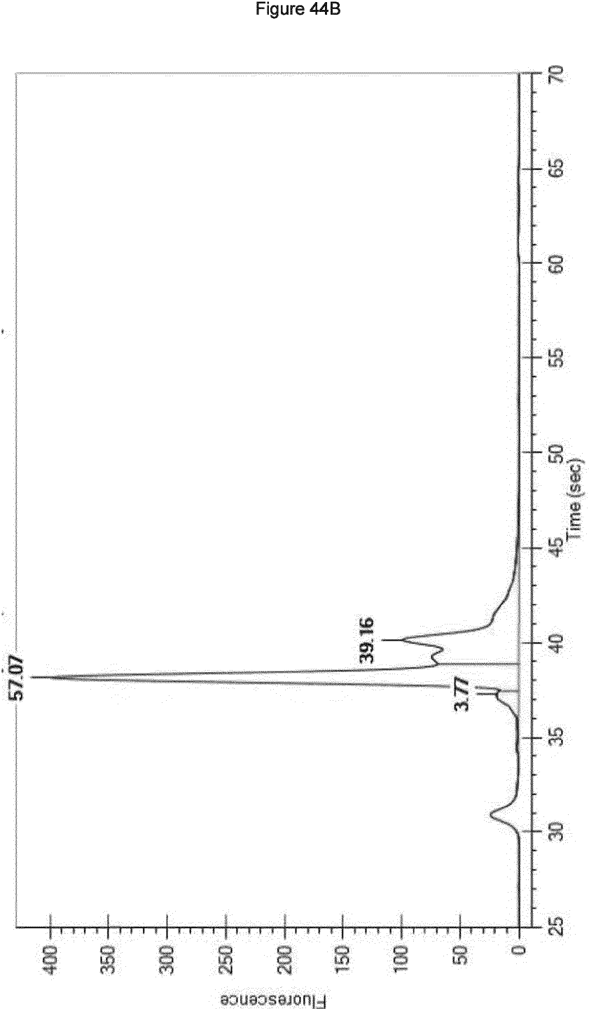

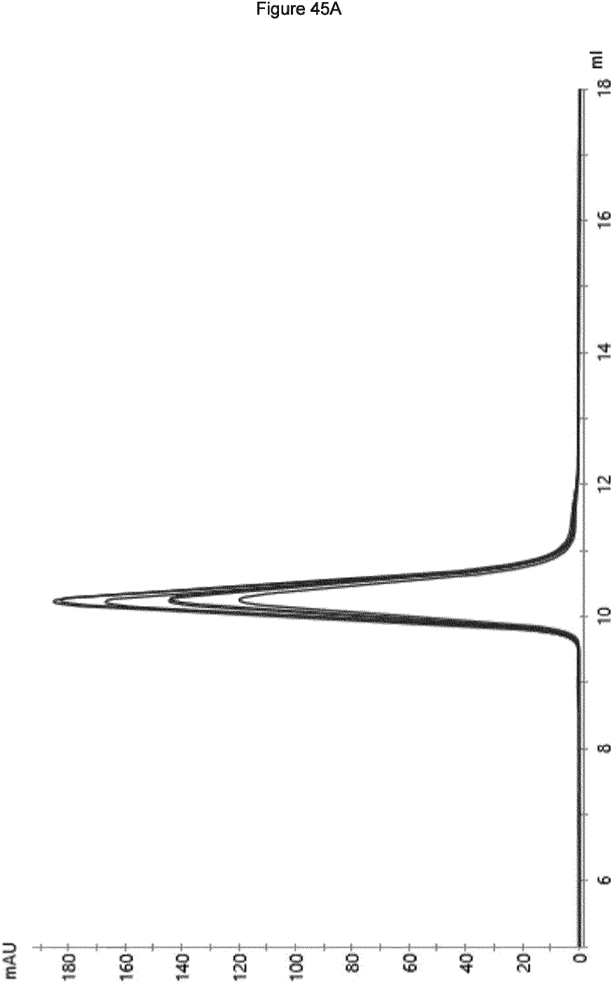

Charge variant analysis - Charge heterogeneity analysis is important in the characterisation of monoclonal antibodies because it provides important information about product quality and stability. Heterogeneity can be caused by enzymatic post-translational modifications (glycosylation, lysine truncation) or chemical modifications during purification and storage (oxidation or deamidation). Charge variant profiling for the provided test articles was performed by a commercial Charge Variant Assay. The charge variant profiles of the IgG² Her47 LHL-LHL (FIG. 44A) and Fab² Her47 LHL-LHL (FIG. 44B) both displayed a homogeneous profile, with one main isoform (50-57 % of total), a major acidic isoform (40-48 % of total) and one minor basic isoform (approx. 3%).

Retention on HIC - Overall hydrophobicity is an indicator of a protein’s tendency to self-associate, which can be a significant risk factor for aggregation and viscosity during bioprocessing. Proteins have hydrophobic ‘patches’ on their surface, generated by the presence of the side chains of hydrophobic or non-polar amino-acids. Depending on their number, size and distribution, the resulting surface hydrophobicity will be a specific characteristic for each protein. HIC separates proteins based on differences in their surface hydrophobicity, utilising reversible binding between the protein and the hydrophobic surface of the HIC resin. IgG² Her47 LHL-LHL and Fab² Her47 LHL-LHL exhibited HIC column retention times of 5.4 and 5.0 mins, respectively. These values are towards the lower range of hydrophobicity in comparison to those obtained for clinical monoclonal antibodies, such as Adalimumab (4.5 mins), Cetuximab (5.9 minutes), Brentuximab (6.3 mins) and Golimumab (8.1 mins). Indeed, these values suggest an aggregation propensity and stability similar to that of anti-Her2 Trastuzumab (5.4 mins retention), from which the Her2 binding domains in both IgG² Her47 LHL-LHL and Fab² Her47 LHL-LHL are derived.

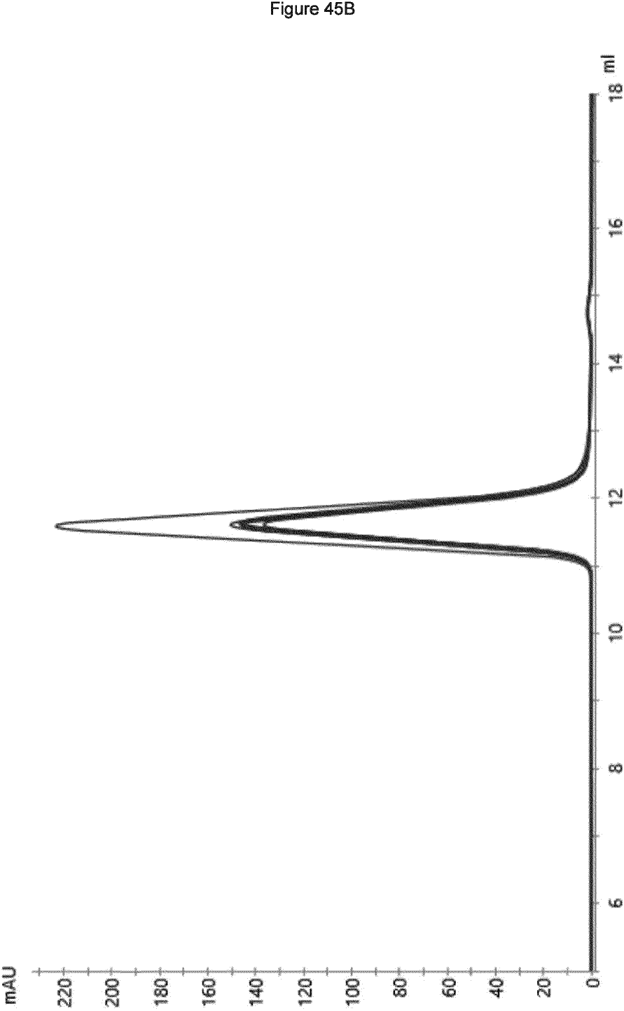

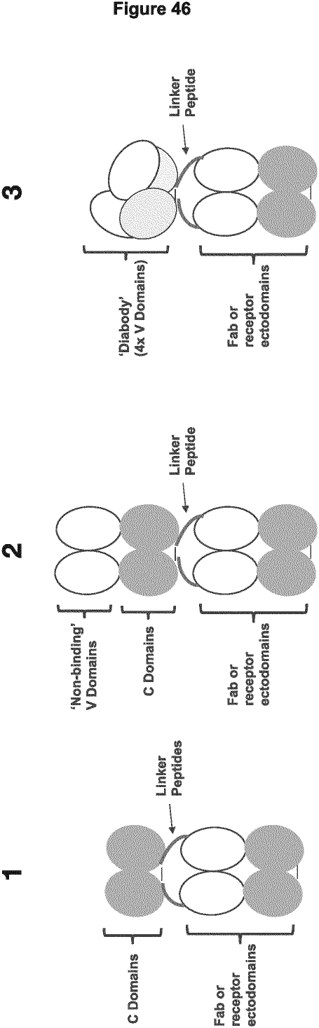

Freeze-thaw stability analyses - Instability of proteins during freeze-thaw steps is an indicator of difficulty in manufacturing and bioprocessing, as increased aggregation or fragmentation of the protein is a risk for reduction in product quality. To assess this risk for proteins containing Fab² structures, IgG² Her47 LHL-LHL and Fab² Her47 LHL-LHL proteins were subjected to 5 rounds of Freeze-thaw , followed by SEC analyses after each round. These analyses showed that neither IgG² Her47 LHL-LH L (FIG. 45A), nor Fab² Her47 LHL-LH L (FIG. 45B) proteins exhibited any change in monodispersity (no aggregation or breakdown products observed) over the 5 rounds of freezing.

Collectively, these findings suggested that both IgG² Her47 LHL-LHL and Fab² Her47 LHL-LHL proteins had low aggregation risk, low hydrophobicity, low charge heterogeneity and low propensity towards oxidation.

### Biacore® Analyses of Affinity of IgG Variants for Human Fc Receptors

Antibodies targeting receptors on diseased cells must bind to Fcy receptors if they are to mediate ADCC and ADCP activities. To examine whether these binding functions were retained in Fab2-based constructs, IgG² Her47 LHL-LHL and Fab² Her47 LHL-LHL proteins were examined for binding affinity to all human and mouse Fc receptors via surface plasmon resonance analyses. These analyses demonstrated that both the isotype control human IgG1 and IgG4 exhibited the expected strong and weak binding affinities (respectively) for all human Fcγ receptors, including both the high and low affinity variants of FcyRIIA and FcyRIIIA (Table 23). Similarly, isotype control mouse IgG2a and IgG1 exhibited the expected strong and weak binding affinities (respectively) for mouse FcγRI, FcγRIII and FcyRIV receptors. For IgG² Her47 LHL-LHL and Fab² Her47 LHL-LHL, the binding to each of the human and mouse Fc receptors tested is highly similar to that observed for the isotype control human IgG1. These data suggest that both IgG² and Fab² proteins that are built on an IgG1 Fc should be capable of binding to Fc receptors, when bound at the surface of diseased cells, in both humans and mice.

### Further Protein Construct Designs

Further protein constructs are envisioned (FIG. 46). The constructs may contain: 1. Only constant domains in the ‘upper fab’ position, meaning that the activity of the ‘lower Fab’ is prevented from binding its target, but may become active in an appropriate proteolytic environment. 2. Dummy ‘non-binding’ variable domains in the ‘upper fab’. These dummy variable domains would be proven not to bind any known target in the body, so only the ‘lower fab’ exhibits potential drug target-binding ability, and only after activation by proteolysis of one of the linker domains. 3. The ‘upper Fab’ is replaced by a ‘diabody’ structure containing 4 variable domains. This diabody structure may or may not contain disulphide linkage, as found in ‘DART’ proteins. Diabody structures may facilitate the binding of 2 copies of the same target, or two separate targets, with the activity of the ‘lower Fab’ being prevented from binding its target, until rendered active in an appropriate proteolytic environment. In any of the constructs envisaged here, or demonstrated above, the Fab² structures may be free, or fused to another functionalising structure, such as an Fc fragment, a small domain or peptide that extends half-life such as an albumin binding moiety. They may also be chemically conjugated to small molecules, peptides or other proteins that mediate further biological functions.

### Cell Proliferation Analyses for Her2CD47 Proteins Using Her2-High BT-474 Cells

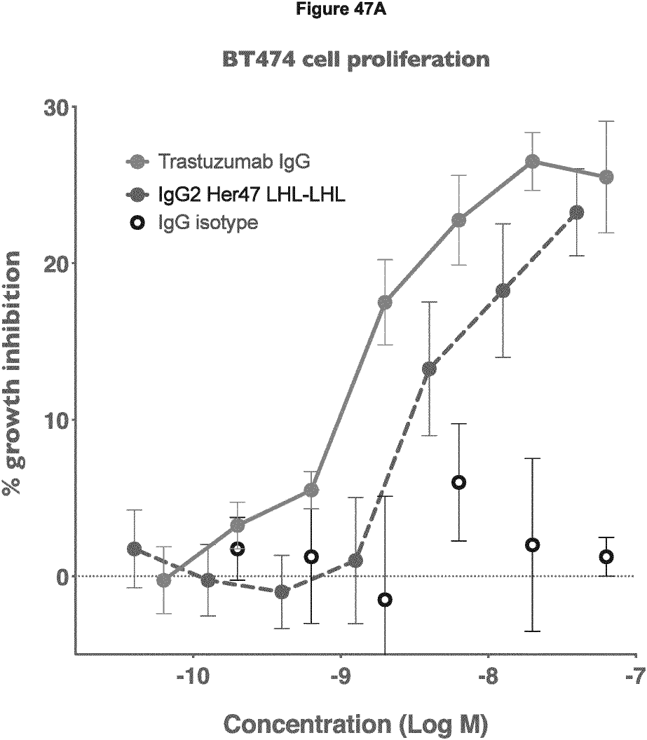

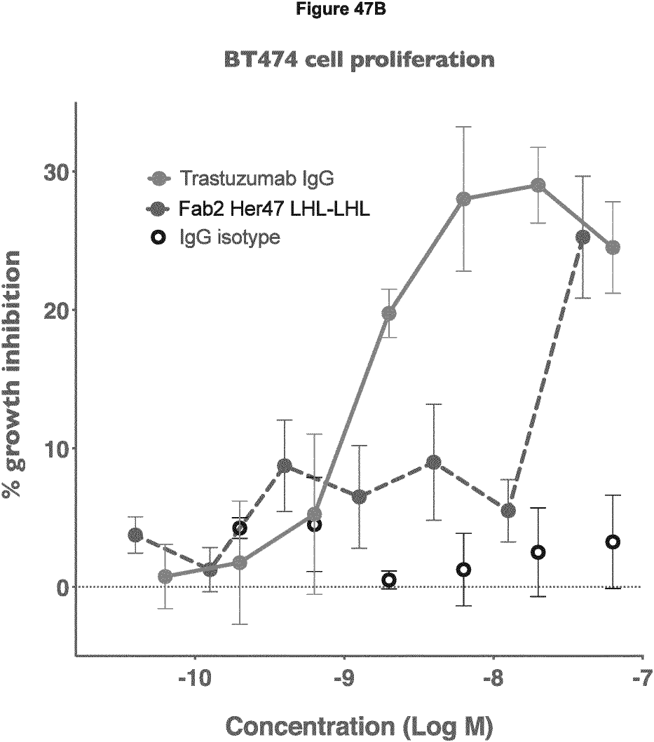

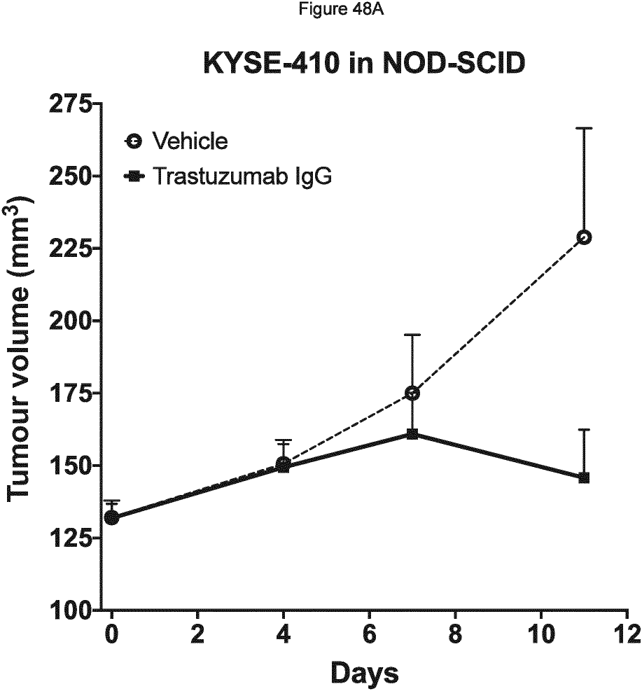

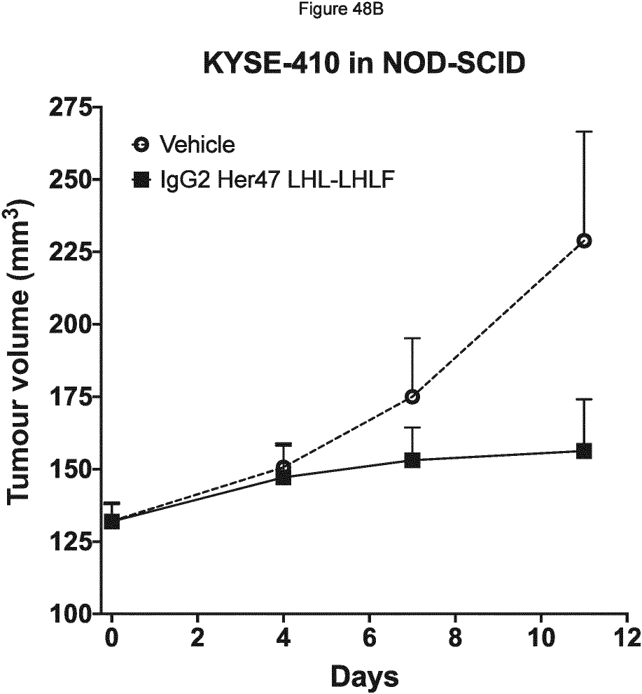

As the Her2 binding domains of the upper Fab of IgG² Her47 and Fab² Her47 proteins are capable of mediating inhibition of the kinase activity of this receptor, a cell proliferation analysis was performed. This assay used BT-474 cells as they are a cell line with a known sensitive response to Her2 inhibition. Trastuzumab, Isotype control IgG1, IgG² Her47 LHL-LHL (FIG. 47A) and Fab² Her47 LHL-LHL (FIG. 47B), were applied to the BT-474 cells over a 72h incubation period and cell proliferation measured. Data was represented as percent inhibition of cell growth (FIG. 47). These analyses showed that while Trastuzumab demonstrated strong, concentration-dependent inhibition of BT-474 cell proliferation, IgG² Her47 LHL-LHL (FIG. 47A) demonstrated slightly lower potency and Fab² Her47 LHL-LHL (FIG. 47B) was lower again in potency (reflecting its monovalent binding capacity).

### In Vivo Efficacy Analyses of Her47 Molecules in Tumour Xenograft-bearing NOD-SCID Mice (KYSE-410 Model)

In in vivo multi-dose efficacy studies, Trastuzumab, IgG² Her47 LHL-LHL, IgG² Her47 LHL-LHLF, Fab² Her47 LHL-LHL and Fab² Her47 LHL-LHLF (Fab² structures containing a human IgG1 Fc, as in FIG. 3B) were each dosed four times (intravenously, on days 0, 5, 10, 15) in NOD-SCID mice bearing tumours generated by sub-cutaneous inoculation with the Her2-expressing oesophageal cancer cell line KYSE-410. Dosing began once tumours were established at >125 mm². IgG² proteins were dosed at 14 mg/kg on day 0 and 7 mg/kg on days 5, 10 and 15. Fab² proteins were dosed at 8 mg/kg on day 0 and 4 mg/kg on days 5, 10 and 15. Trastuzumab was dosed at 8 mg/kg on day 0 and 4 mg/kg on days 5, 10 and 15. Tumour volumes were measured by caliper measurements.

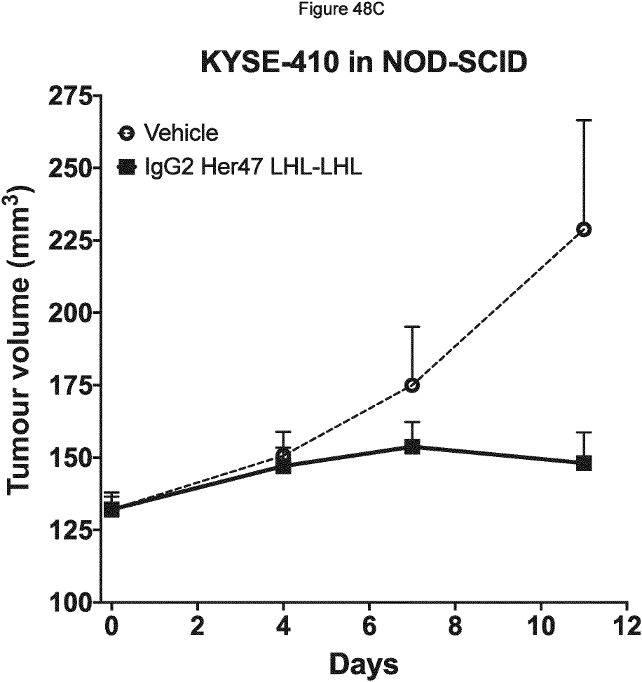

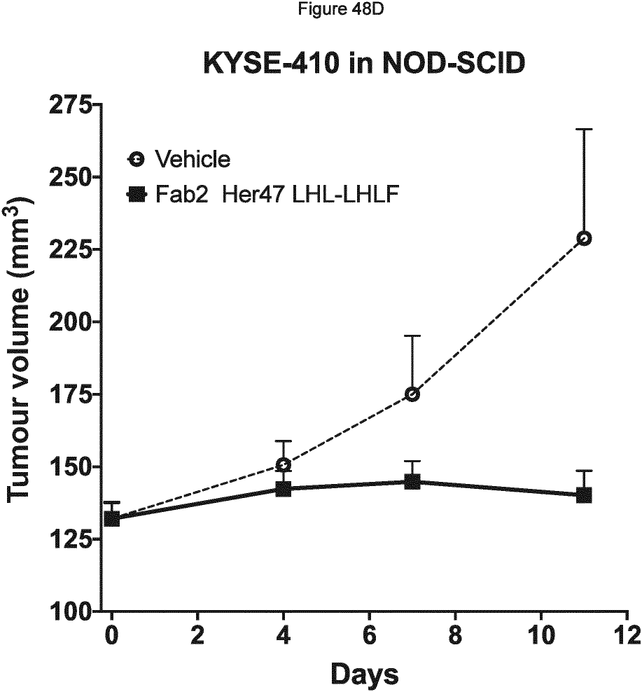

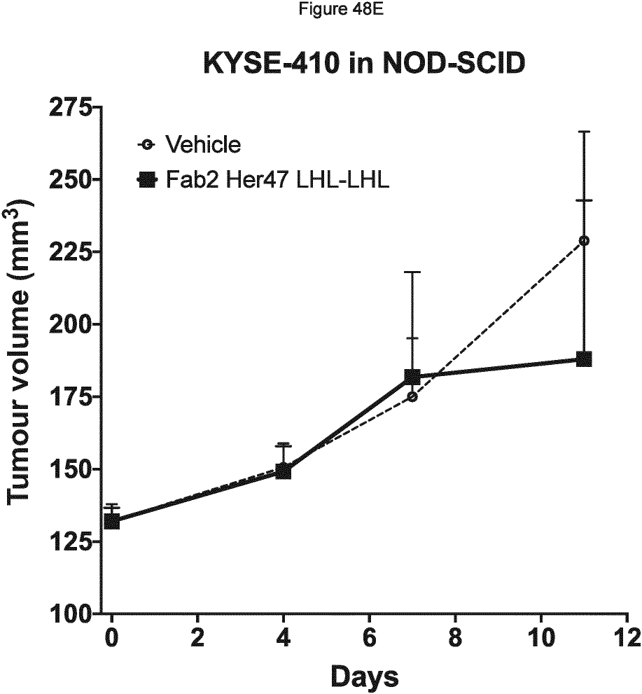

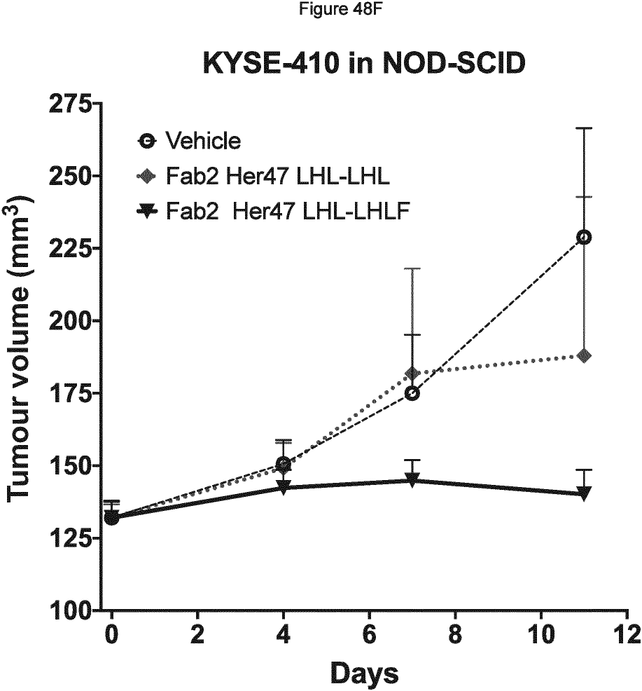

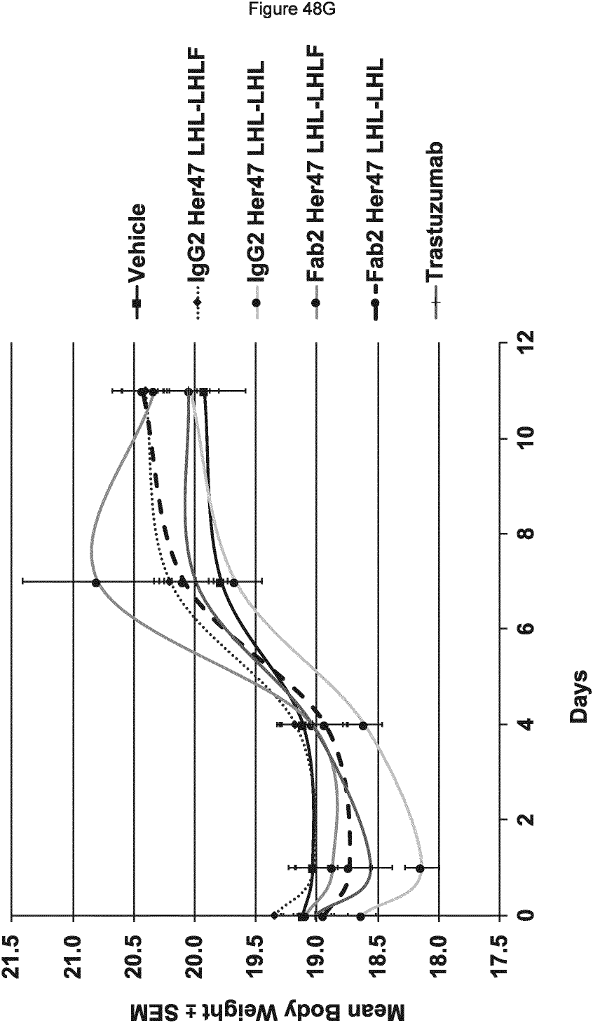

After 3 doses, on day 11, Trastuzumab (FIG. 48A), IgG² Her47 LHL-LHLF (FIG. 48B), IgG² Her47 LHL-LHL (FIG. 48C) and Fab² Her47 LHL-LHLF (FIG. 48D) all demonstrated significantly reduced tumour growth in comparison to vehicle (2-way ANOVA: p = 0.005, 0.005, 0.019 and 0.003, respectively). In contrast, Fab² Her47 LHL-LHL (FIG. 48E) did not significantly reduce tumour growth by day 11 (2-way ANOVA: p = 0.66). Importantly, the sequences of Fab² Her47 LHL-LHL and Fab² Her47 LHL-LHLF are identical (apart from the two point mutations in the LHLF linker that accelerate and broaden protease sensitivity versus the LHL linker, as shown above), but lead to significant differences in potency (FIG. 48F). In addition, the data in FIG. 47 demonstrates that the 1-arm Fab² structure leads to weaker Her2-driven inhibition of cell proliferation than observed for Trastuzumab, which contains identical Her2-binding VH and VL domain sequences to those found in both Fab² Her47 LHL-LHL and Fab² Her47 LHL-LHLF. As a result, it follows that: 1. The approximately equivalent potency at day 11 observed for both Trastuzumab (FIG. 48A) and Fab² Her47 LHL-LHLF (FIG. 48D) cannot be driven by high Her2 kinase activity in Fab² Her47 LHL-LHLF. 2. The high potency of Fab² Her47 LHL-LHLF is therefore most likely driven by protease activation in the KYSE-410 tumour microenvironment, leading to CD47 blockade and innate immune engagement.

In aggregate, the findings outlined above therefore demonstrate that the Fab² structure allows efficient ablation of ‘lower Fab’ activities, with both CD47 and CD3 binding domains acting as examples. Binding capacity of the ‘upper fab’ domains is fully maintained, but significant activity in the lower fab domains is only observed after activation by proteases such as MMPs and Cathepsins, that are associated with high activity in diseased tissues such as tumours and fibrotic tissues. The modulation of linker sequences in the Fab² structure allows the tuning of lower fab activation to be maximised in the disease microenvironment and to avoid both peripheral sink problems and toxicities, as exemplified by the performance of the Her47 molecules, in vitro and in vivo.

### In Vivo Pharmacokinetics of Her47 Molecules in NOD-SCID Mice

In an in vivo multi-dose PK study, IgG² Her47 LHL-LHL, IgG² Her47 LHL-LHLF, Fab² Her47 LHL-LHL and Fab² Her47 LHL-LHLF would each be dosed once (intravenously) in NOD-SCID mice. IgG² proteins would be dosed at 14 mg/kg and Fab² proteins would be dosed at 8 mg/kg. Blood samples would be taken at 15 mins, 30 mins, 1h, 3h, 6h and 24 h and serum antibody levels measured using an anti-human IgG1 ELISA.

### In Vivo Efficacy Analyses of Her47 Molecules in Tumour Xenograft-bearing NOD-SCID Mice (SKOV-3, JIMT-1 and NUGC-4 Models)

In in vivo multi-dose efficacy studies, IgG² Her47 LHL-LHL, IgG² Her47 LHL-LHLF, Fab² Her47 LHL-LHL and Fab² Her47 LHL-LHLF would be each dosed four times (intravenously) in NOD-SCID mice bearing tumours generated by sub-cutaneous inoculation with the cell lines SKOV-3, JIMT-1 and NUGC-4. IgG² proteins would be dosed at 14 mg/kg on day 0 and 7 mg/kg on days 5, 10 and 15. Fab² proteins would be dosed at 8 mg/kg on day 0 and 4 mg/kg on days 5, 10 and 15. Trastuzumab would be dosed at 8 mg/kg on day 0 and 4 mg/kg on days 5, 10 and 15. Tumour volumes would be measured by caliper measurements.

### In Vivo Analyses of Tolerability and Pharmacokinetics in Cynomolgus Monkey

To examine the tolerability and pharmacokinetics of IgG² and/or Fab² in the context of CD47 or CD3 as the lower Fab domain, multiple example molecules would be studied in cynomolgus monkeys. For example, IgG² and/or Fab² might each be dosed once, twice or three times (intravenously) at concentrations of 2 mg/kg or above. Blood samples would be collected from each animal according to a bleeding schedule. Analyses of serum antibody concentration would be measured to calculate PK and to assess the risk of TMDD). To sample the effects of the dosed proteins more broadly, full haematological panels would also be examined at a series of days after dosing. These analyses would measure reticulocyte, erythrocyte (RBC), haemoglobin, mean corpuscular haemoglobin concentration (MCHC), mean corpuscular volume (MCV), leukocyte, monocyte, lymphocyte, basophil, eosinophil and/or neutrophil levels.

### Biosensor Measurements of Target Co-Engagement

To examine the effect of activation on Fab² binding affinity to both Her2 and CD47 (or CD3), a biosensor assay would be established which could sample both Her2 and CD47 (or CD3) binding on the same chip surface, such as a Dynamic Biosensors instrument. In this assay, control antibodies and IgG² or Fab² proteins (undigested or activated for e.g. 2, 4, 8 or 24 hours with an MMP or Cathepsin enzyme) would be applied to a sensor chip surface that had been differentially labelled with purified Her2 and CD47 (or CD3) ectodomain proteins separately, or together at different densities. Affinity for Her2 and CD47 (or CD3) would be measured to ascertain the influence of multivalent interaction on functional affinity for both targets separately, and on the same surface.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

No limitation with respect to the specific embodiments disclosed herein and set forth in the appended claims is intended nor should any be inferred. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term’s definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

### NUMBERED EMBODIMENTS

Notwithstanding the appended claims, the disclosure sets forth the following numbered embodiments:

1. A protein comprising a first moiety and a second moiety and a peptide linker between the first moiety and the second moiety,
wherein the peptide linker comprises an amino acid sequence from a human immunoglobulin hinge region or an amino acid sequence or an amino acid sequence having from 1 to about 7 amino acid substitutions compared to a human immunoglobulin hinge region;
wherein the peptide linker is cleavable by a protease expressed in a diseased tissue;
wherein the second moiety is capable of specifically binding to a molecule expressed in the diseased tissue; and
wherein the binding of the second moiety to the molecule expressed in the diseased tissue is reduced or inhibited when the peptide linker is uncleaved.

2. The protein of embodiment 1, wherein the peptide linker is between about 5 and about 15 amino acids in length.

3. The protein of embodiment 1 or 2, wherein the peptide linker comprises or consists of the amino acid sequence of SEQ ID NO:1, SEQ ID NO:2, SEQ ID NO:3, SEQ ID NO:4, SEQ ID NO:5, SEQ ID NO:69, SEQ ID NO:70, SEQ ID NO:71, SEQ ID NO:72, SEQ ID NO:81, SEQ ID NO:82, SEQ ID NO:83, SEQ ID NO:84, SEQ ID NO:85, SEQ ID NO:86, or SEQ ID NO:87.

4. The protein of any one of embodiments 1-3, wherein the protease is a human matrix metalloprotease (MMP), a human cathepsin, human enterokinase, human thrombin, human tPA, human Granzyme B, human uPA, or human ADAMTs-5.

5. The protein of any one of embodiments 1-4, wherein the peptide linker comprises a human MMP cleavage site, a human cathepsin, human enterokinase, human thrombin, human tPA, human Granzyme B, human uPA, or human ADAMTs-5 cleavage site.

6. The protein of any one of embodiments 1-5, wherein the human MMP is MMP-2, MMP-3, MMP-7, MMP-8, MMP-9, MMP-10, MMP-12, MMP-13 or MMP14.

7. The protein of any one of embodiments 1-6, wherein the level or the activity of the human MMP is elevated in the diseased tissue compared to the level or the activity of the human MMP in a non-diseased tissue.

8. The protein of any one of embodiments 1-5, wherein the human cathepsin is Cathepsin A, Cathepsin C, Cathepsin D, Cathepsin G, Cathepsin L or Cathepsin K.

9. The protein of any one of embodiments 1-5 and 8, wherein the level or the activity of the human cathepsin is elevated in the diseased tissue compared to the level or the activity of the human cathepsin in a non-diseased tissue.

10. The protein of any one of embodiments 1-9, wherein the first moiety comprises an antibody, an antigen-binding portion of an antibody or a receptor ectodomain.

11. The protein of embodiment 10, wherein the first moiety is a Fab, a single-chain Fab, a VH domain, a VL domain, an immunoglobulin new antigen receptor (IgNAR), a single-chain variable fragment (scFv), a diabody, or a T cell receptor domain.

12. The protein of any one of embodiments 1-11, wherein the first moiety specifically binds to a molecule expressed in a diseased tissue.

13. The protein of any one of embodiments 1-12, wherein the first moiety specifically binds to a first molecule expressed in a diseased tissue and the second moiety is capable of specifically binding to a second molecule expressed in a diseased tissue, wherein the first molecule expressed in a diseased tissue and the second molecule expressed in a diseased tissue are different molecules.

14. The protein of embodiment 13, wherein the first molecule expressed in a diseased tissue and the second molecule expressed in a diseased tissue are expressed by the same cell.

15. The protein of embodiment 13, wherein the first molecule expressed in a diseased tissue and the second molecule expressed in a diseased tissue are expressed by different cells.

16. The protein of embodiment 13, wherein the first molecule expressed in a diseased tissue and/or the second molecule expressed in a diseased tissue is expressed on the surface of a cell.

17. The protein of embodiment 13, wherein the first molecule expressed in a diseased tissue and/or the second molecule expressed in a diseased tissue is a soluble molecule.

18. The protein of any one of embodiments 1-17, wherein the first moiety binds specifically to human EGFR, human HER2, human HER3, human CD105, human C-KIT, human PD1, human PD-L1, human PSMA, human EpCAM, human Trop2, human EphA2, human CD20, human BCMA, human GITR, human OX40, human CSF1R, human Lag3 or human cMET.

19. The protein of any one of embodiments 1-17, wherein the second moiety specifically binds to a molecule expressed by a human immune cell.

20. The protein of embodiment 19, wherein the molecule expressed by a human immune cell is human CD3, human CD16A, human CD16B, human CD28, human CD89, human CTLA4, human NKG2D, human SIRPα, human SIRPγ, human PD1, human Lag3, human 4-1BB, human OX40, or human GITR.

21. The protein of any one of embodiments 1-20, wherein the first moiety comprises a heavy chain variable (VH) region and a light chain variable (VL) region.

22. The protein of any one of embodiments 1-21, wherein the first moiety comprises an immunoglobulin constant region or a portion of an immunoglobulin constant region.

23. The protein of embodiment 22, wherein the immunoglobulin constant region is IgG, IgE, IgM, IgD, IgA, or IgY.

24. The protein of embodiment 22, wherein the immunoglobulin constant region is IgG1, IgG2, IgG3, IgG4, IgA1, or IgA2.

25. The protein of embodiment 22, wherein the immunoglobulin constant region is immunologically inert.

26. The protein of embodiment 22, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A and L235A, a human IgG1 constant region comprising the amino acid substitutions L234A, L235A and G237A, a human IgG1 constant region comprising the amino acid substitutions L234A, L235A, G237A and P331 S, or a wild-type human IgG2 constant region.

27. The protein of any one of embodiments 1-26, wherein the second moiety comprises an antibody, an antigen-binding portion of an antibody or a receptor ectodomain.

28. The protein of embodiment 27, wherein the second moiety is a Fab, a single-chain Fab, a VH domain, a VL domain, an immunoglobulin new antigen receptor (IgNAR), a single-chain variable fragment (scFv), or a T cell receptor domain.

29. The protein of any one of embodiments 1-28, wherein the second moiety binds specifically to human CD47.

30. The protein of any one of embodiments 1-28, wherein the second moiety binds specifically to human CD3 or human PD-L1.

31. The protein of any one of embodiments 1-30, wherein the second moiety comprises a heavy chain variable (VH) region and a light chain variable (VL) region.

32. The protein of any one of embodiments 1-31, wherein the second moiety comprises an immunoglobulin constant region or a portion of an immunoglobulin constant region.

33. The protein of embodiment 32, wherein the immunoglobulin constant region is IgG, IgE, IgM, IgD, IgA or IgY.

34. The protein of embodiment 32, wherein the immunoglobulin constant region is IgG1, IgG2, IgG3, IgG4, IgA1 or IgA2.

35. The protein of embodiment 32, wherein the immunoglobulin constant region is immunologically inert.

36. The protein of embodiment 32, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A and L235A, a human IgG1 constant region comprising the amino acid substitutions L234A, L235A and G237A, a human IgG1 constant region comprising the amino acid substitutions L234A, L235A, G237A and P331 S, or a wild-type human IgG2 constant region.

37. The protein of embodiment 1, wherein the protein has an immune effector function or two, three or more immune effector functions.

38. The protein of embodiment 37, wherein the immune effector function is ADCC, CDC or ADCP.

39. The protein of any one of embodiments 1-38, wherein the first moiety prevents or reduces specific binding of the second moiety to the molecule expressed in the diseased tissue.

40. The protein of any one of embodiments 1-39, wherein the peptide linker is cleaved in the vicinity of the diseased tissue or inside the diseased tissue.

41. The protein of any one of embodiments 1-40, wherein the peptide linker is cleaved in the vicinity of the diseased tissue or inside the diseased tissue, wherein the first moiety dissociates from the second moiety in the vicinity of the diseased tissue or inside the diseased tissue and wherein the second moiety specifically binds to the molecule expressed in the diseased tissue in the vicinity of the diseased tissue or inside the diseased tissue.

42. The protein of any one of embodiments 1-41, wherein the diseased tissue is a tumour or an inflamed tissue.

43. The protein of embodiment 1, wherein the first moiety binds specifically to human cMET, wherein the second moiety binds specifically to human CD47, and wherein the protein comprises a first polypeptide chain comprising or consisting of the amino acid sequence of SEQ ID NO:16 and a second polypeptide chain comprising or consisting of the amino acid sequence of SEQ ID NO:17.

44. The protein of embodiment 1, wherein the first moiety binds specifically to human HER2, wherein the second moiety binds specifically to human CD3, and wherein the protein comprises a first polypeptide chain comprising or consisting of the amino acid sequence of SEQ ID NO:26 and a second polypeptide chain comprising or consisting of the amino acid sequence of SEQ ID NO:27.

45. The protein of embodiment 1, wherein the first moiety binds specifically to human HER2, wherein the second moiety binds specifically to human CD47, and wherein the protein comprises a first polypeptide chain comprising or consisting of the amino acid sequence of SEQ ID NO:34 and a second polypeptide chain comprising or consisting of the amino acid sequence of SEQ ID NO:35.

46. The protein of embodiment 1, wherein the first moiety binds specifically to human cMET, wherein the second moiety binds specifically to human CD47, and wherein the protein comprises a first polypeptide chain comprising or consisting of the amino acid sequence of SEQ ID NO:36 and a second polypeptide chain comprising or consisting of the amino acid sequence of SEQ ID NO:37.

47. The protein of embodiment 1, wherein the first moiety binds specifically to human Her2, wherein the second moiety binds specifically to human CD3, and wherein the protein comprises a first polypeptide chain comprising or consisting of the amino acid sequence of SEQ ID NO:38 and a second polypeptide chain comprising or consisting of the amino acid sequence of SEQ ID NO:39.

48. The protein of embodiment 1, wherein the first moiety binds specifically to human Her2, wherein the second moiety binds specifically to human CD3, and wherein the protein comprises a first polypeptide chain comprising or consisting of the amino acid sequence of SEQ ID NO:40 and a second polypeptide chain comprising or consisting of the amino acid sequence of SEQ ID NO:41.

49. The protein of embodiment 1, wherein the first moiety binds specifically to human Her2, wherein the second moiety binds specifically to human CD47, and wherein the protein comprises a first polypeptide chain and a second polypeptide chain, wherein:
(a) the first polypeptide chain comprises or consists of the amino acid sequence of SEQ ID NO:42, and the second polypeptide chain comprises or consists of the amino acid sequence of SEQ ID NO:43; or
(b) the first polypeptide chain comprises or consists of the amino acid sequence of SEQ ID NO:44, and the second polypeptide chain comprises or consists of the amino acid sequence of SEQ ID NO:45; or
(c) the first polypeptide chain comprises or consists of the amino acid sequence of SEQ ID NO:46, and the second polypeptide chain comprises or consists of the amino acid sequence of SEQ ID NO:47; or
(d) the first polypeptide chain comprises or consists of the amino acid sequence of SEQ ID NO:48, and the second polypeptide chain comprises or consists of the amino acid sequence of SEQ ID NO:49; or
(e) the first polypeptide chain comprises or consists of the amino acid sequence of SEQ ID NO:50, and the second polypeptide chain comprises or consists of the amino acid sequence of SEQ ID NO:51; or
(f) the first polypeptide chain comprises or consists of the amino acid sequence of SEQ ID NO:52, and the second polypeptide chain comprises or consists of the amino acid sequence of SEQ ID NO:53; or
(g) the first polypeptide chain comprises or consists of the amino acid sequence of SEQ ID NO:54, and the second polypeptide chain comprises or consists of the amino acid sequence of SEQ ID NO:55; or
(h) the first polypeptide chain comprises or consists of the amino acid sequence of SEQ ID NO:88, and the second polypeptide chain comprises or consists of the amino acid sequence of SEQ ID NO:89; or
(i) the first polypeptide chain comprises or consists of the amino acid sequence of SEQ ID NO:90, and the second polypeptide chain comprises or consists of the amino acid sequence of SEQ ID NO:91; or
(j) the first polypeptide chain comprises or consists of the amino acid sequence of SEQ ID NO:44, and the second polypeptide chain comprises or consists of the amino acid sequence of SEQ ID NO:92; or
(k) the first polypeptide chain comprises or consists of the amino acid sequence of SEQ ID NO:44, and the second polypeptide chain comprises or consists of the amino acid sequence of SEQ ID NO:93; or
(l) the first polypeptide chain comprises or consists of the amino acid sequence of SEQ ID NO:44, and the second polypeptide chain comprises or consists of the amino acid sequence of SEQ ID NO:94; or
(m) the first polypeptide chain comprises or consists of the amino acid sequence of SEQ ID NO:44, and the second polypeptide chain comprises or consists of the amino acid sequence of SEQ ID NO:95; or
(n) the first polypeptide chain comprises or consists of the amino acid sequence of SEQ ID NO:44, and the second polypeptide chain comprises or consists of the amino acid sequence of SEQ ID NO:96; or
(o) the first polypeptide chain comprises or consists of the amino acid sequence of SEQ ID NO:44, and the second polypeptide chain comprises or consists of the amino acid sequence of SEQ ID NO:97.

50. The protein of embodiment 1, wherein the first moiety binds specifically to human Her2, wherein the second moiety binds specifically to human CD3, and wherein the protein comprises a first polypeptide chain comprising or consisting of the amino acid sequence of SEQ ID NO:73 and a second polypeptide chain comprising or consisting of the amino acid sequence of SEQ ID NO:74.

51. The protein of embodiment 1, wherein the first moiety binds specifically to human cMET, wherein the second moiety binds specifically to human cMET, and wherein the protein comprises a first polypeptide chain comprising or consisting of the amino acid sequence of SEQ ID NO:75 and a second polypeptide chain comprising or consisting of the amino acid sequence of SEQ ID NO:76.

52. The protein of embodiment 1, wherein the first moiety binds specifically to human Her2, wherein the second moiety binds specifically to human CD3, and wherein the protein comprises a first polypeptide chain and a second polypeptide chain, wherein:
(a) the first polypeptide chain comprises or consists of the amino acid sequence of SEQ ID NO:98, and the second polypeptide chain comprises or consists of the amino acid sequence of SEQ ID NO:99; or
(b) the first polypeptide chain comprises or consists of the amino acid sequence of SEQ ID NO: 100, and the second polypeptide chain comprises or consists of the amino acid sequence of SEQ ID NO:101; or
(c) the first polypeptide chain comprises or consists of the amino acid sequence of SEQ ID NO:102, and the second polypeptide chain comprises or consists of the amino acid sequence of SEQ ID NO:103; or
(d) the first polypeptide chain comprises or consists of the amino acid sequence of SEQ ID NO:104, and the second polypeptide chain comprises or consists of the amino acid sequence of SEQ ID NO:105.

53. An immunoconjugate comprising the protein of any one of embodiments 1-52 linked to a therapeutic agent.

54. The immunoconjugate of embodiment 53, wherein the therapeutic agent is a cytotoxin, a radioisotope, a chemotherapeutic agent, an immunomodulatory agent, an anti-angiogenic agent, an antiproliferative agent, a pro-apoptotic agent, a cytostatic enzyme, a cytolytic enzymes, a therapeutic nucleic acid, an anti-angiogenic agent, an anti-proliferative agent, or a pro-apoptotic agent.

55. A pharmaceutical composition comprising the protein of any one of embodiments 1-52 or the immunoconjugate of embodiment 53 or 54, and a pharmaceutically acceptable carrier, diluent or excipient.

56. A nucleic acid molecule encoding the protein or a portion of the protein of any one of embodiments 1-52.

57. A nucleic acid molecule encoding the first polypeptide chain, the second polypeptide chain, or both the first polypeptide chain and the second polypeptide chain of the protein of any one of embodiments 43-52.

58. An expression vector comprising the nucleic acid molecule of embodiment 56 or 57.

59. A recombinant host cell comprising the nucleic acid molecule of embodiment 56 or 57 or the expression vector of embodiment 58.

60. A method of producing a protein, the method comprising:
culturing a recombinant host cell comprising the expression vector of embodiment 57 under conditions whereby the nucleic acid molecule is expressed, thereby producing the protein; and
isolating the protein from the host cell or culture.

61. A method for enhancing an anti-cancer immune response in a subject, comprising administering to the subject a therapeutically effective amount of the protein of any one of embodiments 1-52, the immunoconjugate of embodiment 53 or 54, or the pharmaceutical composition of embodiment 55.

62. A method of treating cancer, an autoimmune disease, an inflammatory disease, a cardiovascular disease or a fibrotic disease in a subject, comprising administering to the subject a therapeutically effective amount of the protein of any one of embodiments 1-52, the immunoconjugate of embodiment 53 or 54, or the pharmaceutical composition of embodiment 55.

63. The method of embodiment 62, wherein the cancer is Gastrointestinal Stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma or cancer of hematological tissues.

64. The method of embodiment 62, wherein the autoimmune disease or the inflammatory disease is arthritis, asthma, multiple sclerosis, psoriasis, Crohn’s disease, inflammatory bowel disease, lupus, Grave’s disease, Hashimoto’s thyroiditis or ankylosing spondylitis.

65. The method of embodiment 62, wherein the cardiovascular disease is coronary heart disease, or atherosclerosis or stroke.

66. The method of embodiment 62, wherein the fibrotic disease is myocardial infarction, angina, osteoarthritis, pulmonary fibrosis, cystic fibrosis, bronchitis or asthma.

67. The protein of any one of embodiments 1-52, the immunoconjugate of embodiment 53 or 54, or the pharmaceutical composition of embodiment 55 for use in the treatment of cancer, an autoimmune disease, an inflammatory disease, a cardiovascular disease or a fibrotic disease.

68. The protein or the pharmaceutical composition for use according to embodiment 67, wherein the cancer is Gastrointestinal Stromal cancer (GIST), pancreatic cancer, skin cancer, melanoma, breast cancer, lung cancer, bronchial cancer, colorectal cancer, prostate cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma or cancer of hematological tissues.

69. The protein or the pharmaceutical composition for use according to embodiment 67, wherein the autoimmune disease or the inflammatory disease is arthritis, asthma, multiple sclerosis, psoriasis, Crohn’s disease, inflammatory bowel disease, lupus, Grave’s disease, Hashimoto’s thyroiditis or ankylosing spondylitis.

70. The protein or the pharmaceutical composition for use according to embodiment 67, wherein the cardiovascular disease is coronary heart disease, atherosclerosis, or stroke.

71. The protein or the pharmaceutical composition for use according to embodiment 67, wherein the fibrotic disease is myocardial infarction, angina, osteoarthritis, pulmonary fibrosis, cystic fibrosis, bronchitis or asthma.

72. The protein of any one of embodiments 1-52, the immunoconjugate of embodiment 53 or 54, or the pharmaceutical composition of embodiment 55, for use as a medicament.

TABLE 1

Peptide linker sequences.

| Constant domain | Linker name | Unique linker sequence | Unique Linker length (bp) |
|---|---|---|---|
| CH1 | L1 | GGGGS (SEQ ID NO:15) | 5 |
| | L2 | GGGGSGGGGS (SEQ ID NO:32) | 10 |
| | L3 | GGGGSGGGGSGGGGS (SEQ ID NO:33) | 15 |
| | LH | GPAPELL (SEQ ID NO:1) | 7 |
| | LHL | GPAPELLGGGS (SEQ ID NO:2) | 11 |
| | LHL-F | GPAPLGLGGGS (SEQ ID NO:3) | 11 |
| | LHL-M | PPCPAPELLGGGS (SEQ ID NO:4) | 13 |
| | LHL-MF | PPCPAPLGLGGGS (SEQ ID NO:5) | 13 |
| CL (Kappa or Lambda) | L1 | GGGGS (SEQ ID NO:15) | 5 |
| | L2 | GGGGSGGGGS (SEQ ID NO:32) | 10 |
| | L3 | GGGGSGGGGSGGGGS (SEQ ID NO:33) | 15 |
| | LH | GPAPELL (SEQ ID NO:1) | 7 |
| | LHL | GPAPELLGGGS (SEQ ID NO:2) | 11 |
| | LHL-F | GPAPLGLGGGS (SEQ ID NO:3) | 11 |
| | LHL-M | PPCPAPELLGGGS (SEQ ID NO:4) | 13 |
| | LHL-MF | PPCPAPLGLGGGS (SEQ ID NO:5) | 13 |

Underlined linker peptide sequences are human IgG1 germline.
Non-underlined peptide sequences in bold (LG) are mutations that instate the rapidly-digested MMP peptide substrate sequence ‘PLGL’ (SEQ ID NO:12).

TABLE 2

Protein clone numbers, names (ID) and observed expression characteristics.

| Clone # | Antibody ID | Protein yield* | Percentage Main Peak(SEC) | Expected** Molecular Weight (kDa) | |
|---|---|---|---|---|---|
| | | | | non-reduced | reduced |
| 1 | Fab2 cMETCD47-L1-L1 | 2.7 | 52 | 146.49 | 72.96; 73.55 |
| 2 | Fab2 cMETCD47-L2-L2 | 1.1 | 66 | 147.12 | 73.28; 73.86 |
| 3 | Fab2 cMETCD47-L3-L3 | 5.7 | 45 | 147.75 | 73.59; 74.18 |
| 4 | Fab2 cMETCD47-L1-LH | 2.3 | 49 | 146.85 | 72.96; 73.91 |
| 5 | Fab2 cMETCD47-LH-L1 | 3.4 | 49 | 146.85 | 73.33; 73.55 |
| 6 | Fab2 cMETCD47-LHL-LHL | 3.4 | 66 | 147.73 | 73.58; 74.17 |
| 7 | Fab2 Her2CD3-L2-L2 | 3.1 | 61 | 146.55 | 74.09; 72.47 |
| 8 | Fab2 Her2CD3-L1-LH | 3.6 | 73 | 146.28 | 73.78; 72.52 |
| 9 | Fab2 Her2CD3-LH-L1 | 0.2 | ND | 146.28 | 74.14; 72.16 |
| 10 | Fab2 Her2CD3-LHL-LHL | 1.3 | 58 | 147.16 | 74.40; 72.78 |
| 11 | IgG2 Her2CD47-L1-LH | 2.4 | 29 | 241.56 | 72.66; 48.15 |
| 12 | IgG2 Her2CD47-LH-L1 | 8.1 | 49 | 241.56 | 73.02; 47.78 |
| 13 | IgG2 Her2CD47-LH-LH | 1.96 | 62 | 242.28 | 73.02; 48.15 |
| 14 | IgG2 Her2CD47-LHL-LHL | 18 | 90 | 243.32 | 73.28; 48.40 |
| 15 | FabCD47-Only | 1.5 | 82 | 98.49 | 49.13; 49.37 |

* Total protein after Protein A column affinity purification
** Based on amino acid sequence only
ND = not done

TABLE 3

Sequences of bispecific proteins binding to cMet and CD47.

| Construct Type: | Fab2 CMET/CD47 | | |
|---|---|---|---|
| Clone Name: | Met47-L1-L1 | | |
| Chain name | Heavy chain amino acid sequence | Chain name | Light chain amino acid sequence |
| Met47 knob-L1 | QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQVQLVQSGAEVKKPGESLKISCKGSGYTFTNYYIFWVRQMPGKGLEWMG IINPVDGDTNYNPSFQGQVTISADKSISTAYLQWSSLKASDTAMYYCARGGYTMRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG (SEQ ID NO:6) | Met47 hole-L1 | EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPELLGGPSVFLFPPKPKDTL MISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLS LSPG (SEQ ID NO:7) |
| Clone name: | Met47-L2-L2 | | |
| Chain name | Heavy chain amino acid sequence | Chain name | Light chain amino acid sequence |
| Met47 knob-L2 | QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGESLKISCKGSGYTFTNYYIFWVRQMPGKG LEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHSNTKVDKKVEPKSCDKGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG (SEQ ID NO:8) | Met47 hole-L2 | EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAL PAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG (SEQ ID NO:9) |
| Met47 knob-L3 | QVQLVQSGAEVKKPGASVKVSCKASGY TFTSYTMHWVRQAPGQGLEWMGIINP SGGSTSYAQKFQGRVTMTRDTSTSTVY MELSSLRSEDTAVYYCARQEITTEFDYW GQGTLVTVSSASTKGPSVFPLAPSSKSTS GGTAALGCLVKDYFPEPVTVSWNSGAL TSGVHTFPAVLQSSGLYSLSSVVTVPSSS LGTQTYICNVNHKPSNTKVDKKVEPKSC GGGGSGGGGSGGGGSQVQLVQSGAE VKKPGESLKISCKGSGYTFTNYYIFWVRQ MPGKGLEWMGIINPVDGDTNYNPSFQ GQVTISADKSISTAYLQWSSLKASDTAM YYCARGGYTMDRWGQGTLVTVSSAST KGPSVFPLAPSSKSTSGGTAALGCLVKD YFPEPVTVSWNSGALTSGVHTFPAVLQS SGLYSLSSVVTVPSSSLGTQTYICNVNHK PSNTKVDKKVEPKSCDKGGGGSGGGGS CPPCPAPELLGGPSVFLFPPKPKDTLMIS RTPEVTCVVVDVSHEDPEVKFNWYVDG VEVHNAKTKPREEQYNSTYRVVSVLTVL HQDWLNGKEYKCKVSNKALPAPIEKTIS KAKGQPREPQVYTLPPSRDELTKNQVSL WCLVKGFYPSDIAVEWESNGQPENNYK TTPPVLDSDGSFFLYSKLTVDKSRWQQG NVFSCSVMHEALHNHYTQKSLSLSPG (SEQ ID NO:10) | Met47 hole-L3 | EIVLTQSPGTLSLSPGERATLSCRASQSV SSYANSYLHWYQQKPGQAPRLLIYRGST RESGIPDRFSGSGSGTDFTLTISRLEPEDF AVYYCQQSKSEPLTFGGGTKVEIKRTVA APSVFIFPPSDEQLKSGTASVVCLLNNFY PREAKVQWKVDNALQSGNSQESVTEQ DSKDSTYSLSSTLTLSKADYEKHKVYACE VTHQGLSSPVTKSFNRGECGGGGSGGG GSGGGGSDIVMTQSPLSLPVTPGEPASI SCRSSQSLLHSNGYNYLHWYLQKPGQS PQLLlYKVSNRLSGVPDRFSGSGSGTDFT LKISRVEAEDVGVYYCFQNTHTPRTFGG GTKVRTVAAPSVFIFPPSDEQLKSGTASV VCLLNNFYPREAKVQWKVDNALQSGN SQESVTEQDSKDSTYSLSSTLTLSKADYE KHKVYACEVTHQGLSSPVTKSFNRGEC GGGGSGGGGSCPPCPAPELLGGPSVFL FPPKPKDTLMISRTPEVTCVVVDVSHED PEVKFNWYVDGVEVHNAKTKPREEQY NSTYRVVSVLTVLHQDWLNGKEYKCKV SNKALPAPIEKTISKAKGQPREPQVYTLP PSRDELTKNQVSLSCAVKGFYPSDIAVE WESNGQPENNYKTTPPVLDSDGSFFLV SKLTVDKSRWQQGNVFSCSVMHEALH NHYTQKSLSLSPG (SEQ ID NO:11) |
| Met47 knob-L1 | QVQLVQSGAEVKKPGASVKVSCKASGY TFTSYTMHWVRQAPGQGLEWMGIINP SGGSTSYAQKFQGRVTMTRDTSTSTVY MELSSLRSEDTAVYYCARQEITTEFDYW GQGTLVTVSSASTKGPSVFPLAPSSKSTS GGTAALGCLVKDYFPEPVTVSWNSGAL TSGVHTFPAVLQSSGLYSLSSVVTVPSSS LGTQTYICNVNHKPSNTKVDKKVEPKSC GGGGSQVQLVQSGAEVKKPGESLKISCK GSGYTFTNYYIFWVRQMPGKGLEWMG IINPVDGDTNYNPSFQGQVTISADKSIST AYLQWSSLKASDTAMYYCARGGYTM D RWGQGTLVTVSSASTKGPSVFPLAPSSK STSGGTAALGCLVKDYFPEPVTVSWNS GALTSGVHTFPAVLQSSGLYSLSSVVTVP SSSLGTQTYICNVN H KPSNTKVDKKVE P KSCDKGGGGSGGGGSCPPCPAPELLGG PSVFLFPPKPKDTLMISRTPEVTCVVVDV SHEDPEVKFNWYVDGVEVHNAKTKPRE EQYNSTYRVVSVLTVLHQDWLNGKEYK CKVSNKALPAPIEKTISKAKGQPREPQVY TLPPSRDELTKNQVSLWCLVKGFYPSDI AVEWESNGQPENNYKTTPPVLDSDGSFFLYSKLTVDKSRWQQGNVFSCSVMHEA LHNHYTQKSLSLSPG (SEQ ID NO:6) | Met47 hole-LH | EIVLTQSPGTLSLSPGERATLSCRASQSV SSYANSYLHWYQQKPGQAPRLLIYRGST RESGIPDRFSGSGSGTDFTLTISRLEPEDF AVYYCQQSKSEPLTFGGGTKVEIKRTVA APSVFIFPPSDEQLKSGTASVVCLLNNFY PREAKVQWKVDNALQSGNSQESVTEQ DSKDSTYSLSSTLTLSKADYEKHKVYACE VTHQGLSSPVTKSFNRGECGPAPELLDI VMTQSPLSLPVTPGEPASISCRSSQSLLH SNGYNYLHWYLQKPGQSPQLLIYKVSN RLSGVPDRFSGSGSGTDFTLKISRVEAED VGVYYCFQNTHTPRTFGGGTKVEIKRTV AAPSVFIFPPSDEQLKSGTASVVCLLNNF YPREAKVQWKVDNALQSGNSQESVTE QDSKDSTYSLSSTLTLSKADYEKHKVYAC EVTHQGLSSPVTKSFNRGECGGGGSGG GGSCPPCPAPELLGGPSVFLFPPKPKDTL MISRTPEVTCVVVDVSHEDPEVKFNWY VDGVEVHNAKTKPREEQYNSTYRVVSV LTVLHQDWLNGKEYKCKVSNKALPAPIE KTISKAKGQPREPQVYTLPPSRDELTKN QVSLSCAVKGFYPSDIAVEWESNGQPE NNYKTTPPVLDSDGSFFLVSKLTVDKSRWQQGNVFSCSVMHEALHNHYTQKSLS LSPG (SEQ ID NO:13) |
| Met47 knob-LH | QVQLVQSGAEVKKPGASVKVSCKASGY TFTSYTMHWVRQAPGQGLEWMGIINP SGGSTSYAQKFQGRVTMTRDTSTSTVY MELSSLRSEDTAVYYCARQEITTEFDYW GQGTLVTVSSASTKGPSVFPLAPSSKSTS GGTAALGCLVKDYFPEPVTVSWNSGAL TSGVHTFPAVLQSSGLYSLSSVVTVPSSS LGTQTYICNVNHKPSNTKVDKKVEPKSC GPAPELLQVQLVQSGAEVKKPGESLKIS CKGSGYTFTNYYIFWVRQMPGKGLEW MGIINPVDGDTNYNPSFQGQVTISADK SISTAYLQWSSLKASDTAMYYCARGGYT MDRWGQGTLVTVSSASTKGPSVFPLAP SSKSTSGGTAALGCLVKDYFPEPVTVSW NSGALTSGVHTFPAVLQSSGLYSLSSVVT VPSSSLGTQTYICNVN H KPSNTKVDKKV EPKSCDKGGGGSGGGGSCPPCPAPELL GGPSVFLFPPKPKDTLMISRTPEVTCVV VDVSHEDPEVKFNWYVDGVEVHNAKT KPREEQYNSTYRVVSVLTVLHQDWLNG KEYKCKVSNKALPAPIEKTISKAKGQPRE PQVYTLPPSRDELTKNQVSLWCLVKGFY PSDIAVEWESNGQPENNYKTTPPVLDS DGSFFLYSKLTVDKSRWQQGNVFSCSV MHEALHNHYTQKSLSLSPG (SEQ ID NO:14) | Met47 hole-L1 | EIVLTQSPGTLSLSPGERATLSCRASQSV SSYANSYLHWYQQKPGQAPRLLIYRGST RESGIPDRFSGSGSGTDFTLTISRLEPEDF AVYYCQQSKSEPLTFGGGTKVEIKRTVA APSVFIFPPSDEQLKSGTASVVCLLNNFY PREAKVQWKVDNALQSGNSQESVTEQ DSKDSTYSLSSTLTLSKADYEKHKVYACE VTHQGLSSPVTKSFNRGECGGGGSDIV MTQSPLSLPVTPGEPASISCRSSQSLLHS NGYNYLHWYLQKPGQSPQLLIYKVSNR LSGVPDRFSGSGSGTDFTLKISRVEAEDV GVYYCFQNTHTPRTFGGGTKVEIKRTVA APSVFIFPPSDEQLKSGTASVVCLLNNFY PREAKVQWKVDNALQSGNSQESVTEQ DSKDSTYSLSSTLTLSKADYEKHKVYACE VTHQGLSSPVTKSFNRGECGGGGSGGG GSCPPCPAPELLGGPSVFLFPPKPKDTL MISRTPEVTCVVVDVSHEDPEVKFNWY VDGVEVHNAKTKPREEQYNSTYRVVSV LTVLHQDWLNGKEYKCKVSNKALPAPIE KTISKAKGQPREPQVYTLPPSRDELTKN QVSLSCAVKGFYPSDIAVEWESNGQPE NNYKTTPPVLDSDGSFFLVSKLTVDKSR WQQGNVFSCSVMHEALHNHYTQKSLS LSPG (SEQ ID NO:7) |
| Met47 knob-LHL | QVQLVQSGAEVKKPGASVKVSCKASGY TFTSYTMHWVRQAPGQGLEWMGIINP SGGSTSYAQKFQGRVTMTRDTSTSTVY MELSSLRSEDTAVYYCARQEITTEFDYW GQGTLVTVSSASTKGPSVFPLAPSSKSTS GGTAALGCLVKDYFPEPVTVSWNSGAL TSGVHTFPAVLQSSGLYSLSSVVTVPSSS LGTQTYICNVNHKPSNTKVDKKVEPKSC GPAPELLGGGSQVQLVQSGAEVKKPGE SLKISCKGSGYTFTNYYIFWVRQMPGKG LEWMGIINPVDGDTNYNPSFQGQVTIS ADKSISTAYLQWSSLKASDTAMYYCARG GYTMDRWGQGTLVTVSSASTKGPSVFP LAPSSKSTSGGTAALGCLVKDYFPEPVTV SWNSGALTSGVHTFPAVLQSSGLYSLSS VVTVPSSSLGTQTYICNVN H KPSNTKVD KKVEPKSCDKGGGGSGGGGSCPPCPAP ELLGGPSVFLFPPKPKDTLMISRTPEVTC VVVDVSHEDPEVKFNWYVDGVEVHNA KTKPREEQYNSTYRVVSVLTVLHQDWL NGKEYKCKVSNKALPAPIEKTISKAKGQP REPQVYTLPPSRDELTKNQVSLWCLVKG FYPSDIAVEWESNGQPENNYKTTPPVL DSDGSFFLYSKLTVDKSRWQQGNVFSCSVMHEALHNHYTQKSLSLSPG (SEQ ID NO:16) | Met47 hole-LHL | EIVLTQSPGTLSLSPGERATLSCRASQSV SSYANSYLHWYQQKPGQAPRLLIYRGST RESGIPDRFSGSGSGTDFTLTISRLEPEDF AVYYCQQSKSEPLTFGGGTKVEIKRTVA APSVFIFPPSDEQLKSGTASVVCLLNNFY PREAKVQWKVDNALQSGNSQESVTEQ DSKDSTYSLSSTLTLSKADYEKHKVYACE VTHQGLSSPVTKSFNRGECGPAPELLGG GSDIVMTQSPLSLPVTPGEPASISCRSSQ SLLHSNGYNYLHWYLQKPGQSPQLLIYK VSNRLSGVPDRFSGSGSGTDFTLKISRVE AEDVGVYYCFQNTHTPRTFGGGTKVEIK RTVAAPSVFIFPPSDEQLKSGTASVVCLL NNFYPREAKVQWKVDNALQSGNSQES VTEQDSKDSTYSLSSTLTLSKADYEKHKV YACEVTHQGLSSPVTKSFNRGECGGGG SGGGGSCPPCPAPELLGGPSVFLFPPKP KDTLMISRTPEVTCVVVDVSHEDPEVKF NWYVDGVEVHNAKTKPREEQYNSTYR VVSVLTVLHQDWLNGKEYKCKVSNKAL PAPIEKTISKAKGQPREPQVYTLPPSRDE LTKNQVSLSCAVKGFYPSDIAVEWESNG QPENNYKTTPPVLDSDGSFFLVSKLTVDKSRWQQGNVFSCSVMHEALHNHYTQ KSLSLSPG (SEQ ID NO:17) |
| | QVQLVQSGAEVKKPGESLKISCKGSGYT FTNYYIFWVRQMPGKGLEWMGIINPV DGDTNYNPSFQGQVTISADKSISTAYLQ WSSLKASDTAMYYCARGGYTMDRWG QGTLVTVSSASTKGPSVFPLAPSSKSTSG GTAALGCLVKDYFPEPVTVSWNSGALTS GVHTFPAVLQSSGLYSLSSVVTVPSSSLG TQTYICNVNHKPSNTKVDKKVEPKSCDK GGGGSGGGGSCPPCPAPELLGGPSVFL FPPKPKDTLMISRTPEVTCVVVDVSHED PEVKFNWYVDGVEVHNAKTKPREEQY NSTYRVVSVLTVLHQDWLNGKEYKCKV SNKALPAPIEKTISKAKGQPREPQVYTLP PSRDELTKNQVSLWCLVKGFYPSDIAVE WESNGQPENNYKTTPPVLDSDGSFFLY SKLTVDKSRWQQGNVFSCSVMHEALH NHYTQKSLSLSPG (SEQ ID NO:18) | | DIVMTQSPLSLPVTPGEPASISCRSSQSL LHSNGYNYLHWYLQKPGQSPQLLIYKVS NRLSGVPDRFSGSGSGTDFTLKISRVEAE DVGVYYCFQNTHTPRTFGGGTKVEIKRT VAAPSVFIFPPSDEQLKSGTASVVCLLNN FYPREAKVQWKVDNALQSGNSQESVT EQDSKDSTYSLSSTLTLSKADYEKHKVYA CEVTHQGLSSPVTKSFNRGECGGGGSG GGGSCPPCPAPELLGGPSVFLFPPKPKD TLMISRTPEVTCVVVDVSHEDPEVKFN WYVDGVEVHNAKTKPREEQYNSTYRV VSVLTVLHQDWLNGKEYKCKVSNKALP APIEKTISKAKGQPREPQVYTLPPSRDEL TKNQVSLSCAVKGFYPSDIAVEWESNG QPENNYKTTPPVLDSDGSFFLVSKLTVD KSRWQQGNVFSCSVMHEALHNHYTQ KSLSLSPG (SEQ ID NO:19) |

TABLE 4

Sequences of bispecific proteins binding to Her2 and CD3

| Construct Type: | Fab2 Her2/CD3 | | |
|---|---|---|---|
| Clone Name: | Her23-L2-L2 | | |
| Chain name | Heavy chain amino acid sequence | Chain name | Light chain amino acid sequence |
| Her23 knob-L2 | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVN H KPSNTKVDKKVE P KSCGGGGSGGGGSQVQLQQSGAELAR PGASVKMSCKASGYTFTRYTM HWVKQ RPGQGLEWIGYINPSRGYTNYNQKFKD KATLTTDKSSSTAYM QLSSLTSE DSAVYY CARYYDDHYCLDYWGQGTTLTVSSAST KGPSVFPLAPSSKSTSGGTAALGCLVKD YFPEPVTVSWNSGALTSGVHTFPAVLQS SGLYSLSSVVTVPSSSLGTQTYICNVNHK PSNTKVDKKVEPKSCDKGGGGSGGGGS CPPCPAPEAAGAPSVFLFPPKPKDTLMI SRTPEVTCVVVDVSHEDPEVKFNWYVD GVEVHNAKTKPREEQYNSTYRVVSVLTV LHQDWLNGKEYKCKVSNKALPAPIEKTI SKAKGQPREPQVYTLPPSRDELTKNQVS LWCLVKGFYPSDIAVEWESNGQPENNY KTTPPVLDSDGSFFLYSKLTVDKSRWQQ GNVFSCSVMHEALHNHYTQKSLSLSPG (SEQ ID NO:20) | Her23 hole-L2 | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECGGGGSGGGGS QIVLTQSPAIMSASPGEKVTMTCSASSS VSYMNWYQQKSGTSPKRWIYDTSKLAS GVPAHFRGSGSGTSYSLTISGMEAEDAA TYYCQQWSSNPFTFGSGTKLEINRTVAA PSVFIFPPSDEQLKSGTASVVCLLNNFYP REAKVQWKVDNALQSGNSQESVTEQD SKDSTYSLSSTLTLSKADYEKH KVYACEV THQGLSSPVTKSFNRGECGGGGSGGG GSCPPCPAPEAAGAPSVFLFPPKPKDTL MISRTPEVTCVVVDVSHEDPEVKFNWY VDGVEVHNAKTKPREEQYNSTYRVVSV LTVLHQDWLNGKEYKCKVSNKALPAPIE KTISKAKGQPREPQVYTLPPSRDELTKN QVSLSCAVKGFYPSDIAVEWESNGQPE N NYKTTPPVLDSDGSFFLVSKLTVDKSR WQQG NVFSCSVM H EALH N HYTQKSLS LSPG (SEQ ID NO:21) |
| Her23 knob-L1 | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCGGGGSQVQLQQSGAELARPGASV KMSCKASGYTFTRYTMHWVKQRPGQG LEWIGYINPSRGYTNYNQKFKDKATLTT DKSSSTAYMQLSSLTSEDSAVYYCARYY DDHYCLDYWGQGTTLTVSSASTKGPSV FPLAPSSKSTSGGTAALGCLVKDYFPEPV TVSWNSGALTSGVHTFPAVLQSSGLYSL SSVVTVPSSSLGTQTYICNVNHKPSNTK VDKKVEPKSCDKGGGGSGGGGSCPPCP APEAAGAPSVFLFPPKPKDTLMISRTPEV TCVVVDVSHEDPEVKFNWYVDGVEVH NAKTKPREEQYNSTYRVVSVLTVLHQD WLNGKEYKCKVSNKALPAPIEKTISKAK GQPREPQVYTLPPSRDELTKNQVSLWC LVKGFYPSDIAVEWESNGQPENNYKTT PPVLDSDGSFFLYSKLTVDKSRWQQGN VFSCSVMHEALHNHYTQKSLSLSPG (SEQ ID NO:22) | Her23 hole-LH | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPELLQIVLT QSPAIMSASPGEKVTMTCSASSSVSYM NWYQQKSGTSPKRWIYDTSKLASGVPA H FRGSGSGTSYSLTISGMEAEDAATYYC QQWSSNPFTFGSGTKLEINRTVAAPSVF IFPPSDEQLKSGTASVVCLLNNFYPREAK VQWKVDNALQSGNSQESVTEQDSKDS TYSLSSTLTLSKADYEKHKVYACEVTHQG LSSPVTKSFNRGECGGGGSGGGGSCPP CPAPEAAGAPSVFLFPPKPKDTLMISRT PEVTCVVVDVSHEDPEVKFNWYVDGV EVHNAKTKPREEQYNSTYRVVSVLTVLH QDWLNGKEYKCKVSNKALPAPIEKTISK AKGQPREPQVYTLPPSRDELTKNQVSLS CAVKGFYPSDIAVEWESNGQPENNYKT TPPVLDSDGSFFLVSKLTVDKSRWQQG NVFSCSVMHEALHNHYTQKSLSLSPG (SEQ ID NO:23) |
| Her23 knob-LH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVE P KSCGPAPELLQVQLQQSGAELARPGAS VKMSCKASGYTFTRYTMHWVKQRPGQ GLEWIGYINPSRGYTNYNQKFKDKATLT TDKSSSTAYMQLSSLTSEDSAVYYCARYY DDHYCLDYWGQGTTLTVSSASTKGPSV FPLAPSSKSTSGGTAALGCLVKDYFPEPV TVSWNSGALTSGVHTFPAVLQSSGLYSL SSVVTVPSSSLGTQTYICNVNHKPSNTK VDKKVEPKSCDKGGGGSGGGGSCPPCP APEAAGAPSVFLFPPKPKDTLMISRTPEV TCVVVDVSHEDPEVKFNWYVDGVEVH NAKTKPREEQYNSTYRVVSVLTVLHQD WLNGKEYKCKVSNKALPAPIEKTISKAK GQPREPQVYTLPPSRDELTKNQVSLWC LVKGFYPSDIAVEWESNGQPENNYKTT PPVLDSDGSFFLYSKLTVDKSRWQQGN VFSCSVMHEALHNHYTQKSLSLSPG (SEQ ID NO:24) | Her23 hole-L1 | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGGGGSQIVLT QSPAIMSASPGEKVTMTCSASSSVSYM NWYQQKSGTSPKRWIYDTSKLASGVPA HFRGSGSGTSYSLTISGMEAEDAATYYC QQWSSNPFTFGSGTKLEINRTVAAPSVF IFPPSDEQLKSGTASVVCLLNNFYPREAK VQWKVDNALQSGNSQESVTEQDSKDS TYSLSSTLTLSKADYEKHKVYACEVTHQG LSSPVTKSFNRGECGGGGSGGGGSCPP CPAPEAAGAPSVFLFPPKPKDTLMISRT PEVTCVVVDVSHEDPEVKFNWYVDGV EVHNAKTKPREEQYNSTYRVVSVLTVLH QDWLNGKEYKCKVSNKALPAPIEKTISK AKGQPREPQVYTLPPSRDELTKNQVSLS CAVKGFYPSDIAVEWESNGQPENNYKT TPPVLDSDGSFFLVSKLTVDKSRWQQG NVFSCSVMHEALHNHYTQKSLSLSPG (SEQ ID NO:25) |
| Her23 knob-LHL | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCGPAPELLGGGSQVQLQQSGAELAR PGASVKMSCKASGYTFTRYTMHWVKQ RPGQGLEWIGYINPSRGYTNYNQKFKD KATLTTDKSSSTAYMQLSSLTSEDSAVYY CARYYDDHYCLDYWGQGTTLTVSSAST KGPSVFPLAPSSKSTSGGTAALGCLVKD YFPEPVTVSWNSGALTSGVHTFPAVLQS SGLYSLSSVVTVPSSSLGTQTYICNVNHK PSNTKVDKKVEPKSCDKGGGGSGGGGS CPPCPAPEAAGAPSVFLFPPKPKDTLMI SRTPEVTCVVVDVSHEDPEVKFNWYVD GVEVHNAKTKPREEQYNSTYRVVSVLTV LHQDWLNGKEYKCKVSNKALPAPIEKTI SKAKGQPREPQVYTLPPSRDELTKNQVS LWCLVKGFYPSDIAVEWESNGQPENNY KTTPPVLDSDGSFFLYSKLTVDKSRWQQ GNVFSCSVMHEALHNHYTQKSLSLSPG (SEQ ID NO:26) | Her23 hole-LHL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECGPAPELLGGGS QIVLTQSPAIMSASPGEKVTMTCSASSS VSYMNWYQQKSGTSPKRWIYDTSKLAS GVPAH FRGSGSGTSYSLTISGMEAEDAA TYYCQQWSSNPFTFGSGTKLEINRTVAA PSVFIFPPSDEQLKSGTASVVCLLNNFYP REAKVQWKVDNALQSGNSQESVTEQD SKDSTYSLSSTLTLSKADYEKHKVYACEV THQGLSSPVTKSFNRGECGGGGSGGG GSCPPCPAPEAAGAPSVFLFPPKPKDTL MISRTPEVTCVVVDVSHEDPEVKFNWY VDGVEVHNAKTKPREEQYNSTYRVVSV LTVLHQDWLNGKEYKCKVSNKALPAPIE KTISKAKGQPREPQVYTLPPSRDELTKN QVSLSCAVKGFYPSDIAVEWESNGQPE NNYKTTPPVLDSDGSFFLVSKLTVDKSR WQQGNVFSCSVMHEALHNHYTQKSLS LSPG (SEQ ID NO:27) |

TABLE 5

Sequences of bispecific proteins binding to Her2 and CD47.

| Construct Type: | IgG Her2/CD47 | | |
|---|---|---|---|
| Clone Name: | Her47 -L1-LH | | |
| Chain name | Heavy chain amino acid sequence | Chain name | Light chain amino acid sequence |
| Her47 L1-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCGGGGSQVQLVQSGAEVKKPGESLKI SCKGSGYTFTNYYIFWVRQMPGKGLEW MGIINPVDGDTNYNPSFQGQVTISADK SISTAYLQWSSLKASDTAMYYCARGGYT MDRWGQGTLVTVSS (SEQ ID NO:28) | Her47 LH-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPELLDIVM TQSPLSLPVTPGEPASISCRSSQSLLHSN GYNYLHWYLQKPGQSPQLLIYKVSNRLS GVPDRFSGSGSGTDFTLKISRVEAEDVG VYYCFQNTHTPRTFGGGTKVEIK (SEQ ID NO:29) |
| Clone name: | Her47 -LH-L1 | | |
| Chain name | Heavy chain amino acid sequence | Chain name | Light chain amino acid sequence |
| Her47 LH-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS | Her47 L1-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR |
| | KSTSGGTAALGCLVKDYFPEPVTVSWNS GALTSGVHTFPAVLQSSGLYSLSSVVTVP SSSLGTQTYICNVNHKPSNTKVDKKVEP KSCGPAPELLQVQLVQSGAEVKKPGESL KISCKGSGYTFTNYYIFWVRQMPGKGLE WMGIINPVDGDTNYNPSFQGQVTISAD KSISTAYLQWSSLKASDTAMYYCARGGY TMDRWGQGTLVTVSS (SEQ ID NO:30) | | EAKVQWKVDNALQSGNSQESVTEQDS KDSTYSLSSTLTLSKADYEKH KVYACEVT HQGLSSPVTKSFNRGECGGGSDIVMTQ SPLSLPVTPGEPASISCRSSQSLLHSNGY NYLHWYLQKPGQSPQLLIYKVSNRLSGV PDRFSGSGSGTDFTLKISRVEAEDVGVY YCFQNTHTPRTFGGGTKVEIK (SEQ ID NO:31) |
| Her47 LH-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVE P KSCGPAPELLQVQLVQSGAEVKKPGESL KISCKGSGYTFTNYYIFWVRQMPGKGLE WMGIINPVDGDTNYNPSFQGQVTISAD KSISTAYLQWSSLKASDTAMYYCARGGY TMDRWGQGTLVTVSS (SEQID NO:30) | Her47 LH-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECGPAPELLDIVM TQSPLSLPVTPGEPASISCRSSQSLLHSN GYNYLHWYLQKPGQSPQLLIYKVSNRLS GVPDRFSGSGSGTDFTLKISRVEAEDVG VYYCFQNTHTPRTFGGGTKVEIK (SEQ ID NO:29) |
| Her47 LHL-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCGPAPELLGGGSQVQLVQSGAEVKK PGESLKISCKGSGYTFTNYYIFWVRQMP GKGLEWMGIINPVDGDTNYNPSFQGQ VTISADKSISTAYLQWSSLKASDTAMYY CARGGYTMDRWGQGTLVTVSS (SEQ ID NO:34) | Her47 LHL-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECGPAPELLGGGS DIVMTQSPLSLPVTPGEPASISCRSSQSL LHSNGYNYLHWYLQKPGQSPQLLIYKVS NRLSGVPDRFSGSGSGTDFTLKISRVEAE DVGVYYCFQNTHTPRTFGGGTKVEIK (SEQ ID NO:35) |

TABLE 6

Sequences of bispecific protein binding to cMET and CD47.

| Construct Type: | Fab2 CMET/CD47 “One Arm” style | | |
|---|---|---|---|
| Clone Name: | Met47-LHL-LHL | | |
| Chain name | Heavy chain amino acid sequence | Chain name | Light chain amino acid sequence |
| Met47 knob-LHL | QVQLVQSGAEVKKPGASVKVSCKASGY TFTSYTMHWVRQAPGQGLEWMGIINP SGGSTSYAQKFQGRVTMTRDTSTSTVY MELSSLRSEDTAVYYCARQEITTEFDYW GQGTLVTVSSASTKGPSVFPLAPSSKSTS GGTAALGCLVKDYFPEPVTVSWNSGAL TSGVHTFPAVLQSSGLYSLSSVVTVPSSS | Met47 hole-LHL | EIVLTQSPGTLSLSPGERATLSCRASQSV SSYANSYLHWYQQKPGQAPRLLIYRGST RESGIPDRFSGSGSGTDFTLTISRLEPEDF AVYYCQQSKSEPLTFGGGTKVEIKRTVA APSVFIFPPSDEQLKSGTASVVCLLNNFY PREAKVQWKVDNALQSGNSQESVTEQ DSKDSTYSLSSTLTLSKADYEKHKVYACE |
| | LGTQTYICNVNHKPSNTKVDKKVEPKSC GPAPELLGGGSQVQLVQSGAEVKKPGE SLKISCKGSGYTFTNYYIFWVRQMPGKG LEWMGIINPVDGDTNYNPSFQGQVTIS ADKSISTAYLQWSSLKASDTAMYYCARG GYTMDRWGQGTLVTVSS (SEQ ID NO:36) | | VTHQGLSSPVTKSFNRGECGPAPELLGG GSDIVMTQSPLSLPVTPGEPASISCRSSQ SLLHSNGYNYLHWYLQKPGQSPQLLIYK VSNRLSGVPDRFSGSGSGTDFTLKISRVE AEDVGVYYCFQNTHTPRTFGGGTKVEIK (SEQ ID NO:37) |

TABLE 7

Sequences of bispecific protein binding to Her2 and CD3.

| Construct Type: | Fab2 Her2/CD3 “One Arm” style | | |
|---|---|---|---|
| Clone Name: | Her23-LHL-LHL | | |
| Chain name | Heavy chain amino acid sequence | Chain name | Light chain amino acid sequence |
| Her23 knob-LHL | EVQLVESGGGLVQPGGSLRLSCAASGF NIKDTYIHWVRQAPG KG LEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVN H KPSNTKVDKKVE P KSCGPAPELLGGGSQVQLQQSGAELAR PGASVKMSCKASGYTFTRYTM HWVKQ RPGQGLEWIGYINPSRGYTNYNQKFKD KATLTTDKSSSTAYM QLSSLTSE DSAVYY CARYYDDHYCLDYWGQGTTLTVSS (SEQ ID NO:38) | Her23-LHL (underlined terminal K residue replaces an N that may cause N-link heterogeneity) | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECGPAPELLGGGS QIVLTQSPAIMSASPGEKVTMTCSASSS VSYMNWYQQKSGTSPKRWIYDTSKLAS GVPAH FRGSGSGTSYSLTISG M EAE DAA TYYCQQWSSNPFTFGSGTKLEIK (SEQ ID NO:39) |

TABLE 8

Sequences of bispecific protein binding to Her2 and CD3.

| Construct Type: | Fab2 Her2/CD3(34) “One Arm” style | | |
|---|---|---|---|
| Clone Name: | Her23(34)-LHL-LHL | | |
| Chain name | Heavy chain amino acid sequence | Chain name | Light chain amino acid sequence |
| Her23-LHL | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVN H KPSNTKVDKKVE P KSCGPAPELLGGGSEVKLLESGGGLVQP KGSLKLSCAASG FTFNTYAM NWVRQAP GKGLEWVARIRSKYNNYATYYADSVKD RFTISRDDSQSILYLQMNNLKTEDTAMY YCVRHGNFGNSYVSWFAYWGQGTLVT VSS (SEQ ID NO:40) | Her23-LHL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECGPAPELLGGGS QAVVTQESALTTSPGETVTLTCRSSTGA VTTSNYANWVQEKPDHLFTGLlGGTNK RAPGVPARFSGSLIGDKAALTITGAQTE DEAIYFCALWYSNLWVFGGGTKLTVL (SEQ ID NO:41) |

TABLE 9

Sequences of bispecific proteins binding to Her2 and CD47.

| Construct Type: | IgG2 Her2/CD47 | | |
|---|---|---|---|
| Clone Name: | Her47-LHLF-LHL | | |
| Chain name | Heavy chain amino acid sequence | Chain name | Light chain amino acid sequence |
| Her47 LHLF-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVN H KPSNTKVDKKVEP KSCGPAPLGLGGGSQVQLVQSGAEVK KPGESLKISCKGSGYTFTNYYIFWVRQM PGKGLEWMGIINPVDGDTNYNPSFQG QVTISADKSISTAYLQWSSLKASDTAMY YCARGGYTMDRWGQGTLVTVSS (SEQ ID NO:42) | Her47 LHL-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECGPAPELLGGGS DIVMTQSPLSLPVTPGEPASISCRSSQSL LHSNGYNYLHWYLQKPGQSPQLLIYKVS NRLSGVPDRFSGSGSGTDFTLKISRVEAE DVGVYYCFQNTHTPRTFGGGTKVEIK (SEQ ID NO:43 |
| Clone name: | Her47-LHL-LHLF | | |
| Chain name | Heavy chain amino acid sequence | Chain name | Light chain amino acid sequence |
| Her47 LHL-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVN H KPSNTKVDKKVE P KSCGPAPELLGGGSQVQLVQSGAEVKK PGESLKISCKGSGYTFTNYYIFWVRQMP GKGLEWMGIINPVDGDTNYNPSFQGQ VTISADKSISTAYLQWSSLKASDTAMYY CARGGYTMDRWGQGTLVTVSS (SEQ ID NO:44) | Her47 LHLF-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPLGLGG GSDIVMTQSPLSLPVTPGEPASISCRSSQ SLLHSNGYNYLHWYLQKPGQSPQLLIYK VSNRLSGVPDRFSGSGSGTDFTLKISRVE AEDVGVYYCFQNTHTPRTFGGGTKVEIK (SEQ ID NO:45) |
| Clone name: | Her47-LHLF-LHLF | | |
| Chain name | Heavy chain amino acid sequence | Chain name | Light chain amino acid sequence |
| Her47 LHLF-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVN H KPSNTKVDKKVE P KSCGPAPLGLGGGSQVQLVQSGAEVK KPGESLKISCKGSGYTFTNYYIFWVRQM PGKGLEWMGIINPVDGDTNYNPSFQG QVTISADKSISTAYLQWSSLKASDTAMY YCARGGYTMDRWGQGTLVTVSS (SEQ ID NO:46) | Her47 LHLF-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECGPAPLGLGG GSDIVMTQSPLSLPVTPGEPASISCRSSQ SLLHSNGYNYLHWYLQKPGQSPQLLIYK VSNRLSGVPDRFSGSGSGTDFTLKISRVE AEDVGVYYCFQNTHTPRTFGGGTKVEIK (SEQ ID NO:47) |
| Clone name: | Her47-LHLM-LHLM | | |
| Chain name | Heavy chain amino acid sequence | Chain name | Light chain amino acid sequence |
| Her47 LHLM-VH | EVQLVESGGGLVQPGGSLRLSCAASGF NIKDTYIHWVRQAPGKGLEWVARIYPT NGYTRYADSVKGRFTISADTSKNTAYLQ | Her47 LHLM-VL | DIQMTQSPSSLSASVGDRVTITCRASQD VNTAVAWYQQKPGKAPKLLIYSASFLYS GVPSRFSGSRSGTDFTLTISSLQPEDFAT |
| | MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCPPCPAPELLGGGSQVQLVQSGAEV KKPGESLKISCKGSGYTFTNYYIFWVRQ MPGKGLEWMGIINPVDGDTNYNPSFQ GQVTISADKSISTAYLQWSSLKASDTAM YYCARGGYTMDRWGQGTLVTVSS (SEQ ID NO:48) | | YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECPPCPAPELLG GGSDIVMTQSPLSLPVTPGEPASISCRSS QSLLHSNGYNYLHWYLQKPGQSPQLLIY KVSNRLSGVPDRFSGSGSGTDFTLKISRV EAEDVGVYYCFQNTHTPRTFGGGTKVEI K (SEQ ID NO:49) |
| Her47 LHL-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCPPCPAPELLGGGSQVQLVQSGAEV KKPGESLKISCKGSGYTFTNYYIFWVRQ MPGKGLEWMGIINPVDGDTNYNPSFQ GQVTISADKSISTAYLQWSSLKASDTAM YYCARGGYTMDRWGQGTLVTVSS (SEQ ID NO:50) | Her47 LHLF-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECPPCPAPLGL GGGSDIVMTQSPLSLPVTPGEPASISCRS SQSLLHSNGYNYLHWYLQKPGQSPQLLI YKVSNRLSGVPDRFSGSGSGTDFTLKISR VEAEDVGVYYCFQNTHTPRTFGGGTKV EIK (SEQ ID NO:51) |
| Her47 LHLMF-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCPPCPAPLGLGGGSQVQLVQSGAE VKKPGESLKISCKGSGYTFTNYYIFWVRQ MPGKGLEWMGIINPVDGDTNYNPSFQ GQVTISADKSISTAYLQWSSLKASDTAM YYCARGGYTMDRWGQGTLVTVSS (SEQ ID NO:52) | Her47 LHLM-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECPPCPAPELLG GGSDIVMTQSPLSLPVTPGEPASISCRSS QSLLHSNGYNYLHWYLQKPGQSPQLLIY KVSNRLSGVPDRFSGSGSGTDFTLKISRV EAEDVGVYYCFQNTHTPRTFGGGTKVEI K (SEQ ID NO:53) |
| Her47 LHLF-VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNH KPSNTKVDKKVE P KSCPPCPAPLGLGGGSQVQLVQSGAE VKKPGESLKISCKGSGYTFTNYYIFWVRQ | Her47 LHLF-VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKH KVYACEVT HQGLSSPVTKSFNRGECPPCPAPLGL GGGSDIVMTQSPLSLPVTPGEPASISCRS SQSLLHSNGYNYLHWYLQKPGQSPQLLI |
| | MPGKGLEWMGIINPVDGDTNYNPSFQ GQVTISADKSISTAYLQWSSLKASDTAM YYCARGGYTMDRWGQGTLVTVSS (SEQ ID NO:54) | | YKVSNRLSGVPDRFSGSGSGTDFTLKISR VEAEDVGVYYCFQNTHTPRTFGGGTKV EIK (SEQ ID NO:55) |

TABLE 10

Examples of immunoglobulin Fc region amino acid sequences.
Human IgG4 wild type
ASTKGPSVFPLAPCSRSTSESTAALGCLVKDYFPEPVTVSWNSGALTSGVHTFPAVLQSS GLYSLSSVVTVPSSSLGTKTYTCNVDHKPSNTKVDKRVESKYGPPCPSCPAPEFLGGPSV FLFPPKPKDTLMISRTPEVTCWVDVSQEDPEVQFNUVYVDGVEVHNAKTKPREEQFNST YRVVSVLTVLHQDWLNGKEYKCKVSNKGLPSSIEKTISKAKGQPREPQVYTLPPSQEEMT KNQVSLTCLVKGFYPSDIAVEWESNGQPENNYKTTPPVLDSDGSFFLYSRLTVDKSRWQ EGNVFSCSVMHEALHNHYTQKSLSLSLGK (SEQ ID NO:56)
Human IgG4(S228P)
ASTKGPSVFPLAPCSRSTSESTAALGCLVKDYFPEPVTVSWNSGALTSGVHTFPAVLQSS GLYSLSSVVTVPSSSLGTKTYTCNVDHKPSNTKVDKRVESKYGPPCPPCPAPEFLGGPSV FLFPPKPKDTLMISRTPEVTCWVDVSQEDPEVQFNUVYVDGVEVHNAKTKPREEQFNST YRWSVLTVLHQDWLNGKEYKCKVSNKGLPSSIEKTISKAKGQPREPQVYTLPPSQEEMT KNQVSLTCLVKGFYPSDIAVEWESNGQPENNYKTTPPVLDSDGSFFLYSRLTVDKSRWQ EGNVFSCSVMHEALHNHYTQKSLSLSLGK (SEQ ID NO:57)
Human IgG1 wild type
ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PSVFLFPPKPKDTLMISRTPEVTCVWDVSHEDPEVKFNWYVDGVEVHNAKTKPREEQYN STYRVVSVLTVLHQDWLNGKEYKCKVSNKALPAPIEKTISKAKGQPREPQVYTLPPSRDEL TKNQVSLTCLVKGFYPSDIAVEWESNGQPENNYKTTPPVLDSDGSFFLYSKLTVDKSRW QQGNVFSCSVMHEALHNHYTQKSLSLSPGK (SEQ ID NO:58)
Human IgG1-3M
ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPEAAGA PSVFLFPPKPKDTLMISRTPEVTCVWDVSHEDPEVKFNWYVDGVEVHNAKTKPREEQYN STYRVVSVLTVLHQDWLNGKEYKCKVSNKALPAPIEKTISKAKGQPREPQVYTLPPSRDEL TKNQVSLTCLVKGFYPSDIAVEWESNGQPENNYKTTPPVLDSDGSFFLYSKLTVDKSRW QQGNVFSCSVMHEALHNHYTQKSLSLSPGK (SEQ ID NO:59)
Human IgG2 wild type
ASTKGPSVFPLAPCSRSTSESTAALGCLVKDYFPEPVTVSWNSGALTSGVHTFPAVLQSS GLYSLSSVVTVPSSNFGTQTYTCNVDHKPSNTKVDKTVERKCCVECPPCPAPPVAGPSV FLFPPKPKDTLMISRTPEVTCVWDVSHEDPEVQFNWYVDGVEVHNAKTKPREEQFNSTF RVVSVLTWHQDWLNGKEYKCKVSNKGLPAPIEKTISKTKGQPREPQVYTLPPSREEMTK NQVSLTCLVKGFYPSDISVEWESNGQPENNYKTTPPMLDSDGSFFLYSKLTVDKSRWQQ GNVFSCSVMHEALHNHYTQKSLSLSPGK (SEQ ID NO:60)
Human IgG1 wild type “REEM” allotype
ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PSVFLFPPKPKDTLMISRTPEVTCVWDVSHEDPEVKFNWYVDGVEVHNAKTKPREEQYN STYRVVSVLTVLHQDWLNGKEYKCKVSNKALPAPIEKTISKAKGQPREPQVYTLPPSREE MTKNQVSLTCLVKGFYPSDIAVEWESNGQPENNYKTTPPVLDSDGSFFLYSKLTVDKSR WQQGNVFSCSVMHEALHNHYTQKSLSLSPGK (SEQ ID NO:61)
Human IgG1-3M “REEM” allotype
ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPEAAGA PSVFLFPPKPKDTLMISRTPEVTCVWDVSHEDPEVKFNWYVDGVEVHNAKTKPREEQYN STYRVVSVLTVLHQDWLNGKEYKCKVSNKALPAPIEKTISKAKGQPREPQVYTLPPSREE MKNQVSLTCLVKGFYPSDIAVEWESNGQPENNYKTTPPVLDSDGSFFLYSKLTVDKSRW QQGNVFSCSVMHEALHNHYTQKSLSLSPGK (SEQ ID NO:62)

TABLE 11

Examples of CD47 protein amino acid sequences.
Human CD47 sequence
MWPLVAALLLGSACCGSAQLLFNKTKSVEFTFCNDTVVIPCFVTNMEAQNTTEVYVKWKFKGRD IYTFDGALNKSTVPTDFSSAKIEVSQLLKGDASLKMDKSDAVSHTGNYTCEVTELTREGETIIE LKYRVVSWFSPNENILIVIFPIFAILLFWGQFGIKTLKYRSGGMDEKTIALLVAGLVITVIVIV GAILFVPGEYSLKNATGLGLIVTSTGILILLHYYVFSTAIGLTSFVIAILVIQVIAYILAWGL SLCIAACIPMHGPLLISGLSILALAQLLGLVYMKFVASNQKTIQPPRKAVEEPLNAFKESKGMM NDE (SEQ ID NO:63)
Cynomolgus monkey CD47 sequence
MWPLVAALLLGSACCGSAQLLFNKTKSVEFTFCNDTVVIPCFVTNMEAQNTTEVYVKWKFKGRD IYTFDGALNKSTAPANFSSAKIEVSQLLKGDASLKMDKSDAVSHTGNYTCEVTELTREGETIIE LKYRVVSWFSPNENILIVIFPIFAILLFWGQFGIKTLKYRSGGMDEKTIALLVAGLMITVIVIV GAILFVPGEYSLKNATGLGLIVTSTGILILLHYYVFSTAIGLTSFVIAILVIQVIAYILAWGL SLCIAACIPMHGPLLISGLSILALAQLLGLVYMKFVASNQKTIQPPRKAVEEPLNAFKESKGMM NDE (SEQ ID NO:64)

TABLE 12

Examples of cMET protein amino acid sequences.
Human cMET sequence
MKAPAVLAPGILVLLFTLVQRSNGECKEALAKSEMNVNMKYQLPNFTAETPIQNVILHEH HIFLGATNYIYVLNEEDLQKVAEYKTGPVLEHPDCFPCQDCSSKANLSGGVWKDNINMAL VVDTYYDDQLISCGSVNRGTCQRHVFPHNHTADIQSEVHCIFSPQIEEPSQCPDCVVSAL GAKVLSSVKDRFINFFVGNTINSSYFPDHPLHSISVRRLKETKDGFMFLTDQSYIDVLPE FRDSYPIKYVHAFESNNFIYFLTVQRETLDAQTFHTRIIRFCSINSGLHSYMEMPLECIL TEKRKKRSTKKEVFNILQAAYVSKPGAQLARQIGASLNDDILFGVFAQSKPDSAEPMDRS AMCAFPIKYVNDFFNKIVNKNNVRCLQHFYGPNHEHCFNRTLLRNSSGCEARRDEYRTEF TTALQRVDLFMGQFSEVLLTSISTFIKGDLTIANLGTSEGRFMQVVVSRSGPSTPHVNFL LDSHPVSPEVIVEHTLNQNGYTLVITGKKITKIPLNGLGCRHFQSCSQCLSAPPFVQCGW CHDKCVRSEECLSGTWTQQICLPAIYKVFPNSAPLEGGTRLTICGWDFGFRRNNKFDLKK TRVLLGNESCTLTLSESTMNTLKCTVGPAMNKHFNMSIIISNGHGTTQYSTFSYVDPVIT SISPKYGPMAGGTLLTLTGNYLNSGNSRHISIGGKTCTLKSVSNSILECYTPAQTISTEF AVKLKIDLANRETSIFSYREDPIVYEIHPTKSFISGGSTITGVGKNLNSVSVPRMVINVH EAGRNFTVACQHRSNSEIICCTTPSLQQLNLQLPLKTKAFFMLDGILSKYFDLIYVHNPV FKPFEKPVMISMGNENVLEIKGNDIDPEAVKGEVLKVGNKSCENIHLHSEAVLCTVPNDL LKLNSELNIEWKQAISSTVLGKVIVQPDQNFTGLIAGVVSISTALLLLLGFFLWLKKRKQ IKDLGSELVRYDARVHTPHLDRLVSARSVSPTTEMVSNESVDYRATFPEDQFPNSSQNGS CRQVQYPLTDMSPILTSGDSDISSPLLQNTVHIDLSALNPELVQAVQHVVIGPSSLIVHF NEVIGRGHFGCVYHGTLLDNDGKKIHCAVKSLNRITDIGEVSQFLTEGIIMKDFSHPNVL SLLGICLRSEGSPLVVLPYMKHGDLRNFIRNETHNPTVKDLIGFGLQVAKGMKYLASKKF VHRDLAARNCMLDEKFTVKVADFGLARDMYDKEYYSVHNKTGAKLPVKWMALESLQTQKFTTKS DVWSFGVLLWELMTRGAPPYPDVNTFDITVYLLQGRRLLQPEYCPDPLYEVMLKCWHPKAEMRP SFSELVSRISAIFSTFIGEHYVHVNATYVNVKCVAPYPSLLSSEDNADDEVD TRPASFWETS (SEQ ID NO:67)
Cynomolgus Monkey cMET sequence
mkapavlvpg ilvllftlvq rsngeckeal aksemnvnmk yqlpnftaet aiqnvilheh hiflgatnyi yvlneedlqk vaeyktgpvl ehpdcfpcqd csskanlsgg vwkdninmal vvdtyyddql iscgsvnrgt cqrhvfphnh tadiqsevhc ifspqieepn qcpdcvvsal gakvlssvkd rfinffvgnt inssyfphhp lhsisvrrlk etkdgfmflt dqsyidvlpe frdsypikyi hafesnnfiy fltvqretln aqtfhtriir fcslnsglhs ymemplecil tekrkkrstk kevfnilqaa yvskpgaqla rqigaslndd ilfgvfaqsk pdsaepmdrs amcafpikyv ndffnkivnk nnvrclqhfy gpnhehcfnr tllrnssgce arrdeyraef ttalqrvdlf mgqfsevllt sistfvkgdl tianlgtseg rfmqvvvsrs gpstphvnfl ldshpvspev ivehplnqng ytlvvtgkki tkiplnglgc rhfqscsqcl sappfvqcgw chdkcvrsee cpsgtwtqqi clpaiykvfp tsapleggtr lticgwdfgf rrnnkfdlkk trvllgnesc tltlsestmn tlkctvgpam nkhfnmsiii snghgttqys tfsyvdpiit sispkygpma ggtlltltgn ylnsgnsrhi siggktctlk svsnsilecy tpaqtistef avklkidlan retsifsyre dpivyeihpt ksfisggsti tgvgknlhsv svprmvinvh eagrnftvac qhrsnseiic cttpslqqln lqlplktkaf fmldgilsky fdliyvhnpv fkpfekpvmi smgnenvlei kgndidpeav kgevlkvgnk scenihlhse avlctvpndl lklnselnie wkqaisstvl gkvivqpdqn ftgliagvvs isialllllg lflwlkkrkq ikdlgselvr ydarvhtphl drlvsarsvs pttemvsnes vdyratfped qfpnssqngs crqvqypltd mspiltsgds disspllqnt vhidlsalnp elvqavqhvv igpsslivhf nevigrghfg cvyhgtlldn dgkkihcavk slnritdige vsqfltegii mkdfshpnvl sllgiclrse gsplvvlpym khgdlrnfir nethnptvkd ligfglqvak gmkylaskkf vhrdlaarnc mldekftvkv adfglardmy dkeyysvhnk tgaklpvkwm aleslqtqkf ttksdvwsfg vllwelmtrg appypdvntf ditvyllqgr rllqpeycpd plyevmlkcw hpkaemrpsf selvsrisai fstfigehyv hvnatyvnvk cvapypslls sednaddevdt (SEQ ID NO:68)

TABLE 13

Sequences of bispecific protein binding to Her2 and CD3.

| Construct Type: | Fc-Her2/CD3(34) | | |
|---|---|---|---|
| Clone Name: | Fc-Her23(34) | | |
| Chain name | Heavy chain amino acid sequence | Chain name | Light chain amino acid sequence |
| FC-Her23(34) (Hole) | CPPCPAPEAAGAPSVFLFPPKPKDTLMI SRTPEVTCVVVDVSHEDPEVKFNWYVD GVEVHNAKTKPREEQYNSTYRVVSVLTV LHQDWLNGKEYKCKVSNKALPAPIEKTI SKAKGQPREPQVYTLPPSRDELTKNQVS LSCAVKGFYPSDIAVEWESNGQPENNY KTTPPVLDSDGSFFLVSKLTVDKSRWQQ GNVFSCSVMHEALHNHYTQKSLSLSPG GPAPLGLGGGSEVQLVESGGGLVQPGG SLRLSCAASGFNIKDTYIHWVRQAPGKG LEWVARIYPTNGYTRYADSVKGRFTISA DTSKNTAYLQMNSLRAEDTAVYYCSRW GGDGFYAMDYWGQGTLVTVSSASTKG PSVFPLAPSSKSTSGGTAALGCLVKDYFP EPVTVSWNSGALTSGVHTFPAVLQSSG LYSLSSVVTVPSSSLGTQTYICNVNHKPS NTKVDKKVEPKSCGPAPLGLGGGSEVKL LESGGGLVQPKGSLKLSCAASGFTFNTY AMNWVRQAPGKGLEWVARIRSKYNN YATYYADSVKDRFTISRDDSQSILYLQM NNLKTEDTAMYYCVRHGNFGNSYVSW FAYWGQGTLVTVSSASTKGPSVFPLAPS SKSTSGGTAALGCLVKDYFPEPVTVSWN SGALTSGVHTFPAVLQSSGLYSLSSVVTV PSSSLGTQTYICNVNHKPSNTKVDKKVE PKSC (SEQ ID NO:73) | FC-Her23(34) (Knob) | CPPCPAPEAAGAPSVFLFPPKPKDTLMI SRTPEVTCVVVDVSHEDPEVKFNWYVD GVEVHNAKTKPREEQYNSTYRVVSVLTV LHQDWLNGKEYKCKVSNKALPAPIEKTI SKAKGQPREPQVYTLPPSRDELTKNQVS LWCLVKGFYPSDIAVEWESNGQPENNY KTTPPVLDSDGSFFLYSKLTVDKSRWQQ GNVFSCSVMHEALHNHYTQKSLSLSPG GPAPLGLGGGSDIQMTQSPSSLSASVG DRVTITCQASQDISNYLNWYQQKPGKA PKLLIYDASNLETGVPSRFSGSGSGTDFT FTISSLQPEDIATYFCQHFDHLPLAFGGG TKVEIKRTVAAPSVFIFPPSDEQLKSGTA SVVCLLNNFYPREAKVQWKVDNALQSG NSQESVTEQDSKDSTYSLSSTLTLSKADY EKHKVYACEVTHQGLSSPVTKSFNRGEC GPAPELLGGGSQAVVTQESALTTSPGET VTLTCRSSTGAVTTSNYANWVQEKPDH LFTGLIGGTNKRAPGVPARFSGSLIGDKA ALTITGAQTEDEAIYFCALWYSNLWVFG GGTKLTVLGQPKAAPSVTLFPPSSEELQ ANKATLVCLISDFYPGAVTVAWKADSSP VKAGVETTTPSKQSNNKYAASSYLSLTP EQWKSHRSYSCQVTHEGSTVEKTVAPT ECS (SEQ ID NO:74) |

TABLE 14

Sequences of Fab2-based protein binding to cMET and cMET.

| Construct Type: | Fab2 CMET/CMET “One Arm” style | | |
|---|---|---|---|
| Clone Name: | MetMet-LHL-LHL | | |
| Chain name | Heavy chain amino acid sequence | Chain name | Light chain amino acid sequence |
| MetMet-LHL-LHL VH | QVQLVQSGAEVKKPGASVKVSCKASGY TFTSYTMHWVRQAPGQGLEWMGIINP SGGSTSYAQKFQGRVTMTRDTSTSTVY MELSSLRSEDTAVYYCARQEITTEFDYW GQGTLVTVSSASTKGPSVFPLAPSSKSTS GGTAALGCLVKDYFPEPVTVSWNSGAL TSGVHTFPAVLQSSGLYSLSSVVTVPSSS LGTQTYICNVNHKPSNTKVDKKVEPKSC GPAPELLGGGSQVQLVQSGAEVKKPGA SVKVSCKASGYTFTSYTM HWVRQAPG QGLEWMGIINPSGGSTSYAQKFQGRVT MTRDTSTSTVYM E LSSLRSEDTAVYYCA RQEITTEFDYWGQGTLVTVSS (SEQ ID NO:75) | Met47 hole-LHL VL | EIVLTQSPGTLSLSPGERATLSCRASQSV SSYANSYLHWYQQKPGQAPRLLIYRGST RESGIPDRFSGSGSGTDFTLTISRLEPEDF AVYYCQQSKSEPLTFGGGTKVEIKRTVA APSVFIFPPSDEQLKSGTASVVCLLNNFY PREAKVQWKVDNALQSGNSQESVTEQ DSKDSTYSLSSTLTLSKADYEKHKVYACE VTHQGLSSPVTKSFNRGECGPAPELLGG GSEIVLTQSPGTLSLSPGERATLSCRASQ SVSSYANSYLHWYQQKPGQAPRLLIYR GSTRESGIPDRFSGSGSGTDFTLTISRLEP EDFAVYYCQQSKSEPLTFGGGTKVEIK (SEQ ID NO:76) |

TABLE 15

Examples of Her2 protein amino acid sequences.
HUMAN Her2 (erbB-2) sequence
MELAALCRWGLLLALLPPGAASTQVCTGTDMKLRLPASPETHLDMLRHLYQGCQWQGN LELTYLPTNASLSFLQDIQEVQGYVLIAHNQVRQVPLQRLRIVRGTQLFEDNYALAVLDNG DPLNNTTPVTGASPGGLRELQLRSLTEILKGGVLIQRNPQLCYQDTILWKDIFHKNNQLALT LIDTNRSRACHPCSPMCKGSRCWGESSEDCQSLTRTVCAGGCARCKGPLPTDCCHEQC AAGCTGPKHSDCLACLHFNHSGICELHCPALVTYNTDTFESMPNPEGRYTFGASCVTACP YNYLSTDVGSCTLVCPLHNQEVTAEDGTQRCEKCSKPCARVCYGLGMEHLREVRAVTSA NIQEFAGCKKIFGSLAFLPESFDGDPASNTAPLQPEQLQVFETLEEITGYLYISAWPDSLPD LSVFQNLQVIRGRILHNGAYSLTLQGLGISWLGLRSLRELGSGLALIHHNTHLCFVHTVPW DQLFRNPHQALLHTANRPEDECVGEGLACHQLCARGHCWGPGPTQCVNCSQFLRGQE CVEECRVLQGLPREYVNARHCLPCHPECQPQNGSVTCFGPEADQCVACAHYKDPPFCV ARCPSGVKPDLSYMPIWKFPDEEGACQPCPINCTHSCVDLDDKGCPAEQRASPLTSIISA VVGILLVWLGWFGILIKRRQQKIRKYTMRRLLQETELVEPLTPSGAMPNQAQMRILKETE LRKVKVLGSGAFGTVYKGIWIPDGENVKIPVAIKVLRENTSPKANKEILDEAYVMAGVGSP YVSRLLGICLTSTVQLVTQLMPYGCLLDHVRENRGRLGSQDLLNWCMQIAKGMSYLEDV RLVHRDLAARNVLVKSPNHVKITDFGLARLLDIDETEYHADGGKVPIKWMALESILRRRFT HQSDVWSYGVTVWELMTFGAKPYDGIPAREIPDLLEKGERLPQPPICTIDVYMIMVKCWM IDSECRPRFRELVSEFSRMARDPQRFWIQNEDLGPASPLDSTFYRSLLEDDDMGDLVDA EEYLVPQQGFFCPDPAPGAGGMVHHRHRSSSTRSGGGDLTLGLEPSEEEAPRSPLAPS EGAGSDVFDGDLGMGAAKGLQSLPTHDPSPLQRYSEDPTVPLPSETDGYVAPLTCSPQP EYVNQPDVRPQPPSPREGPLPAARPAGATLERPKTLSPGKNGVVKDVFAFGGAVENPEY LTPQGGAAPQPHPPPAFSPAFDNLYYWDQDPPERGAPPSTFKGTPTAENPEYLGLDVPV (SEQ ID NO:77)
Cynomolgus monkey Her2 (erbB2) sequence
MAESPASAFRDSLRKSVRTAAGNPGVPELGGTHPGLREEREKVKLGVATPRLVGMQLEG ASWERACSQSQEEEEVEEEGCLRKYKNEWELRFPSIGTGETRGAPWAAVRPFPRGSFR RRAPGPHPSPHPAPHALPAGSSRSHGAGAAVSTMELAAWYRWGLLLALLPPGATGTQV CTGTDMKLRLPASPETHLDMLRHLYQGCQWQGNLELTYLPTNASLSFLQDIQEVQGYVL IAHNQVRQVPLQRLRIVRGTQLFEDNYALAVLDNGDPLNNTTPVTGASPGGLRELQLRSL TEILKGGVLIQRNPQLCYQDTILWKDIFHKNNQLALTLIDTNRSRACHPCSPVCKGSRCWG ESSEDCQSLTRTVCAGGCARCKGPLPTDCCHEQCAAGCTGPKHSDCLACLHFNHSGICE LHCPALVTYNTDTFESMPNPEGRYTFGASCVTACPYNYLSTDVGSCTLVCPLHNQEVTAE DGTQRCEKCSKPCARVCYGLGMEHLREVRAVTSANIQEFAGCKKIFGSLAFLPESFDGDP ASNTAPLQPEQLRVFETLEEITGYLYISAWPDSLPDLSVLQNLQVIRGRILHNGAYSLTLQG LGISWLGLRSLRELGSGLALIHHNTRLCFVHTVPWDQLFRNPHQALLHTANRPEDECVGE GLACHQLCARGHCWGPGPTQCVNCSQFLRGQECVEECRVLQGLPREYVNARHCLPCH PECQPQNGSVTCFGPEADQCVACAHYKDPPFCVARCPSGVKPDLSYMPIWKFPDEEGT CQSCPI NCTHSCVDLDDKGCP AEQRASPL TSIISA WGILL VWLGVVFGILlKRRQQKIRKY TMRRLLQETELVEPLTPSGAMPNQAQMRILKETELRKVKVLGSGAFGTVYKGIWIPDGEN VKIPVAIKVLRENTSPKANKEILDEAYVMAGVGSPYVSRLLGICLTSTVQLVTQLMPYGCLL DHVRENRGRLGSQDLLNWCMQIAKGMSYLEDVRLVHRDLAARNVLVKSPNHVKITDFGL ARLLDIDETEYHADGGKVPIKWMALESILRRRFTHQSDVWSYGVTVWELMTFGAKPYDGI PAREIPDLLEKGERLPQPPICTIDVYMIMVKCWMIDSECRPRFRELVSEFSRMARDPQRFV VIQNEDLGPASPLDSTFYRSLLEDDDMGDLVDAEEYLVPQQGFFCPDPAPGTGGMVHHR HRSSSTRSGGGDLTLGLEPSEEEAPRSPRAPSEGTGSDVFDGDLGMGAAKGLQSLPAH DPSPLQRYSEDPTVPLPSETDGYVAPLTCSPQPEYVNQPDVRPQPPSPQEGPLSPARPT GATLERPKTLSPGKNGVVKDVFAFGGAVENPEYLAPRGGAAPQPHLPPAFSPAFDNLYY WDQDPSERGAPPSTFKGTPTAENPEYLGLDVPV (SEQ ID NO:78)

TABLE 16

Examples of CD3 epsilon domain amino acid sequences
Human CD3 epsilon sequence
MQSGTHWRVLGLCLLSVGVWGQDGNEEMGGITQTPYKVSISGTTVILTCPQYPGSEILQHNDKN IGGDEDDKNIGSDEDHLSLKEFSELEQSGYYVCYPRGSKPEDANFYLYLRARVCENCMEMDVMS VATIVIVDICITGGLLLLVYYWSKNRKAKAKPVTRGAGAGGRQRGQNKERPPPVPNPDYE PIRK GQRDLYSGLNQRRI (SEQ ID NO:79)

Cynomolgus monkey CD3 epsilon sequence
MQSGTRWRVLGLCLLSIGVWGQDGNEEMGSITQTPYQVSISGTTVILTCSQHLGSEAQWQHNGK NKEDSGDRLFLPEFSEMEQSGYYVCYPRGSNPEDASHHLYLKARVCENCMEMDVMAVATIVIVD ICITLGLLLLVYYWSKNRKAKAKPVTRGAGAGGRQRGQNKERPPPVPNPDYEPIRKGQQDLYSG LNQRRI (SEQ ID NO:80)

TABLE 17

Biacore binding values for control anti-Her2 and anti-CD47 antibodies

| Clone ID | Analyte | ka (⅟Ms) | kd (⅟s) | Rmax (RU) | Concentration range (nM) | KD (M) | Chi² | KD (nM) |
|---|---|---|---|---|---|---|---|---|
| Trastuzumab IgG1 (beginning) | hHer2 | 2.34E+05 | 5.06E-04 | 265.80 | 100-12.5 | 2.16E-09 | 3.40 | 2.16 |
| Trastuzumab IgG1 (end) | hHer2 | 2.52E+05 | 6.18E-04 | 144.13 | 100-12.5 | 2.45E-09 | 1.73 | 2.45 |
| A-D5 IgG1 (beginning) | hCD47 | 8.33E+05 | 1.07E-03 | 122.87 | 100-25 | 1.29E-09 | 8.58 | 1.29 |
| A-D5 IgG1 (end) | hCD47 | 7.75E+05 | 1.03E-03 | 85.51 | 100-25 | 1.32E-09 | 5.34 | 1.32 |

TABLE 18

Biacore binding for Her2 and CD47 during MMP12 activation of IgG² Her47-LHL-LHLF protein.

| Digestion Time Point | Analyte | ka (⅟Ms) | kd (⅟s) | Rmax (RU) | Concentration range (nM) | KD (M) | Chi² | KD (nM) |
|---|---|---|---|---|---|---|---|---|
| Undigested | hHer2 | 3.62E+05 | 3.67E-04 | 172.38 | 100-12.5 | 1.02E-09 | 2.64 | 1.02 |
| 2 hours | | 3.71E+05 | 3.39E-04 | 156.08 | 100-12.5 | 9.14E-10 | 2.38 | 0.91 |
| 4 hours | | 4.52E+05 | 3.56E-04 | 128.53 | 50-12.5 | 7.87E-10 | 0.62 | 0.79 |
| 8 hours | | 3.83E+05 | 3.67E-04 | 125.18 | 100-12.5 | 9.59E-10 | 2.05 | 0.96 |
| 24 hours | | 3.89E+05 | 4.19E-04 | 103.96 | 100-12.5 | 1.08E-09 | 1.83 | 1.08 |
| Undigested | hCD47 | N/A | N/A | N/A | N/A | N/A | N/A | N/A |
| 2 hours | | 2.95E+05 | 7.12E-03 | 8.3 | 400-25 | 2.42E-08 | 0.02 | 24.16 |
| 4 hours | | 4.57E+05 | 5.92E-03 | 12.09 | 400-25 | 1.30E-08 | 0.1 | 12.96 |
| 8 hours | | 4.81E+05 | 4.10E-03 | 17.27 | 200-25 | 8.52E-09 | 0.32 | 8.52 |
| 24 hours | | 8.00E+05 | 3.97E-03 | 23.93 | 200-25 | 4.97E-09 | 0.64 | 4.97 |

N/A = Not Applicable. No binding signal observed.

TABLE 19

Sequences of bispecific proteins binding to Her2 and CD47.

| Construct Type: | IgG2 ‘IgG1-DAA’ Her2/CD47 | | |
|---|---|---|---|
| Clone Name: | Her47 LHLF-LHL IgG1-2hDAA | | |
| Chain name | Heavy chain amino acid sequence | Chain name | Light chain amino acid sequence |
| Her47 LHLF-LHL IgG1-2hDAA VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCGPAPLGLGGGSQVQLVQSGAEVKK PGESLKISCKGSGYTFTNYYIFWVRQMP GKGLEWMGIINPVDGDTNYNPSFQGQ VTISADKSISTAYLQWSSLKASDTAMYY CARGGYTMDRWGQGTLVTVSSASTKG PSVFPLAPSSKSTSGGTAALGCLVKDYFP EPVTVSWNSGALTSGVHTFPAVLQSSG LYSLSSVVTVPSSSLGTQTYICNVNHKPS NTKVDKKVEPKSCDKTHTCPPCPAPPVA GPDVFLFPPKPKDTLMISRTPEVTCVVV DVSHEDPEVKFNWYVDGVEVHNAKTK PREEQYNSTYRVVSVLTVLHQDWLNGK EYKCKVSNAALPAPIAKTISKAKGQPREP QVYTLPPSRDELTKNQVSLTCLVKGFYPS DIAVEWESNGQPENNYKTTPPVLDSDG SFFLYSKLTVDKSRWQQGNVFSCSVMH EALHNHYTQKSLSLSPG (SEQ ID NO:88) | Her47 LHLF-LHL IgGl-2hDAA VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPELLGGGS DIVMTQSPLSLPVTPGEPASISCRSSQSL LHSNGYNYLHWYLQKPGQSPQLLIYKVS NRFSGVPDRFSGSGSGTDFTLKISRVEAE DVGVYYCFQNTHTPRTFGGGTKVEIKRT VAAPSVFIFPPSDEQLKSGTASVVCLLNN FYPREAKVQWKVDNALQSGNSQESVT EQDSKDSTYSLSSTLTLSKADYEKHKVYA CEVTHQGLSSPVTKSFNRGEC (SEQ ID NO: 89) |
| Clone name: | Her47 LHL-LHLF IgG1-2hDAA | | |
| Chain name | Heavy chain amino acid sequence | Chain name | Light chain amino acid sequence |
| Her47 LHL-LHLF IgG1-2hDAA VH | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCGPAPELLGGGSQVQLVQSGAEVKK PGESLKISCKGSGYTFTNYYIFWVRQMP GKGLEWMGIINPVDGDTNYNPSFQGQ VTISADKSISTAYLQWSSLKASDTAMYY CARGGYTMDRWGQGTLVTVSSASTKG PSVFPLAPSSKSTSGGTAALGCLVKDYFP EPVTVSWNSGALTSGVHTFPAVLQSSG LYSLSSVVTVPSSSLGTQTYICNVNHKPS NTKVDKKVEPKSCDKTHTCPPCPAPPVA GPDVFLFPPKPKDTLM ISRTPEVTCVVV DVSHEDPEVKFNWYVDGVEVHNAKTK PREEQYNSTYRVVSVLTVLHQDWLNGK EYKCKVSNAALPAPIAKTISKAKGQPREP QVYTLPPSRDELTKN QVSLTCLVKGFYPS | Her47 LHL-LHLF IgGl-2hDAA VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPLGLGGG SDIVMTQSPLSLPVTPGEPASISCRSSQS LLHSNGYNYLHWYLQKPGQSPQLLIYKV SNRFSGVPDRFSGSGSGTDFTLKISRVEA EDVGVYYCFQNTHTPRTFGGGTKVEIKR TVAAPSVFIFPPSDEQLKSGTASVVCLLN NFYPREAKVQWKVDNALQSGNSQESV TEQDSKDSTYSLSSTLTLSKADYEKHKVY ACEVTHQGLSSPVTKSFNRGEC (SEQ ID NO:91) |
| | DIAVEWESNGQPENNYKTTPPVLDSDG SFFLYSKLTVDKSRWQQGNVFSCSVMH EALHNHYTQKSLSLSPG (SEQ ID NO:90) | | |

TABLE 20

Sequences of bispecific proteins binding to Her2 and CD47.

| Construct Type: | IgG2 Her2/CD47 | | |
|---|---|---|---|
| Clone Name: | Her47 LHL-LHL-EK | | |
| Chain name | Heavy chain amino acid sequence | Chain name | Light chain amino acid sequence |
| Her47 LHL VH | (SEQ ID NO:44) | Her47 LHL-EK VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPADDDDKSG SDIVMTQSPLSLPVTPGEPASISCRSSQS LLHSNGYNYLHWYLQKPGQSPQLLIYKV SNRFSGVPDRFSGSGSGTDFTLKISRVEA EDVGVYYCFQNTHTPRTFGGGTKVEIK (SEQ ID NO:92) |
| Clone name: | Her47-LHL-LHL-Thr | | |
| Chain name | Heavy chain amino acid sequence | Chain name | Light chain amino acid sequence |
| Her47 LHL VH | (SEQ ID NO:44) | Her47 LHL-Thr VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPALVPRGSGS DIVMTQSPLSLPVTPGEPASISCRSSQSL LHSNGYNYLHWYLQKPGQSPQLLIYKVS NRFSGVPDRFSGSGSGTDFTLKISRVEAE DVGVYYCFQNTHTPRTFGGGTKVEIK (SEQ ID NO:93) |
| Clone name: | Her47-LHL-LHL-tPA | | |
| Chain name | Heavy chain amino acid sequence | Chain name | Light chain amino acid sequence |
| Her47 LHL VH | (SEQ ID NO:44) | Her47 LHL-tPA VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPGPFGRSAG GPDIVMTQSPLSLPVTPGEPASISCRSSQ SLLHSNGYNYLHWYLQKPGQSPQLLIYK VSNRFSGVPDRFSGSGSGTDFTLKISRVE AEDVGVYYCFQNTHTPRTFGGGTKVEIK (SEQ ID NO:94) |
| Her47 LHL VH | (SEQ ID NO:44) | Her47 LHL-GrB VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPLEADAG SDIVMTQSPLSLPVTPGEPASISCRSSQS LLHSNGYNYLHWYLQKPGQSPQLLIYKV SNRFSGVPDRFSGSGSGTDFTLKISRVEA EDVGVYYCFQNTHTPRTFGGGTKVEIK (SEQ ID NO:95) |
| Her47 LHL VH | (SEQ ID NO:44) | Her47 LHL-uPA VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPEARRGG SDIVMTQSPLSLPVTPGEPASISCRSSQS LLHSNGYNYLHWYLQKPGQSPQLLIYKV SNRFSGVPDRFSGSGSGTDFTLKISRVEA EDVGVYYCFQNTHTPRTFGGGTKVEIK (SEQ ID NO:96) |
| Her47 LHL VH | (SEQ ID NO:44) | Her47 LHL-A5 VL | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPEGEARG SDIVMTQSPLSLPVTPGEPASISCRSSQS LLHSNGYNYLHWYLQKPGQSPQLLIYKV SNRFSGVPDRFSGSGSGTDFTLKISRVEA EDVGVYYCFQNTHTPRTFGGGTKVEIK (SEQ ID NO:97) |

TABLE 21

Sequences of linker peptides containing protease cleavage motifs

| | |
|---|---|
| Protease | Linker sequence |
| Enterokinase (EK) | GPADDDDKSGS (SEQ ID NO: 82) |
| Thrombin (Thr) | GPALVPRGSGS (SEQ ID NO: 83) |
| tPA | GPGPFGRSAGGP (SEQ ID NO: 84) |
| Granzyme B (GrB) | GPAPLEADAGS (SEQ ID NO: 85) |
| uPA | GPAPEARRGGS (SEQ ID NO: 86) |
| ADAMTs-5 (A5) | GPAPEGEARGS (SEQ ID NO: 87) |

TABLE 22

Sequences of bispecific proteins binding to Her2 and CD3 or to EpCAM and CD3.

| Construct Type: | Fab2 Her2/CD3 | | |
|---|---|---|---|
| Clone Name: | Fab2 Her23 LHL-LHLF | | |
| Chain name | Heavy chain amino acid sequence | Chain name | Light chain amino acid sequence |
| | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCGPAPELLGGGSQVQLQQSGAELAR PGASVKMSCKASGYTFTRYTMHWVKQ RPGQGLEWIGYINPSRGYFNYNQKFKD KATLTTDKSSSTAYMQLSSLTSEDSAVYY CARYYDDHYCLDYWGQGTTLTVSSAST KGPSVFPLAPSSKSTSGGTAALGCLVKD YFPEPVTVSWNSGALTSGVHTFPAVLQS SGLYSLSSVVTVPSSSLGTQTYICNVNHK PSNTKVDKKVEPKSCGPAPEAAGAPSVF LFPPKPKDTLMISRTPEVTCVVVDVSHE DPEVKFNWYVDGVEVHNAKTKPREEQ YNSTYRVVSVLTVLHQDWLNGKEYKCK VSNKALPAPIEKTISKAKGQPREPQVYTL PPSRDELTKNQVSLSCAVKGFYPSDIAVE WESNGQPENNYKTTPPVLDSDGSFFLV SKLTVDKSRWQQGNVFSCSVMHEALH NHYTQKSLSLSPG (SEQ ID NO:98) | | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPLGLGGG SQIVLTQSPAIMSASPGEKVTMTCSASS SVSYMNWYQQKSGTSPKRWIYDTSKLA SGVPAHFRGSGSGTSYSLTISGMEAEDA ATYYCQQWSSNPFTFGSGTKLEIKRTVA APSVFIFPPSDEQLKSGTASVVCLLNNFY PREAKVQWKVDNALQSGNSQESVTEQ DSKDSTYSLSSTLTLSKADYEKHKVYACE VTHQGLSSPVTKSFNRGECGPAPEAAG APSVFLFPPKPKDTLMISRTPEVTCVVVD VSHEDPEVKFNWYVDGVEVHNAKTKP REEQYNSTYRVVSVLTVLHQDWLNGKE YKCKVSNKALPAPIEKTISKAKGQPREPQ VYTLPPSRDELTKNQVSLWCLVKGFYPS DIAVEWESNGQPENNYKTTPPVLDSDG SFFLYSKLTVDKSRWQQGNVFSCSVMH EALHNHYTQKSLSLSPG (SEQ ID NO:99) |
| Clone name: | Fab2 Her23 LHL-LHL | | |
| Chain name | Heavy chain amino acid sequence | Chain name | Light chain amino acid sequence |
| | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP | | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPELLGGGS |
| | KSCGPAPELLGGGSQVQLQQSGAELAR PGASVKMSCKASGYTFTRYTMHWVKQ RPGQG LEWIGYINPSRGYFNYNQKFKD KATLTTDKSSSTAYMQLSSLTSEDSAVYY CARYYDDHYCLDYWGQGTTLTVSSAST KGPSVFPLAPSSKSTSGGTAALGCLVKD YFPEPVTVSWNSGALTSGVHTFPAVLQS SGLYSLSSVVTVPSSSLGTQTYICNVNHK PSNTKVDKKVEPKSCGPAPEAAGAPSVF LFPPKPKDTLMISRTPEVTCVVVDVSHE DPEVKFN WYVDGVEVH NAKTKPREEQ YNSTYRVVSVLTVLHQDWLNGKEYKCK VSNKALPAPIEKTISKAKGQPREPQVYTL PPSRDELTKNQVSLSCAVKGFYPSDIAVE WESNGQPENNYKTTPPVLDSDGSFFLV SKLTVDKSRWQQGNVFSCSVMHEALH NHYTQKSLSLSPG (SEQ ID NO:100) | | QIVLTQSPAIMSASPGEKVTMTCSASSS VSYMNWYQQKSGTSPKRWIYDTSKLAS GVPAHFRGSGSGTSYSLTISGMEAEDAA TYYCQQWSSNPFTFGSGTKLEIKRTVAA PSVFIFPPSDEQLKSGTASVVCLLNNFYP REAKVQWKVDNALQSGNSQESVTEQD SKDSTYSLSSTLTLSKADYEKHKVYACEV THQGLSSPVTKSFNRGECGPAPEAAGA PSVFLFPPKPKDTLMISRTPEVTCVVVDV SHEDPEVKFNWYVDGVEVHNAKTKPR EEQYNSTYRVVSVLTVLHQDWLNGKEY KCKVSNKALPAPIEKTISKAKGQPREPQV YTLPPSRDELTKNQVSLWCLVKGFYPSDI AVEWESNGQPENNYKTTPPVLDSDGSF FLYSKLTVDKSRWQQGNVFSCSVMHEA LHNHYTQKSLSLSPG (SEQ ID NO:101) |
| | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPLGLGGG SQVQLQQSGAELARPGASVKMSCKAS GYTFTRYTMHWVKQRPGQGLEWIGYI NPSRGYTNYNQKFKDKATLTTDKSSSTA YMQLSSLTSEDSAVYYCARYYDDHYCLD YWGQGTTLTVSSASTKGPSVFPLAPSSK STSGGTAALGCLVKDYFPEPVTVSWNS GALTSGVHTFPAVLQSSGLYSLSSVVTVP SSSLGTQTYICNVN HKPSNTKVDKKVEP KSCGPAPEAAGAPSVFLFPPKPKDTLMI SRTPEVTCVVVDVSHEDPEVKFNWYVD GVEVHNAKTKPREEQYNSTYRVVSVLTV LHQDWLNGKEYKCKVSNKALPAPIEKTI SKAKGQPREPQVYTLPPSRDELTKNQVS LSCAVKGFYPSDIAVEWESNGQPENNY KTTPPVLDSDGSFFLVSKLTVDKSRWQQ GNVFSCSVMHEALHNHYTQKSLSLSPG (SEQ ID NO:102) | | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPEPVTVSWNS GALTSGVHTFPAVLQSSGLYSLSSVVTVP SSSLGTQTYICNVNHKPSNTKVDKKVEP KSCGPAPELLGGGSQIVLTQSPAIMSAS PGEKVTMTCSASSSVSYMNWYQQKSG TSPKRWIYDTSKLASGVPAHFRGSGSGT SYSLTISGMEAEDAATYYCQQWSSNPF TFGSGTKLEIKRTVAAPSVFIFPPSDEQLK SGTASVVCLLNNFYPREAKVQWKVDNA LQSGNSQESVTEQDSKDSTYSLSSTLTLS KADYEKHKVYACEVTHQGLSSPVTKSFN RGECGPAPEAAGAPSVFLFPPKPKDTL MISRTPEVTCVVVDVSHEDPEVKFNWY VDGVEVHNAKTKPREEQYNSTYRVVSV LTVLHQDWLNGKEYKCKVSNKALPAPIE KTISKAKGQPREPQVYTLPPSRDELTKN QVSLWCLVKGFYPSDIAVEWESNGQPE NNYKTTPPVLDSDGSFFLYSKLTVDKSR WQQGNVFSCSVMHEALHNHYTQKSLS LSPG (SEQ ID NO:103) |
| | DIQMTQSPSSLSASVGDRVTITCRASQD VNTAVAWYQQKPGKAPKLLIYSASFLYS GVPSRFSGSRSGTDFTLTISSLQPEDFAT YYCQQHYTTPPTFGQGTKVEIKRTVAAP SVFIFPPSDEQLKSGTASVVCLLNNFYPR EAKVQWKVDNALQSGNSQESVTEQDS KDSTYSLSSTLTLSKADYEKHKVYACEVT HQGLSSPVTKSFNRGECGPAPELLGGGS QVQLQQSGAELARPGASVKMSCKASG YTFTRYTMHWVKQRPGQGLEWIGYIN | | EVQLVESGGGLVQPGGSLRLSCAASGF NIKDTYIHWVRQAPGKGLEWVARIYPT NGYTRYADSVKGRFTISADTSKNTAYLQ MNSLRAEDTAVYYCSRWGGDGFYAM DYWGQGTLVTVSSASTKGPSVFPLAPSS KSTSGGTAALGCLVKDYFPE PVTVSWNS GALTSGVHTFPAVLQSSGLYSLSSVVTVP SSSLGTQTYICNVNHKPSNTKVDKKVEP KSCGPAPELLGGGSQIVLTQSPAIMSAS PGEKVTMTCSASSSVSYMNWYQQKSG |
| | PSRGYTNYNQKFKDKATLTTDKSSSTAY MQLSSLTSEDSAVYYCARYYDDHYCLDY WGQGTTLTVSSASTKGPSVFPLAPSSKS TSGGTAALGCLVKDYFPEPVTVSWNSG ALTSGVHTFPAVLQSSGLYSLSSVVTVPS SSLGTQTYICNVNHKPSNTKVDKKVEPK SCGPAPEAAGAPSVFLFPPKPKDTLMIS RTPEVTCVVVDVSHEDPEVKFNWYVDG VEVHNAKTKPREEQYNSTYRVVSVLTVL HQDWLNGKEYKCKVSNKALPAPIEKTIS KAKGQPREPQVYTLPPSRDELTKNQVSL SCAVKGFYPSDIAVEWESNGQPENNYK TTPPVLDSDGSFFLVSKLTVDKSRWQQ GNVFSCSVMHEALHNHYTQKSLSLSPG (SEQ ID NO:104) | | TSPKRWIYDTSKLASGVPAHFRGSGSGT SYSLTISGMEAEDAATYYCQQWSSNPF TFGSGTKLEIKRTVAAPSVFIFPPSDEQLK SGTASVVCLLNNFYPREAKVQWKVDNA LQSGNSQESVTEQDSKDSTYSLSSTLTLS KADYEKHKVYACEVTHQGLSSPVTKSFN RGECGPAPEAAGAPSVFLFP PKPKDTL MISRTPEVTCVVVDVSHEDPEVKFNWY VDGVEVHNAKTKPREEQYNSTYRVVSV LTVLHQDWLNGKEYKCKVSNKALPAPIE KTISKAKGQPREPQVYTLPPSRDELTKN QVSLWCLVKGFYPSDIA VEWESNGQPE NNYKTTPPVLDSDGSFFLYSKLTVDKSR WQQGNVFSCSVMHEALHNHYTQKSLS LSPG (SEQ ID NO:105) |
| | DIQMTQSPASLSASLGETVSIECLASEGIS NDLAWYQQKSGKSPQLLIYATSRLQDG VPSRFSGSGSGTRYSLKISGMQPEDEAD YFCQQSYKYPWTFGGGTKLELKRTVAA PSVFIFPPSDEQLKSGTASVVCLLNNFYP REAKVQWKVDNALQSGNSQESVTEQD SKDSTYSLSSTLTLSKADYEKH KVYACEV THQGLSSPVTKSFNRGECGPAPLGLGG GSQVQLQQSGAELARPGASVKMSCKA SGYTFTRYTMHWVKQRPGQGLEWIGYI NPSRGYTNYNQKFKDKATLTTDKSSST A YMQLSSLTSEDSAVYYCARYYDDHYCLD YWGQGTTLTVSSASTKGPSVFPLAPSSK STSGGTAALGCLVKDYFPEPVTVSWNS GALTSGVHTFPAVLQSSGLYSLSSVVTVP SSSLGTQTYICNVNHKPSNTKVDKKVEP KSCGPAPEAAGAPSVFLFPPKPKDTLMI SRTPEVTCVVVDVSHEDPEVKFNWYVD GVEVHNAKTKPREEQYNSTYRVVSVLTV LHQDWLNGKEYKCKVSNKALPAPIEKTI SKAKGQPREPQVYTLPPSRDELTKNQVS LSCAVKGFYPSDIAVEWESNGQPENNY KTTPPVLDSDGSFFLVSKLTVDKSRWQQ GNVFSCSVMHEALHNHYTQKSLSLSPG (SEQ ID NO:106) | | EVQLAESGGGLVQPGRSMKLSCAASGF TFSNFPMAWVRQAPTKGLEWVATISTS GGSTYYRDSVKGRFTISRDNAKSTLYLQ MNSLRSEDTATYYCTRTLYILRVFYFDY WGQGVMVTVSSASTKGPSVFPLAPSSK STSGGTAALGCLVKDYFPEPVTVSWNS GALTSGVHTFPAVLQSSGLYSLSSVVTVP SSSLGTQTYICNVNHKPSNTKVDKKVEP KSCGPAPELLGGGSQIVLTQSPAIMSAS PG EKVTMTCSASSSVSYMNWYQQKSG TSPKRWIYDTSKLASGVPAHFRGSGSGT SYSLTISGMEAEDAATYYCQQWSSNPF TFGSGTKLEIKRTVAAPSVFIFPPSDEQLK SGTASVVCLLNNFYPREAKVQWKVDNA LQSGNSQESVTEQDSKDSTYSLSSTLTLS KADYEKHKVYACEVTHQGLSSPVTKSFN RG ECG PAPEAAGAPSVFLFPPKPKDTL MISRTPEVTCVVVDVSHEDPEVKFNWY VDGVEVHNAKTKPREEQYNSTYRVVSV LTVLHQDWLNGKEYKCKVSNKALPAPIE KTISKAKGQPREPQVYTLPPSRDELTKN QVSLWCLVKGFYPSDIAVEWESNGQPE NNYKTTPPVLDSDGSFFLYSKLTVDKSR WQQGNVFSCSVMHEALHNHYTQKSLS LSPG (SEQ ID NO:107) |
| | DIQMTQSPASLSASLGETVSIECLASEGIS NDLAWYQQKSGKSPQLLIYATSRLQDG VPSRFSGSGSGTRYSLKISGMQPEDEAD YFCQQSYKYPWTFGGGTKLELKRTVAA PSVFIFPPSDEQLKSGTASVVCLLNNFYP REAKVQWKVDNALQSGNSQESVTEQD SKDSTYSLSSTLTLSKADYEKH KVYACEV THQGLSSPVTKSFNRGECGPAPELLGGG SQVQLQQSGAELARPGASVKMSCKAS GYTFTRYTMHWVKQRPGQGLEWIGYI NPSRGYTNYNQKFKDKATLTTDKSSSTA YMQLSSLTSEDSAVYYCARYYDDHYCLD | | EVQLAESGGGLVQPGRSMKLSCAASGF TFSNFPMAWVRQAPTKGLEWVATISTS GGSTYYRDSVKGRFTISRDNAKSTLYLQ MNSLRSEDTATYYCTRTLYILRVFYFDY WGQGVMVTVSSASTKGPSVFPLAPSSK STSGGTAALGCLVKDYFPEPVTVSWNS GALTSGVHTFPAVLQSSGLYSLSSVVTVP SSSLGTQTYICNVNHKPSNTKVDKKVEP KSCGPAPELLGGGSQIVLTQSPAIMSAS PG EKVTMTCSASSSVSYMNWYQQKSG TSPKRWIYDTSKLASGVPAHFRGSGSGT SYSLTISGMEAEDAATYYCQQWSSNPF |
| | YWGQGTTLTVSSASTKGPSVFPLAPSSK STSGGTAALGCLVKDYFPEPVTVSWNS GALTSGVHTFPAVLQSSGLYSLSSVVTVP SSSLGTQTYICNVNHKPSNTKVDKKVEP KSCGPAPEAAGAPSVFLFPPKPKDTLMI SRTPEVTCVVVDVSHEDPEVKFNWYVD GVEVHNAKTKPREEQYNSTYRVVSVLTV LHQDWLNGKEYKCKVSNKALPAPIEKTI SKAKGQPREPQVYTLPPSRDELTKNQVS LSCAVKGFYPSDIAVEWESNGQPENNY KTTPPVLDSDGSFFLVSKLTVDKSRWQQ GNVFSCSVMHEALHNHYTQKSLSLSPG (SEQ ID NO:108) | | TFGSGTKLEIKRTVAAPSVFIFPPSDEQLK SGTASVVCLLNNFYPREAKVQWKVDNA LQSGNSQESVTEQDSKDSTYSLSSTLTLS KADYEKHKVYACEVTHQGLSSPVTKSFN RGECGPAPEAAGAPSVFLFPPKPKDTL MISRTPEVTCVVVDVSHEDPEVKFNWY VDGVEVHNAKTKPREEQYNSTYRVVSV LTVLHQDWLNGKEYKCKVSNKALPAPIE KTISKAKGQPREPQVYTLPPSRDELTKN QVSLWCLVKGFYPSDIAVEWESNGQPE NNYKTTPPVLDSDGSFFLYSKLTVDKSR WQQGNVFSCSVMHEALHNHYTQKSLS LSPG (SEQ ID NO:109) |

TABLE 23

Binding affinities to human and murine Fc receptors by Biacore.

| | | | | | | |
|---|---|---|---|---|---|---|
| FcγR | Isotype hIgG1 | Isotype hIgG4 | Isotype mIgG1 | Isotype mIgG2a | IgG² Her47 LHL-LHL | Fab² Her47 LHL-LHL |
| hFcγRI | 3.9 × 10⁻⁹ | 1.2 × 10⁻⁸ | ND | ND | 5.8 × 10⁻⁹ | 5.5 × 10⁻⁹ |
| hFcγRIIA 167 Arg | 8.1 × 10⁻⁶ | NB | ND | ND | 1.5 ×10⁻⁵ | 6.8 × 10⁻⁶ |
| hFcγRIIA 167 His | 4.4 × 10⁻⁶ | NB | ND | ND | 3.5 × 10⁻⁶ | 4.2 × 10⁻⁶ |
| hFcγRIIB | 1.3 × 10⁻⁵ | 2.2 × 10⁻⁵ | ND | ND | 2.3 × 10⁻⁵ | 1.2 × 10⁻⁵ |
| hFcγRIIIA 176 Val | 6.4 × 10⁻⁶ | NB | ND | ND | 1.7 × 10⁻⁶ | 1.8 × 10⁻⁶ |
| hFcγRIIIA 176 Phe | 2.6 × 10⁻⁶ | NB | ND | ND | 8.9 × 10⁻⁷ | 7.3 × 10⁻⁷ |
| hFcγRIIIB | 6.8 × 10⁻⁶ | NB | ND | ND | 3.4 × 10⁻⁶ | 3.9 × 10⁻⁶ |
| mFcγRI | 1.1 × 10⁻⁷ | 2.7 × 10⁻⁷ | NB | 2.6 × 10⁻⁸ | 1.9 × 10⁻⁷ | 1.6 × 10⁻⁷ |
| mFcγRIIB | 1.3 × 10⁻⁵ | NB | ND | ND | 1.2 × 10⁻⁵ | 1.0 × 10⁻⁵ |
| mFcγRIII | 1.8 × 10⁻⁵ | NB | 3.2 × 10⁻⁵ | 1.7 × 10⁻⁵ | 2.3 × 10⁻⁵ | 1.5 × 10⁻⁵ |
| mFcγRIV | 1.8 × 10⁻⁶ | NB | NB | 1.2 × 10⁻⁶ | 2.0 × 10⁻⁶ | 1.1 × 10⁻⁶ |

ND = Not Done
NB = No Binding

1. A protein comprising a first moiety and a second moiety and a peptide linker between the first moiety and the second moiety,
wherein the first moiety comprises an antibody, an antigen-binding portion of an antibody, or a receptor ectodomain, wherein the first moiety is capable of specifically binding to a molecule expressed in a diseased tissue;
wherein the peptide linker comprises an amino acid sequence from a human immunoglobulin hinge region or an amino acid sequence having from 1 to about 7 amino acid substitutions compared to a human immunoglobulin hinge region;
wherein the peptide linker is cleavable by a protease expressed in a diseased tissue;
wherein the second moiety comprises an antibody, an antigen-binding portion of an antibody, or a receptor ectodomain, wherein the second moiety is capable of specifically binding to a molecule expressed in the diseased tissue;
wherein the binding of the second moiety to the molecule expressed in the diseased tissue is reduced or inhibited when the peptide linker is uncleaved; and
wherein the peptide linker cleavage unmasks the second moiety in the diseased tissue and allows binding of the second moiety to the molecule expressed in a diseased tissue selectively in the diseased tissue.

2. The protein of claim 1, wherein the peptide linker is between about 5 and about 15 amino acids in length.

3. The protein of claim 1, wherein the peptide linker comprises or consists of the amino acid sequence of SEQ ID NO:1, SEQ ID NO:2, SEQ ID NO:3, SEQ ID NO:4, SEQ ID NO:5, SEQ ID NO:69, SEQ ID NO:70, SEQ ID NO:71, SEQ ID NO:72, SEQ ID NO:81, SEQ ID NO:82, SEQ ID NO:83, SEQ ID NO:84, SEQ ID NO:85, SEQ ID NO:86, or SEQ ID NO:87.

4. The protein of claim 1, wherein the protease is a human matrix metalloprotease (MMP), a human cathepsin, human enterokinase, human thrombin, human tPA, human Granzyme B, human uPA, or human ADAMTs-5.

5. The protein of claim 1, wherein the peptide linker comprises a cleavage site for a human MMP a human cathepsin, human enterokinase, human thrombin, human tPA, human Granzyme B, human uPA, or human ADAMTs-5.

6-10. (canceled)

11. The protein of claim 1, wherein the first moiety is a Fab, a single-chain Fab, a VH domain, a VL domain, an immunoglobulin new antigen receptor (IgNAR), a single-chain variable fragment (scFv), a diabody, or a T cell receptor domain.

12. (canceled)

13. The protein of claim 1, wherein the first moiety specifically binds to a first molecule expressed in a diseased tissue and the second moiety is capable of specifically binding to a second molecule expressed in a diseased tissue, wherein the first molecule expressed in a diseased tissue and the second molecule expressed in a diseased tissue are different molecules.

14-17. (canceled)

18. The protein of claim 1, wherein the first moiety binds specifically to human EGFR, human HER2, human HER3, human CD105, human C-KIT, human PD1, human PD-L1, human PSMA, human EpCAM, human Trop2, human EphA2, human CD20, human BCMA, human GITR, human OX40, human CSF1R, human Lag3 or human cMET.

19. The protein of claim 1, wherein the second moiety is capable of specifically binding to a molecule expressed by a human immune cell.

20. The protein of claim 19, wherein the molecule expressed by a human immune cell is human CD3, human CD16A, human CD16B, human CD28, human CD89, human CTLA4, human NKG2D, human SIRPα, human SIRPγ, human PD1, human Lag3, human 4-1BB, human OX40, or human GITR.

21. The protein of claim 1, wherein the first moiety comprises a heavy chain variable (VH) region and a light chain variable (VL) region.

22. The protein of claim 1, wherein the first moiety comprises an immunoglobulin constant region or a portion of an immunoglobulin constant region.

23. The protein of claim 22, wherein the immunoglobulin constant region is IgG, IgE, IgM, IgD, IgA, or IgY.

24. The protein of claim 22, wherein the immunoglobulin constant region is IgG1, IgG2, IgG3, IgG4, IgA1, or IgA2.

25. The protein of claim 22, wherein the immunoglobulin constant region is immunologically inert.

26. The protein of claim 22, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A and L235A, a human IgG1 constant region comprising the amino acid substitutions L234A, L235A and G237A, a human IgG1 constant region comprising the amino acid substitutions L234A, L235A, G237A and P331S, or a wild-type human IgG2 constant region.

27. (canceled)

28. The protein of claim 1, wherein the second moiety is a Fab, a single-chain Fab, a VH domain, a VL domain, an immunoglobulin new antigen receptor (IgNAR), a single-chain variable fragment (scFv), or a T cell receptor domain.

29. The protein of claim 1, wherein the second moiety is capable of binding specifically to human CD47, human CD3 or human PD-L1.

30. (canceled)

31. The protein of claim 1, wherein the second moiety comprises a heavy chain variable (VH) region and a light chain variable (VL) region.

32. The protein of claim 1, wherein the second moiety comprises an immunoglobulin constant region or a portion of an immunoglobulin constant region.

33. The protein of claim 32, wherein the immunoglobulin constant region is IgG, IgE, IgM, IgD, IgA or IgY.

34. The protein of claim 32, wherein the immunoglobulin constant region is IgG1, IgG2, IgG3, IgG4, IgA1 or IgA2.

35. The protein of claim 32, wherein the immunoglobulin constant region is immunologically inert.

36. The protein of claim 32, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A and L235A, a human IgG1 constant region comprising the amino acid substitutions L234A, L235A and G237A, a human IgG1 constant region comprising the amino acid substitutions L234A, L235A, G237A and P331S, or a wild-type human IgG2 constant region.

37-41. (canceled)

42. The protein of claim 1, wherein the diseased tissue is a tumour or an inflamed tissue.

43. The protein of claim 1, wherein the first moiety binds specifically to human cMET, wherein the second moiety binds specifically to human CD47, and wherein the protein comprises a first polypeptide chain comprising or consisting of the amino acid sequence of SEQ ID NO: 16 and a second polypeptide chain comprising or consisting of the amino acid sequence of SEQ ID NO: 17.

44. The protein of claim 1, wherein the first moiety binds specifically to human HER2, wherein the second moiety binds specifically to human CD3, and wherein the protein comprises a first polypeptide chain comprising or consisting of the amino acid sequence of SEQ ID NO:26 and a second polypeptide chain comprising or consisting of the amino acid sequence of SEQ ID NO:27.

45. The protein of claim 1, wherein the first moiety binds specifically to human HER2, wherein the second moiety binds specifically to human CD47, and wherein the protein comprises a first polypeptide chain comprising or consisting of the amino acid sequence of SEQ ID NO:34 and a second polypeptide chain comprising or consisting of the amino acid sequence of SEQ ID NO:35.

46. The protein of claim 1, wherein the first moiety binds specifically to human cMET, wherein the second moiety binds specifically to human CD47, and wherein the protein comprises a first polypeptide chain comprising or consisting of the amino acid sequence of SEQ ID NO:36 and a second polypeptide chain comprising or consisting of the amino acid sequence of SEQ ID NO:37.

47. The protein of claim 1, wherein the first moiety binds specifically to human Her2, wherein the second moiety binds specifically to human CD3, and wherein the protein comprises a first polypeptide chain comprising or consisting of the amino acid sequence of SEQ ID NO:38 and a second polypeptide chain comprising or consisting of the amino acid sequence of SEQ ID NO:39.

48. The protein of claim 1, wherein the first moiety binds specifically to human Her2, wherein the second moiety binds specifically to human CD3, and wherein the protein comprises a first polypeptide chain comprising or consisting of the amino acid sequence of SEQ ID NO:40 and a second polypeptide chain comprising or consisting of the amino acid sequence of SEQ ID NO:41.

49. The protein of claim 1, wherein the first moiety binds specifically to human Her2, wherein the second moiety binds specifically to human CD47, and wherein the protein comprises a first polypeptide chain and a second polypeptide chain, wherein:
(a) the first polypeptide chain comprises or consists of the amino acid sequence of SEQ ID NO:42, and the second polypeptide chain comprises or consists of the amino acid sequence of SEQ ID NO:43; or
(b) the first polypeptide chain comprises or consists of the amino acid sequence of SEQ ID NO:44, and the second polypeptide chain comprises or consists of the amino acid sequence of SEQ ID NO:45; or
(c) the first polypeptide chain comprises or consists of the amino acid sequence of SEQ ID NO:46, and the second polypeptide chain comprises or consists of the amino acid sequence of SEQ ID NO:47; or
(d) the first polypeptide chain comprises or consists of the amino acid sequence of SEQ ID NO:48, and the second polypeptide chain comprises or consists of the amino acid sequence of SEQ ID NO:49; or
(e) the first polypeptide chain comprises or consists of the amino acid sequence of SEQ ID NO:50, and the second polypeptide chain comprises or consists of the amino acid sequence of SEQ ID NO:51; or
(f) the first polypeptide chain comprises or consists of the amino acid sequence of SEQ ID NO:52, and the second polypeptide chain comprises or consists of the amino acid sequence of SEQ ID NO:53; or
(g) the first polypeptide chain comprises or consists of the amino acid sequence of SEQ ID NO:54, and the second polypeptide chain comprises or consists of the amino acid sequence of SEQ ID NO:55; or
(h) the first polypeptide chain comprises or consists of the amino acid sequence of SEQ ID NO:88, and the second polypeptide chain comprises or consists of the amino acid sequence of SEQ ID NO: 89; or
(i) the first polypeptide chain comprises or consists of the amino acid sequence of SEQ ID NO:90, and the second polypeptide chain comprises or consists of the amino acid sequence of SEQ ID N0:91; or
(j) the first polypeptide chain comprises or consists of the amino acid sequence of SEQ ID NO:44, and the second polypeptide chain comprises or consists of the amino acid sequence of SEQ ID NO: 92; or
(k) the first polypeptide chain comprises or consists of the amino acid sequence of SEQ ID NO:44, and the second polypeptide chain comprises or consists of the amino acid sequence of SEQ ID NO:93; or
(1) the first polypeptide chain comprises or consists of the amino acid sequence of SEQ ID NO:44, and the second polypeptide chain comprises or consists of the amino acid sequence of SEQ ID NO: 94; or
(m) the first polypeptide chain comprises or consists of the amino acid sequence of SEQ ID NO:44, and the second polypeptide chain comprises or consists of the amino acid sequence of SEQ ID NO: 95; or
(n) the first polypeptide chain comprises or consists of the amino acid sequence of SEQ ID NO:44, and the second polypeptide chain comprises or consists of the amino acid sequence of SEQ ID NO: 96; or
(o) the first polypeptide chain comprises or consists of the amino acid sequence of SEQ ID NO:44, and the second polypeptide chain comprises or consists of the amino acid sequence of SEQ ID NO:97.

50. The protein of claim 1, wherein the first moiety binds specifically to human Her2, wherein the second moiety binds specifically to human CD3, and wherein the protein comprises a first polypeptide chain comprising or consisting of the amino acid sequence of SEQ ID NO:73 and a second polypeptide chain comprising or consisting of the amino acid sequence of SEQ ID NO:74.

51. The protein of claim 1, wherein the first moiety binds specifically to human cMET, wherein the second moiety binds specifically to human cMET, and wherein the protein comprises a first polypeptide chain comprising or consisting of the amino acid sequence of SEQ ID NO:75 and a second polypeptide chain comprising or consisting of the amino acid sequence of SEQ ID NO:76.

52. The protein of claim 1, wherein the first moiety binds specifically to human Her2, wherein the second moiety binds specifically to human CD3, and wherein the protein comprises a first polypeptide chain and a second polypeptide chain, wherein:
(a) the first polypeptide chain comprises or consists of the amino acid sequence of SEQ ID NO:98, and the second polypeptide chain comprises or consists of the amino acid sequence of SEQ ID NO: 99; or
(b) the first polypeptide chain comprises or consists of the amino acid sequence of SEQ ID NO: 100, and the second polypeptide chain comprises or consists of the amino acid sequence of SEQ ID NO: 101; or
(c) the first polypeptide chain comprises or consists of the amino acid sequence of SEQ ID NO: 102, and the second polypeptide chain comprises or consists of the amino acid sequence of SEQ ID NO: 103; or
(d) the first polypeptide chain comprises or consists of the amino acid sequence of SEQ ID NO: 104, and the second polypeptide chain comprises or consists of the amino acid sequence of SEQ ID NO: 105.

53. An immunoconjugate comprising the protein of claim 1 linked to a therapeutic agent.

54. The immunoconjugate of claim 53, wherein the therapeutic agent is a cytotoxin, a radioisotope, a chemotherapeutic agent, an immunomodulatory agent, a cytostatic enzyme, a cytolytic enzyme, a therapeutic nucleic acid, an anti-angiogenic agent, an anti-proliferative agent, or a pro-apoptotic agent.

55. A pharmaceutical composition comprising the protein of claim 1, and a pharmaceutically acceptable carrier, diluent or excipient.

56. A nucleic acid molecule encoding the protein or a portion of the protein of claim 1.

57. (canceled)

58. An expression vector comprising the nucleic acid molecule of claim 56.

59. A recombinant host cell comprising the nucleic acid molecule of claim 56.

60. A method of producing a protein, the method comprising:
culturing a recombinant host cell comprising the expression vector of claim 58 under conditions whereby the nucleic acid molecule is expressed, thereby producing the protein; and
isolating the protein from the host cell or culture.

61. A method for enhancing an anti-cancer immune response in a subject, comprising administering to the subject a therapeutically effective amount of the protein of claim 1.

62. A method of treating cancer, an autoimmune disease, an inflammatory disease, a cardiovascular disease or a fibrotic disease in a subject, comprising administering to the subject a therapeutically effective amount of the protein of claim 1.

63-72. (canceled)


SEQUENCE LISTING (112 sequences)

SEQ ID NO: 1 - protein, 7 aa, Artificial Sequence
  peptide linker
GPAPELL

SEQ ID NO: 2 - protein, 11 aa, Artificial Sequence
  peptide linker
GPAPELLGGGS

SEQ ID NO: 3 - protein, 11 aa, Artificial Sequence
  peptide linker
GPAPLGLGGGS

SEQ ID NO: 4 - protein, 13 aa, Artificial Sequence
  peptide linker
PPCPAPELLGGGS

SEQ ID NO: 5 - protein, 13 aa, Artificial Sequence
  peptide linker
PPCPAPLGLGGGS

SEQ ID NO: 6 - protein, 678 aa, Artificial Sequence
  Fab2 CMET/CD47 Heavy chain
QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 673 aa, Artificial Sequence
  Fab2 CMET/CD47 Light chain
EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 8 - protein, 683 aa, Artificial Sequence
  Fab2 CMET/CD47 Heavy chain
QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 678 aa, Artificial Sequence
  Fab2 CMET/CD47 Light chain
EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 10 - protein, 688 aa, Artificial Sequence
  Fab2 CMET/CD47 Heavy chain
QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 680 aa, Artificial Sequence
  Fab2 CMET/CD47 Light chain
EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 12 - protein, 4 aa, Unknown
  MMP peptide substrate sequence
PLGL

SEQ ID NO: 13 - protein, 675 aa, Artificial Sequence
  Fab2 CMET/CD47 Light chain
EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 14 - protein, 680 aa, Artificial Sequence
  Fab2 CMET/CD47 Heavy chain
QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 5 aa, Artificial Sequence
  peptide linker
GGGGS

SEQ ID NO: 16 - protein, 684 aa, Artificial Sequence
  Fab2 CMET/CD47 Heavy chain
QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 679 aa, Artificial Sequence
  Fab2 CMET/CD47 Light chain
EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 18 - protein, 452 aa, Artificial Sequence
  Fab2 CMET/CD47 Heavy chain
QVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 450 aa, Artificial Sequence
  Fab2 CMET/CD47 Light chain
DIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 20 - protein, 688 aa, Artificial Sequence
  Fab2 Her2/CD3 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 668 aa, Artificial Sequence
  Fab2 Her2/CD3 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 22 - protein, 683 aa, Artificial Sequence
  Fab2 Her2/CD3 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 665 aa, Artificial Sequence
  Fab2 Her2/CD3 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 24 - protein, 685 aa, Artificial Sequence
  Fab2 Her2/CD3 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 663 aa, Artificial Sequence
  Fab2 Her2/CD3 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 26 - protein, 689 aa, Artificial Sequence
  Fab2 Her2/CD3 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 669 aa, Artificial Sequence
  Fab2 Her2/CD3 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 28 - protein, 344 aa, Artificial Sequence
  IgG Her2/CD47 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSS

SEQ ID NO: 29 - protein, 333 aa, Artificial Sequence
  IgG Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIK

SEQ ID NO: 30 - protein, 346 aa, Artificial Sequence
  IgG Her2/CD47 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSS

SEQ ID NO: 31 - protein, 330 aa, Artificial Sequence
  IgG Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIK

SEQ ID NO: 32 - protein, 10 aa, Artificial Sequence
  peptide linker
GGGGSGGGGS

SEQ ID NO: 33 - protein, 15 aa, Artificial Sequence
  peptide linker
GGGGSGGGGSGGGGS

SEQ ID NO: 34 - protein, 350 aa, Artificial Sequence
  IgG Her2/CD47 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSS

SEQ ID NO: 35 - protein, 337 aa, Artificial Sequence
  IgG Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIK

SEQ ID NO: 36 - protein, 348 aa, Artificial Sequence
  Fab2 CMET/CD47 One Arm style Heavy chain
QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSS

SEQ ID NO: 37 - protein, 341 aa, Artificial Sequence
  Fab2 CMET/CD47 One Arm style Light chain
EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIK

SEQ ID NO: 38 - protein, 353 aa, Artificial Sequence
  Fab2 Her2/CD3 One Arm style Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 39 - protein, 331 aa, Artificial Sequence
  Fab2 Her2/CD3 One Arm style Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIK

SEQ ID NO: 40 - protein, 359 aa, Artificial Sequence
  Fab2 Her2/CD3(34) One Arm style Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 41 - protein, 334 aa, Artificial Sequence
  Fab2 Her2/CD3(34) One Arm style Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVL

SEQ ID NO: 42 - protein, 350 aa, Artificial Sequence
  IgG2 Her2/CD47 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSS

SEQ ID NO: 43 - protein, 337 aa, Artificial Sequence; identical to SEQ ID NO: 35
  IgG2 Her2/CD47 Light chain

SEQ ID NO: 44 - protein, 350 aa, Artificial Sequence; identical to SEQ ID NO: 34
  IgG2 Her2/CD47 Heavy chain

SEQ ID NO: 45 - protein, 337 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIK

SEQ ID NO: 46 - protein, 350 aa, Artificial Sequence; identical to SEQ ID NO: 42
  IgG2 Her2/CD47 Heavy chain

SEQ ID NO: 47 - protein, 337 aa, Artificial Sequence; identical to SEQ ID NO: 45
  IgG2 Her2/CD47 Light chain

SEQ ID NO: 48 - protein, 352 aa, Artificial Sequence
  IgG2 Her2/CD47 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPELLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSS

SEQ ID NO: 49 - protein, 339 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPELLGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIK

SEQ ID NO: 50 - protein, 352 aa, Artificial Sequence; identical to SEQ ID NO: 48
  IgG2 Her2/CD47 Heavy chain

SEQ ID NO: 51 - protein, 339 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECPPCPAPLGLGGGSDIVMTQSPLSLPVTPGEPASISCRSSQSLLHSNGYNYLHWYLQKPGQSPQLLIYKVSNRLSGVPDRFSGSGSGTDFTLKISRVEAEDVGVYYCFQNTHTPRTFGGGTKVEIK

SEQ ID NO: 52 - protein, 352 aa, Artificial Sequence
  IgG2 Her2/CD47 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPPCPAPLGLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSS

SEQ ID NO: 53 - protein, 339 aa, Artificial Sequence; identical to SEQ ID NO: 49
  IgG2 Her2/CD47 Light chain

SEQ ID NO: 54 - protein, 352 aa, Artificial Sequence; identical to SEQ ID NO: 52
  IgG2 Her2/CD47 Heavy chain

SEQ ID NO: 55 - protein, 339 aa, Artificial Sequence; identical to SEQ ID NO: 51
  IgG2 Her2/CD47 Light chain

SEQ ID NO: 56 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 327 aa, Artificial Sequence
  Human IgG4(S228P) immunoglobulin Fc region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 330 aa, Homo sapiens [sequence omitted]

SEQ ID NO: 59 - protein, 330 aa, Artificial Sequence
  Human IgG1-3M immunoglobulin Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 61 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 329 aa, Artificial Sequence
  Human IgG1-3M REEM allotype immunoglobulin Fc region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 64 - protein, 323 aa, Macaca fascicularis
MWPLVAALLLGSACCGSAQLLFNKTKSVEFTFCNDTVVIPCFVTNMEAQNTTEVYVKWKFKGRDIYTFDGALNKSTAPANFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 65 - protein, 5 aa, Artificial Sequence
  immunoglobulin constant region
RDELT

SEQ ID NO: 66 - protein, 4 aa, Artificial Sequence
  immunoglobulin constant region
REEM

SEQ ID NO: 67 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 68 - protein, 1381 aa, Macaca fascicularis
MKAPAVLVPGILVLLFTLVQRSNGECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISIALLLLLGLFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDT

SEQ ID NO: 69 - protein, 11 aa, Artificial Sequence
  peptide linker
GPAPELLGGPS

SEQ ID NO: 70 - protein, 11 aa, Artificial Sequence
  peptide linker
GPAPLGLGGPS

SEQ ID NO: 71 - protein, 13 aa, Artificial Sequence
  peptide linker
PPCPAPELLGGPS

SEQ ID NO: 72 - protein, 13 aa, Artificial Sequence
  peptide linker
PPCPAPLGLGGPS

SEQ ID NO: 73 - protein, 694 aa, Artificial Sequence
  Fc-Her2/CD3(34) Heavy chain
CPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGPAPLGLGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLGGGSEVKLLESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 74 - protein, 672 aa, Artificial Sequence
  Fc-Her2/CD3(34) Light chain
CPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGPAPLGLGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 75 - protein, 350 aa, Artificial Sequence
  Fab2 CMET/CMET One Arm style Heavy chain
QVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGASVKVSCKASGYTFTSYTMHWVRQAPGQGLEWMGIINPSGGSTSYAQKFQGRVTMTRDTSTSTVYMELSSLRSEDTAVYYCARQEITTEFDYWGQGTLVTVSS

SEQ ID NO: 76 - protein, 340 aa, Artificial Sequence
  Fab2 CMET/CMET One Arm style Light chain
EIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSEIVLTQSPGTLSLSPGERATLSCRASQSVSSYANSYLHWYQQKPGQAPRLLIYRGSTRESGIPDRFSGSGSGTDFTLTISRLEPEDFAVYYCQQSKSEPLTFGGGTKVEIK

SEQ ID NO: 77 - protein, 1255 aa, Homo sapiens [sequence omitted]

SEQ ID NO: 78 - protein, 1406 aa, Macaca fascicularis
MAESPASAFRDSLRKSVRTAAGNPGVPELGGTHPGLREEREKVKLGVATPRLVGMQLEGASWERACSQSQEEEEVEEEGCLRKYKNEVVELRFPSIGTGETRGAPWAAVRPFPRGSFRRRAPGPHPSPHPAPHALPAGSSRSHGAGAAVSTMELAAWYRWGLLLALLPPGATGTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPVCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLRVFETLEEITGYLYISAWPDSLPDLSVLQNLQVIRGRILHNGAYSLTLQGLGISWLGLRSLRELGSGLALIHHNTRLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGTCQSCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGTGGMVHHRHRSSSTRSGGGDLTLGLEPSEEEAPRSPRAPSEGTGSDVFDGDLGMGAAKGLQSLPAHDPSPLQRYSEDPTVPLPSETDGYVAPLTCSPQPEYVNQPDVRPQPPSPQEGPLSPARPTGATLERPKTLSPGKNGVVKDVFAFGGAVENPEYLAPRGGAAPQPHLPPAFSPAFDNLYYWDQDPSERGAPPSTFKGTPTAENPEYLGLDVPV

SEQ ID NO: 79 - protein, 206 aa, Homo sapiens
MQSGTHWRVLGLCLLSVGVWGQDGNEEMGGITQTPYKVSISGTTVILTCPQYPGSEILQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 80 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 81 - protein, 11 aa, Artificial Sequence
  peptide linker
GPAPEAAGAGS

SEQ ID NO: 82 - protein, 11 aa, Artificial Sequence
  peptide linker
GPADDDDKSGS

SEQ ID NO: 83 - protein, 11 aa, Artificial Sequence
  peptide linker
GPALVPRGSGS

SEQ ID NO: 84 - protein, 12 aa, Artificial Sequence
  peptide linker
GPGPFGRSAGGP

SEQ ID NO: 85 - protein, 11 aa, Artificial Sequence
  peptide linker
GPAPLEADAGS

SEQ ID NO: 86 - protein, 11 aa, Artificial Sequence
  peptide linker
GPAPEARRGGS

SEQ ID NO: 87 - protein, 11 aa, Artificial Sequence
  peptide linker
GPAPEGEARGS

SEQ ID NO: 88 - protein, 678 aa, Artificial Sequence
  IgG2 IgG1-DAA Her2/CD47 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPLGLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPDVFLFPPKPKDTLMISRTPEVTCVVVDVSHEDPEVKFNWYVDGVEVHNAKTKPREEQYNSTYRVVSVLTVLHQDWLNGKEYKCKVSNAALPAPIAKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 444 aa, Artificial Sequence
  IgG2 IgG1-DAA Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 678 aa, Artificial Sequence
  IgG2 IgG1-DAA Her2/CD47 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLVQSGAEVKKPGESLKISCKGSGYTFTNYYIFWVRQMPGKGLEWMGIINPVDGDTNYNPSFQGQVTISADKSISTAYLQWSSLKASDTAMYYCARGGYTMDRWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPDVFLFPPKPKDTLMISRTPEVTCVVVDVSHEDPEVKFNWYVDGVEVHNAKTKPREEQYNSTYRVVSVLTVLHQDWLNGKEYKCKVSNAALPAPIAKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 444 aa, Artificial Sequence
  IgG2 IgG1-DAA Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 337 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPADDDDKSGSDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIK

SEQ ID NO: 93 - protein, 337 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPALVPRGSGSDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIK

SEQ ID NO: 94 - protein, 338 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPGPFGRSAGGPDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIK

SEQ ID NO: 95 - protein, 337 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLEADAGSDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIK

SEQ ID NO: 96 - protein, 337 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPEARRGGSDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIK

SEQ ID NO: 97 - protein, 337 aa, Artificial Sequence
  IgG2 Her2/CD47 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPEGEARGSDIVMTQSPLSLPVTPGEPASISCRSSQSLLHSNGYNYLHWYLQKPGQSPQLLIYKVSNRFSGVPDRFSGSGSGTDFTLKISRVEAEDVGVYYCFQNTHTPRTFGGGTKVEIK

SEQ ID NO: 98 - protein, 674 aa, Artificial Sequence
  Fab2 Her2/CD3 Heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 99 - protein, 656 aa, Artificial Sequence
  Fab2 Her2/CD3 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 674 aa, Artificial Sequence; identical to SEQ ID NO: 98
  Fab2 Her2/CD3 Heavy chain

SEQ ID NO: 101 - protein, 656 aa, Artificial Sequence
  Fab2 Her2/CD3 Light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 665 aa, Artificial Sequence
  Fab2 Her2/CD3 Heavy chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 103 - protein, 665 aa, Artificial Sequence
  Fab2 Her2/CD3 Light chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 665 aa, Artificial Sequence
  Fab2 Her2/CD3 Heavy chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 105 - protein, 665 aa, Artificial Sequence; identical to SEQ ID NO: 103
  Fab2 Her2/CD3 Light chain

SEQ ID NO: 106 - protein, 665 aa, Artificial Sequence
  Fab2 Her2/CD3 Heavy chain
DIQMTQSPASLSASLGETVSIECLASEGISNDLAWYQQKSGKSPQLLIYATSRLQDGVPSRFSGSGSGTRYSLKISGMQPEDEADYFCQQSYKYPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPLGLGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 107 - protein, 666 aa, Artificial Sequence
  Fab2 Her2/CD3 Light chain
EVQLAESGGGLVQPGRSMKLSCAASGFTFSNFPMAWVRQAPTKGLEWVATISTSGGSTYYRDSVKGRFTISRDNAKSTLYLQMNSLRSEDTATYYCTRTLYILRVFYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPELLGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 108 - protein, 665 aa, Artificial Sequence
  Fab2 Her2/CD3 Heavy chain
DIQMTQSPASLSASLGETVSIECLASEGISNDLAWYQQKSGKSPQLLIYATSRLQDGVPSRFSGSGSGTRYSLKISGMQPEDEADYFCQQSYKYPWTFGGGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGPAPELLGGGSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 109 - protein, 666 aa, Artificial Sequence; identical to SEQ ID NO: 107
  Fab2 Her2/CD3 Light chain

SEQ ID NO: 110 - protein, 7 aa, Unknown
  intact MMP-cleavage site
SCGPAPE

SEQ ID NO: 111 - protein, 6 aa, Unknown
  cleaved MMP-cleavage site
SCGPAP

SEQ ID NO: 112 - protein, 4 aa, Artificial Sequence
  hinge sequence
ELLG